SEQUENCE LISTING (137 sequences)

SEQ ID NO: 1 - protein, 160 aa, Escherichia coli [sequence omitted]

SEQ ID NO: 2 - protein, 160 aa, synthetic construct
FACKTASGTAIPIGGGSANVYVNLAPAVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 3 - protein, 160 aa, synthetic construct
IACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 4 - protein, 160 aa, synthetic construct
LACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 5 - protein, 160 aa, synthetic construct
VACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 6 - protein, 160 aa, synthetic construct
MACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 7 - protein, 160 aa, synthetic construct
YACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 8 - protein, 160 aa, synthetic construct
WACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 9 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 10 - protein, 160 aa, synthetic construct
FACKTASGTAIPIGAGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 11 - protein, 160 aa, synthetic construct
FACKTASGTAIPIGPGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 12 - protein, 160 aa, synthetic construct
FACKTASGTAIPIGGASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 13 - protein, 160 aa, synthetic construct
FACKTASGTAIPIGGPSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 14 - protein, 160 aa, synthetic construct
FACKTASGTAIPIGAASANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 15 - protein, 160 aa, synthetic construct
FACKTASGTAIPIGGGSANVYVNLAPVVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 16 - protein, 160 aa, synthetic construct
FACKTASGTAIPIGGGSANVYVNLAPVVNVGQNLVVDLSTQIFCHNDYPETITDYVTLQRGSAYAGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 17 - protein, 160 aa, synthetic construct
FACKTASGTAICIGGGSCNVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 18 - protein, 160 aa, synthetic construct
FACKTASGTAIPICGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQCVWNIYANNDVVVPTGG

SEQ ID NO: 19 - protein, 160 aa, synthetic construct
FACKTASGTAIPIGGGSANVYVNLACVVNVGQNLCVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 20 - protein, 160 aa, synthetic construct
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDCVVPTGG

SEQ ID NO: 21 - protein, 160 aa, synthetic construct
FACKTASGTAIPIGGGSANVYVNLACVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVCPTGG

SEQ ID NO: 22 - protein, 160 aa, synthetic construct
FACKTASGTAIPIGGGSANVYVNLAPCVNVGQNCVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 23 - protein, 160 aa, synthetic construct
FACKTASGTAIPIGGGSANVYVNLAPVCNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 24 - protein, 160 aa, synthetic construct
FACKTASGTAIPIGGGSANVYVNLAPVCNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 25 - protein, 160 aa, synthetic construct
FACKTASGTAIPIGGGSANVYVNLAPVVNVGCNLVVDLSTQIFCHNDYPETITDYVTLQRGSAYGGVLSSFSGTVKYSGSSYPFPTTSETPRVVYNSRTDKPWPVALCLTPVSSAGGVAIKAGSLIAVLILRQTNNYNSDDFQFVWNIYANNDVVVPTGG

SEQ ID NO: 26 - protein, 160 aa, synthetic construct
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 27 - protein, 160 aa, synthetic construct
FACKTASGTAIPIGGGSANVYVNLAPVVNVGQCLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVCTGG

SEQ ID NO: 28 - protein, 160 aa, synthetic construct
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVACYLTPVSSAGGVAIKAGSLIAVLILRQTNNYNSDDFQFVWNIYANNDVVVPTGG

SEQ ID NO: 29 - protein, 160 aa, synthetic construct
FACKTASGTAIPIGGGSANVYVNLAPVVNVGQNLCVDLSTQIFCHNDYPETITDYVTLQRGSAYGGVLSSFSGTVKYSGSSYPFPTTSETPRVVYNSRTDKPWPVALYCTPVSSAGGVAIKAGSLIAVLILRQTNNYNSDDFQFVWNIYANNDVVVPTGG

SEQ ID NO: 30 - protein, 160 aa, synthetic construct
FACKTASGTAIPIGGGSANVYVNLAPVVNVGQNLVVDLSTQIFCHNDYPETITDYVTLQRGCAYGGVLSSFSGTVKYSGSSYPFPCTSETPRVVYNSRTDKPWPVALYLTPVSSAGGVAIKAGSLIAVLILRQTNNYNSDDFQFVWNIYANNDVVVPTGG

SEQ ID NO: 31 - protein, 160 aa, synthetic construct
FACKTASGTAIPIGGGSANVYVNLAPVVNVGQNLVVDLSTQIFCHNDYPETITDYVTLQRGCAYGGVLSSFSGTVKYSGSSYPFPTTSETPRVVYNSRTDKPWPVALYLTPVSSAGGVAIKAGSLIAVCILRQTNNYNSDDFQFVWNIYANNDVVVPTGG

SEQ ID NO: 32 - protein, 160 aa, synthetic construct
FACKTASGTAIPIGGGSANVYVNLAPVVNVGQNLVVDLSTQIFCHNDYPETITDYVTLQRGSACGGVCSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 33 - protein, 160 aa, synthetic construct
FACKTASGTAIPIGGGSANVYVNLAPVVNVGQNLVVDLSTQIFCHNDYPETITDYVTLQRGSACGGVLSSFSGTVKYSGSSYPFPTTSETPRVVYNSRTDKPWPVALYLTPVSSAGGVAIKAGSLICVLILRQTNNYNSDDFQFVWNIYANNDVVVPTGG

SEQ ID NO: 34 - protein, 160 aa, synthetic construct
FACKTASGTAIPIGGGSANVYVNLAPVVNVGQNLVVDLSTQIFCHNDYPETITDYVTLQRGSAYGGVCSSCSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 35 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPCSSAGGVAIKAGSLIAVLILRQTNNYNSDDFQFVWNIYANNDVVVPCGG

SEQ ID NO: 36 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSACGVAIKAGSLIAVLILRQTNNYNSDDFQFVWNIYANNDVVVPTGG

SEQ ID NO: 37 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVCSAGGVAIKAGSLIAVLILRQTNNYNSDDFQFVWNIYANNDVVVPCGG

SEQ ID NO: 38 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGCAIKAGSLIAVLILRQTNNYNSDDFQFVWNIYANNDVVCPTGG

SEQ ID NO: 39 - protein, 160 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVCIKAGSLIAVLILRQTNNYNSDDFQFVWNIYANNDVCVPTGG

SEQ ID NO: 40 - protein, 160 aa, synthetic construct
FACKTASGTAIPIGGGSANVYVNLAPAVNVGQNNVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 41 - protein, 160 aa, synthetic construct
FACKTASGTAIPIGGGSANVYVNLAPAVNVGQNSVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 42 - protein, 160 aa, synthetic construct
FACKTASGTAIPIGGGSANVYVNLAPAVNVGQNTVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 43 - protein, 160 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVNIKAGSLIAVLILRQTNNYNSDDFQFVWNIYANNDVVVPTGG

SEQ ID NO: 44 - protein, 160 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVSIKAGSLIAVLILRQTNNYNSDDFQFVWNIYANNDVVVPTGG

SEQ ID NO: 45 - protein, 160 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVTIKAGSLIAVLILRQTNNYNSDDFQFVWNIYANNDVVVPTGG

SEQ ID NO: 46 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSVGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 47 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDISARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 48 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTIYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 49 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADITITVNGKVVAK

SEQ ID NO: 50 - protein, 160 aa, synthetic construct
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 51 - protein, 160 aa, synthetic construct
FACKTASGTAIPIGGASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 52 - protein, 160 aa, synthetic construct
FACKTASGTAIPIGPGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 53 - protein, 160 aa, synthetic construct
FACKTASGTAIPIGGPSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 54 - protein, 160 aa, synthetic construct
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 55 - protein, 160 aa, synthetic construct
FACKTASGTAIPIGGGSANVYVNLAPAVNVGQNLVVDLSTQIFCHNDYPETITDYVTLQPGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 56 - protein, 160 aa, synthetic construct; identical to SEQ ID NO: 2

SEQ ID NO: 57 - protein, 160 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 58 - protein, 160 aa, synthetic construct
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGG

SEQ ID NO: 59 - protein, 300 aa, synthetic construct
FACKTASGTAIPIGGGSANVYVNLAPVVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 60 - protein, 300 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 61 - protein, 300 aa, synthetic construct
FACKTASGTAIPIGAGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 62 - protein, 300 aa, synthetic construct
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 63 - protein, 300 aa, synthetic construct
FACKTASGTAIPIGGGSANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 64 - protein, 300 aa, synthetic construct
FACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRKTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRQGTIIPANNTVSLGAVGTSAVSLGLTANYARTGGQVTAGNVQSIIGVTFVYQGGSSGGGADVTITVNGKVVAK

SEQ ID NO: 65 - protein, 19 aa, synthetic construct
ETDTLLLWVLLLWVPGSTG

SEQ ID NO: 66 - RNA, 1363 nt, synthetic construct
  source 1..1363: mol_type = other RNA
aggaaataagagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - RNA, 1356 nt, synthetic construct
  source 1..1356: mol_type = other RNA
agaagagaacctcgtcgagtcctggtagtagtaatcctagaggagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - RNA, 1343 nt, synthetic construct
  source 1..1343: mol_type = other RNA
aggagggtaattcgcttagcgatagtactatcgaagcgtacagagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggtgtgtggaggacaccctgaaccccccgctttcaaacaagttttcaaattgtttgaggtcaggatttctcaaactgattcctttctttgcatatgagtatttgaaaataaatattttcccaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - RNA, 1357 nt, synthetic construct
  source 1..1357: mol_type = other RNA
aggaggactgcgcgaacctgcatagtgatcataaggtcatgatagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - RNA, 1368 nt, synthetic construct
  source 1..1368: mol_type = other RNA
aggaaataagagaaagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - RNA, 1509 nt, synthetic construct
  source 1..1509: mol_type = other RNA
aggaaataagaaagaagacagaagaagacagaagaagaaccagagaaggacaagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagatgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgataggctcgctttcttgctgtccaatttctattaaaggttcctttgttccctaagtccaactactaaactgggggatattatgaagggccttgagcatctggattctgcctaataaaaaacatttattttcattgcaaccctcgactggtactgcatgcacgcaatgctagctgcccctttcccgtcctgggtaccccgagtctcccccgacctcgggtcccaggtatgctcccacctccacctgccccactcaccacctctgctagttccagacacctccaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 72 - RNA, 943 nt, synthetic construct
  source 1..943: mol_type = other RNA
aggaaataagagaggataagacgactaaggagacatacagaataagaggcagccaccatggagaccgacacactgctgctgtgggtgcttttgttgtgggtgcccggttctaccggatttgcgtgtaagaccgcctctgggactgcgatacccatcggtgctgcatccgctaacgtgtatgtgaatctcgccccagccgtcaacgtaggtcaaaacttggttgttgacttgtctacgcagatattttgtcacaatgattacccagaaacgattaccgactatgttacactccaacggggcagcgcctatggcggtgtactcagcagtttcagtggtacagtgaaatattctggcagcagttatccatttcccacaactagcgaaacccctagagttgtatataactcacgaacggacaagccttggccggtggcgctctatctgaccccggttagctcagcagggggagtggcaattaaggcggggagtttgatcgccgtgcttatactgcgccaaaccaacaattacaatagtgacgattttcaatttgtctggaacatatacgccaataacgacgtcgttgtgccaactggaggtagttctggtggcggtcccaacaaagggagtggaacaacatccgggactacgcgattgctttccggccatacttgctttacactgactggcctgttggggacgctcgttacgatgggtctgctcacctgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 73 - RNA, 1237 nt, synthetic construct
  source 1..1237: mol_type = other RNA
aggaaataagagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 74 - DNA, 672 bp, synthetic construct
  source 1..672: mol_type = other DNA
atggagaccgacacactgctgctgtgggtgcttttgttgtgggtgcccggttctaccggatttgcgtgtaagaccgcctctgggactgcgatacccatcggtgctgcatccgctaacgtgtatgtgaatctcgccccagccgtcaacgtaggtcaaaacttggttgttgacttgtctacgcagatattttgtcacaatgattacccagaaacgattaccgactatgttacactccaacggggcagcgcctatggcggtgtactcagcagtttcagtggtacagtgaaatattctggcagcagttatccatttcccacaactagcgaaacccctagagttgtatataactcacgaacggacaagccttggccggtggcgctctatctgaccccggttagctcagcagggggagtggcaattaaggcggggagtttgatcgccgtgcttatactgcgccaaaccaacaattacaatagtgacgattttcaatttgtctggaacatatacgccaataacgacgtcgttgtgccaactggaggtagttctggtggcggtcccaacaaagggagtggaacaacatccgggactacgcgattgctttccggccatacttgctttacactgactggcctgttggggacgctcgttacgatgggtctgctcacctgatga

SEQ ID NO: 75 - protein, 222 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGSSGGGPNKGSGTTSGTTRLLSGHTCFTLTGLLGTLVTMGLLT

SEQ ID NO: 76 - DNA, 966 bp, synthetic construct
  source 1..966: mol_type = other DNA
atggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatga

SEQ ID NO: 77 - protein, 320 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQGGSSGGGADVTITVNGKVVAK

SEQ ID NO: 78 - DNA, 1092 bp, synthetic construct
  source 1..1092: mol_type = other DNA
atggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtcccaacaaagggagtggaacaacatccgggactacgcgattgctttccggccatacttgctttacactgactggcctgttggggacgctcgttacgatgggtctgctcacctgatga

SEQ ID NO: 79 - protein, 362 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQGGSSGGGADVTITVNGKVVAKSSGGGPNKGSGTTSGTTRLLSGHTCFTLTGLLGTLVTMGLLT

SEQ ID NO: 80 - RNA, 1363 nt, synthetic construct
  source 1..1363: mol_type = other RNA
aggaaataagagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 81 - protein, 350 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQADVTITVNGKVVAKGSSGSGSSSGTTRLLSGHTCFTLTGLLGTLVTMGLLT

SEQ ID NO: 82 - RNA, 1241 nt, synthetic construct
  source 1..1241: mol_type = other RNA
aggaaataagagaaagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - protein, 320 aa, synthetic construct; identical to SEQ ID NO: 77

SEQ ID NO: 84 - RNA, 1367 nt, synthetic construct
  source 1..1367: mol_type = other RNA
aggaaataagagaaagaggataagacgactaaggagacatacagaataagaggcagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 85 - protein, 362 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQGGSSGGGADVTITVNGKVVAKSSGGGGSSGSGSSSGTTRLLSGHTCFTLTGLLGTLVTMGLLT

SEQ ID NO: 86 - RNA, 1230 nt, synthetic construct
  source 1..1230: mol_type = other RNA
aggaggactggtcgaacctgcatagtgatcataaggtcagcatagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 87 - protein, 313 aa, synthetic construct
METDTLLLWVLLLWVPGSTGFACKTASGTAIPIGAASANVYVNLAPAVNVGQNLVVDLSTQIFCHNDYPETITDYVTLQRGSAYGGVLSSFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRSGTIIPASNTVSLGAVGTSAVSLGLTANYARTGGQVTAGNVQSIIGVTFVYQADVTITVNGKVVAK

SEQ ID NO: 88 - RNA, 1356 nt, synthetic construct
  source 1..1356: mol_type = other RNA
aggaggactggtcgaacctgcatagtgatcataaggtcagcatagccaccatggagaccgacacactgctgctgtgggtgctgctgctgtgggtgcccggctccaccggcttcgcttgcaagacagccagcggaaccgccatccctatcggcgcagcaagcgccaacgtctacgtgaatctggctcccgcagtgaacgtgggacagaatctggtggtggatctgagcacccagatcttctgccacaatgactaccccgagaccatcacagactacgtgacactgcagagaggaagcgcctacggcggcgtgctgagcagcttcagcggaaccgtgaaatacagcggctccagctaccccttccccacaaccagcgagacccctagagtggtctataactctagaacagacaagccttggcccgtggctctgtatctgacccccgtgtccagcgctggaggagtggccatcaaggccggcagcctcatcgccgtcctcattctgaggcagaccaacaactacaacagcgacgacttccagttcgtgtggaacatctacgccaacaacgacgtggtggtccccaccggcggatgtgacgtgtccgccagagacgtgaccgtgacactgcccgattaccccggaagcgtccctatccctctgacagtgtactgcgccaagagccaaaatctgggctactatctgtccggaaccacagccgacgccggaaactccatcttcaccaacaccgccagcttttcccccgcccaaggagtgggagtccagctgacaagaagcggcaccatcatccccgccagcaacacagtgtctctgggcgctgtgggcacatccgctgtgtctctgggactgacagctaattatgccagaaccggaggccaagtgaccgctggaaatgtgcagagcattattggggtgaccttcgtgtaccagggcggaagtagcggaggcggtgccgacgtgaccatcaccgtgaacggcaaggtggtggccaagagttctggtggcggtggttcaagtggtagtggcagttcaagtgggacaacacgactgttgagcgggcatacgtgttttacgctgacaggtcttctgggcacgctggttactatgggcttgcttacgtgatgagctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 89 - protein, 362 aa, synthetic construct; identical to SEQ ID NO: 85

SEQ ID NO: 90 - DNA, 82 bp, synthetic construct
  source 1..82: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 91 - RNA, 109 nt, synthetic construct
  source 1..109: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaa

SEQ ID NO: 92 - protein, 8 aa, synthetic construct
GSSGSGSS

SEQ ID NO: 93 - RNA, 57 nt, synthetic construct
  source 1..57: mol_type = other RNA
aggaaataagagaggataagacgactaaggagacatacagaataagaggcagccacc

SEQ ID NO: 94 - RNA, 50 nt, synthetic construct
  source 1..50: mol_type = other RNA
agaagagaacctcgtcgagtcctggtagtagtaatcctagaggagccacc

SEQ ID NO: 95 - RNA, 50 nt, synthetic construct
  source 1..50: mol_type = other RNA
aggagggtaattcgcttagcgatagtactatcgaagcgtacagagccacc

SEQ ID NO: 96 - RNA, 50 nt, synthetic construct
  source 1..50: mol_type = other RNA
aggaggactgcgcgaacctgcatagtgatcataaggtcatgatagccacc

SEQ ID NO: 97 - RNA, 61 nt, synthetic construct
  source 1..61: mol_type = other RNA
aggaaataagagaaagaggataagacgactaaggagacatacagaataagaggcagccacc

SEQ ID NO: 98 - RNA, 59 nt, synthetic construct
  source 1..59: mol_type = other RNA
aggaaataagaaagaagacagaagaagacagaagaagaaccagagaaggacaagccacc

SEQ ID NO: 99 - RNA, 50 nt, synthetic construct
  source 1..50: mol_type = other RNA
aggaggactggtcgaacctgcatagtgatcataaggtcagcatagccacc

SEQ ID NO: 100 - RNA, 132 nt, synthetic construct
  source 1..132: mol_type = other RNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 101 - RNA, 120 nt, synthetic construct
  source 1..120: mol_type = other RNA
gtgtgtggaggacaccctgaaccccccgctttcaaacaagttttcaaattgtttgaggtcaggatttctcaaactgattcctttctttgcatatgagtatttgaaaataaatattttccc

SEQ ID NO: 102 - RNA, 277 nt, synthetic construct
  source 1..277: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaaccctcgactggtactgcatgcacgcaatgctagctgcccctttcccgtcctgggtaccccgagtctcccccgacctcgggtcccaggtatgctcccacctccacctgccccactcaccacctctgctagttccagacacctcc

SEQ ID NO: 103 - protein, 325 aa, Escherichia coli
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQEQVTKPQVQQTEDVTQDTLFLLGSEALESMIKHEATRPLVFSSNYYQTRQNLLDIESLKVDDLDIHAYRYVMKPTLPIRRDSPKKAITLILAVLLGGMVGAGIVLGRNALRNYNAK

SEQ ID NO: 104 - protein, 326 aa, Escherichia coli
MRVENNNVSGQNNDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQDEPEKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPTLPIRRDSPKKAITLILAVLLGGMVGAGIVLGRNALRNYNAK

SEQ ID NO: 105 - protein, 326 aa, Escherichia coli
MRVENNNVSGQNHDPEQIDLIDLLVQLWRGKMTIIISVVVAIALAIGYLAVAKEKWTSTAIITQPDVGQIAGYNNAMNVIYGQAAPKVSDLQETLIGRFSFAFSALAETLDNQKEPEKLTIEPSVKNQQLPLTVSYVGQTAEDAQMKLAQYIQQVDDKVNQELEKDLKDNLALGRKNLQDSLRTQEVVAQEQKDLRIRQIQEALQYANQAQVTKPQIQQTGEDITQDTLFLLGSEALESMIKHEATRPLVFSPNYYQTRQNLLDIENLKVDDLDIHAYRYVMKPTLPIRRDSPKKAITLILAVLLGGMVGAGIVLGRNALRNYNSK

SEQ ID NO: 106 - protein, 326 aa, Escherichia coli
MRVENNNVSGQNHDPEQIDLIDLLVQLWRGKMTIIISVIVAIALAIGYLAVAKEKWTSTAIITQPDVGQIAGYNNAMNVIYGQAAPKVSDLQETLIGRFSSAFSALAETLDNQEEREKLTIEPSVKNQQLPLTVSYVGQTAEGAQMKLAQYIQQVDDKVNQELEKDLKDNIALGRKNLQDSLRTQEVVAQEQKDLRIRQIQEALQYANQAQVTKPQIQQTGEDITQDTLFLLGSEALESMIKHEATRPLVFSPNYYQTRQNLLDIESLKVDDLDIHAYRYVMKPMLPIRRDSPKKAITLILAVLLGGMVGAGIVLGRNALRNYNAK

SEQ ID NO: 107 - protein, 327 aa, Salmonella enterica
MTVDSNTSSGRGNDPEQIDLIELLLQLWRGKMTIIVAVIIAILLAVGYLMIAKEKWTSTAIITQPDAAQVATYTNALNVLYGGNAPKISEVQANFISRFSSAFSALSEVLDNQKEREKLTIEQSVKGQALPLSVSYVSTTAEGAQRRLAEYIQQVDEEVAKELEVDLKDNITLQTKTLQESLETQEVVAQEQKDLRIKQIEEALRYADEAKITQPQIQQTQDVTQDTMFLLGSDALKSMIQNEATRPLVFSPAYYQTKQTLLDIKNLKVTADTVHVYRYVMKPTLPVRRDSPKTAITLVLAVLLGGMIGAGIVLGRNALRSYKPKAL

SEQ ID NO: 108 - protein, 377 aa, Escherichia coli
MSSLNIKQGSDAHFPDYPLASPSNNEIDLLNLISVLWRAKKTVMAVVFAFACAGLLISFILPQKWTSAAVVTPPEPVQWQELEKSFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISTLVVKESLENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKAHVNDVNFTPFKYQLSPSLPVKKDGPGKAIIVILSALIGGMVACGGVLLRYAMASRKQDAMMADHLV

SEQ ID NO: 109 - protein, 377 aa, Escherichia coli
MSSLNIKQGSEAHFPEYPLASPSNNEIDLLNLIEVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDPLDLHRAIVALSEKMKAVDDNASKKKDESALYTSWTLSFTAPTSEEAQKVLAGYIDYISALVVKESIENVRNKLEIKTQFEKEKLAQDRIKTKNQLDANIQRLNYSLDIANAAGIKKPVYSNGQAVKDDPDFSISLGADGIERKLEIEKAVTDVAELNGELRNRQYLVEQLTKTNINDVNFTPFKYQLRPSLPVKKDGQGKAIIVILSALVGGMVACGGVLLRHAMASRKQDAMMADHLV

SEQ ID NO: 110 - protein, 377 aa, Escherichia coli; identical to SEQ ID NO: 108

SEQ ID NO: 111 - protein, 377 aa, Escherichia coli
MSSLNIKQGSDAHFPDYPLASPSNNEIDLLNLISVLWRAKKTVMAVVFAFACAGLLISFILPQKWTSAAVVTPPEPVQWQELEKTFTKLRVLDLDIKIDRTEAFNLFIKKFQSVSLLEEYLRSSPYVMDQLKEAKIDELDLHRAIVALSEKMKAVDDNASKKKDEPSLYTSWTLSFTAPTSEEAQTVLSGYIDYISALVVKESIENVRNKLEIKTQFEKEKLAQDRIKMKNQLDANIQRLNYSLDIANAAGIKKPVYSNGQAVKDDPDFSISLGADGIERKLEIEKAVTDVAELNGELRNRQYLVEQLTKANINDVNFTPFKYQLSPSLPVKKDGPGKAIIVILSALIGGMVACGSVLLRYAMASRKQDAMMADHLV

SEQ ID NO: 112 - protein, 378 aa, Salmonella enterica
MPSLNVKQEKNQSFAGYSLPPANSHEIDLFSLIEVLWQAKRRILATVFAFACVGLLLSFLLPQKWTSQAIVTPAESVQWQGLERTLTALRVLDMEVSVDRGSVFNLFIKKFSSPSLLEEYLRSSPYVMDQLKGAQIDEQDLHRAIVLLSEKMKAVDSNVGKKNETSLFTSWTLSFTAPTREEAQKVLAGYIQYISDIVVKETLENIRNQLEIKTRYEQEKLAMDRVRLKNQLDANIQRLHYSLEIANAAGIKRPVYSNGQAVKDDPDFSISLGADGISRKLEIEKGVTDVAEIDGDLRNRQYHVEQLAAMNVSDVKFTPFKYQLSPSLPVKKDGPGKAIIIILAALIGGMMACGGVLLRHAMVSRKMENALAIDERLV

SEQ ID NO: 113 - protein, 950 aa, synthetic construct
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 114 - protein, 949 aa, synthetic construct
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 115 - RNA, 21 nt, synthetic construct
  source 1..21: mol_type = other RNA
ggggacgacgtcgtggggggg

SEQ ID NO: 116 - RNA, 21 nt, synthetic construct
  source 1..21: mol_type = other RNA
tcgtcgtttttcggtgctttt

SEQ ID NO: 117 - RNA, 22 nt, synthetic construct
  source 1..22: mol_type = other RNA
tgactgtgaacgttcgagatga

SEQ ID NO: 118 - RNA, 24 nt, synthetic construct
  source 1..24: mol_type = other RNA
tcgtcgttttgtcgttttgtcgtt

SEQ ID NO: 119 - RNA, 21 nt, synthetic construct
  source 1..21: mol_type = other RNA
tcgtcgtttttcggtcgtttt

SEQ ID NO: 120 - RNA, 20 nt, synthetic construct
  source 1..20: mol_type = other RNA
tccatgacgttcctgacgtt

SEQ ID NO: 121 - RNA, 24 nt, synthetic construct
  source 1..24: mol_type = other RNA
tcgtcgtttcgtcgttttgtcgtt

SEQ ID NO: 122 - RNA, 24 nt, synthetic construct
  source 1..24: mol_type = other RNA
tcgtcgttttgtcgtttttttcga

SEQ ID NO: 123 - RNA, 21 nt, synthetic construct
  source 1..21: mol_type = other RNA
  modified_base 1..21: mod_base = OTHER; note = phosphorothioate bond linkage
tcgtcgtttttcggtgctttt

SEQ ID NO: 124 - RNA, 22 nt, synthetic construct
  source 1..22: mol_type = other RNA
tcgcgtcgttcggcgcgcgccg

SEQ ID NO: 125 - RNA, 23 nt, synthetic construct
  source 1..23: mol_type = other RNA
tcgtcgacgttcggcgcgcgccg

SEQ ID NO: 126 - RNA, 21 nt, synthetic construct
  source 1..21: mol_type = other RNA
tcggacgttcggcgcgcgccg

SEQ ID NO: 127 - RNA, 19 nt, synthetic construct
  source 1..19: mol_type = other RNA
tcggacgttcggcgcgccg

SEQ ID NO: 128 - RNA, 20 nt, synthetic construct
  source 1..20: mol_type = other RNA
tcgcgtcgttcggcgcgccg

SEQ ID NO: 129 - RNA, 20 nt, synthetic construct
  source 1..20: mol_type = other RNA
tcgacgttcggcgcgcgccg

SEQ ID NO: 130 - RNA, 18 nt, synthetic construct
  source 1..18: mol_type = other RNA
tcgacgttcggcgcgccg

SEQ ID NO: 131 - RNA, 18 nt, synthetic construct
  source 1..18: mol_type = other RNA
tcgcgtcgttcggcgccg

SEQ ID NO: 132 - RNA, 22 nt, synthetic construct
  source 1..22: mol_type = other RNA
tcgcgacgttcggcgcgcgccg

SEQ ID NO: 133 - RNA, 22 nt, synthetic construct
  source 1..22: mol_type = other RNA
tcgtcgttttcggcgcgcgccg

SEQ ID NO: 134 - RNA, 22 nt, synthetic construct
  source 1..22: mol_type = other RNA
tcgtcgttttcggcggccgccg

SEQ ID NO: 135 - RNA, 24 nt, synthetic construct
  source 1..24: mol_type = other RNA
tcgtcgttttacggcgccgtgccg

SEQ ID NO: 136 - RNA, 23 nt, synthetic construct
  source 1..23: mol_type = other RNA
tcgtcgttttcggcgcgcgccgt

SEQ ID NO: 137 - RNA, 23 nt, synthetic construct
  source 1..23: mol_type = other RNA
tcgtcgacgatcggcgcgcgccg

(19) United States
(12) Patent Application Publication
(10) Pub. No.: US 2025/0281590 A1
(43) Pub. Date: Sep. 11, 2025

(54) IMMUNOGENIC COMPOSITIONS COMPRISING CONJUGATED ESCHERICHIA COLI SACCHARIDES AND USES THEREOF

(71) Applicant: Pfizer Inc., New York, NY (US)

(72) Inventors: Catherine Alex, Westwood, NJ (US); Robert George Konrad Donald, South Orange, NJ (US); Michaela Kathryn Feeney, Bayside, NY (US); Navdeep Kaur, Old Tappan, NJ (US); Jin-hwan Kim, Suffern, NY (US); Srinivas Kodali, Hillsborough, NJ (US); Rosalind Pan, Morris Plains, NJ (US); Axay Manojbhai Patel, Passaic, NJ (US); Suddham Singh, Hillsborough, NJ (US); Abhishek Ravindra Vartak, Elmsford, NY (US); YuYing Yang, Stamford, CT (US)

(21) Appl. No.: 19/074,851

(22) Filed: Mar. 10, 2025

Related U.S. Application Data
(60) Provisional application No. 63/563,748, filed on Mar. 11, 2024.
(60) Provisional application No. 63/569,359, filed on Mar. 25, 2024.
(60) Provisional application No. 63/755,767, filed on Feb. 7, 2025.
(60) Provisional application No. 63/758,638, filed on Feb. 14, 2025.

(51) Int. Cl. A61K 39/108; A61K 39/00; A61K 39/385; A61K 47/64; A61P 31/14

(52) U.S. Cl. CPC A61K 39/0258; A61K 39/0266; A61K 39/385; A61K 47/646; A61P 31/14; A61K 2039/53; A61K 2039/545; A61K 2039/55555; A61K 2039/55561; A61K 2039/575; A61K 2039/6037; A61K 2039/6068

(57) ABSTRACT

In one aspect, the present disclosure relates to an immunogenic composition comprising conjugated O-polysaccharide molecules derived from E. coli lipopolysaccharides. In one embodiment, the O-polysaccharide molecules are conjugated to streptococcal C5a peptidase (SCP). In some embodiments, the O-polysaccharide molecules are conjugated to SCP using click chemistry.

### RELATED APPLICATIONS

This application claims priority to U.S. Provisional Application No. 63/563,748 filed Mar. 11, 2024, U.S. Provisional Application No. 63/569,359 filed Mar. 25, 2024, U.S. Provisional Application No. 63/755,767 filed Feb. 7, 2025, and U.S. Provisional Application No. 63/758,638 filed Feb. 14, 2025. The entire content of each of the foregoing applications is herein incorporated by reference in its entirety.

### REFERENCE TO SEQUENCE LISTING

This application is being filed electronically via EFS-Web and includes an electronically submitted sequence listing in .xml format. The .xml file contains a sequence listing entitled “PC073039A Sequence Listing.xml” created on Feb. 25, 2025 and having a size of 157 KB. The sequence listing contained in this .xml file is part of the specification and is incorporated herein by reference in its entirety.

### FIELD OF THE DISCLOSURE

The present disclosure relates to conjugated saccharide antigens (glycoconjugates), immunogenic compositions comprising said glycoconjugates, and uses thereof. Immunogenic compositions of the present disclosure will typically comprise glycoconjugates, wherein the saccharides are derived from Escherichia coli (E. coli). The disclosure also relates to vaccination of human subjects against E. coli infections using said glycoconjugates.

### BACKGROUND OF THE DISCLOSURE

The cell wall of Gram-negative bacteria includes an outer membrane and a peptidoglycan layer on the inside of the outer membrane. The outer membrane includes phospholipids, lipopolysaccharides (LPS), lipoproteins, and membrane proteins. A lipopolysaccharide is found in an outer layer of the membrane and a phospholipid in an inner layer thereof.

The LPS includes a lipid A membrane anchor that links a core oligosaccharide to a polymer of O-polysaccharides containing repeated saccharide monomer units, which form short, long or very long O-chains. While the core oligosaccharide is mostly conserved within individual bacterial species, the O-polysaccharide can be variable amongst serotypes.

E. coli is a Gram-negative bacterium known to cause life-threatening bacterial sepsis. Both capsular (K) and lipopolysaccharide (LPS) O-antigens are important virulence factors.

There is marked interest in the use of O-polysaccharides as the basis of vaccines for E. coli. However, previous attempts to develop E. coli glycoconjugate vaccines using conventional chemical conjugation or bioconjugation approaches have failed to generate robust functional immune responses for all serotypes. Accordingly, there exists an unmet need for immunogenic compositions against E. coli that generate robust functional immune responses.

### SUMMARY OF THE DISCLOSURE

The present disclosure relates to E. coli lipopolysaccharide-associated O-antigens (O-antigens) conjugated to a streptococcal C5a peptidase (SCP) carrier protein that result in improved immunogenicity, compositions comprising the O-antigens conjugated to SCP, and uses thereof. For example, in one aspect the present disclosure provides a composition comprising a glycoconjugate, wherein the glycoconjugate comprises a streptococcal C5a peptidase (SCP) carrier protein covalently bound to a saccharide, the saccharide comprising a structure selected from the group consisting of: O1A, O2, O6, and O25b. In some embodiments, the composition comprises a glycoconjugate for each of O1A, O2, O6, and O25b. In a particular embodiment, a saccharide comprising the structure of O25b is conjugated to SCP. In some embodiments, each of O1A, O2, O6, and O25b are conjugated to SCP.

In some embodiments, n is an integer consisting of 1 to 100 in the Formula thereof for each saccharide molecule. In an exemplary embodiment, n is an integer consisting of 31 to 100 in the Formula thereof for each saccharide molecule.

In one aspect, the SCP is from Group B streptococcus (SCPB). In some embodiments, the SCP or SCPB is enzymatically inactive. In some embodiments, the SCP or SCPB is a fragment of the full-length protein. In particular embodiments, the SCP fragment comprises the sequence of SEQ ID NO: 113 or 114.

In another aspect, the O-antigen is conjugated to SCP in a click chemistry reaction. In some embodiments, the O-antigen is conjugated to SCP by an azide-alkyne cycloaddition reaction. In some embodiments, the O-antigen is conjugated to SCP in a click chemistry reaction mediated by copper.

In yet another aspect, the present disclosure provides a method of producing a glycoconjugate comprising a SCP carrier protein, and a saccharide disclosed herein, comprising the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate. In some embodiments, the cycloaddition reaction is mediated by Cu+1.

In a further aspect, the present disclosure provides a pharmaceutical composition comprising (i) an O-antigen conjugated to a SCP carrier as disclosed herein and (ii) a pharmaceutically acceptable carrier.

In another aspect, the present disclosure provides an immunogenic composition comprising an O-antigen conjugated to a SCP carrier as disclosed herein. In some embodiments, the immunogenic composition further comprises at least one additional antigen, such as a polysaccharide, a glycoconjugate, a protein, or a nucleic acid. In some embodiments, the immunogenic composition comprises each of O1A, O2, O6, and O25b, wherein at least one O-antigen is conjugated to SCP. In some embodiments, the immunogenic composition further comprises at least one adjuvant. In a particular embodiment, the adjuvant is LiNA-2, described herein.

The present disclosure further provides a method for (i) inducing an immune response in a subject against extra-intestinal pathogenic E. coli, or (ii) inducing the production of opsonophagocytic and/or neutralizing antibodies in a subject that are specific to extra-intestinal pathogenic E. coli, wherein the method comprises administering to the subject an effective amount of a composition as disclosed herein. In one example, the subject is at risk of developing a urinary tract infection. In a further example, the subject is at risk of developing bacteremia. In a further example, the subject is at risk of developing sepsis.

The present disclosure further provides a method of eliciting an immune response against E. coli in a mammal, comprising administering to the mammal an effective amount of a composition as disclosed herein. In one example, the immune response comprises opsonophagocytic and/or neutralizing antibodies against E. coli. In a further example, the immune response protects the mammal from an E. coli infection.

The present disclosure further provides a method of preventing, treating or ameliorating a bacterial infection, disease or condition in a subject, comprising administering to the subject an immunologically effective amount of a composition as disclosed herein.

### BRIEF DESCRIPTION OF THE FIGURES

FIG. 1 schematically depicts the preparation of the conjugated E. coli O-polysaccharide molecules of the present disclosure using click chemistry, described in Example 1. The reactants include O-antigen polysaccharide activated with an azido linker (azido-polysaccharide) and activated alkyne-SCP (A-SCP).

FIG. 2 presents a schematic of the schedule of immunization for the study of Example 2, as well as a table of the components administered. In this study, a comparison of the effect of O25b-SCP and O25b-CRM₁₉₇ platform chemistries on immune response was completed. CD-1 mice were provided subcutaneous (SQ) injections of the O25b-containing immunogenic compositions at week 0, week 5, and week 13.

FIG. 3 graphically depicts OPA titers at post-dose 2 (PD2) and post-dose 3 (PD3) for mice treated with a 0.2 pg dose of either O25b-SCP or O25b-CRM197 in the study of Example 2.

FIG. 4 graphically depicts OPA titers at PD2 and PD3 for mice treated with a 2 pg dose of either O25b-SCP or O25b-CRM197 in the study of Example 2.

FIG. 5A-C graphically depict antibody titers to O1A (2 μg), O2 (2 μg), O6 (2 μg), and O25b (4 μg) antigens conjugated to SCP at post-dose 1 (PD1) for non-human primates (NHPs) treated with O-antigens only (FIG. 5A), O-antigens+LiNA-2 (FIG. 5B), and O-antigens+FimH modRNA LNP (FIG. 5C). These results demonstrate that LiNA-2 or FimH modRNA LNP enhance antibody titers to 4V O-antigen SCP-conjugates after a single dose. For each respective O-antigen, the titer values are presented as baseline first (left side) and week 2 after PD1 second (right side).

### SEQUENCE IDENTIFIERS

SEQ ID NO: 1 sets forth an amino acid sequence for wild type E. coli FimH_{LD} (FimHLD_WT).

SEQ ID NO: 2 sets forth an amino acid sequence for the mutant E. coli FimHLD_G65A_V27A.

SEQ ID NO: 3 sets forth an amino acid sequence for the mutant E. coli FimHLD_F1I.

SEQ ID NO: 4 sets forth an amino acid sequence for the mutant E. coli FimHLD_F1L.

SEQ ID NO: 5 sets forth an amino acid sequence for the mutant E. coli FimHLD_F1V.

SEQ ID NO: 6 sets forth an amino acid sequence for the mutant E. coli FimHLD_F1M.

SEQ ID NO: 7 sets forth an amino acid sequence for the mutant E. coli FimHLD_F1Y.

SEQ ID NO: 8 sets forth an amino acid sequence for the mutant E. coli FimHLD_F1W.

SEQ ID NO: 9 sets forth an amino acid sequence for the mutant E. coli FimHLD_Q133K.

SEQ ID NO: 10 sets forth an amino acid sequence for the mutant E. coli FimHLD_G15A.

SEQ ID NO: 11 sets forth an amino acid sequence for the mutant E. coli FimHLD_G15P.

SEQ ID NO: 12 sets forth an amino acid sequence for the mutant E. coli FimHLD_G16A.

SEQ ID NO: 13 sets forth an amino acid sequence for the mutant E. coli FimHLD_G16P.

SEQ ID NO: 14 sets forth an amino acid sequence for the mutant E. coli FimHLD_G15A_G16A.

SEQ ID NO: 15 sets forth an amino acid sequence for the mutant E. coli FimHLD_R60P.

SEQ ID NO: 16 sets forth an amino acid sequence for the mutant E. coli FimHLD_G65A.

SEQ ID NO: 17 sets forth an amino acid sequence for the mutant E. coli FimHLD_P12C_A18C.

SEQ ID NO: 18 sets forth an amino acid sequence for the mutant E. coli FimHLD_G14C_F144C.

SEQ ID NO: 19 sets forth an amino acid sequence for the mutant E. coli FimHLD_P26C_V35C.

SEQ ID NO: 20 sets forth an amino acid sequence for the mutant E. coli FimHLD_P26C_V154C.

SEQ ID NO: 21 sets forth an amino acid sequence for the mutant E. coli FimHLD_P26C_V156C.

SEQ ID NO: 22 sets forth an amino acid sequence for the mutant E. coli FimHLD_V27C_L34C.

SEQ ID NO: 23 sets forth an amino acid sequence for the mutant E. coli FimHLD_V28C_N33C.

SEQ ID NO: 24 sets forth an amino acid sequence for the mutant E. coli FimHLD_V28C_P157C.

SEQ ID NO: 25 sets forth an amino acid sequence for the mutant E. coli FimHLD_Q32C_Y108C.

SEQ ID NO: 26 sets forth an amino acid sequence for the mutant E. coli FimHLD_N33C_L109C.

SEQ ID NO: 27 sets forth an amino acid sequence for the mutant E. coli FimHLD_N33C_P157C.

SEQ ID NO: 28 sets forth an amino acid sequence for the mutant E. coli FimHLD_V35C_L107C.

SEQ ID NO: 29 sets forth an amino acid sequence for the mutant E. coli FimHLD_V35C_L109C.

SEQ ID NO: 30 sets forth an amino acid sequence for the mutant E. coli FimHLD_S62C_T86C.

SEQ ID NO: 31 sets forth an amino acid sequence for the mutant E. coli FimHLD_S62C_L129C.

SEQ ID NO: 32 sets forth an amino acid sequence for the mutant E. coli FimHLD_Y64C_L68C.

SEQ ID NO: 33 sets forth an amino acid sequence for the mutant E. coli FimHLD_Y64C_A127C.

SEQ ID NO: 34 sets forth an amino acid sequence for the mutant E. coli FimHLD_L68C_F71C.

SEQ ID NO: 35 sets forth an amino acid sequence for the mutant E. coli FimHLD_V112C_T158C.

SEQ ID NO: 36 sets forth an amino acid sequence for the mutant E. coli FimHLD_S113C_G116C.

SEQ ID NO: 37 sets forth an amino acid sequence for the mutant E. coli FimHLD_S113C_T158C.

SEQ ID NO: 38 sets forth an amino acid sequence for the mutant E. coli FimHLD_V118C_V156C.

SEQ ID NO: 39 sets forth an amino acid sequence for the mutant E. coli FimHLD_A119C_V155C.

SEQ ID NO: 40 sets forth an amino acid sequence for the mutant E. coli FimHLD_L34N_V27A.

SEQ ID NO: 41 sets forth an amino acid sequence for the mutant E. coli FimHLD_L34S_V27A.

SEQ ID NO: 42 sets forth an amino acid sequence for the mutant E. coli FimHLD_L34T_V27A.

SEQ ID NO: 43 sets forth an amino acid sequence for the mutant E. coli FimHLD_A119N_V27A.

SEQ ID NO: 44 sets forth an amino acid sequence for the mutant E. coli FimHLD_A119S_V27A.

SEQ ID NO: 45 sets forth an amino acid sequence for the mutant E. coli FimHLD_A119T_V27A.

SEQ ID NO: 46 sets forth an amino acid sequence for the mutant E. coli FimH-DSG_A115V.

SEQ ID NO: 47 sets forth an amino acid sequence for the mutant E. coli FimH-DSG_V163I.

SEQ ID NO: 48 sets forth an amino acid sequence for the mutant E. coli FimH-DSG_V185I.

SEQ ID NO: 49 sets forth an amino acid sequence for the mutant E. coli FimH-DSG_DSG_V3I.

SEQ ID NO: 50 sets forth an amino acid sequence for the mutant E. coli FimHLD_G15A_V27A.

SEQ ID NO: 51 sets forth an amino acid sequence for the mutant E. coli FimHLD_G16A_V27A.

SEQ ID NO: 52 sets forth an amino acid sequence for the mutant E. coli FimHLD_G15P_V27A.

SEQ ID NO: 53 sets forth an amino acid sequence for the mutant E. coli FimHLD_G16P_V27A.

SEQ ID NO: 54 sets forth an amino acid sequence for the mutant E. coli FimHLD_G15A_G16A_V27A.

SEQ ID NO: 55 sets forth an amino acid sequence for the mutant E. coli FimHLD_V27A_R60P.

SEQ ID NO: 56 sets forth an amino acid sequence for the mutant E. coli FimHLD_G65A_V27A.

SEQ ID NO: 57 sets forth an amino acid sequence for the mutant E. coli FimHLD_V27A_Q133K.

SEQ ID NO: 58 sets forth an amino acid sequence for the mutant E. coli FimHLD_G15A_G16A_V27A_Q133K.

SEQ ID NO: 59 sets forth an amino acid sequence for wild type E. coli full-length FimH, including the donor strand FimG peptide connected through a linker (FimH-DSG_WT).

SEQ ID NO: 60 sets forth an amino acid sequence for the mutant E. coli FimH-DSG_V27A.

SEQ ID NO: 61 sets forth an amino acid sequence for the mutant E. coli FimH-DSG_G15A_V27A.

SEQ ID NO: 62 sets forth an amino acid sequence for the mutant E. coli FimH_{DSG}_G15A_G16A_V27A.

SEQ ID NO: 63 sets forth an amino acid sequence for the mutant E. coli FimH_{DSG}_V27A_Q133K.

SEQ ID NO: 64 sets forth an amino acid sequence for the mutant E. coli FimH_{DSG}_G15A_G16A_V27A_Q133K.

SEQ ID NO: 65 sets forth an amino acid sequence for the mouse Ig Kappa signal peptide sequence.

SEQ ID NO: 66 sets forth the nucleic acid sequence for BMD2/FimH_{DSG}-GPI/hHBB_80 pA.

SEQ ID NO: 67 sets forth the nucleic acid sequence for BMD70/FimH_{DSG-}GPI/hHBB_80 pA.

SEQ ID NO: 68 sets forth the nucleic acid sequence for BMD91/FimH_{DSG-}GPI/CYP2E1_80 pA.

SEQ ID NO: 69 sets forth the nucleic acid sequence for BMD105/FimH_{DSG-}GPI/hHBB_80 pA.

SEQ ID NO: 70 sets forth the nucleic acid sequence for BMD562/FimH_{DSG-}GPI/hHBB_80 pA.

SEQ ID NO: 71 sets forth the nucleic acid sequence for BMD3/FimH_{DSG-}GPI/hHBB-AES_80 pA.

SEQ ID NO: 72 sets forth the nucleic acid sequence for BMD2/FimH_{LD-}GPI/hHBB_80 pA.

SEQ ID NO: 73 sets forth the nucleic acid sequence of BMD2/FimH_{DSG-}Sec/hHBB_80 pA.

SEQ ID NO: 74 sets forth the nucleic acid sequence of FimH_{LD-CtDAFGPI.}

SEQ ID NO: 75 sets forth the amino acid sequence of FimH_{LD}-CtDAFGPI set forth in SEQ ID NO: 76.

SEQ ID NO: 76 sets forth the nucleic acid sequence of FimH_{LD}-CtDAFGPI.

SEQ ID NO: 77 sets forth the amino acid sequence of FimH_{LD}-CtDAFGPI set forth in SEQ ID NO: 78.

SEQ ID NO: 78 sets forth the nucleic acid sequence of FimH_{DSG}-CtDAFGPI.

SEQ ID NO: 79 sets forth amino acid sequence of FimH_{DSG}-CtDAFGPI set forth in SEQ ID NO: 80.

SEQ ID NO: 80 sets forth the nucleic acid sequence for BMD2/FimH_{DSG-}SerGlyGPI/hHBB_80 pA.

SEQ ID NO: 81 sets forth the amino acid sequence for BMD2/FimH_{DSG-}SerGlyGPI/hHBB_80 pA set forth in SEQ ID NO: 82.

SEQ ID NO: 82 sets forth the nucleic acid sequence for BMD562/FimH_{DSG-}Sec/hHBB_80 pA.

SEQ ID NO: 83 sets forth the amino acid sequence for BMD562/FimH_{DSG-}Sec/hHBB_80 pA set forth in SEQ ID NO: 84.

SEQ ID NO: 84 sets forth the nucleic acid sequence for BMD562/FimH_{DSG}-SerGlyGPI/hHBB_80 pA.

SEQ ID NO: 85 sets forth the amino acid sequence for BMD562/FimH_{DSG-}SerGlyGPI/hHBB_80 pA set forth in SEQ ID NO: 86.

SEQ ID NO: 86 sets forth the nucleic acid sequence for BMD576/FimH_{DSG-}Sec/hHBB_80 pA.

SEQ ID NO: 87 sets forth the amino acid sequence for BMD576/FimH_{DSG-}Sec/hHBB_80 pA set forth in SEQ ID NO: 88.

SEQ ID NO: 88 sets forth the nucleic acid sequence for BMD576/FimH_{DSG-}SerGlyGPI/hHBB_80 pA.

SEQ ID NO: 89 sets forth the amino acid sequence for BMD576/FimH_{DSG-}SerGlyGPI/hHBB_80 pA set forth in SEQ ID NO: 90.

SEQ ID NO: 90 sets forth the nucleic acid sequence for a 80A polyA tail.

SEQ ID NO: 91 sets forth the nucleic acid sequence for a split polyA tail, which is referred to as the “30L70” polyA tail.

SEQ ID NO: 92 sets forth the amino acid sequence of an eight amino acid Glycine-Serine linker substitution in the DAF GPI anchor.

SEQ ID NO: 93 sets forth the nucleic acid sequence for 5′ UTR_BMD2.

SEQ ID NO: 94 sets forth the nucleic acid sequence for 5′ UTR_BMD70.

SEQ ID NO: 95 sets forth the nucleic acid sequence for 5′ UTR_BMD91.

SEQ ID NO: 96 sets forth the nucleic acid sequence for 5′UTR_BMD105.

SEQ ID NO: 97 sets forth the nucleic acid sequence for 5′UTR_BMD562.

SEQ ID NO: 98 sets forth the nucleic acid sequence for 5′UTR_BMD3.

SEQ ID NO: 99 sets forth the nucleic acid sequence for 5′ UTR_BMD576.

SEQ ID NO: 100 sets forth the nucleic acid sequence for 3′ UTR_hHBB.

SEQ ID NO: 101 sets forth the nucleic acid sequence for 3′ UTR_CYP2E1.

SEQ ID NO: 102 sets forth the nucleic acid sequence for 3′ UTR_hHBB-AES.

SEQ ID NO: 103 sets forth a O25b 2401 WzzB amino acid sequence.

SEQ ID NO: 104 sets forth a O25a:K5:H1 WzzB amino acid sequence.

SEQ ID NO: 105 sets forth a O25a ETEC ATCC WzzB amino acid sequence.

SEQ ID NO: 106 sets forth a K12 W3110 WzzB amino acid sequence.

SEQ ID NO: 107 sets forth a Salmonella LT2 WzzB amino acid sequence.

SEQ ID NO: 108 sets forth a O25b 2401 FepE amino acid sequence.

SEQ ID NO: 109 sets forth a O25a:K5:H1 FepE amino acid sequence.

SEQ ID NO: 110 sets forth a O25a ETEC ATCC FepE amino acid sequence.

SEQ ID NO: 111 sets forth a O157 FepE amino acid sequence.

SEQ ID NO: 112 sets forth a Salmonella LT2 FepE amino acid sequence.

SEQ ID NO: 113 sets forth an enzymatically inactive fragment of SCP that contains 950 amino acids.

SEQ ID NO: 114 sets forth an enzymatically inactive fragment of SCP that contains 949 amino acids.

SEQ ID NO: 115 sets forth the nucleic acid sequence for an exemplary class A CpG oligonucleotide.

SEQ ID NO: 116 sets forth the nucleic acid sequence for CpG 24555.

SEQ ID NO: 117 sets forth the nucleic acid sequence for CpG 1018.

SEQ ID NO: 118 sets forth the nucleic acid sequence for CpG 7909.

SEQ ID NO: 119 sets forth the nucleic acid sequence for CpG 10103.

SEQ ID NO: 120 sets forth the nucleic acid sequence for CpG 1826.

SEQ ID NO: 121 sets forth the nucleic acid sequence for an exemplary class B CpG oligonucleotide.

SEQ ID NO: 122 sets forth the nucleic acid sequence for an exemplary class B CpG oligonucleotide.

SEQ ID NO: 123 sets forth the nucleic acid sequence for CpG 24555 wherein each of the internucleotide linkages are phosphorothioate linkages.

SEQ ID NO: 124 sets forth the nucleic acid sequence for an exemplary class C CpG oligonucleotide.

SEQ ID NO: 125 sets forth the nucleic acid sequence for an exemplary class C CpG oligonucleotide.

SEQ ID NO: 126 sets forth the nucleic acid sequence for an exemplary class C CpG oligonucleotide.

SEQ ID NO: 127 sets forth the nucleic acid sequence for an exemplary class C CpG oligonucleotide.

SEQ ID NO: 128 sets forth the nucleic acid sequence for an exemplary class C CpG oligonucleotide.

SEQ ID NO: 129 sets forth the nucleic acid sequence for an exemplary class C CpG oligonucleotide.

SEQ ID NO: 130 sets forth the nucleic acid sequence for an exemplary class C CpG oligonucleotide.

SEQ ID NO: 131 sets forth the nucleic acid sequence for an exemplary class C CpG oligonucleotide.

SEQ ID NO: 132 sets forth the nucleic acid sequence for an exemplary class C CpG oligonucleotide.

SEQ ID NO: 133 sets forth the nucleic acid sequence for an exemplary class C CpG oligonucleotide.

SEQ ID NO: 134 sets forth the nucleic acid sequence for an exemplary class C CpG oligonucleotide.

SEQ ID NO: 135 sets forth the nucleic acid sequence for an exemplary class C CpG oligonucleotide.

SEQ ID NO: 136 sets forth the nucleic acid sequence for an exemplary class C CpG oligonucleotide.

SEQ ID NO: 137 sets forth the nucleic acid sequence for an exemplary class P CpG oligonucleotide.

### DETAILED DESCRIPTION

The present disclosure relates to E. coli lipopolysaccharide-associated O-antigens (O-antigens) conjugated to a streptococcal C5a peptidase (SCP) carrier protein that result in improved immunogenicity, compositions comprising the O-antigens conjugated to SCP, methods for producing the compositions, and methods of using said compositions.

Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to further illustrate the disclosure and does not pose a limitation on the scope of the claims. No language in the specification should be construed as indicating any non-claimed element essential to the practice of the disclosure.

Several documents are cited throughout the text of this disclosure. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

Unless otherwise defined herein, scientific and technical terms used in connection with the present invention have the meanings that are commonly understood by those of ordinary skill in the art.

Throughout this application, the term “about” is used according to its plain and ordinary meaning in the area of cell and molecular biology to indicate a deviation of ±10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, modRNA and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a p-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease. In specific aspects, the pathogenic are peptide or protein antigens derived from E. coli FimH. Conditions and/or diseases that may be treated with RNA disclosed herein include, but are not limited to, those caused and/or impacted by bacterial infection. Such bacteria include, but are not limited to, E. coli.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule.

In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. In one aspect, the variant polypeptide or nucleic acid sequence has from 1 to about 4 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence. The term “mutant” of a wild-type E. coli FimH protein, “mutant” of a E. coli FimH protein, “E. coli FimH protein mutant,” or “modified E. coli FimH protein” refers to a polypeptide that displays introduced mutations relative to a wild-type FimH protein and is immunogenic against the wild-type FimH protein.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a bacteria). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more therapeutic regimens (e.g., two or more therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity).

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

As used herein, the term ‘glycoconjugate’ indicates a saccharide (in particular a bacterial saccharide) linked to a carrier protein.

### Compositions and Formulations

In one aspect, the disclosure includes a composition that includes an E. coli O-antigen conjugated to a carrier protein. In some embodiments, the composition elicits an immune response, including antibodies, that may confer immunity to pathogenic species of E. coli.

In some embodiments, the composition includes the E. coli O-antigen conjugated to a carrier protein and an additional E. coli antigen. In some embodiments, the composition includes the E. coli O-antigen conjugated to a carrier protein and a FimH protein, variant, or functional fragment thereof. In some embodiments, the composition includes the E. coli O-antigen conjugated to a carrier protein and a nucleic acid encoding FimH, a variant, or a functional fragment thereof. In some embodiments, the composition includes the E. coli O-antigen conjugated to a carrier protein and an RNA encoding FimH, a variant, or a functional fragment thereof.

In some embodiments, the composition includes the E. coli O-antigen conjugated to a carrier protein and a Klebsiella pneumoniae antigen.

In some embodiments, the composition includes a polypeptide, or a functional fragment thereof, that is derived from E. coli FimH. In some embodiments, the composition includes a polypeptide derived from E. coli FimC, or a functional fragment thereof. In some embodiments, the composition includes a polypeptide derived from E. coli FimH, or a functional fragment thereof, and a polypeptide derived from E. coli FimC or a fragment thereof.

In one aspect, the disclosure includes a composition including a polypeptide derived from E. coli FimH, or a functional fragment thereof, and a glycoconjugate, wherein the saccharide within the glycoconjugate comprises a structure selected from the group consisting of Formula O1, Formula O1A, Formula O1A1, Formula O1B, Formula O1C, Formula O2, Formula O3, Formula O4, Formula O4:K52, Formula O4:K6, Formula O5, Formula O5ab, Formula O5ac, Formula O6, Formula O6:K2, Formula O6:K13, Formula O6:K15, Formula O6:K54, Formula O7, Formula O8, Formula O9, Formula O9a, Formula O10, Formula O11, Formula O12, Formula O13, Formula O15, Formula O16, Formula O17, Formula O18, Formula O18A, Formula O18ac, Formula O18A1, Formula O18B, Formula O18B1, Formula O19, Formula O19ab, Formula O20, Formula O20ab, Formula O20ac, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O35, Formula O36, Formula O37, Formula O38, Formula O39, Formula O40, Formula O41, Formula O42, Formula O43, Formula O44, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D₁, Formula O63, Formula O64, Formula O65, Formula O66, Formula O68, Formula O69, Formula O70, Formula O71, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O112ab, Formula O112ac, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O174ab, Formula O174ac, Formula O175, Formula O176, Formula O177, Formula O178, Formula O179, Formula O180, Formula O181, Formula O182, Formula O183, Formula O184, Formula O185, Formula O186, Formula O187, and Formula O188, wherein n is an integer consisting of 1 to 100 in the Formula thereof for each saccharide molecule.

In some embodiments, the composition includes any one of the saccharides disclosed herein. In particular embodiments, the composition includes any one of the conjugates disclosed herein.

In some embodiments, the composition includes at least one glycoconjugate from E. coli serotype O25, for example serotype O25b. In one embodiment, the composition includes at least one glycoconjugate from E. coli serotype O1, for example serotype O1A. In one embodiment, the composition includes at least one glycoconjugate from E. coli serotype O2. In one embodiment, the composition includes at least one glycoconjugate from E. coli serotype O6.

In one embodiment, the composition comprises at least one glycoconjugate selected from any one of the following E. coli serotypes: O25, O1, O2, and O6, for example O25b, O1a, O2, and O6. In one embodiment, the composition comprises at least two glycoconjugates selected from any one of the following E. coli serotypes: O25, O1, O2, and O6, for example O25b, O1a, O2, and O6. In one embodiment, the composition comprises at least three glycoconjugates selected from any one of the following E. coli serotypes: O25, O1, O2, and O6, for example O25b, O1a, O2, and O6. In one embodiment, the composition comprises a glycoconjugate from each of the following E. coli serotypes: O25, O1, O2, and O6, for example O25b, O1a, O2, and O6.

In a particular embodiment, the glycoconjugate of any of the above compositions is individually conjugated to SCP. In some embodiments, the glycoconjugate of any of the above compositions is individually conjugated to CRM₁₉₇.

In one aspect, the composition includes a saccharide from at least one E. coli serotype. In a particular embodiment, the composition includes a saccharide from more than 1 E. coli serotype. For example, the composition may include a saccharide from two different E. coli serotypes (or “v”, valences) to 20 different serotypes (20v), or more. In one embodiment, the composition includes a saccharide from 3 different E. coli serotypes. In one embodiment, the composition includes a saccharide from 4, 5, 6, 7, 8, 9, 10, 11, 12, 13, 14, 15, 16, 17, 18, 19, 20, or more different E. coli serotypes. In a particular embodiment, the composition includes a saccharide from 4, 5, 6, 7, 8, 9, 10, or more different E. coli serotypes, wherein each saccharide is conjugated to a carrier protein to form a glycoconjugate as described herein.

Accordingly, in some embodiments, the composition includes an O-polysaccharide from at least one E. coli serotype. In a particular embodiment, the composition includes an O-polysaccharide from more than 1 E. coli serotype. For example, the composition may include an O-polysaccharide from two different E. coli serotypes (or “v”, valences) to 20 different serotypes (20v), or more. In one embodiment, the composition includes an O-polysaccharide from 3 different E. coli serotypes. In one embodiment, the composition includes an O-polysaccharide from 4, 5, 6, 7, 8, 9, 10, 11, 12, 13, 14, 15, 16, 17, 18, 19, 20, or more different E. coli serotypes. In a particular embodiment, the composition includes an O-polysaccharide from 4, 5, 6, 7, 8, 9, 10, or more different E. coli serotypes, wherein each O-polysaccharide is conjugated to a carrier protein to form a glycoconjugate as described herein.

In some embodiments, the O-polysaccharide includes the O-antigen and core saccharide. In a particular embodiment, the composition includes an O-polysaccharide from 4, 5, 6, 7, 8, 9, 10, or more different E. coli serotypes, wherein the O-polysaccharide is conjugated to a carrier protein, and wherein the O-polysaccharide includes the O-antigen and core saccharide.

In an exemplary embodiment, the composition includes an O-polysaccharide conjugated to SCP, wherein the O-polysaccharide includes Formula O25b, wherein n is at least 31. In another embodiment, the composition further includes an O-polysaccharide conjugated to SCP or CRM₁₉₇, wherein the O-polysaccharide includes Formula O1A, wherein n is at least 31. In another embodiment, the composition further includes an O-polysaccharide conjugated to SCP or CRM₁₉₇, wherein the O-polysaccharide includes Formula O2, wherein n is at least 31. In another embodiment, the composition further includes an O-polysaccharide conjugated to SCP or CRM₁₉₇, wherein the O-polysaccharide includes Formula O6, wherein n is at least 31.

In another embodiment, the composition further includes an O-polysaccharide conjugated to SCP or CRM₁₉₇, wherein the O-polysaccharide includes Formula O4, wherein n is at least 31. In another embodiment, the composition further includes an O-polysaccharide conjugated to SCP or CRM₁₉₇, wherein the O-polysaccharide includes Formula O11, wherein n is at least 31. In another embodiment, the composition further includes an O-polysaccharide conjugated to SCP or CRM₁₉₇, wherein the O-polysaccharide includes Formula O13, wherein n is at least 31. In another embodiment, the composition further includes an O-polysaccharide conjugated to SCP or CRM₁₉₇, wherein the O-polysaccharide includes Formula O15, wherein n is at least 31. In another embodiment, the composition further includes an O-polysaccharide conjugated to SCP or CRM₁₉₇, wherein the O-polysaccharide includes Formula O16, wherein n is at least 31. In another embodiment, the composition further includes an O-polysaccharide conjugated to SCP or CRM₁₉₇, wherein the O-polysaccharide includes Formula O17, wherein n is at least 31. In another embodiment, the composition further includes an O-polysaccharide conjugated to SCP or CRM₁₉₇, wherein the O-polysaccharide includes Formula O18, wherein n is at least 31. In another embodiment, the composition further includes an O-polysaccharide conjugated to SCP or CRM₁₉₇, wherein the O-polysaccharide includes Formula O21, wherein n is at least 31. In another embodiment, the composition further includes an O-polysaccharide conjugated to SCP or CRM₁₉₇, wherein the O-polysaccharide includes Formula O75, wherein n is at least 31. In another embodiment, the composition further includes an O-polysaccharide conjugated to SCP or CRM₁₉₇, wherein the O-polysaccharide includes Formula O86, wherein n is at least 31.

In another embodiment, the composition further includes an O-polysaccharide conjugated to SCP or CRM₁₉₇, wherein the O-polysaccharide includes Formula O8. In another embodiment, the composition further includes an O-polysaccharide conjugated to SCP or CRM₁₉₇, wherein the O-polysaccharide includes Formula O9 or Formula O9a.

As described above, the composition may include any combination of conjugated O-polysaccharides (antigens). In one exemplary embodiment, the composition includes a polysaccharide that includes Formula O25b, a polysaccharide that includes Formula O1A, a polysaccharide that includes Formula O2, and a polysaccharide that includes Formula O6. More specifically, such as a composition that includes: (i) an O-polysaccharide conjugated to SCP, wherein the O-polysaccharide includes Formula O25b, wherein n is at least 31; (ii) an O-polysaccharide conjugated to SCP, wherein the O-polysaccharide includes Formula O1a, wherein n is at least 31; (iii) an O-polysaccharide conjugated to SCP, wherein the O-polysaccharide includes Formula O2, wherein n is at least 31; and (iv) an O-polysaccharide conjugated to SCP, wherein the O-polysaccharide includes Formula O6, wherein n is at least 31.

In another exemplary embodiment, the composition includes: (i) an O-polysaccharide conjugated to SCP, wherein the O-polysaccharide includes Formula O25b, wherein n is at least 31; (ii) an O-polysaccharide conjugated to CRM₁₉₇, wherein the O-polysaccharide includes Formula O1a, wherein n is at least 31; (iii) an O-polysaccharide conjugated to CRM₁₉₇, wherein the O-polysaccharide includes Formula O2, wherein n is at least 31; and (iv) an O-polysaccharide conjugated to CRM₁₉₇, wherein the O-polysaccharide includes Formula O6, wherein n is at least 31.

In exemplary embodiments, the use of a particular protein carrier within a glycoconjugate induces a higher immune response versus the use of another protein carrier within a glycoconjugate. Immune response can be measured by OPA titer, for example geometric mean titer. In some embodiments, the use of a particular protein carrier within a glycoconjugate induces a higher OPA titer versus the use of another protein carrier within a glycoconjugate. In particular embodiments, the use of a SCP carrier within a glycoconjugate induces a higher OPA titer versus the use of another protein carrier within a glycoconjugate, for example CRM₁₉₇. In other embodiments, the use of a SCP carrier within a glycoconjugate induces a higher OPA titer versus the use of another protein carrier within a glycoconjugate (i.e., CRM₁₉₇) after administration of one dose of the glycoconjugate to a subject. In an exemplary embodiment, the use of a SCP carrier within a glycoconjugate induces a higher OPA titer versus the use of another protein carrier within a glycoconjugate (i.e., CRM₁₉₇) after administration of two doses of the glycoconjugate to a subject. In yet another exemplary embodiment, the use of a SCP carrier within a glycoconjugate induces a higher OPA titer versus the use of another protein carrier within a glycoconjugate (i.e., CRM₁₉₇) after administration of three doses of the glycoconjugate to a subject.

In one embodiment, the immunogenic composition elicits IgG antibodies in humans, said antibodies being capable of binding an E. coli serotype O25B polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the disclosure can be conducted and compared for their response to serotype O25B to assess the potential increase of responders. In one embodiment, the immunogenic composition elicits IgG antibodies in humans, said antibodies being capable of killing E. coli serotype O25B as determined by in vitro opsonophagocytic assay. In one embodiment, the immunogenic composition elicits functional antibodies in humans, said antibodies being capable of killing E. coli serotype O25B as determined by in vitro opsonophagocytic assay. In one embodiment, the immunogenic composition of the disclosure increases the proportion of responders against E. coli serotype O25B (i.e., individual with a serum having a titer of at least 1:8 as determined by in vitro OPA) as compared to the pre-immunized population. In one embodiment, the immunogenic composition elicits a titer of at least 1:8 against E. coli serotype O25B in at least 50% of the subjects as determined by in vitro opsonophagocytic killing assay. In one embodiment, the immunogenic composition of the disclosure elicits a titer of at least 1:8 against E. coli serotype O25B in at least 60%, 70%, 80%, or at least 90% of the subjects as determined by in vitro opsonophagocytic killing assay. In one embodiment, the immunogenic composition of the disclosure significantly increases the proportion of responders against E. coli serotypes O25B (i.e., individual with a serum having a titer of at least 1:8 as determined by in vitro OPA) as compared to the pre-immunized population. In one embodiment, the immunogenic composition of the disclosure significantly increases the OPA titers of human subjects against E. coli serotype O25B as compared to the pre-immunized population.

In one embodiment, the immunogenic composition elicits IgG antibodies in humans, said antibodies being capable of binding an E. coli serotype O1A polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the disclosure can be conducted and compared for their response to serotype O1A to assess the potential increase of responders. In one embodiment, the immunogenic composition elicits IgG antibodies in humans, said antibodies being capable of killing E. coli serotype O1A as determined by in vitro opsonophagocytic assay. In one embodiment, the immunogenic composition elicits functional antibodies in humans, said antibodies being capable of killing E. coli serotype O1A as determined by in vitro opsonophagocytic assay. In one embodiment, the immunogenic composition of the disclosure increases the proportion of responders against E. coli serotype O1A (i.e., individual with a serum having a titer of at least 1:8 as determined by in vitro OPA) as compared to the pre-immunized population. In one embodiment, the immunogenic composition elicits a titer of at least 1:8 against E. coli serotype O1A in at least 50% of the subjects as determined by in vitro opsonophagocytic killing assay. In one embodiment, the immunogenic composition of the disclosure elicits a titer of at least 1:8 against E. coli serotype O1A in at least 60%, 70%, 80%, or at least 90% of the subjects as determined by in vitro opsonophagocytic killing assay. In one embodiment, the immunogenic composition of the disclosure significantly increases the proportion of responders against E. coli serotypes O1A (i.e., individual with a serum having a titer of at least 1:8 as determined by in vitro OPA) as compared to the pre-immunized population. In one embodiment, the immunogenic composition of the disclosure significantly increases the OPA titers of human subjects against E. coli serotype O1A as compared to the pre-immunized population.

In one embodiment, the immunogenic composition elicits IgG antibodies in humans, said antibodies being capable of binding an E. coli serotype O2 polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the disclosure can be conducted and compared for their response to serotype O2 to assess the potential increase of responders. In one embodiment, the immunogenic composition elicits IgG antibodies in humans, said antibodies being capable of killing E. coli serotype O2 as determined by in vitro opsonophagocytic assay. In one embodiment, the immunogenic composition elicits functional antibodies in humans, said antibodies being capable of killing E. coli serotype O2 as determined by in vitro opsonophagocytic assay. In one embodiment, the immunogenic composition of the disclosure increases the proportion of responders against E. coli serotype O2 (i.e., individual with a serum having a titer of at least 1:8 as determined by in vitro OPA) as compared to the pre-immunized population. In one embodiment, the immunogenic composition elicits a titer of at least 1:8 against E. coli serotype O2 in at least 50% of the subjects as determined by in vitro opsonophagocytic killing assay. In one embodiment, the immunogenic composition of the disclosure elicits a titer of at least 1:8 against E. coli serotype O2 in at least 60%, 70%, 80%, or at least 90% of the subjects as determined by in vitro opsonophagocytic killing assay. In one embodiment, the immunogenic composition of the disclosure significantly increases the proportion of responders against E. coli serotypes O2 (i.e., individual with a serum having a titer of at least 1:8 as determined by in vitro OPA) as compared to the pre-immunized population. In one embodiment, the immunogenic composition of the disclosure significantly increases the OPA titers of human subjects against E. coli serotype O2 as compared to the pre-immunized population.

In one embodiment, the immunogenic composition elicits IgG antibodies in humans, said antibodies being capable of binding an E. coli serotype O6 polysaccharide at a concentration of at least 0.2 pg/ml, 0.3 pg/ml, 0.35 pg/ml, 0.4 pg/ml or 0.5 pg/ml as determined by ELISA assay. Therefore, comparison of OPA activity of pre- and post-immunization serum with the immunogenic composition of the disclosure can be conducted and compared for their response to serotype O6 to assess the potential increase of responders. In one embodiment, the immunogenic composition elicits IgG antibodies in humans, said antibodies being capable of killing E. coli serotype O6 as determined by in vitro opsonophagocytic assay. In one embodiment, the immunogenic composition elicits functional antibodies in humans, said antibodies being capable of killing E. coli serotype O6 as determined by in vitro opsonophagocytic assay. In one embodiment, the immunogenic composition of the disclosure increases the proportion of responders against E. coli serotype O6 (i.e., individual with a serum having a titer of at least 1:8 as determined by in vitro OPA) as compared to the pre-immunized population. In one embodiment, the immunogenic composition elicits a titer of at least 1:8 against E. coli serotype O6 in at least 50% of the subjects as determined by in vitro opsonophagocytic killing assay. In one embodiment, the immunogenic composition of the disclosure elicits a titer of at least 1:8 against E. coli serotype O6 in at least 60%, 70%, 80%, or at least 90% of the subjects as determined by in vitro opsonophagocytic killing assay. In one embodiment, the immunogenic composition of the disclosure significantly increases the proportion of responders against E. coli serotypes O6 (i.e., individual with a serum having a titer of at least 1:8 as determined by in vitro OPA) as compared to the pre-immunized population. In one embodiment, the immunogenic composition of the disclosure significantly increases the OPA titers of human subjects against E. coli serotype O6 as compared to the pre-immunized population.

### Saccharides

In one embodiment, the saccharide is produced by expression (not necessarily overexpression) of different Wzz proteins (e.g., WzzB) to control of the size of the saccharide.

As used herein, the term “saccharide” refers to a single sugar moiety or monosaccharide unit as well as combinations of two or more single sugar moieties or monosaccharide units covalently linked to form disaccharides, oligosaccharides, and polysaccharides. The saccharide may be linear or branched.

In one embodiment, the saccharide is produced in a recombinant Gram-negative bacterium. In one embodiment, the saccharide is produced in a recombinant E. coli cell. In one embodiment, the saccharide is produced in a recombinant Salmonella cell. Exemplary bacteria include E. coli O25K5H1, E. coli BD559, E. coli GAR2831, E. coli GAR865, E. coli GAR868, E. coli GAR869, E. coli GAR872, E. coli GAR878, E. coli GAR896, E. coli GAR1902, E. coli O25a ETC NR-5, E. coli O157:H7:K−, Salmonella enterica serovar Typhimurium strain LT2, E. coli GAR2401, Salmonella enterica serotype Enteritidis CVD 1943, Salmonella enterica serotype Typhimurium CVD 1925, Salmonella enterica serotype Paratyphi A CVD 1902, and Shigella flexneri CVD 1208S. In one embodiment, the bacterium is not E. coli GAR2401. This genetic approach towards saccharide production allows for efficient production of O-polysaccharides and O-antigen molecules as vaccine components.

The term “wzz protein,” as used herein, refers to a chain length determinant polypeptide, such as, for example, wzzB, wzz, wzz_{SF}, wZZ_{ST}, fepE, wzz_{fepE}, wzzl and wzz2. The GenBank accession numbers for the exemplary wzz gene sequences are AFO11910 for E4991/76, AFO11911 for F186, AF011912 for M70/1-1, AFO11913 for 79/311, AFO11914 for Bi7509-41, AF011915 for C664-1992, AF011916 for C258-94, AF011917 for C722-89, and AF011919 for EDL933. The GenBank accession numbers for the G7 and Bi316-41 wzz genes sequences are U39305 and U39306, respectively. Further GenBank accession numbers for exemplary wzz gene sequences are NP_459581 for Salmonella enterica subsp. Enterica serovar Typhimurium str. LT2 FepE; AIG66859 for E. coli O157:H7 Strain EDL933 FepE; NP_461024 for Salmonella enterica subsp. Enterica serovar Typhimurium str. LT2 WzzB. NP_416531 for E. coli K-12 substr. MG1655 WzzB, NP_415119 for E. coli K-12 substr. MG1655 FepE. In some aspects, the wzz family protein is any one of wzzB, wzz, wzz_{SF}, wzz_{ST}, fepE, wzz_{fepE}, wzz1 and wzz2, such as wzzB, in particular fepE.

Exemplary wzzB sequences include sequences set forth in SEQ ID NOs: 103-107. Exemplary FepE sequences include sequences set forth in SEQ ID NOs: 108-112.

In some aspects, a modified saccharide (modified as compared to the corresponding wild-type saccharide) may be produced by expressing (not necessarily overexpressing) a wzz family protein (e.g., fepE) from a Gram-negative bacterium in a Gram-negative bacterium and/or by switching off (i.e., repressing, deleting, removing) a second wzz gene (e.g., wzzB) to generate high molecular weight saccharides, such as lipopolysaccharides, containing intermediate or long O-antigen chains, which have an increased number of repeating units as compared to the corresponding wild-type O-polysaccharide. For example, the modified saccharides may be produced by expressing (not necessarily overexpressing) wzz2 and switching off wzzl. Or, in the alternative, the modified saccharides may be produced by expressing (not necessarily overexpressing) wzz/fepE and switching off wzzB. In another embodiment, the modified saccharides may be produced by expressing (not necessarily overexpressing) wzzB but switching off wzz/fepE. In another embodiment, the modified saccharides may be produced by expressing fepE. For example, the wzz family protein is derived from a strain that is heterologous to the host cell. Methods of determining the length of saccharides are known in the art. Such methods include, but are not limited to, nuclear magnetic resonance, mass spectroscopy, and size exclusion chromatography. Methods for producing the high molecular weight saccharides described herein, such as lipopolysaccharides, containing intermediate or long O-antigen chains, are described in PCT Intl. Publication No. WO2020/039359 and corresponding US Publication No. US2020/0061177, which are each incorporated herein by reference in their entireties.

### Exemplary Wzz Sequences are Shown Below:

SEQ ID NO: 103 sets forth a O25b 2401 WzzB amino acid sequence:
MRVENNNVSGQNHDPEQIDLIDLLVQLWRGKMTIIISVIVAIALAIGYLAVAKEKWTSTA
IITQPDVGQIAGYNNAMNVIYGQAAPKVSDLQETLIGRFSSAFSALAETLDNQEEPEKLT
IEPSVKNQQLPLTVSYVGQTAEGAQMKLAQYIQQVDDKVNQELEKDLKDNIALGRKNLQD
SLRTQEVVAQEQKDLRIRQIQEALQYANQEQVTKPQVQQTEDVTQDTLFLLGSEALESMI
KHEATRPLVFSSNYYQTRQNLLDIESLKVDDLDIHAYRYVMKPTLPIRRDSPKKAITLIL
AVLLGGMVGAGIVLGRNALRNYNAK
SEQ ID NO: 104 sets forth a O25a:K5:H1 WzzB amino acid sequence:
MRVENNNVSGQNNDPEQIDLIDLLVQLWRGKMTIIISVIVAIALAIGYLAVAKEKWTSTA
IITQPDVGQIAGYNNAMNVIYGQAAPKVSDLQETLIGRFSSAFSALAETLDNQDEPEKLT
IEPSVKNQQLPLTVSYVGQTAEGAQMKLAQYIQQVDDKVNQELEKDLKDNIALGRKNLQD
SLRTQEVVVAQEQKDLRIRQIQEALQYANQAQVTKPQIQQTGEDITQDTLFLLGSEALESM
IKHEATRPLVFSPNYYQTRQNLLDIESLKVDDLDIHAYRYVMKPTLPIRRDSPKKAITLI
LAVLLGGMVGAGIVLGRNALRNYNAK
SEQ ID NO: 105 sets forth a O25a ETEC ATCC WzzB amino acid sequence:
MRVENNNVSGQNHDPEQIDLIDLLVQLWRGKMTIIISVWVAIALAIGYLAVAKEKWTSTA
IITQPDVGQIAGYNNAMNVIYGQAAPKVSDLQETLIGRFSFAFSALAETLDNQKEPEKLT
IEPSVKNQQLPLTVSYVGQTAEDAQMKLAQYIQQVDDKVNQELEKDLKDNLALGRKNLQD
SLRTQEVVAQEQKDLRIRQIQEALQYANQAQVTKPQIQQTGEDITQDTLFLLGSEALESM
IKHEATRPLVFSPNYYQTRQNLLDIENLKVDDLDIHAYRYVMKPTLPIRRDSPKKAITLI
LAVLLGGMVGAGIVLGRNALRNYNSK
SEQ ID NO: 106 sets forth a K12 W3110 WzzB amino acid sequence:
MRVENNNVSGQNHDPEQIDLIDLLVQLWRGKMTIIISVIVAIALAIGYLAVAKEKWTSTA
IITQPDVGQIAGYNNAMNVIYGQAAPKVSDLQETLIGRFSSAFSALAETLDNQEEREKLT
IEPSVKNQQLPLTVSYVGQTAEGAQMKLAQYIQQVDDKVNQELEKDLKDNIALGRKNLQD
SLRTQEVVAQEQKDLRIRQIQEALQYANQAQVTKPQIQQTGEDITQDTLFLLGSEALESM
IKHEATRPLVFSPNYYQTRQNLLDIESLKVDDLDIHAYRYVMKPMLPIRRDSPKKAITLI
LAVLLGGMVGAGIVLGRNALRNYNAK
SEQ ID NO: 107 sets forth a Salmonella LT2 WzzB amino acid sequence:
MTVDSNTSSGRGNDPEQIDLIELLLQLWRGKMTIIVAVIIAILLAVGYLMIAKEKWTSTA
IITQPDAAQVATYTNALNVLYGGNAPKISEVQANFISRFSSAFSALSEVLDNQKEREKLT
IEQSVKGQALPLSVSYVSTTAEGAQRRLAEYIQQVDEEVAKELEVDLKDNITLQTKTLQE
SLETQEVVAQEQKDLRIKQIEEALRYADEAKITQPQIQQTQDVTQDTMFLLGSDALKSMI
QNEATRPLVFSPAYYQTKQTLLDIKNLKVTADTVHVYRYVMKPTLPVRRDSPKTAITLVL
AVLLGGMIGAGIVLGRNALRSYKPKAL
SEQ ID NO: 108 sets forth a O25b 2401 FepE amino acid sequence:
MSSLNIKQGSDAHFPDYPLASPSNNEIDLLNLISVLWRAKKTVMAVVFAFACAGLLISFI
LPQKWTSAAVVTPPEPVQWQELEKSFTKLRVLDLDIKIDRTEAFNLFIKKFQSVSLLEEY
LRSSPYVMDQLKEAKIDELDLHRAIVALSEKMKAVDDNASKKKDEPSLYTSWTLSFTAPT
SEEAQTVLSGYIDYISTLVVKESLENVRNKLEIKTQFEKEKLAQDRIKTKNQLDANIQRL
NYSLDIANAAGIKKPVYSNGQAVKDDPDFSISLGADGIERKLEIEKAVTDVAELNGELRN
RQYLVEQLTKAHVNDVNFTPFKYQLSPSLPVKKDGPGKAIIVILSALIGGMVACGGVLLR
YAMASRKQDAMMADHLV
SEQ ID NO: 109 sets forth a O25a:K5:H1 FepE amino acid sequence:
MSSLNIKQGSEAHFPEYPLASPSNNEIDLLNLIEVLWRAKKTVMAVVFAFACAGLLISFI
LPQKWTSAAVVTPPEPVQWQELEKTFTKLRVLDLDIKIDRTEAFNLFIKKFQSVSLLEEY
LRSSPYVMDQLKEAKIDPLDLHRAIVALSEKMKAVDDNASKKKDESALYTSWTLSFTAPT
SEEAQKVLAGYIDYISALVVKESIENVRNKLEIKTQFEKEKLAQDRIKTKNQLDANIQRL
NYSLDIANAAGIKKPVYSNGQAVKDDPDFSISLGADGIERKLEIEKAVTDVAELNGELRN
RQYLVEQLTKTNINDVNFTPFKYQLRPSLPVKKDGQGKAIIVILSALVGGMVACGGVLLR
HAMASRKQDAMMADHLV
SEQ ID NO: 110 sets forth a O25a ETEC ATCC FepE amino acid sequence:
MSSLNIKQGSDAHFPDYPLASPSNNEIDLLNLISVLWRAKKTVMAVVFAFACAGLLISFI
LPQKWTSAAVVTPPEPVQWQELEKSFTKLRVLDLDIKIDRTEAFNLFIKKFQSVSLLEEY
LRSSPYVMDQLKEAKIDELDLHRAIVALSEKMKAVDDNASKKKDEPSLYTSWTLSFTAPT
SEEAQTVLSGYIDYISTLVVKESLENVRNKLEIKTQFEKEKLAQDRIKTKNQLDANIQRL
NYSLDIANAAGIKKPVYSNGQAVKDDPDFSISLGADGIERKLEIEKAVTDVAELNGELRN
RQYLVEQLTKAHVNDVNFTPFKYQLSPSLPVKKDGPGKAIIVILSALIGGMVACGGVLLR
YAMASRKQDAMMADHLV
SEQ ID NO: 111 sets forth a O157 FepE amino acid sequence:
MSSLNIKQGSDAHFPDYPLASPSNNEIDLLNLISVLWRAKKTVMAVVFAFACAGLLISFI
LPQKWTSAAVVTPPEPVQWQELEKTFTKLRVLDLDIKIDRTEAFNLFIKKFQSVSLLEEY
LRSSPYVMDQLKEAKIDELDLHRAIVALSEKMKAVDDNASKKKDEPSLYTSWTLSFTAPT
SEEAQTVLSGYIDYISALVVKESIENVRNKLEIKTQFEKEKLAQDRIKMKNQLDANIQRL
NYSLDIANAAGIKKPVYSNGQAVKDDPDFSISLGADGIERKLEIEKAVTDVAELNGELRN
RQYLVEQLTKANINDVNFTPFKYQLSPSLPVKKDGPGKAIIVILSALIGGMVACGSVLLR
YAMASRKQDAMMADHLV
SEQ ID NO: 112 sets forth a Salmonella LT2 FepE amino acid sequence:
MPSLNVKQEKNQSFAGYSLPPANSHEIDLFSLIEVLWQAKRRILATVFAFACVGLLLSFL
LPQKWTSQAIVTPAESVQWQGLERTLTALRVLDMEVSVDRGSVFNLFIKKFSSPSLLEEY
LRSSPYVMDQLKGAQIDEQDLHRAIVLLSEKMKAVDSNVGKKNETSLFTSWTLSFTAPTR
EEAQKVLAGYIQYISDIVVKETLENIRNQLEIKTRYEQEKLAMDRVRLKNQLDANIQRLH
YSLEIANAAGIKRPVYSNGQAVKDDPDFSISLGADGISRKLEIEKGVTDVAEIDGDLRNR
QYHVEQLAAMNVSDVKFTPFKYQLSPSLPVKKDGPGKAIIIILAALIGGMMACGGVLLRH
AMVSRKMENALAIDERLV

In some embodiments, the saccharide is produced by expressing a wzz family protein having an amino acid sequence that is at least 30%, 50%, 70%, 75%, 80%, 85%, 90%, 95%, 98%, 99% or 100% sequence identity to any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, and SEQ ID NO: 112. In one embodiment, the wzz family protein includes a sequence selected from any one of SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, and SEQ ID NO: 112. For example, the wzz family protein has at least 30%, 50%, 70%, 75%, 80%, 85%, 90%, 95%, 98%, 99% or 100% sequence identity to SEQ ID NO: 103, SEQ ID NO: 104, SEQ ID NO: 105, SEQ ID NO: 106, SEQ ID NO: 107. In some embodiments, the saccharide is produced by expressing a protein having an amino acid sequence that is at least 30%, 50%, 70%, 75%, 80%, 85%, 90%, 95%, 98%, 99% or 100% sequence identity to an fepE protein.

In one aspect, the disclosure relates to saccharides produced by expressing a wzz family protein, such as fepE, in a Gram-negative bacterium to generate high molecular weight saccharides containing intermediate or long O-antigen chains, which have an increase of at least 1, 2, 3, 4, or 5 repeating units, as compared to the corresponding wild-type O-polysaccharide. In one aspect, the disclosure relates to saccharides produced by a Gram-negative bacterium in culture that expresses (not necessarily overexpresses) a wzz family protein (e.g., wzzB) from a Gram-negative bacterium to generate high molecular weight saccharides containing intermediate or long O-antigen chains, which have an increase of at least 1, 2, 3, 4, or 5 repeating units, as compared to the corresponding wild-type O-antigen. See description of O-polysaccharides and O-antigens below for additional exemplary saccharides having increased number of repeat units, as compared to the corresponding wild-type saccharides. A desired chain length is the one which produces improved or maximal immunogenicity in the context of a given vaccine construct.

In another embodiment, the saccharide includes any one Formula selected from Table 1, wherein the number of repeat units n in the saccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. For example, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide. Methods of determining the length of saccharides are known in the art. Such methods include nuclear magnetic resonance, mass spectroscopy, and size exclusion chromatography.

In a particular embodiment, the disclosure relates to a saccharide produced in a recombinant E. coli host cell, wherein the gene for an endogenous wzz O-antigen length regulator (e.g., wzzB) is deleted and is replaced by a (second) wzz gene from a Gram-negative bacterium heterologous to the recombinant E. coli host cell (e.g., Salmonella fepE) to generate high molecular weight saccharides, such as lipopolysaccharides, containing intermediate or long O-antigen chains. In some embodiments, the recombinant E. coli host cell includes a wzz gene from Salmonella, such as from Salmonella enterica.

In one embodiment, the host cell includes the heterologous gene for a wzz family protein as a stably maintained plasmid vector. In another embodiment, the host cell includes the heterologous gene for a wzz family protein as an integrated gene in the chromosomal DNA of the host cell. Methods of stably expressing a plasmid vector in an E. coli host cell and methods of integrating a heterologous gene into the chromosome of an E. coli host cell are known in the art. In one embodiment, the host cell includes the heterologous genes for an O-antigen as a stably maintained plasmid vector. In another embodiment, the host cell includes the heterologous genes for an O-antigen as an integrated gene in the chromosomal DNA of the host cell. Methods of stably expressing a plasmid vector in an E. coli host cell and a Salmonella host cell are known in the art. Methods of integrating a heterologous gene into the chromosome of an E. coli host cell and a Salmonella host cell are known in the art.

In another embodiment, the modified saccharide (as compared to the corresponding wild-type saccharide) described herein is synthetically produced, for example, in vitro. Synthetic production or synthesis of the saccharides may facilitate the avoidance of cost- and time-intensive production processes. In one embodiment, the saccharide is synthetically synthesized, such as, for example, by using sequential glycosylation strategy or a combination of sequential glycosylations and [3+2] block synthetic strategy from suitably protected monosaccharide intermediates. For example, thioglycosides and glycosyl trichloroacetimidate derivatives may be used as glycosyl donors in the glycosylations. In one embodiment, a saccharide that is synthetically synthesized in vitro has the identical structure to a saccharide produced by recombinant means, such as by manipulation of a wzz family protein described above.

The individual polysaccharides are typically purified (enriched with respect to the amount of polysaccharide-protein conjugate) through methods known in the art, such as, for example, dialysis, concentration operations, diafiltration operations, tangential flow filtration, precipitation, elution, centrifugation, precipitation, ultra-filtration, depth filtration, and/or column chromatography (ion exchange chromatography, multimodal ion exchange chromatography, DEAE, and hydrophobic interaction chromatography). For example, the polysaccharides are purified through a method that includes tangential flow filtration.

Purified polysaccharides may be activated (e.g., chemically activated) to make them capable of reacting (e.g., with a linker) and then incorporated into glycoconjugates, as further described herein.

For the purposes of this disclosure the term ‘glycoconjugate’ indicates a saccharide linked to a carrier protein.

In general, conjugation of saccharides to carriers enhances the immunogenicity of saccharides as it converts them from T-independent antigens to T-dependent antigens, thus allowing priming for immunological memory.

In one particular embodiment, the saccharide of the disclosure is derived from an E. coli serotype, wherein the serotype is O25b. In another particular embodiment, the serotype is O1A. In another particular embodiment, the serotype is O2. In another particular embodiment, the serotype is O6.

As used herein, reference to any of the serotypes listed above, refers to a serotype that encompasses a repeating unit structure (O-unit, as described below) known in the art and is unique to the corresponding serotype. For example, the term “O25b” serotype refers to a serotype that encompasses Formula O25b shown in Table 1.

As used herein, the serotypes are referred generically herein unless specified otherwise such that, for example, the term Formula “O18” refers generically to encompass Formula O18A Formula O18ac, Formula 18A1, Formula O18B, and Formula O18B1.

As used herein, the term “O1” refers generically to encompass the species of Formula that include the generic term “O1” in the Formula name according to Table 1, such as any one of Formula O1A, Formula O1A1, Formula O1B, and Formula O1C, each of which is shown in Table 1. Accordingly, an “O1 serotype” refers generically to a serotype that encompasses any one of Formula O1A, Formula O1A1, Formula O1B, and Formula O1C.

As used herein, the term “O6” refers generically to species of Formula that include the generic term “O6” in the Formula name according to Table 1, such as any one of Formula O6:K2; K13; K15; and O6:K54, each of which is shown in Table 1. Accordingly, an “O6 serotype” refers generically to a serotype that encompasses any one of Formula O6:K2; K13; K15; and O6:K54.

Other examples of terms that refer generically to species of a Formula that include the generic term in the Formula name according to Table 1 include: “O4”, “O5”, “O18”, and “O45”.

As used herein, the term “O2” refers to Formula O2 shown in Table 1. The term “O2 O-antigen” refers to a saccharide that encompasses Formula O2 shown in Table 1.

As used herein, reference to an O-antigen from a serotype listed above refers to a saccharide that encompasses the formula labeled with the corresponding serotype name. For example, the term “O25B O-antigen” refers to a saccharide that encompasses Formula O25B shown in Table 1.

As another example, the term “O1 O-antigen” generically refers to a saccharide that encompasses a Formula including the term “O1,” such as the Formula O1A, Formula O1A1, Formula O1B, and Formula O1C, each of which are shown in Table 1.

As another example, the term “O6 O-antigen” generically refers to a saccharide that encompasses a Formula including the term “O6,” such as Formula O6:K2; Formula O6:K13; Formula O6:K15 and Formula O6:K54, each of which are shown in Table 1.

As used herein, the term “O-polysaccharide” refers to any structure that includes an O-antigen, provided that the structure does not include a whole cell or Lipid A. For example, in one embodiment, the O-polysaccharide includes a lipopolysaccharide wherein the Lipid A is not bound. The step of removing Lipid A is known in the art and includes, as an example, heat treatment with addition of an acid. An exemplary process includes treatment with 1% acetic acid at 100° C. for 90 minutes. This process is combined with a process of isolating Lipid A as removed. An exemplary process for isolating Lipid A includes ultracentrifugation.

In one embodiment, the O-polysaccharide refers to a structure that consists of the O-antigen, in which case, the O-polysaccharide is synonymous with the term O-antigen. In one particular embodiment, the O-polysaccharide refers to a structure that includes repeating units of the O-antigen, without the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety. In another particular embodiment, the O-polysaccharide refers to a structure that includes an O-antigen and a core saccharide. In another embodiment, the O-polysaccharide refers to a structure that includes an O-antigen, a core saccharide, and a KDO moiety.

Methods of purifying an O-polysaccharide, which includes the core oligosaccharide, from LPS are known in the art. For example, after purification of LPS, purified LPS may be hydrolyzed by heating in 1% (v/v) acetic acid for 90 minutes at 100 degrees Celsius, followed by ultracentrifugation at 142,000×g for 5 hours at 4 degrees Celsius. The supernatant containing the O-polysaccharide is freeze-dried and stored at 4 degrees Celsius. In certain embodiments, deletion of capsule synthesis genes to enable simple purification of O-polysaccharide is described.

The O-polysaccharide can be isolated by methods including, but not limited to mild acid hydrolysis to remove lipid A from LPS. Other embodiments may include use of hydrazine as an agent for O-polysaccharide preparation. Preparation of LPS can be accomplished by known methods in the art.

In certain embodiments, the O-polysaccharides purified from wild-type, modified, or attenuated Gram-negative bacterial strains that express (not necessarily overexpress) a Wzz protein (e.g., wzzB) are provided for use in conjugate vaccines. In exemplary embodiments, the O-polysaccharide chain is purified from the Gram-negative bacterial strain expressing (not necessarily overexpressing) wzz protein for use as a vaccine antigen either as a conjugate or complexed vaccine.

In another embodiment, the O-polysaccharide includes any one Formula selected from Table 1, wherein the number of repeat units n in the O-polysaccharide is greater than the number of repeat units in the corresponding wild-type O-polysaccharide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units. For example, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide.

### O-Antigen

The O-antigen is part of the lipopolysaccharide (LPS) in the outer membrane of Gram-negative bacteria. The O-antigen is on the cell surface and is a variable cell constituent. The variability of the O-antigen provides a basis for serotyping of Gram-negative bacteria. The current E. coli serotyping scheme includes O-polysaccharides 1 to 188.

The O-antigen includes oligosaccharide repeating units (O-units), the wild type structure of which usually contains two to eight residues from a broad range of sugars. The O-units of exemplary E. coli O-antigens are described Table A and in PCT Intl. Publication No. WO2021/084429, published May 6, 2021, which is incorporated herein by reference in its entirety. In some embodiments, the present disclosure includes a composition comprising at least one FimH mutant polypeptide and at least one of the O-antigens as described Table A and in PCT Intl. Publication No. WO2021/084429, published May 6, 2021, which is incorporated herein by reference in its entirety.

In one embodiment, the saccharide of the disclosure may be one oligosaccharide unit. In one embodiment, the saccharide of the disclosure is one repeating oligosaccharide unit of the relevant serotype. In such embodiments, the saccharide may include a structure selected from any one of Formula O1a, Formula O2, Formula O6, Formula O8, Formula O9a, Formula O9, Formula O20ab, Formula O20ac, Formula O25b, Formula O52, Formula O97, and Formula O101. In a further embodiment, the saccharide may include a structure selected from any one of Formula O1a, Formula O2, Formula O6, and Formula O25b.

In one embodiment, the saccharide of the disclosure may be oligosaccharides. Oligosaccharides have a low number of repeat units (typically 5-15 repeat units) and are typically derived synthetically or by hydrolysis of polysaccharides. In such embodiments, the saccharide may include a structure selected from any one of Formula O1a, Formula O2, Formula O6, Formula O8, Formula O9a, Formula O9, Formula O20ab, Formula O20ac, Formula O25b, Formula O52, Formula O97, and Formula O101. In a further embodiment, the saccharide may include a structure selected from any one of Formula O1a, Formula O2, Formula O6, and Formula O25b.

In particular embodiments, all of the saccharides of the present disclosure and in the immunogenic compositions of the present disclosure are polysaccharides. High molecular weight polysaccharides may induce certain antibody immune responses due to the epitopes present on the antigenic surface. The isolation and purification of high molecular weight polysaccharides are contemplated for use in the conjugates, compositions and methods of the present disclosure.

In some embodiments, the number of repeat O units in each individual O-antigen polymer (and therefore the length and molecular weight of the polymer chain) depends on the wzz chain length regulator, an inner membrane protein. Different wzz proteins confer different ranges of modal lengths (4 to >100 repeat units). The term “modal length” refers to the number of repeating O-units. Gram-negative bacteria often have two different Wzz proteins that confer two distinct Oag modal chain lengths, one longer and one shorter. The expression (not necessarily the overexpression) of wzz family proteins (e.g., wzzB) in Gram-negative bacteria may allow for the manipulation of O-antigen length, to shift or to bias bacterial production of O-antigens of certain length ranges, and to enhance production of high-yield large molecular weight lipopolysaccharides. In one embodiment, a “short” modal length as used herein refers to a low number of repeat O-units, e.g., 1-20. In one embodiment, a “long” modal length as used herein refers to a number of repeat O-units greater than 20 and up to a maximum of 40. In one embodiment, a “very long” modal length as used herein refers to greater than 40 repeat O-units.

In one embodiment, the saccharide produced has an increase of at least 10 repeating units, 15 repeating units, 20 repeating units, 25 repeating units, 30 repeating units, 35 repeating units, 40 repeating units, 45 repeating units, 50 repeating units, 55 repeating units, 60 repeating units, 65 repeating units, 70 repeating units, 75 repeating units, 80 repeating units, 85 repeating units, 90 repeating units, 95 repeating units, or 100 repeating units, as compared to the corresponding wild-type O-polysaccharide.

In another embodiment, the saccharide of the disclosure has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide. In particular embodiments, the saccharide includes an increase of at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 repeat units, as compared to the corresponding wild-type O-polysaccharide. Methods of determining the length of saccharides are known in the art. Such methods include nuclear magnetic resonance, mass spectroscopy, and size exclusion chromatography.

Methods of determining the number of repeat units in the saccharide are also known in the art. For example, the number of repeat units (or “n” in the Formula) may be calculated by dividing the molecular weight of the polysaccharide (without the molecular weight of the core saccharide or KDO residue) by the molecular weight of the repeat unit (i.e., molecular weight of the structure in the corresponding Formula, shown for example in Table 1, which may be theoretically calculated as the sum of the molecular weight of each monosaccharide within the Formula). The molecular weight of each monosaccharide within the Formula is known in the art. The molecular weight of a repeat unit of Formula O25b, for example, is about 862 Da. The molecular weight of a repeat unit of Formula O1a, for example, is about 845 Da. The molecular weight of a repeat unit of Formula O2, for example, is about 829 Da. The molecular weight of a repeat unit of Formula O6, for example, is about 893 Da. When determining the number of repeat units in a conjugate, the carrier protein molecular weight and the protein:polysaccharide ratio is factored into the calculation. As defined herein, “n” refers to the number of repeating units (represented in brackets in Table 1) in a polysaccharide molecule. As is known in the art, in biological macromolecules, repeating structures may be interspersed with regions of imperfect repeats, such as, for example, missing branches. In addition, it is known in the art that polysaccharides isolated and purified from natural sources such as bacteria may be heterogenous in size and in branching. In such a case, n may represent an average or median value for n for the molecules in a population.

In one embodiment, the O-polysaccharide has an increase of at least one repeat unit of an O-antigen, as compared to the corresponding wild-type O-polysaccharide. The repeat units of O-antigens are shown in Table 1. In one embodiment, the O-polysaccharide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more total repeat units. In particular embodiments, the saccharide has a total of at least 3 to at most 80 repeat units. In another embodiment, the O-polysaccharide has an increase of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more repeat units, as compared to the corresponding wild-type O-polysaccharide.

In one embodiment, the saccharide includes an O-antigen wherein n in any of the O-antigen formulas (such as, for example, the Formulas shown in Table 1 (see also FIG. 9A-9C and FIG. 10A-10B)) is an integer of at least 1, 2, 3, 4, 5, 10, 20, 21, 22, 23, 24, 25, 26, 27, 28, 29, 30, 31, 32, 33, 34, 35, 36, 37, 38, 39, 40, and at most 200, 100, 99, 98, 97, 96, 95, 94, 93, 92, 91, 90, 89, 88, 87, 86, 81, 80, 79, 78, 77, 76, 75, 74, 73, 72, 71, 70, 69, 68, 67, 66, 65, 60, 59, 58, 57, 56, 55, 54, 53, 52, 51, or 50. Any minimum value and any maximum value may be combined to define a range. Exemplary ranges include, for example, at least 1 to at most 1000; at least 10 to at most 500; and at least 20 to at most 80, such as at most 90. In particular embodiments, n is at least 31 to at most 90. In another particular embodiment, n is 40 to 90, such as 60 to 85.

In one embodiment, the saccharide includes an O-antigen wherein n in any one of the O-antigen Formulas is at least 1 and at most 200. In one embodiment, n in any one of the O-antigen Formulas is at least 5 and at most 200. In one embodiment, n in any one of the O-antigen Formulas is at least 10 and at most 200. In one embodiment, n in any one of the O-antigen Formulas is at least 25 and at most 200. In one embodiment, n in any one of the O-antigen Formulas is at least 50 and at most 200. In one embodiment, n in any one of the O-antigen Formulas is at least 75 and at most 200. In one embodiment, n in any one of the O-antigen Formulas is at least 100 and at most 200. In one embodiment, n in any one of the O-antigen Formulas is at least 125 and at most 200. In one embodiment, n in any one of the O-antigen Formulas is at least 150 and at most 200. In one embodiment, n in any one of the O-antigen Formulas is at least 175 and at most 200. In one embodiment, n in any one of the O-antigen Formulas is at least 1 and at most 100. In one embodiment, n in any one of the O-antigen Formulas is at least 5 and at most 100. In one embodiment, n in any one of the O-antigen Formulas is at least 10 and at most 100. In one embodiment, n in any one of the O-antigen Formulas is at least 25 and at most 100. In one embodiment, n in any one of the O-antigen Formulas is at least 50 and at most 100. In one embodiment, n in any one of the O-antigen Formulas is at least 75 and at most 100. In one embodiment, n in any one of the O-antigen Formulas is at least 1 and at most 75. In one embodiment, n in any one of the O-antigen Formulas is at least 5 and at most 75. In one embodiment, n in any one of the O-antigen Formulas is at least 10 and at most 75. In one embodiment, n in any one of the O-antigen Formulas is at least 20 and at most 75. In one embodiment, n in any one of the O-antigen Formulas is at least 25 and at most 75. In one embodiment, n in any one of the O-antigen Formulas is at least 30 and at most 75. In one embodiment, n in any one of the O-antigen Formulas is at least 40 and at most 75. In one embodiment, n in any one of the O-antigen Formulas is at least 50 and at most 75. In one embodiment, n in any one of the O-antigen Formulas is at least 30 and at most 90. In one embodiment, n in any one of the O-antigen Formulas is at least 35 and at most 85. In one embodiment, n in any one of the O-antigen Formulas is at least 35 and at most 75. In one embodiment, n in any one of the O-antigen Formulas is at least 35 and at most 70. In one embodiment, n in any one of the O-antigen Formulas is at least 35 and at most 60. In one embodiment, n in any one of the O-antigen Formulas is at least 35 and at most 50. In one embodiment, n in any one of the O-antigen Formulas is at least 35 and at most 49. In one embodiment, n in any one of the O-antigen Formulas is at least 35 and at most 48. In one embodiment, n in any one of the O-antigen Formulas is at least 35 and at most 47. In one embodiment, n in any one of the O-antigen Formulas is at least 35 and at most 46. In one embodiment, n in any one of the O-antigen Formulas is at least 36 and at most 45. In one embodiment, n in any one of the O-antigen Formulas is at least 37 and at most 44. In one embodiment, n in any one of the O-antigen Formulas is at least 38 and at most 43. In one embodiment, n in any one of the O-antigen Formulas is at least 39 and at most 42. In one embodiment, n in any one of the O-antigen Formulas is at least 39 and at most 41.

For example, in one embodiment, n in the saccharide is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90, for example 40. In another embodiment, n is at least 35 to at most 60. For example, in one embodiment, n is any one of 35, 36, 37, 38, 39, 40, 41, 42, 43, 44, 45, 46, 47, 48, 49, 50, 51, 52, 53, 54, 55, 56, 57, 58, 59, and 60, such as 50. In another particular embodiment, n is at least 55 to at most 75. For example, in one embodiment, n is 55, 56, 57, 58, 59, 60, 61, 62, 63, 64, 65, 66, 67, 68, or 69, such as 60.

The saccharide structure may be determined by methods and tools known art, such as, for example, NMR, including 1D, 1H, and/or 13C, 2D TOCSY, DQF-COSY, NOESY, and/or HMQC.

In some embodiments, the purified polysaccharide before conjugation has a molecular weight of between 5 kDa and 400 kDa. In other such embodiments, the saccharide has a molecular weight of between 10 kDa and 400 kDa; between 5 kDa and 400 kDa; between 5 kDa and 300 kDa; between 5 kDa and 200 kDa; between 5 kDa and 150 kDa; between 10 kDa and 100 kDa; between 10 kDa and 75 kDa; between 10 kDa and 60 kDa; between 10 kDa and 40 kDa; between 10 kDa and 100 kDa; 10 kDa and 200 kDa; between 15 kDa and 150 kDa; between 12 kDa and 120 kDa; between 12 kDa and 75 kDa; between 12 kDa and 50 kDa; between 12 and 60 kDa; between 35 kDa and 75 kDa; between 40 kDa and 60 kDa; between 35 kDa and 60 kDa; between 20 kDa and 60 kDa; between 12 kDa and 20 kDa; or between 20 kDa and 50 kDa. In further embodiments, the polysaccharide has a molecular weight of between 7 kDa to 15 kDa; 8 kDa to 16 kDa; 9 kDa to 25 kDa; 10 kDa to 100; 10 kDa to 60 kDa; 10 kDa to 70 kDa; 10 kDa to 160 kDa; 15 kDa to 600 kDa; 20 kDa to 1000 kDa; 20 kDa to 600 kDa; 20 kDa to 400 kDa; 30 kDa to 1,000 Kda; 30 kDa to 60 kDa; 30 kDa to 50 kDa or 5 kDa to 60 kDa. Any whole number integer within any of the above ranges is contemplated as an embodiment of the disclosure.

As used herein, the term “molecular weight” of polysaccharide or of carrier protein-polysaccharide conjugate refers to molecular weight calculated by size exclusion chromatography (SEC) combined with multiangle laser light scattering detector (MALLS).

A polysaccharide can become slightly reduced in size during normal purification procedures. Additionally, as described herein, polysaccharide can be subjected to sizing techniques before conjugation. Mechanical or chemical sizing maybe employed. Chemical hydrolysis may be conducted using acetic acid. Mechanical sizing may be conducted using High Pressure Homogenization Shearing. The molecular weight ranges mentioned above refer to purified polysaccharides before conjugation (e.g., before activation).

The structure of E. coli saccharide structures are known to those skilled in the art. Exemplary E. coli saccharide structures are shown in Table 1, below. Additional E. coli saccharide structures can be found with reference to Rojas-Macias et al. “Development of the ECODAB into a relational database for Escherichia coli O-antigens and other bacterial polysaccharides Glycobiology” (2015) 25:341-347, citing the ECOBAD database, available at https://nevyn.organ.su.se/ECODAB/.

TABLE 1
E. coli serogroups/serotypes and O-unit moieties

| | | Moiety |
|---|---|---|
| | | structure |
| | | referred to |
| Serogroup/ | | herein |
| Serotype | Moiety Structure (O-unit) | as: |
| O1A, | [→3)-α-L-Rha-(1→3)-α-L-Rha-(1→3)-βL-Rha-(1→4)-β-D-GlcNAc-(1→\| | Formula |
| O1A1 | β-D-ManNAc-(1→2)]ₙ | O1A |
| O1B | [→3)-α-L-Rha-(1→2)-α-L-Rha-(1→2)-α-D-Gal-(1→3)-β-D-GlcNAc-(1→\|β- | Formula |
| | D-ManNAc-(1→2)]ₙ | O1B |
| O1C | [→3)-α-L-Rha-(1→2)-α-L-Rha-(1→3)-α-D-Gal-(1→3)-β-D-GlcNAc-(1→\|β- | Formula |
| | D-ManNAc-(1→2)]ₙ | O1C |
| O2 | [→3)-α-L-Rha-(1→2)-α-L-Rha-(1→3)-β-L-Rha-(1→4)-β-D-GlcNAc-(1→\| | Formula |
| | α-D-Fuc3Nac-(1→2)]ₙ | O2 |
| O3 | [β-L-RhaNAc(1→4)α-D-Glc-(1→4)\|\|→3)-β-D-GlcNAc-(1→3)-α-D-Gal- | Formula |
| | (1→3)-β-D-GlcNAc-(1→]ₙ | O3 |
| O4:K52 | [→2)-α-L-Rha-(1→6)-α-D-Glc-(1→3)-α-L-FucNAc-(1→3)-β-D- | Formula |
| | GlcNAc(1→]ₙ | O4:K52 |
| O4:K6 | [α-D-Glc-(1→3)\|→2)-α-L-Rha-(1→6)-α-D-Glc-(1→3)-α-L-FucNAc- | Formula |
| | (1→3)-β-D-GlcNAc(1→]ₙ | O4:K6 |
| O5ab | [→4)-β-D-Qui3Nac-(1→3)-β-D-Ribf-(1→4)-β-D-Gal-(1→3)-α-D-GalNAc(1→]ₙ | Formula |
| | | O5ab |
| O5ac | [→2)-β-D-Qui3Nac-(1→3)-β-D-Ribf-(1→4)-β-D-Gal-(1→3)-α-D- | Formula |
| (strain | GalNAc(1→]ₙ | O5ac |
| 180/C3) | | (strain |
| | | 180/C3) |
| O6:K2; | [→4)-α-D-GalNAc-(1→3)-β-D-Man-(1→4)-β-D-Man-(1→3)-α-D-GlcNAc- | Formula O6: |
| K13; K15 | (1→\|β-D-Glc-(1→2)]ₙ | K2; K13; K15 |
| O6:K54 | [→4)-α-D-GalNAc-(1→3)-β-D-Man-(1→4)-β-D-Man-(1→3)-α-D-GlcNAc- | Formula |
| | (1→\|β-D-GlcNAc-(1→2)]ₙ | O6:K54 |
| O7 | [α-L-Rha-(1→3)\|→3)-β-D-Qui4Nac-(1→2)-α-D-Man-(1→4)-β-D-Gal- | Formula |
| | (1→3)-α-D-GlcNAc-(1→]ₙ | O7 |
| O8 | [→2)-α-D-Man-(1→2)-α-D-Man-(1→3)-β-D-Man-(1→]ₙ | Formula O8 |
| O9a | [→2)-α-D-Man-(1→2)-α-D-Man-(1→3)-α-D-Man-(1→3)-α-D-Man-(1→]ₙ | Formula O9a |
| O9 | [→2)-[α-D-Man-(1→2)]₂-α-D-Man-(1→3)-α-D-Man-(1→3)-α-D-Man- | Formula O9 |
| | (1→]ₙ | |
| O10 | [→3)-α-L-Rha-(1→3)-α-L-Rha-(1→3)-α-D-Gal-(1→3)-β-D-GlcNAc-(1→\| | Formula |
| | α-D-Fuc4Nacyl-(1→2) Acyl=acetyl (60%) or I-3-hydroxybutyryl (40%)]ₙ | O10 |
| O11 | 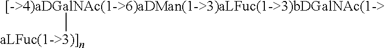 | Formula O11 |
| O12 | [->2)bDGlc(1->6)aDGlcNAc(1->3)aLFucNAc(1->3)bDGlcNAc(1->]ₙ | Formula |
| | | O12 |
| O13 | 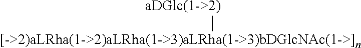 | Formula O13 |
| O15 | [->2)bDGal(1->3)aLFucNAc(1->3)bDGlcNAc(1->]ₙ | Formula |
| | | O15 |
| O16 | [→2)-β-D-Galf-(1→6)-α-D-Glc-(1→3)-α-L-Rha2Ac-(1→3)-α-D-GlcNAc- | Formula |
| | (1→]ₙ | O16 |
| O17 | [α-D-Glc-(1→6)\|→6-α-D-Man-(1→2)-α-D-Man-(1→2)-β-D-Man-(1→3)- | Formula |
| | α-D-GlcNAc(1→]ₙ | O17 |
| O18A, | [→2)-α-L-Rha-(1→6)-α-D-Glc-(1→4)-α-D-Gal-(1→3)-α-D-GlcNAc-(1→\| | Formula |
| O18ac | β-D-GlcNAc-(1→3)]ₙ | O18A, |
| | | Formula |
| | | O18ac |
| O18A1 | [α-D-Glc-(1→6)\|→2)-α-L-Rha-(1→6)-α-D-Glc-(1→4)-α-D-Gal-(1→3)-α- | Formula |
| | D-GlcNAc-(1→\|β-D-GlcNAc-(1→3)]ₙ | O18A1 |
| O18B | [→3)-α-L-Rha-(1→6)-α-D-Glc-(1→4)-α-D-Gal-(1→3)-α-D-GlcNAc-(1→\| | Formula |
| | β-D-Glc-(1→3)]ₙ | O18B |
| O18B1 | [α-D-Glc-(1→4)\|→3)-α-L-Rha-(1→6)-α-D-Glc-(1→4)-α-D-Gal-(1→3)-α- | Formula |
| | D-GlcNAc-(1→\|β-D-Glc-(1→3)]ₙ | O18B1 |
| O19ab | [->2)aLRha(1->2)aLRha(1->2)aLRha(1->2)aDGlc(1->3)aDGlcNAc6Ac(1->]ₙ | Formula |
| | | O19ab |
| O20ab | [→2)-β-D-Ribf-(1→4)-α-D-Gal-(1→]ₙ | Formula |
| | | O20ab |
| O20ac | [α-D-Gal-(1→3)\|→2)-β-D-Ribf-(1→4)-α-D-Gal-(1→]ₙ | Formula |
| | | O20ac |
| O21 | [β-D-Gal-(1→4)\|→3)-β-D-Gal-(1→4)-β-D-Glc-(1→3)-β-D-GalNAc-(1→\| | Formula |
| | β-D-GlcNAc-(1→2)]ₙ | O21 |
| O22 | [→6)-α-D-Glc-(1→4)-β-D-GlcA-(1→4)-β-D-GalNAc3Ac-(1→3)-α-D-Gal- | Formula |
| | (1→3)-β-D-GlcNAc-(1→]ₙ | O22 |
| O23A | [α-D-Glc-(1→6)\|→6)-α-D-Glc-(1→4)-β-D-Gal-(1→3)-α-D-GalNAc- | Formula |
| | (1→3)-β-D-GlcNAc-(1→\|β-D-GlcNAc(1→3)]ₙ | O23A |
| O24 | [→7)-α-Neu5Ac-(2→3)-β-D-Glc-(1→3)-β-D-GalNAc-(1→\|α-D-Glc- | Formula |
| | (1→2)]ₙ | O24 |
| O25/O25a | [β-D-Glc-(1→6)\|→4)-α-D-Glc-(1→3)-α-L-FucNAc-(1→3)-β-D-GlcNAc- | Formula |
| | (1→\|α-L-Rha-(1→3)]ₙ | O25a |
| O25b | 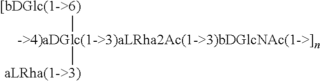 | Formula O25b |
| O26 | [→3)-α-L-Rha-(1→4)-α-L-FucNAc-(1→3)-β-D-GlcNAc-(1→]ₙ | Formula |
| | | O26 |
| O27 | 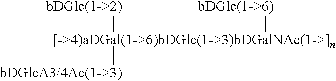 | Formula O27 |
| O28 | [→2)-\|-Gro-1-P→4)-β-D-GlcNAc-(1→3)-β-D-Galf2Ac-(1→3)-α-D- | Formula |
| | GlcNAc-(1→]ₙ | O28 |
| O29 | 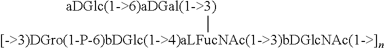 | Formula O29 |
| O30 | 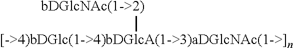 | Formula O30 |
| O32 | [->6)aDGal(1->4)bDGlcA(1->6)bDGal(1->4)bDGal2Ac3Ac(1->4)bDGlcNAc(1->]ₙ | Formula |
| | | O32 |
| O33 | 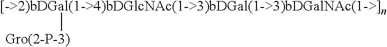 | Formula O33 |
| O35 | [→3)-α-L-Rha-(1→2)-α-L-Rha-(1→3)-α-L-Rha-(1→2)-α-L-Rha-(1→3)-β- | Formula |
| | D-GlcNAc-(1→\|α-D-GalNAcA6N-(1→2)]ₙ | O35 |
| O36 | 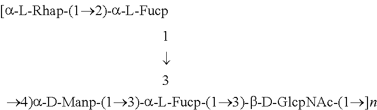 | Formula O36 |
| O37 | 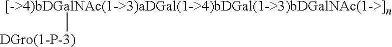 | Formula O37 |
| O38 | 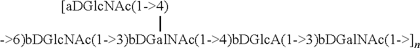 | Formula O38 |
| O39 | 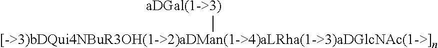 | Formula O39 |
| O40 | [->2)bDGal(1->4)bDMan(1->4)aDGal(1->3)bDGlcNAc(1->]ₙ | Formula |
| | | O40 |
| O41 | 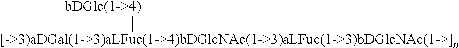 | Formula O41 |
| O42 | 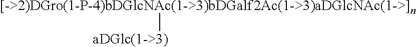 | Formula O42 |
| O43 | 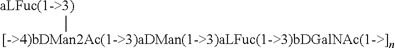 | Formula O43 |
| O44 | [α-D-Glc-(1→4)\|→6)-α-D-Man-(1→2)-α-D-Man-(1→2)-β-D-Man-(1→3)- | Formula |
| | α-D-GlcNAc(1→]ₙ | O44 |
| O45 | [→2)-β-D-Glc-(1→3)-α-L-6dTal2Ac-(1→3)-α-D-FucNAc-(1→]ₙ | Formula |
| | | O45 |
| O45rel | [→2)-β-D-Glc-(1→3)-α-L-6dTal2Ac-(1→3)-β-D-GlcNAc-(1→]ₙ | Formula |
| | | O45rel |
| O46 | [->4)bDG1cA6LThr(1->6)bDGal(1->6)bDGlc(1->3)bDGalNAc(1->]ₙ | Formula |
| | | O46 |
| O48 | 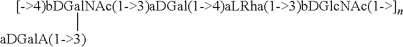 | Formula O48 |
| O49 | [->2)aDQui4NBuS3OH(1->4)bDGalNAc(1->4)aLRha(1->3)bDGlcNAc6Ac(1->]ₙ | Formula |
| | | O49 |
| O50 | [->3)aLRha(1->2)aLRha(1->3)bLRha(1->4)bDGlcNAc(1->]ₙ | Formula |
| | | O50 |
| O51 | 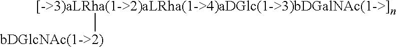 | Formula O52 |
| O52 | [→3)-β-D-Fucf-(1→3)-β-D-6dmanHep2Ac-(1→]ₙ | Formula |
| | | O52 |
| O53 | 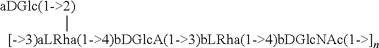 | Formula O53 |
| O54 | [→4)-α-d-GalpA-(1→2)-α-I-Rhap-(1→2)-β-d-Ribf-(1→3)-β-d-Galp- | Formula |
| | (1→3)-β-d-GlcpNAc-(1→]n | O54 |
| O55 | [→6)-β-D-GlcNAc-(1→3)-α-D-Gal-(1→3)-β-D-GalNAc-(1→\|α-Col- | Formula |
| | (1→2)-β-D-Gal-(1→3)]ₙ | O55 |
| O56 | [→7)-α-Neu5Ac-(2→3)-β-D-Glc-(1→3)-β-D-GlcNAc-(1→\|α-D-Gal- | Formula |
| | (1→2)]ₙ | O56 |
| O57 | 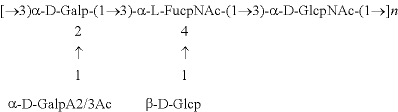 | Formula O57 |
| O58 | [3-O-[I-1-carboxyethyl]-α-L-Rha-(1→3)\|→4)-α-D-Man-(1→4)-α-D- | Formula |
| | Man2Ac-(1→3)-β-D-GlcNAc-(1→]ₙ | O58 |
| O59 | 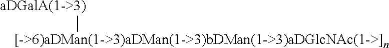 | Formula O59 |
| O60 | 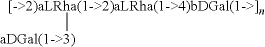 | Formula O60 |
| O61 | [->4)a8eLeg5Ac7Ac8Ac(2->4)bDGlc(1->3)aDGalNAc(1->]ₙ | Formula |
| | | O61 |
| O62 | 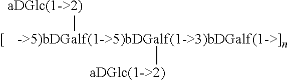 | Formula O62 |
| 62D₁ | [α-D-Gal(1→6)\|→2)-β-D-Qui3Nac-(1→3)-α-L-Rha-(1→3)-β-D-Gal- | Formula |
| Suggested | (1→3)-α-D-FucNAc-(1→]ₙ | 62D₁ |
| as Erwinia | | |
| herbicola | | |
| O63 | [->2)bDQui3NDalloThrAc(1->2)bDRibf(1->4)bDGal(1->3)aDGlcNAc(1->]ₙ | Formula |
| | | O63 |
| O64 | [β-D-Gal-(1→6)\|→3)-α-D-ManNAc-(1→3)-β-D-GlcA-(1→4)-β-D- | Formula |
| | (1→3)-β-D-GlcNAc(1→]ₙ | O64 |
| O65 | [→2)-β-D-Qui3Nac-(1→4)-α-D-GalA6N-(1→4)-α-D-GalNAc-(1→4)-β-D- | Formula |
| | GalA-(1→3)-α-D-GlcNAc-(1→]ₙ | O65 |
| O66 | [→2)-β-D-Man-(1→3)-α-D-GlcNAc-(1→2)-β-D-Glc3Ac-(1→3)-α-L-6dTal- | Formula |
| | (1→3)-α-D-GlcNAc(1→]ₙ | O66 |
| O68 | 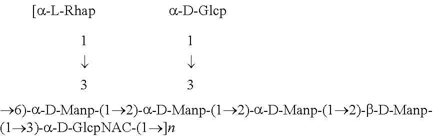 | Formula O68 |
| O69 | [→2)-α-L-Rha-(1→2)-α-L-Rha-(1→2)-α-D-Gal-(1→3)-β-D-GlcNAc- | Formula |
| | (1→]ₙ | O69 |
| O70 | 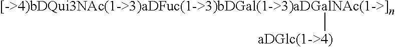 | Formula O70 |
| O71 | [->4)aDQui3NAc(1->3)aLRha(1->4)bDGal(1->3)aDGalNAc(1->]ₙ | Formula |
| | | O71 |
| O73 | [α-D-Glc-(1→3)\|→4)-α-D-Man-(1→2)-α-D-Man-(1→2)-β-D-Man-(1→3)- | Formula |
| (Strain 73- | α-D-GalNAc(1→]ₙ | O73 |
| 1) | | (Strain |
| | | 73-1) |
| O74 | 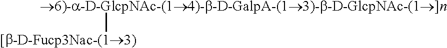 | Formula O74 |
| O75 | [β-D-Man-(1→4)\|→3)-α-D-Gal-(1→4)-α-L-Rha-(1→3)-β-D-GlcNAc- | Formula |
| | (1→]ₙ | O75 |
| O76 | [→4)-β-D-GlcpA-(1→4)-β-D-GalpNAc3Ac-(1→4)-α-D-GalpNAc-(1→3)-β- | Formula |
| | D-GalpNAc-(1→]n | O76 |
| O77 | [→6)-α-D-Man-(1→2)-α-D-Man-(1→2)-β-D-Man-(1→3)-α-D- | Formula |
| | GlcNAc(1→]ₙ | O77 |
| O78 | [→4)-β-D-GlcNAc-(1→4)-β-D-Man-(1→4)-α-D-Man-(1→3)-β-GlcNAc- | Formula |
| | (1→]ₙ | O78 |
| O79 | 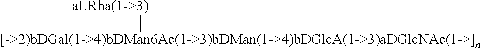 | Formula O79 |
| O80 | 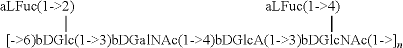 | Formula O80 |
| O81 | 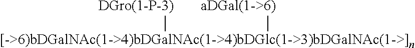 | Formula O81 |
| O82 | 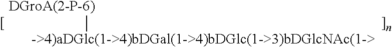 | Formula O82 |
| O83 | [→6)-α-D-Glc-(1→4)-β-D-GlcA-(1→6)-β-D-Gal-(1→4)-β-D-Gal-(1→4)-β- | Formula |
| | D-GlcNAc-(1→]ₙ | O83 |
| O84 | 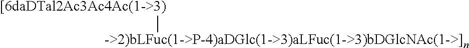 | Formula O84 |
| O85 | 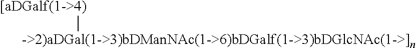 | Formula O85 |
| O86 | [α-D-Gal-(1→3)\|→4)-α-L-Fuc-(1→2)-β-D-Gal-(1→3)-α-D-GalNAc- | Formula |
| | (1→3)-β-D-GalNAc-(1→]ₙ | O86 |
| O87 | [->3)bDGalf(1->4)aDGal(1->3)aDGalA4Ac(1->3)bDGalNAc(1->]ₙ | Formula |
| | | O87 |
| O88 | [α-L-6dTal-(1→3)\|→4)-α-D-Man-(1→3)-α-D-Man-(1→3)-β-D-GlcNAc- | Formula |
| | (1→]ₙ | O88 |
| O90 | [→4)-α-L-Fuc2/3Ac-(1→2)-β-D-Gal-(1→3)-α-D-GalNAc-(1→3)-β-D- | Formula |
| | GalNAc-(1→]ₙ | O90 |
| O91 | [→4)-α-D-Qui3Nacyl-(1→4)-β-D-Gal-(1→4)-β-D-GlcNAc-(1→4)-β-D- | Formula |
| | GlcA6Ngly-(1→3)-β-D-GlcNAc-(1→Acyl=l-3-hydroxybutyryl]ₙ | O91 |
| O92 | [->1)bDFruf3Ac(2->3)bDFruf(2->]ₙ | Formula |
| | | O92 |
| O93 | [->2)bDMan(1->3)bDMan2Ac6Ac(1->4)bDGlcA(1->3)aDGlcNAc(1->]ₙ | Formula |
| | | O93 |
| O95 | [->3)aDFuc(1->3)bDXulf(2->]ₙ | Formula |
| | | O95 |
| O96 | 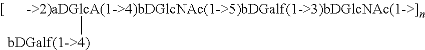 | Formula O96 |
| O97 | [→3)-α-I-Rha-(1→3)-β-I-Rha-(1→\|\|β-d-Xuff-(2→2)β-d-Xulf-(2→2)]n | Formula |
| | | O97 |
| O98 | [→3)-α-L-QuiNAc-(1→4)-α-D-GalNAcA-(1→3)-α-L-QuiNAc-(1→3)-β-D- | Formula |
| | GlcNAc-(1→]ₙ | O98 |
| O99 | 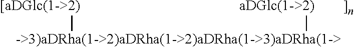 | Formula O99 |
| O100 | 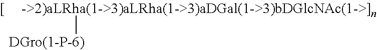 | Formula O100 |
| O101 | [->6)aDGlcNAc(1->4)aDGalNAc(1->]ₙ | Formula |
| | | O101 |
| O102 | 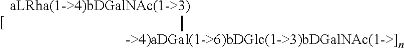 | Formula O102 |
| O103 | [->2)bDGlc(1->2)bDFuc3NBuR3OH(1->6)aDGlcNAc(1->4)aDGalNAc(1->3)bDGlcNAc(1->]ₙ | Formula |
| | | O103 |
| O104 | [→4)-α-D-Gal-(1→4)-α-Neu5,7,9Ac₃-(2→3)-β-D-Gal-(1→3)-β-D-GalNAc- | Formula |
| | (1→]ₙ | O104 |
| O105 | [β-D-Ribf-(1→3)\|→4)-α-D-GlcA2Ac3Ac-(1→2)-α-L-Rha4Ac-(1→3)-β-L- | Formula |
| | Rha-(1→4)-β-L-Rha-(1→3)-β-D-GlcNAc6Ac-(1→]ₙ | O105 |
| O106 | 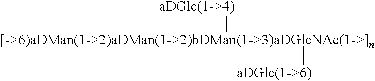 | Formula O106 |
| O107 | [->4)bDGalNAc(1->3)aLRha(1->4)aDGlcNAc(1->4)bDGal(1->3)aDGalNAc(1->]ₙ | Formula |
| | | O107 |
| O108 | [->4)a8eLeg5Ac7Ac(2->6)aDGal(1->3)aLFucNAc(1->3)aDGlcNAc(1->]ₙ | Formula |
| | | O108 |
| O109 | 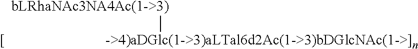 | Formula O109 |
| O110 | [->4)aDGalA6DaThr(1->4)aDGal(1->3)aDGal(1->3)bDGlcNAc(1->]ₙ | Formula |
| | | O110 |
| O111 | [α-Col-(1→6)\|→4)-α-D-Glc-(1→4)-α-D-Gal-(1→3)-β-D-GlcNAc-(1→\|α- | Formula |
| | Col-(1→3)]ₙ | O111 |
| O112ab | 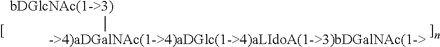 | Formula O112ab |
| O112ac | 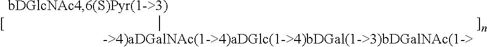 | Formula O112ac |
| O113 | [→4)-α-D-GalNAc-(1→4)-α-D-GalA-(1→3)-α-D-Gal-(1→3)-β-D-GlcNAc- | Formula |
| | (1→\|β-D-Gal-(1→3)]ₙ | O113 |
| O114 | [→4)-β-D-Qui3N(N-acetyl-L-seryl)-(1→3)-β-D-Ribf-(1→4)-β-D-Gal- | Formula |
| | (1→3)-α-D-GlcNAc(1→]ₙ | O114 |
| O115 | 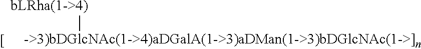 | Formula O115 |
| O116 | [→2)-β-D-Qui4Nac-(1→6)-α-D-GlcNAc-(1→4)-α-D-GalNAc-(1→4)-α-D- | Formula |
| | GalA-(1→3)-β-D-GlcNAc-(1→]ₙ | O116 |
| O117 | [→4)-β-D-GalNAc-(1→3)-α-L-Rha-(1→4)-α-D-Glc-(1→4)-β-D-Gal- | Formula |
| | (1→3)-α-D-GalNAc-(1→]ₙ | O117 |
| O118 | [->3)DRibitol(5-P-6)aDGal(1->3)aLFucNAm(1->3)bDGlcNAc(1->]ₙ | Formula |
| | | O118 |
| O119 | [β-D-RhaNAc3Nfo-(1→3)\|→2)-β-D-Man-(1→3)-α-D-Gal-(1→4)-α-L- | Formula |
| | Rha-(1→3)-α-D-GlcNAc-(1→]ₙ | O119 |
| O120 | 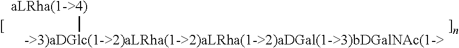 | Formula O120 |
| O121 | [→3)-β-D-Qui4N(N-acetyl-glycyl)-(1→4)-α-D-GalNAc3AcA6N-(1→4)-α- | Formula |
| | D-GalNAcA-(1→3)-α-D-GlcNAc-(1→]ₙ | O121 |
| O123 | [->3)bDQui4NLA1aBuS3OH(1->6)aDGlcNAc(1->3)aLQuiNAc(1->3)aDGlcNAc(1->]ₙ | Formula |
| | | O123 |
| O124 | [4-O-[I-1-carboxyethyl]-β-D-Glc-(1→6)-α-D-Glc(1→4)\|→3)-α-D-Gal- | Formula |
| | (1→6)-β-D-Galf-(1→3)-β-D-GalNAc-(1→]ₙ | O124 |
| O125 | [α-D-Glc-(1→3)\|→4)-β-D-GalNAc-(1→2)-α-D-Man-(1→3)-α-L-Fuc- | Formula |
| | (1→3)-α-D-GalNAc-(1→\|β-D-Gal-(1→3)]ₙ | O125 |
| O126 | [→2)-β-D-Man-(1→3)-β-D-Gal-(1→3)-α-D-GlcNAc-(1→3)-β-D-GlcNAc- | Formula |
| | (1→\|α-L-Fuc-(1→2)]ₙ | O126 |
| O127 | [→2)-α-L-Fuc-(1→2)-β-D-Gal-(1→3)-α-D-GalNAc-(1→3)-α-D-GalNAc- | Formula |
| | (1→]ₙ | O127 |
| O128 | [α-L-Fuc-(1→2)\|→6)-β-D-Gal-(1→3)-β-D-GalNAc-(1→4)-α-D-Gal- | Formula |
| | (1→3)-β-D-GalNAc-(1→]ₙ | O128 |
| O129 | 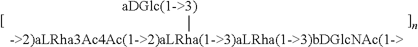 | Formula O129 |
| O130 | 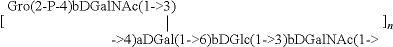 | Formula O130 |
| O131 | [->8)aNeu5Ac(2->6)bDGal(1->6)bDGal(1->3)bDGalNAc(1->]ₙ | Formula |
| | | O131 |
| O132 | [->2)aDGalf(1->3)aLRha2Ac(1->4)aDGlc(1->2)aLRha(1->3)bDGlcNAc(1->]ₙ | Formula |
| | | O132 |
| O133 | 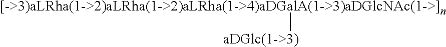 | Formula O133 |
| O134 | [->4)bDGlcA6LThr(1->6)bDGal(1->6)bDGlc(1->3)bDGalNAc(1->]ₙ | Formula |
| | | O134 |
| O135 | 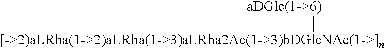 | Formula O135 |
| O136 | [→4)-β-Pse5Ac7Ac-(2→4)-β-D-Gal-(1→4)-β-D-GlcNAc-(1→β- | Formula |
| | Pse5Ac7Ac=5,7-diacetamido-3,5,7,9-tetradeoxy-L-glycero-β-L-manno- | O136 |
| | nonulosonic acid]ₙ | |
| O137 | 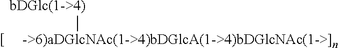 | Formula O137 |
| O138 | [→2)-α-L-Rha-(1→3)-α-L-Rha-(1→4)-α-D-GalNAcA-(1→3)-β-D-GlcNAc- | Formula |
| | (1→]ₙ | O138 |
| O139 | [β-D-Glc-(1→3)\|→3)-α-L-Rha-(1→4)-α-D-GalA-(1→2)-α-L-Rha-(1→3)- | Formula |
| | α-L-Rha-(1→2)-α-L-Rha-(1→3)-α-D-GlcNAc-(1→]ₙ | O139 |
| O140 | 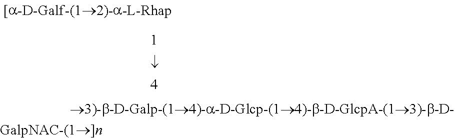 | Formula O140 |
| O141 | [α-L-Rha-(1→3)\|→4)-α-D-Man-(1→3)-α-D-Man6Ac-(1→3)-β-D-GlcNAc- | Formula |
| | (1→\|β-D-GlcA-(1→2)]ₙ | O141 |
| O142 | [→2)-α-L-Rha-(1→6)-α-D-GalNAc-(1→4)-α-D-GalNAc-(1→3)-α-D- | Formula |
| | GalNAc-(1→\|β-D-GlcNAc-(1→3)]ₙ | O142 |
| O143 | [→2)-β-D-GalA6R3,4Ac-(1→3)-α-D-GalNAc-(1→4)-β-D-GlcA-(1→3)-β- | Formula |
| | D-GlcNAc-(1→R=1,3-dihydroxy-2-propylamino]ₙ | O143 |
| O145 | [->4)aNeu5Ac(2->3)aLFucNAm(1->3)bDGlcNAc(1->]ₙ | Formula |
| | | O145 |
| O146 | 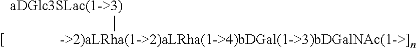 | Formula O146 |
| O147 | [→2)-α-L-Rha-(1→2)-α-L-Rha-(1→4)-β-D-GalA-(1→3)-β-D-GalNAc- | Formula |
| | (1→]ₙ | O147 |
| O148 | [->3)aLRha(1->3)aLRha(1->2)aDG1c(1->3)aDG1cNAc(1->]ₙ | Formula |
| | | O148 |
| O149 | [→3)-β-D-GlcNAc-(S)-4,6Py-(1→3)-β-L-Rha-(1→4)-β-D-GlcNAc-(1→ | Formula |
| | (S)-4,6Py=4,6-O-[(S)-1-carboxyethylidene]-]ₙ | O149 |
| O150 | 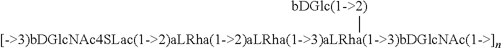 | Formula O150 |
| O151 | 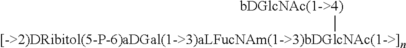 | Formula O151 |
| O152 | [β-L-Rha-(1→4)\|→3)-α-D-GlcNAc-(1-P→6)-α-D-Glc-(1→2)-β-D-Glc- | Formula |
| | (1→3)-β-D-GlcNAc-(1→]ₙ | O152 |
| O153 | [→2)-β-D-Ribf-(1→4)-β-D-Gal-(1→4)-α-D-GlcNAc-(1→4)-β-D-Gal- | Formula |
| | (1→3)-α-D-GlcNAc-(1→]ₙ | O153 |
| O154 | 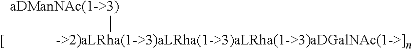 | Formula O154 |
| O155 | 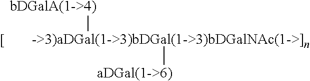 | Formula O155 |
| O156 | 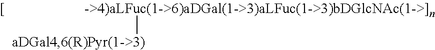 | Formula O156 |
| O157 | [→2)-α-D-Rha4Nac-(1→3)-α-L-Fuc-(1→4)-β-D-Glc-(1→3)-α-D-GalNAc- | Formula |
| | (1→]ₙ | O157 |
| O158 | [α-D-Glc-(1→6)\|→4)-α-D-Glc-(1→3)-α-D-GalNAc-(1→3)-β-D-GalNAc- | Formula |
| | (1→\|α-L-Rha-(1→3)]ₙ | O158 |
| O159 | [α-L-Fuc-(1→4)\|→3)-β-D-GlcNAc-(1→4)-α-D-GalA-(1→3)-β-L-Fuc- | Formula |
| | (1→3)-β-D-GlcNAc-(1→]ₙ | O159 |
| O160 | 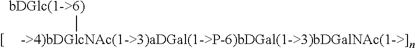 | Formula O160 |
| O161 | [->8)aLeg5Ac7DAla(2->4)bDGlcA(1->3)bDGlcNAc(1->]ₙ | Formula |
| | | O161 |
| O162 | [->6)aDGlcNAc(1->4)aDGalNAc(1->]ₙ | Formula |
| | | O162 |
| O163 | [->2)bDMan(1->4)bDGlcA(1->3)aLQuiNAc(1->3)aDGlcNAc(1->]ₙ | Formula |
| | | O163 |
| O164 | [β-D-Glc-(1→6)-α-D-Glc(1→4)\|→3)-β-D-Gal-(1→6)-β-D-Galf-(1→3)-β- | Formula |
| | D-GalNAc-(1→]ₙ | O164 |
| O165 | [->8)aPse5NBuR3OH(2->6)bDGal(1->4)bDGlc(1->3)aDGlcNAc(1->]ₙ | Formula |
| | | O165 |
| O166 | 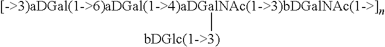 | Formula O166 |
| O167 | [α-D-Galf-(1→4)\|→2)-β-D-GalA6N(L)Ala-(1→3)-α-D-GlcNAc-(1→2)-β- | Formula |
| | D-Galf-(1→5)-β-D-Galf-(1→3)-β-D-GlcNAc-(1→]ₙ | O167 |
| O168 | 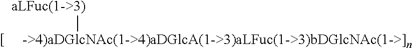 | Formula O168 |
| O169 | 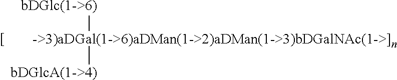 | Formula O169 |
| O170 | 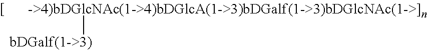 | Formula O170 |
| O171 | [->4)aNeu5Ac7Ac9Ac(2->6)bDGal(1->6)bDGlc(1->3)bDGal(1->3)bDGalNAc(1->]ₙ | Formula |
| | | O171 |
| O172 | [→3)-α-L-FucNAc-(1→4)-α-D-Glc6Ac-(1-P→4)-α-D-Glc-(1→3)-α-L- | Formula |
| | FucNAc-(1→3)-α-D-GlcNAc-(1→]ₙ | O172 |
| O173 | [α-L-Fuc-(1→4)\|→3)-α-D-Glc-(1-P→6)-α-D-Glc-(1→2)-β-D-Glc-(1→3)- | Formula |
| | β-D-GlcNAc-(1→]ₙ | O173 |
| O174ab | 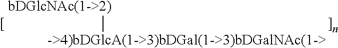 | Formula O174ab |
| O174ac | 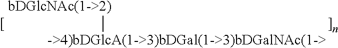 | Formula O174ac |
| O175 | [->2)aDGlc(1->4)aDGlcA(1->3)aDMan(1->2)aDMan(1->3)bDGalNAc(1->]ₙ | Formula |
| | | O175 |
| O176 | [->4)aDMan(1->2)aDMan(1->2)bDMan(1->3)aDGalNAc(1->]ₙ | Formula |
| | | O176 |
| O177 | [->2)aLRha(1->3)aLFucNAc(1->3)aLFucNAc(1->3)bDGlcNAc(1->]ₙ | Formula |
| | | O177 |
| O178 | [->4)aDGlcNAc(1->4)aDGalA(1->2)bDRibf(1->4)bDGal(1->3)bDGalNAc(1->]ₙ | Formula |
| | | O178 |
| O179 | 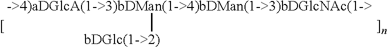 | Formula O179 |
| O180 | [->4)bDManNAc3NAcA(1->2)aLRha(1->3)bLRha(1->4)aDGlcNAc(1->]ₙ | Formula |
| | | O180 |
| O181 | 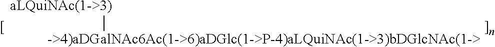 | Formula O181 |
| O182 | 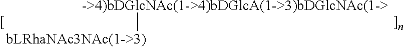 | Formula O182 |
| O183 | 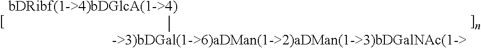 | Formula O183 |
| O184 | [->2)aDFuc3NBuR3OH(1->3)aDMan6Ac(1->3)aLFuc(1->3)aDGalNAc(1->]ₙ | Formula |
| | | O184 |
| O185 | [->4)bDGalNAc(1->2)bDGlcA(1->3)bDRibf(1->4)bDGal(1->3)aDGalNAc(1->]ₙ | Formula |
| | | O185 |
| O186 | [->3)bDQui4NLAlaBuS3OH(1->6)aDGlc(1->3)aLQuiNAc(1->3)aDGlNAc(1->]ₙ | Formula |
| | | O186 |
| O187 | [->2)bDFuc3NAc(1->3)aDGlcNAc(1->4)aDGlcA(1->3)aDMan(1->2)aDMan(1->3)bDGlcNAc(1->]ₙ | Formula |
| | | O187 |
| O188 | 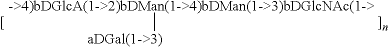 | Formula O188 |

† β-D-6dmanHep2Ac is 2-O-acetyl-6-deoxy-β-D-manno-heptopyranosyl.
‡ B-D-Xulf is β-D-threo-pentofuranosyl.

### Core Oligosaccharide

The core oligosaccharide is positioned between Lipid A and the O-antigen outer region in wild-type E. coli LIPS. More specifically, the core oligosaccharide is the part of the polysaccharide that includes the bond between the O-antigen and the lipid A in wild type E. coli.

This bond includes a ketosidic bond between the hemiketal function of the innermost 3-deoxy-d-manno-oct-2-ulosonic acid (KDO)) residue and a hydroxyl-group of a GlcNAc-residue of the lipid A. The core oligosaccharide region shows a high degree of similarity among wild-type E. coli strains. It usually includes a limited number of sugars. The core oligosaccharide includes an inner core region and an outer core region.

More specifically, the inner core is composed primarily of L-glycero-D-manno-heptose (heptose) and KDO residues. The inner core is highly conserved. A KDO residue includes the following Formula KDO:

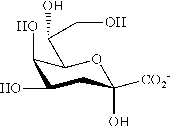

The outer region of the core oligosaccharide displays more variation than the inner core region, and differences in this region distinguish the five chemotypes in E. coli: R1, R2, R3, R4, and K-12. The generalized structures of the carbohydrate backbone of the outer core oligosaccharides of the five known chemotypes are well-known in the art. HeplI is the last residue of the inner core oligosaccharide. While all of the outer core oligosaccharides share a structural theme, with a (hexose)₃ carbohydrate backbone and two side chain residues, the order of hexoses in the backbone and the nature, position, and linkage of the side chain residues can all vary. The structures for the R1 and R4 outer core oligosaccharides are highly similar, differing in only a single β-linked residue.

The core oligosaccharides of wild-type E. coli are categorized in the art based on the structures of the distal oligosaccharide, into five different chemotypes: E. coli R1, E. coli R2, E. coli R3, E. coli R4, and E. coli K12.

In a particular embodiment, the compositions described herein include glycoconjugates in which the O-polysaccharide includes a core oligosaccharide bound to the O-antigen. In one embodiment, the composition induces an immune response against at least any one of the core E. coli chemotypes E. coli R1, E. coli R2, E. coli R3, E. coli R4, and E. coli K12. In another embodiment, the composition induces an immune response against at least two core E. coli chemotypes. In another embodiment, the composition induces an immune response against at least three core E. coli chemotypes. In another embodiment, the composition induces an immune response against at least four core E. coli chemotypes. In another embodiment, the composition induces an immune response against all five core E. coli chemotypes.

In another particular embodiment, the compositions described herein include glycoconjugates in which the O-polysaccharide does not include a core oligosaccharide bound to the O-antigen. In one embodiment, such a composition induces an immune response against at least any one of the core E. coli chemotypes E. coli R1, E. coli R2, E. coli R3, E. coli R4, and E. coli K12, despite the glycoconjugate having an O-polysaccharide that does not include a core oligosaccharide.

E. coli serotypes may be characterized according to one of the five chemotypes. Table 2 lists exemplary serotypes characterized according to chemotype. The serotypes in bold represent the serotypes that are most commonly associated with the indicated core chemotype.

Accordingly, in a particular embodiment, the composition induces an immune response against at least any one of the core E. coli chemotypes E. coli R1, E. coli R2, E. coli R3, E. coli R4, and E. coli K12, which includes an immune response against any one of the respective corresponding E. coli serotypes.

TABLE 2
Core Chemotype and associated E. coli Serotype

| Core | |
|---|---|
| chemotype | Serotype |
| R1 | O25a, O6, O2, O1, O75, O4, O16, O8, O18, O9, O13, |
| | O20, O21, O91, and O163. |
| R2 | O21, O44, O11, O89, O162, O9 |
| R3 | O25b, O15, O153, O21, O17, O11, O159, O22 O86, O93 |
| R4 | O2, O1, O86, O7, O102, O160, O166 |
| K-12 | O25b, O16 |

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O8, Formula O18, Formula O9, Formula O13, Formula O20, Formula O21, Formula O91, and Formula O163, wherein n is 1 to 100. In some embodiments, the saccharide in said composition further includes an E. coli R1 core moiety.

In some embodiments, the composition includes a saccharide that includes a structure derived from a serotype having an R1 chemotype, e.g., selected from a saccharide having Formula O25a, Formula O6, Formula O2, Formula O1, Formula O75, Formula O4, Formula O16, Formula O18, Formula O13, Formula O20, Formula O21, Formula O91, and Formula O163, wherein n is 1 to 100, such as 31 to 100, from 31 to 90, 35 to 90, or 35 to 65. In some embodiments, the saccharide in said composition further includes an E. coli R1 core moiety in the saccharide.

In some embodiments, the composition includes a saccharide that includes a structure derived from a serotype having an R2 chemotype, e.g., selected from a saccharide having Formula O21, Formula O44, Formula O11, Formula O89, Formula O162, and Formula O9, wherein n is 1 to 100, such as 31 to 100, 31 to 90, 35 to 90, or 35 to 65. In some embodiments, the saccharide in said composition further includes an E. coli R2 core moiety.

In some embodiments, the composition includes a saccharide that includes a structure derived from a serotype having an R3 chemotype, e.g., selected from a saccharide having Formula O25b, Formula O15, Formula O153, Formula O21, Formula O17, Formula O11, Formula O159, Formula O22, Formula O86, and Formula O93, wherein n is 1 to 100, such as 31 to 100, 31 to 90, 35 to 90, or 35 to 65. In some embodiments, the saccharide in said composition further includes an E. coli R3 core moiety.

In some embodiments, the composition includes a saccharide that includes a structure derived from a serotype having an R4 chemotype, e.g., selected from a saccharide having Formula O2, Formula O1, Formula O86, Formula O7, Formula O102, Formula O160, and Formula O166, wherein n is 1 to 100, such as 31 to 100, such as from 31 to 90, such as 35 to 90, or such as 35 to 65. In some embodiments, the saccharide in said composition further includes an E. coli R4 core moiety.

In some embodiments, the composition includes a saccharide that includes a structure derived from a serotype having an K-12 chemotype (e.g., selected from a saccharide having Formula O25b and a saccharide having Formula O16), wherein n is 1 to 1000, such as 31 to 100, such as from 31 to 90, such as 35 to 90, such as 35 to 65. In some embodiments, the saccharide in said composition further includes an E. coli K-12 core moiety.

In some embodiments, the saccharide includes the core saccharide. Accordingly, in one embodiment, the O-polysaccharide further includes an E. coli R1 core moiety. In another embodiment, the O-polysaccharide further includes an E. coli R2 core moiety. In another embodiment, the O-polysaccharide further includes an E. coli R3 core moiety. In another embodiment, the O-polysaccharide further includes an E. coli R4 core moiety. In another embodiment, the O-polysaccharide further includes an E. coli K12 core moiety.

In some embodiments, the saccharide does not include the core saccharide. Accordingly, in one embodiment, the O-polysaccharide does not include an E. coli R1 core moiety. In another embodiment, the O-polysaccharide does not include an E. coli R2 core moiety. In another embodiment, the O-polysaccharide does not include an E. coli R3 core moiety. In another embodiment, the O-polysaccharide does not include an E. coli R4 core moiety. In another embodiment, the O-polysaccharide does not include an E. coli K12 core moiety.

### Glycoconjugates

Chemical linkage of O-antigens or O-polysaccharides to protein carriers may improve the immunogenicity of the O-antigens or O-polysaccharides. However, variability in polymer size represents a practical challenge for production. In commercial use, the size of the saccharide can influence the compatibility with different conjugation synthesis strategies, product uniformity, and conjugate immunogenicity. Controlling the expression of a Wzz family protein chain length regulator through manipulation of the O-antigen synthesis pathway allows for production of a desired length of O-antigen chains in a variety of Gram-negative bacterial strains, including E. coli.

In one embodiment, the purified saccharides are chemically activated to produce activated saccharides capable of reacting with the carrier protein. Once activated, each saccharide is separately conjugated to a carrier protein to form a conjugate, namely a glycoconjugate. As used herein, the term ‘glycoconjugate’ indicates a saccharide (in particular a bacterial saccharide) linked to a carrier protein. In one embodiment a saccharide is linked directly to a carrier protein. In another embodiment, a saccharide is linked to a protein through a spacer/linker. Conjugates may be prepared by schemes that bind the carrier to the O-antigen at one or at multiple sites along the O-antigen, or by schemes that activate at least one residue of the core oligosaccharide.

In some embodiments, a glycoconjugate described herein can comprise a saccharide covalently bound to a carrier protein. In a particular embodiment, a glycoconjugate comprises a saccharide covalently bound to a SCP carrier protein.

In other embodiments, a glycoconjugate described herein can comprise a saccharide noncovalently bound to a carrier protein. In one embodiment, a glycoconjugate comprises a saccharide bound to a carrier protein via the noncovalent interaction between biotin and streptavidin. In some embodiments, a glycoconjugate comprises a saccharide bound to a carrier protein via the noncovalent interaction between biotin and streptavidin and the saccharide is biotinylated. In further embodiments, a glycoconjugate comprises a saccharide bound to a carrier protein via the noncovalent interaction between biotin and streptavidin and the carrier protein is expressed as a fusion protein with streptavidin. In additional embodiments, the streptavidin is a streptavidin fragment.

In one embodiment, each saccharide is conjugated to the same carrier protein. If the protein carrier is the same for 2 or more saccharides in the composition, the saccharides may be conjugated to the same molecule of the carrier protein (e.g., carrier molecules having 2 or more different saccharides conjugated to it).

In a particular embodiment, the saccharides are each individually conjugated to different molecules of the protein carrier (each molecule of protein carrier only having one type of saccharide conjugated to it). In said embodiment, the saccharides are said to be individually conjugated to the carrier protein.

In some embodiments, the glycoconjugate of the present disclosure comprises a saccharide wherein the weight average molecular weight (Mw) of said polysaccharide before conjugation is between about 10 kDa and about 2,000 kDa.

The average molecular weight (Mw) of the saccharide before conjugation refers to the average Mw before the activation of the saccharide (i.e., before reacting the saccharide with an activating agent). In an embodiment, the saccharide is activated with a carbonic acid derivative (e.g., CDI or CDT) in combination with an agent comprising an azide (e.g., 3-azido-1-propylamine).

In an embodiment, the glycoconjugate of the present disclosure comprises a saccharide wherein the average Mw of said saccharide before conjugation is between about 10 kDa and about 2,000 kDa. For example, in some embodiments, the average Mw of the saccharide before conjugation is between about 10 kDa and about 1,000 kDa. In still other embodiments, the average Mw of the saccharide before conjugation is between about 10 kDa and about 500 kDa. In particular embodiments, the average Mw of the saccharide before conjugation is between about 10 kDa and about 100 kDa. In other particular embodiments, the average Mw of the saccharide before conjugation is between about 40 kDa and about 60 kDa. For example, in some embodiments, the average Mw of the saccharide before conjugation is about 40, about 41, about 42, about 43, about 44, about 45, about 46, about 47, about 48, about 49, about 50, about 51, about 52, about 53, about 54, about 55, about 56, about 57, about 58, about 59, or about 60 kDa.

In another embodiment, the glycoconjugate of the present disclosure has an average molecular weight (Mw) between about 100 kDa and about 5,000 kDa. For example, in some embodiments, the average Mw of the glycoconjugate is between about 100 kDa and about 2,000 kDa. In still other embodiments, the average Mw of the glycoconjugate is between about 500 kDa and about 1,500 kDa. In particular embodiments, the average Mw of the glycoconjugate is about 400, about 450, about 500, about 550, about 600, about 650, about 700, about 750, about 800, about 850, about 900, about 950, about 1,000, about 1,050, about 1,100, about 1,150, about 1,200, about 1,250, about 1,300, about 1,350, about 1,400, about 1,450 or about 1,500 kDa.

The molecular weight of the glycoconjugate may be measured by SEC-MALLS. Any whole number integer within any of the above ranges is contemplated as an embodiment of the disclosure.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules. Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (Vᵢ), (K_{d}=1). The fraction at which a specified sample attribute is reached (Vₑ), is related to Kd by the expression,

$${K}_{d} = ( {V}_{e} - {V}_{o} ) / ( {V}_{i} - {V}_{0} ) .$$

The chemical activation of the saccharides and subsequent conjugation to the carrier protein can be achieved by the activation and conjugation methods disclosed herein. After conjugation of the polysaccharide to the carrier protein, the glycoconjugates are purified (enriched with respect to the amount of polysaccharide-protein conjugate) by a variety of techniques. These techniques include concentration/diafiltration operations, precipitation/elution, column chromatography, and depth filtration. After the individual glycoconjugates are purified, they are compounded to formulate the immunogenic composition of the present disclosure.

The present disclosure further relates to activated polysaccharides produced from any of the embodiments described herein wherein the polysaccharide is activated with a chemical reagent to produce reactive groups for conjugation to a linker or carrier protein. In some embodiments, the saccharide of the disclosure is activated prior to conjugation to the carrier protein.

In a particular embodiment, the polysaccharide is activated with a carbonic acid derivative and an agent comprising an azide. In another particular embodiment, the polysaccharide is activated with 1,1′-carbonyldiimidazole (CDI) as the carbonic acid derivative. In still another particular embodiment, the polysaccharide is activated with 1,1′-carbonyl-di-(1,2,4-triazole) (CDT) as the carbonic acid derivative. In one particular embodiment, the polysaccharide is activated with 3-azido-1-propylamine as the agent comprising an azide.

In some embodiments, the glycoconjugate is prepared by CDAP chemistry. In another embodiment, the polysaccharide is activated with 1-cyano-4-dimethylamino pyridinium tetrafluoroborate (CDAP) to form a cyanate ester. The activated polysaccharide is then coupled directly or via a spacer (linker) group to an amino group on the carrier protein (for example, CRM₁₉₇, SCP, or tetanus toxoid).

For example, the spacer may be cystamine or cysteamine to give a thiolated polysaccharide which could be coupled to the carrier via a thioether linkage obtained after reaction with a maleimide-activated carrier protein (for example using N—[Y-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA), or succinimidyl 3-[bromoacetamido]proprionate (SBAP)). In one embodiment, the cyanate ester (optionally made by CDAP chemistry) is coupled with hexane diamine or adipic acid dihydrazide (ADH) and the amino-derivatised saccharide is conjugated to the carrier protein using carbodiimide (e.g., EDAC or EDC) chemistry via a carboxyl group on the protein carrier.

Other suitable techniques for conjugation use carbodiimides, hydrazides, active esters, norborane, p-nitrobenzoic acid, N-hydroxysuccinimide, S—NHS, EDC, TSTU. Conjugation may involve a carbonyl linker which may be formed by reaction of a free hydroxyl group of the saccharide with CDI followed by reaction with a protein to form a carbamate linkage. This may involve reduction of the anomeric terminus to a primary hydroxyl group, optional protection/deprotection of the primary hydroxyl group, reaction of the primary hydroxyl group with CDI to form a CDI carbamate intermediate and coupling the CDI carbamate intermediate with an amino group on a protein (CDI chemistry).

Another way to characterize the glycoconjugates of the disclosure is by the number of lysine residues in the carrier protein (e.g., CRM₁₉₇ or SCP) that become conjugated to the saccharide which can be characterized as a range of conjugated lysines (degree of conjugation). The evidence for lysine modification of the carrier protein, due to covalent linkages to the saccharides, can be obtained by amino acid analysis using routine methods known to those of skill in the art. Conjugation results in a reduction in the number of lysine residues recovered compared to the carrier protein starting material used to generate the conjugate materials. In a particular embodiment, the degree of conjugation of the glycoconjugate of the disclosure is between 2 and 15. In an embodiment, the degree of conjugation of the glycoconjugate of the disclosure is between 2 and 13. In an embodiment, the degree of conjugation of the glycoconjugate of the disclosure is between 2 and 10. In an embodiment, the degree of conjugation of the glycoconjugate of the disclosure is between 2 and 8. In an embodiment, the degree of conjugation of the glycoconjugate of the disclosure is between 2 and 6. In an embodiment, the degree of conjugation of the glycoconjugate of the disclosure is between 2 and 5. In an embodiment, the degree of conjugation of the glycoconjugate of the disclosure is between 2 and 4. In an embodiment, the degree of conjugation of the glycoconjugate of the disclosure is between 3 and 15. In an embodiment, the degree of conjugation of the glycoconjugate of the disclosure is between 3 and 13. In an embodiment, the degree of conjugation of the glycoconjugate of the disclosure is between 3 and 10. In an embodiment, the degree of conjugation of the glycoconjugate of the disclosure is between 3 and 8. In an embodiment, the degree of conjugation of the glycoconjugate of the disclosure is between 3 and 6. In an embodiment, the degree of conjugation of the glycoconjugate of the disclosure is between 3 and 5. In an embodiment, the degree of conjugation of the glycoconjugate of the disclosure is between 3 and 4. In an embodiment, the degree of conjugation of the glycoconjugate of the disclosure is between 5 and 15. In an embodiment, the degree of conjugation of the glycoconjugate of the disclosure is between 5 and 10. In an embodiment, the degree of conjugation of the glycoconjugate of the disclosure is between 8 and 15. In an embodiment, the degree of conjugation of the glycoconjugate of the disclosure is between 8 and 12. In an embodiment, the degree of conjugation of the glycoconjugate of the disclosure is between 10 and 15. In an embodiment, the degree of conjugation of the glycoconjugate of the disclosure is between 10 and 12.

In an embodiment, the degree of conjugation of the glycoconjugate of the disclosure is about 2. In an embodiment, the degree of conjugation of the glycoconjugate of the disclosure is about 3. In an embodiment, the degree of conjugation of the glycoconjugate of the disclosure is about 4. In an embodiment, the degree of conjugation of the glycoconjugate of the disclosure is about 5. In an embodiment, the degree of conjugation of the glycoconjugate of the disclosure is about 6. In an embodiment, the degree of conjugation of the glycoconjugate of the disclosure is about 7. In an embodiment, the degree of conjugation of the glycoconjugate of the disclosure is about 8. In an embodiment, the degree of conjugation of the glycoconjugate of the disclosure is about 9. In an embodiment, the degree of conjugation of the glycoconjugate of the disclosure is about 10, about 11. In an embodiment, the degree of conjugation of the glycoconjugate of the disclosure is about 12. In an embodiment, the degree of conjugation of the glycoconjugate of the disclosure is about 13. In an embodiment, the degree of conjugation of the glycoconjugate of the disclosure is about 14. In an embodiment, the degree of conjugation of the glycoconjugate of the disclosure is about 15. In a particular embodiment, the degree of conjugation of the glycoconjugate of the disclosure is between 4 and 7. In some such, embodiments, the carrier protein is SCP. In other such embodiments, the carrier protein is CRM₁₉₇.

The frequency of attachment of the saccharide chain to a lysine on the carrier protein is another parameter for characterizing the glycoconjugates of the disclosure. For example, in some embodiments, at least one linkage between the carrier protein and the polysaccharide for every 4 saccharide repeat units of the polysaccharide. In another embodiment, the linkage between the carrier protein and the polysaccharide occurs at least once in every 10 saccharide repeat units of the polysaccharide. In another embodiment, the linkage between the carrier protein and the polysaccharide occurs at least once in every 15 saccharide repeat units of the polysaccharide. In a further embodiment, the linkage between the carrier protein and the polysaccharide occurs at least once in every 25 saccharide repeat units of the polysaccharide.

In some embodiments, the saccharides of the disclosure are O-acetylated. In some embodiments, the glycoconjugate comprises a saccharide which has a degree of O-acetylation of between 10-100%, between 20-100%, between 30-100%, between 40-100%, between 50-100%, between 60-100%, between 70-100%, between 75-100%, 80-100%, 90-100%, 50-90%, 60-90%, 70-90% or 80-90%. In other embodiments, the degree of O-acetylation is 10%, ≥20%, ≥30%, ≥40%, ≥50%, ≥60%, ≥70%, ≥80%, or ≥90%, or about 100%. By % of O-acetylation it is meant the percentage of a given saccharide relative to 100% (where each repeat unit is fully acetylated relative to its acetylated structure).

The glycoconjugates of the disclosure may also be characterized by the ratio (weight/weight) of saccharide to carrier protein. In some embodiments, the ratio of saccharide to carrier protein in the glycoconjugate (w/w) is between about 0.5 and about 3.0. In other embodiments, the saccharide to carrier protein ratio (w/w) is between about 0.5 and about 2.0. In other embodiments, the saccharide to carrier protein ratio (w/w) is between about 0.5 and about 1.5. In some embodiments, the saccharide to carrier protein ratio (w/w) is between about 0.5 and about 1.0. For examples, in particular embodiments, the saccharide to carrier protein ratio (w/w) is about 0.5, about 0.6, about 0.7, about 0.8, about 0.9, or about 1.0. In some such, embodiments, the carrier protein is SCP. In other such embodiments, the carrier protein is CRM₁₉₇.

The glycoconjugates of the disclosure may also be characterized by the number of linkages between the carrier protein and the saccharide as a function of repeat units of the saccharide. In one embodiment, the glycoconjugate of the disclosure comprises at least one linkage between the carrier protein and the saccharide for between about every 1 and about every 10 repeat units of the saccharide. In other embodiments, the conjugate comprises at least one linkage between the carrier protein and saccharide for every 5 to 10 saccharide repeat units; every 2 to 7 saccharide repeat units; every 3 to 8 saccharide repeat units; every 4 to 9 saccharide repeat units; every 6 to 11 saccharide repeat units; every 7 to 12 saccharide repeat units; every 8 to 13 saccharide repeat units; every 9 to 14 saccharide repeat units; every 10 to 15 saccharide repeat units; every 2 to 6 saccharide repeat units, every 3 to 7 saccharide repeat units; every 4 to 8 saccharide repeat units; every 6 to 10 saccharide repeat units; every 7 to 11 saccharide repeat units; every 8 to 12 saccharide repeat units; every 9 to 13 saccharide repeat units; every 10 to 14 saccharide repeat units; every 10 to 20 saccharide repeat units; every 4 to 25 saccharide repeat units or every 2 to 25 saccharide repeat units. In other embodiments, the glycoconjugate of the disclosure comprises at least one linkage between the carrier protein and the saccharide for between about every 10 and about every 25 repeat units of the saccharide. In another embodiment, at least one linkage between carrier protein and saccharide occurs for every 1, 2, 3, 4, 5, 6, 7, 8, 9, 10, 11, 12, 13, 14, 15, 16, 17, 18, 19, 20, 21, 22, 23, 24 or 25 saccharide repeat units of the polysaccharide. In some such, embodiments, the carrier protein is SCP. In other such embodiments, the carrier protein is CRM₁₉₇.

The glycoconjugates and immunogenic compositions of the disclosure may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In some embodiments, the glycoconjugate comprises less than about 25% of free saccharide compared to the total amount of said saccharide. In some embodiments, the glycoconjugate comprises less than about 10% of free saccharide compared to the total amount of said saccharide. In other embodiments, the glycoconjugate comprises less than about 10%, about 9%, about 8%, about 7%, about 6%, about 5%, about 4%, about 3%, about 2%, or about 1% of free saccharide compared to the total amount of said saccharide.

In some embodiments, the glycoconjugate is prepared by click chemistry, as described herein.

In some embodiments, the glycoconjugate is prepared by reductive amination. In some embodiments, the glycoconjugate is a single-end-linked conjugated saccharide, wherein the saccharide is covalently bound to a carrier protein directly. In some embodiments, the glycoconjugate is covalently bound to a carrier protein through a (2-((2-oxoethyl)thio)ethyl) carbamate (eTEC) spacer.

Reductive Amination: In one embodiment, the saccharide is conjugated to the carrier protein by reductive amination (such as described in U.S. Patent Appl. Pub. Nos. 2006/0228380, 2007/0231340, 2007/0184071 and 2007/0184072, WO 2006/110381, WO 2008/079653, and WO 2008/143709).

Reductive amination includes (1) oxidation of the saccharide, (2) reduction of the activated saccharide and a carrier protein to form a conjugate. Before oxidation, the saccharide is optionally hydrolyzed. Mechanical or chemical hydrolysis may be employed. Chemical hydrolysis may be conducted using acetic acid.

The oxidation step may involve reaction with periodate. The term “periodate” as used herein refers to both periodate and periodic acid. The term also includes both metaperiodate (IO₄⁻) and orthoperiodate (IO₆⁵⁻) and the various salts of periodate (e.g., sodium periodate and potassium periodate). In one embodiment the polysaccharide is oxidized in the presence of metaperiodate, for example in the presence of sodium periodate (NaIO₄). In another embodiment the polysaccharide is oxidized in the presence of orthoperiodate, for example in the presence of periodic acid.

In one embodiment, the oxidizing agent is a stable nitroxyl or nitroxide radical compound, such as piperidine-N-oxy or pyrrolidine-N-oxy compounds, in the presence of an oxidant to selectively oxidize primary hydroxyls. In said reaction, the actual oxidant is the N-oxoammonium salt, in a catalytic cycle. In an aspect, said stable nitroxyl or nitroxide radical compound are piperidine-N-oxy or pyrrolidine-N-oxy compounds. In an aspect, said stable nitroxyl or nitroxide radical compound bears a TEMPO (2,2,6,6-tetramethyl-1-piperidinyloxy) or a PROXYL (2,2,5,5-tetramethyl-1-pyrrolidinyloxy) moiety. In an aspect, said stable nitroxyl radical compound is TEMPO or a derivative thereof. In an aspect, said oxidant is a molecule bearing a N-halo moiety. In an aspect, said oxidant is selected from any one of N-ChloroSuccinimide, N-Bromosuccinimide, N-lodosuccinimide, Dichloroisocyanuric acid, 1,3,5-trichloro-I,3,5-triazinane-2,4,6-trione, Dibromoisocyanuric acid, 1,3,5-tribromo-I,3,5-triazinane-2,4,6-trione, Diiodoisocyanuric acid and 1,3,5-triiodo-I,3,5-triazinane-2,4,6-trione. In a particular embodiment, said oxidant is N-Chlorosuccinimide.

Following the oxidation step of the saccharide, the saccharide is said to be activated. The activated saccharide and the carrier protein may be lyophilised (freeze-dried), either independently (discrete lyophilization) or together (co-lyophilized). In one embodiment the activated saccharide and the carrier protein are co-lyophilized. In another embodiment the activated polysaccharide and the carrier protein are lyophilized independently.

In one embodiment the lyophilization takes place in the presence of a non-reducing sugar, possible non-reducing sugars include sucrose, trehalose, raffinose, stachyose, melezitose, dextran, mannitol, lactitol and palatinit.

The next step of the conjugation process is the reduction of the activated saccharide and a carrier protein to form a conjugate (so-called reductive amination), using a reducing agent. Suitable reducing agents include the cyanoborohydrides, such as sodium cyanoborohydride, sodium triacetoxyborohydride or sodium or zinc borohydride in the presence of Bronsted or Lewis acids), amine boranes such as pyridine borane, 2-Picoline Borane, 2,6-diborane-methanol, dimethylamine-borane, t-BuMe′PrN—BH3, benzylamine-BH3 or 5-ethyl-2-methylpyridine borane (PEMB), borane-pyridine, or borohydride exchange resin. In one embodiment the reducing agent is sodium cyanoborohydride.

In an embodiment, the reduction reaction is carried out in aqueous solvent (e.g., selected from PBS, MES, HEPES, Bis-tris, ADA, PIPES, MOPSO, BES, MOPS, DIPSO, MOBS, HEPPSO, POPSO, TEA, EPPS, Bicine or HEPB, at a pH between 6.0 and 8.5, 7.0 and 8.0, or 7.0 and 7.5), in another embodiment the reaction is carried out in aprotic solvent. In an embodiment, the reduction reaction is carried out in DMSO (dimethylsulfoxide) or in DMF (dimethylformamide) solvent. The DMSO or DMF solvent may be used to reconstitute the activated polysaccharide and carrier protein which has been lyophilized.

At the end of the reduction reaction, there may be unreacted aldehyde groups remaining in the conjugates, these may be capped using a suitable capping agent. In one embodiment this capping agent is sodium borohydride (NaBH₄). Following the conjugation (the reduction reaction and optionally the capping), the glycoconjugates may be purified (enriched with respect to the amount of polysaccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. The glycoconjugates maybe purified by diafiltration and/or ion exchange chromatography and/or size exclusion chromatography. In an embodiment, the glycoconjugates are purified by diafiltration or ion exchange chromatography or size exclusion chromatography. In one embodiment the glycoconjugates are sterile filtered.

In a some embodiments, a glycoconjugate from an E. coli serotype is selected from any one of O1, O2, and O6 is prepared by reductive amination. In some embodiments, the glycoconjugates from E. coli serotypes O1, O2, and O6 are prepared by reductive amination.

Activation and formation of an Aldehyde: In some embodiments, the saccharide of the disclosure is activated and results in the formation of an aldehyde. In such embodiments wherein the saccharide is activated, the percentage (%) of activation (or degree of oxidation (DO)) refers to moles of a saccharide repeat unit per moles of aldehyde of the activated polysaccharide. For example, in some embodiments, the saccharide is activated by periodate oxidation of vicinal diols on a repeat unit of the polysaccharide, resulting in the formation of an aldehyde. Varying the molar equivalents (meq) of sodium periodate relative to the saccharide repeat unit and temperature during oxidation results in varying levels of degree of oxidation (DO).

The saccharide and aldehyde concentrations are typically determined by colorimetric assays. An alternative reagent is TEMPO (2,2,6,6-tetramethylpiperidine 1-oxyl radical)-N-chlorosuccinimide (NCS) combination, which results in the formation of aldehydes from primary alcohol groups.

In some embodiments, the activated saccharide has a degree of oxidation wherein the moles of a saccharide repeat unit per moles of aldehyde of the activated saccharide is between 1-100, such as, for example, between 2-80, between 2-50, between 3-30, and between 4-25. The degree of activation is at least 1, 2, 3, 4, 5, 6, 7, 8, 9, 10, 11, 12, 13, 14, 15, 16, 17, 18, 19, ≥20, ≥30, ≥40, ≥50, ≥60, ≥70, ≥80, or ≥90, or about 100. For example, in some embodiments, the degree of oxidation (DO) is at least 5 and at most 50, e.g., at least 10 and at most 25. In one embodiment, the degree of activation is at least 10 and at most 25. Any minimum value and any maximum value may be combined to define a range. A degree of oxidation value may be represented as percentage (%) of activation. For example, in one embodiment, a DO value of 10 refers to one activated saccharide repeat unit out of a total of 10 saccharide repeat units in the activated saccharide, in which case the DO value of 10 may be represented as 10% activation.

Single-End Linked Conjugates: In some embodiments, the conjugate is single-end-linked conjugated saccharide, wherein the saccharide is covalently bound at one end of the saccharide to a carrier protein. In some embodiments, the single-end-linked conjugated polysaccharide has a terminal saccharide. For example, a conjugate is single-end linked if one of the ends (a terminal saccharide residue) of the polysaccharide is covalently bound to a carrier protein. In some embodiments, the conjugate is single-end linked if a terminal saccharide residue of the polysaccharide is covalently bound to a carrier protein through a linker. Such linkers may include, for example, a cystamine linker, a 3,3′-dithio bis(propanoic dihydrazide) linker, and a 2,2′-dithio-N,N′-bis(ethane-2,1-diyl)bis(2-(aminooxy)acetamide) linker. In some embodiments, the saccharide is conjugated to the carrier protein through a 3-deoxy-d-manno-oct-2-ulosonic acid (KDO) residue to form a single-end linked conjugate.

In some embodiments, the conjugate is not a bioconjugate. The term “bioconjugate” refers to a conjugate between a protein (e.g., a carrier protein) and an antigen, e.g., an O antigen (e.g., O25B) prepared in a host cell background, wherein host cell machinery links the antigen to the protein (e.g., N-links). Glycoconjugates include bioconjugates, as well as sugar antigen (e.g., oligo- and polysaccharides)-protein conjugates prepared by means that do not require preparation of the conjugate in a host cell, e.g., conjugation by chemical linkage of the protein and saccharide.

Thiol Activated Saccharides: In some embodiments, the saccharide of the disclosure is thiol activated. In such embodiments wherein the saccharide is thiol activated, the percentage (%) of activation refers to moles of thiol per saccharide repeat unit of the activated polysaccharide. The saccharide and thiol concentrations are typically determined by Ellman's assay for quantitation of sulfhydryls. For example, in some embodiments, the saccharide includes activation of 2-Keto-3-deoxyoctanoic acid (KDO) with a disulfide amine linker. In some embodiments, the saccharide is covalently bound to a carrier protein through a bivalent, heterobifunctional linker (also referred to herein as a “spacer”). In some embodiments, the linker provides a thioether bond between the saccharide and the carrier protein, resulting in a glycoconjugate referred to herein as a “thioether glycoconjugate.” In some embodiments, the linker further provides carbamate and amide bonds, such as, for example, (2-((2-oxoethyl)thio)ethyl) carbamate (eTEC).

eTec Conjugates: In some embodiments, the glycoconjugates comprise a saccharide derived from E. coli described above covalently conjugated to a carrier protein through a (2-((2-oxoethyl)thio)ethyl)carbamate (eTEC) spacer (as described, for example, in U.S. Pat. No. 9,517,274 and International Patent Application Publication WO2014027302, incorporated by reference herein in their entireties), including immunogenic compositions comprising such glycoconjugates, and methods for the preparation and use of such glycoconjugates and immunogenic compositions. Said glycoconjugates comprise a saccharide covalently conjugated to a carrier protein through one or more eTEC spacers, wherein the saccharide is covalently conjugated to the eTEC spacer through a carbamate linkage, and wherein the carrier protein is covalently conjugated to the eTEC spacer through an amide linkage. The eTEC spacer includes seven linear atoms (i.e., —C(O)NH(CH₂)₂SCH₂C(O)—) and provides stable thioether and amide bonds between the saccharide and carrier protein.

The eTEC linked glycoconjugates of the disclosure may be represented by the general formula (I):

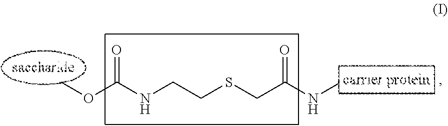

where the atoms that comprise the eTEC spacer are contained in the central box.

In said glycoconjugates of the disclosure, the saccharide may be a polysaccharide or an oligosaccharide.

In another aspect, the disclosure provides a method of making a glycoconjugate comprising a saccharide described herein conjugated to a carrier protein through an eTEC spacer, comprising the steps of a) reacting a saccharide with a carbonic acid derivative in an organic solvent to produce an activated saccharide; b) reacting the activated saccharide with cystamine or cysteamine or a salt thereof, to produce a thiolated saccharide; c) reacting the thiolated saccharide with a reducing agent to produce an activated thiolated saccharide comprising one or more free sulfhydryl residues; d) reacting the activated thiolated saccharide with an activated carrier protein comprising one or more α-haloacetamide groups, to produce a thiolated saccharide-carrier protein conjugate; and e) reacting the thiolated saccharide-carrier protein conjugate with (i) a first capping reagent capable of capping unconjugated α-haloacetamide groups of the activated carrier protein; and/or (ii) a second capping reagent capable of capping unconjugated free sulfhydryl residues of the activated thiolated saccharide; whereby an eTEC linked glycoconjugate is produced.

In frequent embodiments, the carbonic acid derivative is 1,1′-carbonyl-di-(1,2,4-triazole) (CDT) or 1,1′-carbonyldiimidazole (CDI). For example, the carbonic acid derivative is CDT and the organic solvent is a polar aprotic solvent, such as dimethylsulfoxide (DMSO). In some embodiments, the thiolated saccharide is produced by reaction of the activated saccharide with the bifunctional symmetric thioalkylamine reagent, cystamine or a salt thereof. Alternatively, the thiolated saccharide may be formed by reaction of the activated saccharide with cysteamine or a salt thereof. The eTEC linked glycoconjugates produced by the methods of the disclosure may be represented by general Formula (I).

In frequent embodiments, the first capping reagent is N-acetyl-L-cysteine, which reacts with unconjugated α-haloacetamide groups on lysine residues of the carrier protein to form an S-carboxymethylcysteine (CMC) residue covalently linked to the activated lysine residue through a thioether linkage.

In other embodiments, the second capping reagent is iodoacetamide (IAA), which reacts with unconjugated free sulfhydryl groups of the activated thiolated saccharide to provide a capped thioacetamide. Frequently, step e) comprises capping with both a first capping reagent and a second capping reagent. In certain embodiments, step e) comprises capping with N-acetyl-L-cysteine as the first capping reagent and IAA as the second capping reagent.

In some embodiments, the capping step e) further comprises reaction with a reducing agent, for example, DTT, TCEP, or mercaptoethanol, after reaction with the first and/or second capping reagent.

The eTEC linked glycoconjugates and immunogenic compositions of the disclosure may include free sulfhydryl residues. In some instances, the activated thiolated saccharides formed by the methods provided herein will include multiple free sulfhydryl residues, some of which may not undergo covalent conjugation to the carrier protein during the conjugation step. Such residual free sulfhydryl residues are capped by reaction with a athiol-reactive capping reagent, for example, iodoacetamide (IAA), to cap the potentially reactive functionality. Other thiol-reactive capping reagents, e.g., maleimide containing reagents and the like are also contemplated.

In addition, the eTEC linked glycoconjugates and immunogenic compositions of the disclosure may include residual unconjugated carrier protein, which may include activated carrier protein which has undergone modification during the capping process steps.

In some embodiments, step d) further comprises providing an activated carrier protein comprising one or more α-haloacetamide groups prior to reacting the activated thiolated saccharide with the activated carrier protein. In frequent embodiments, the activated carrier protein comprises one or more α-bromoacetamide groups.

In another aspect, the disclosure provides an eTEC linked glycoconjugate comprising a saccharide described herein conjugated to a carrier protein through an eTEC spacer produced according to any of the methods disclosed herein.

In some embodiments, the carrier protein is CRM₁₉₇ and the covalent linkage via an eTEC spacer between the CRM₁₉₇ and the polysaccharide occurs at least once in every 4, 10, 15 or 25 saccharide repeat units of the polysaccharide.

### Carrier Protein of the Glycoconjugates

A component of the glycoconjugate is a carrier protein to which the saccharide is conjugated. The terms “protein carrier” or “carrier protein” or “carrier” may be used interchangeably herein. Carrier proteins should be amenable to standard conjugation procedures.

In some embodiments, the carrier protein of the glycoconjugate of the disclosure is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EPO427347), heat shock proteins (WO 93/17712, WO 94/03208), pertussis proteins (WO 98/58668, EPO471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substitution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In one embodiment, the carrier protein of the glycoconjugate of the disclosure is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382. In an embodiment, the carrier protein of the glycoconjugate of the disclosure is the A chain of CRM₁₉₇ (see CN103495161). In an embodiment, the carrier protein of the glycoconjugate of the disclosure is the A chain of CRM₁₉₇ obtained via expression by genetically recombinant E. coli (see CN103495161).

In a particular embodiment, the carrier protein of the glycoconjugate of the disclosure is SCP (Streptococcal C5a Peptidase). Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

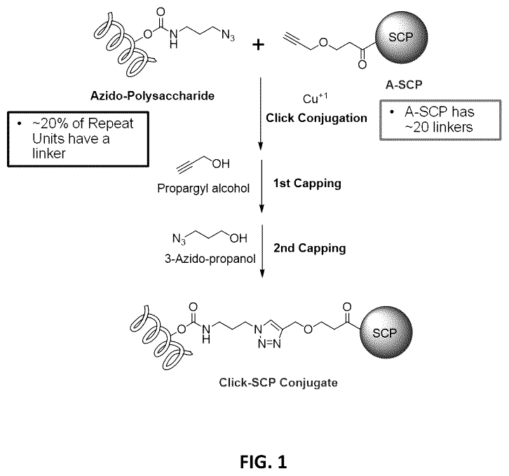

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type III (Fn) domains (Fn1, commonly residues 583/584-712/713; Fn2, commonly residues 712/713-928/929/930; commonly Fn3, residues 929/930-1029/1030/1031) and a cell wall anchor domain (commonly residues 1029/1030/1031 to the C-terminus).

In an embodiment, the carrier protein of the glycoconjugate is an SCP from GBS (SCPB). An example of SCPB is provided at SEQ ID.NO: 3 of WO97/26008. See also SEQ ID NO: 3 of WO00/34487.

In another embodiment, the carrier protein of the glycoconjugate is an SCP from GAS (SCPA). Examples of SCPA can be found at SEQ ID. No. 1 and SEQ ID. No. 2 of WO97/26008. See also SEQ ID NO: 1, 2 and 23 of WO00/34487.

In a particular embodiment, the carrier protein of the glycoconjugate is an enzymatically inactive SCP.

In another particular embodiment, the carrier protein of the glycoconjugate is an enzymatically inactive SCP from GBS (SCPB).

In still another particular embodiment, the carrier protein of the glycoconjugate is an enzymatically inactive SCP from GAS (SCPA).

In another embodiment, the carrier protein of the glycoconjugate is a fragment of an SCP. In an embodiment, the carrier protein of the glycoconjugate is a fragment of an SCPA. In another embodiment, the carrier protein of the glycoconjugate is a fragment of an SCPB.

In an embodiment, the carrier protein of the glycoconjugate is a fragment of an SCP which comprises the protease domain, the protease-associated domain (PA domain) and the three fibronectin type III (Fn) domains but does not comprise the export signal presequence, the pro-sequence, or the cell wall anchor domain.

In an embodiment, the carrier protein of the glycoconjugate is an enzymatically inactive fragment of an SCP which comprises the protease domain, the protease-associated domain (PA domain) and two of the three fibronectin type III (Fn) domains but does not comprise the export signal presequence, the pro-sequence, or the cell wall anchor domain.

In an embodiment, the carrier protein of the glycoconjugate is an enzymatically inactive fragment of an SCP. In an embodiment, said enzymatically inactive fragment of SCP comprises the protease domain, the protease-associated domain (PA domain) and the three fibronectin type III (Fn) domains but does not comprise the export signal presequence, the pro-sequence, or the cell wall anchor domain.

In an embodiment, the carrier protein of the glycoconjugate is an enzymatically inactive fragment of an SCPA. In an embodiment, said enzymatically inactive fragment of an SCPA comprises the protease domain, the protease-associated domain (PA domain) and the three fibronectin type III (Fn) domains but does not comprise the export signal presequence, the pro-sequence, or the cell wall anchor domain.

In another embodiment, the carrier protein of the glycoconjugate is an enzymatically inactive fragment of SCPB. For example, said enzymatically inactive fragment of SCPB can comprise the protease domain, the protease-associated domain (PA domain) and the three fibronectin type III (Fn) domains but not contain the export signal presequence, the pro-sequence, or the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 recited in WO00/34487.

Therefore, in an embodiment, the carrier protein of the glycoconjugate is an enzymatically inactive SCP where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive SCPA where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive SCPB where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive fragment of an SCP where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive fragment of SCP which comprises the protease domain, the protease-associated domain (PA domain) and the three fibronectin type III (Fn) domains but does not comprise the export signal presequence, the pro-sequence, or the cell wall anchor domain, where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive fragment of SCPA which comprises the protease domain, the protease-associated domain (PA domain) and the three fibronectin type III (Fn) domains but does not comprise the export signal presequence, the pro-sequence, or the cell wall anchor domain, where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate is an enzymatically inactive fragment of SCPB which comprises the protease domain, the protease-associated domain (PA domain) and the three fibronectin type III (Fn) domains but does not comprise the export signal presequence, the pro-sequence, or the cell wall anchor domain, where said inactivation is accomplished by replacing at least one amino acid of the wild type sequence. In some embodiments, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the glycoconjugate is an enzymatically inactive SCP where said inactivation is accomplished by replacing at least two amino acids of the wild type sequence. In some embodiments, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In some embodiments, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate is an enzymatically inactive SCPA where said inactivation is accomplished by replacing at least two amino acids of the wild type sequence. In some embodiments, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In some embodiments, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate is an enzymatically inactive SCPB where said inactivation is accomplished by replacing at least two amino acids of the wild type sequence. In some embodiments, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In some embodiments, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate is an enzymatically inactive fragment of an SCP where said inactivation is accomplished by replacing at least two amino acids of the wild type sequence. In some embodiments, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In some embodiments, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the glycoconjugate is an enzymatically inactive SCP where said inactivation is accomplished by replacing at least three amino acids of the wild type sequence. In some embodiments, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate is an enzymatically inactive SCPA where said inactivation is accomplished by replacing at least three amino acids of the wild type sequence. In some embodiments, said replacement of at least three amino acids is in the protease domain. In some embodiments, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate is an enzymatically inactive SCPB where said inactivation is accomplished by replacing at least three amino acids of the wild type sequence. In some embodiments, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate is an enzymatically inactive fragment of an SCP where said inactivation is accomplished by replacing at least three amino acids of the wild type sequence. In some embodiments, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the glycoconjugate is an enzymatically inactive SCP where said inactivation is accomplished by replacing at least four amino acids of the wild type sequence. In some embodiments, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A.

In an embodiment, the carrier protein of the glycoconjugate is an enzymatically inactive SCPA where said inactivation is accomplished by replacing at least four amino acids of the wild type sequence. In some embodiments, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A.

In an embodiment, the carrier protein of the glycoconjugate is an enzymatically inactive SCPB where said inactivation is accomplished by replacing at least four amino acids of the wild type sequence. In some embodiments, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A.

In an embodiment, the carrier protein of the glycoconjugate is an enzymatically inactive fragment of an SCP where said inactivation is accomplished by replacing at least four amino acids of the wild type sequence. In some embodiments, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A.

In another particular embodiment, the carrier protein of the glycoconjugate of the disclosure is an enzymatically inactive fragment of SCP which comprises SEQ ID NO: 113. In another particular embodiment, the carrier protein of the glycoconjugate of the disclosure is an enzymatically inactive fragment of SCP which consists of SEQ ID NO: 113.

In another particular embodiment, the carrier protein of the glycoconjugate of the disclosure is an enzymatically inactive fragment of SCP which comprises SEQ ID NO: 114. In another particular embodiment, the carrier protein of the glycoconjugate of the disclosure is an enzymatically inactive fragment of SCP which consists of SEQ ID NO: 114.

In one embodiment, the carrier protein of the glycoconjugate is an enzymatically inactive fragment of SCP comprising or consisting of a polypeptide having at least 90% identity with SEQ ID NO: 113. In another particular embodiment, the carrier protein of the glycoconjugate is an enzymatically inactive fragment of SCP comprising or consisting of a polypeptide having at least 95% identity with SEQ ID NO: 113. In still another embodiment, the carrier protein of the glycoconjugate is an enzymatically inactive fragment of SCP comprising or consisting of a polypeptide having at least 99% identity with SEQ ID NO: 113. In another embodiment, the carrier protein of the glycoconjugate is an enzymatically inactive fragment of SCP comprising or consisting of a polypeptide having at least 99.5% identity with SEQ ID NO: 113. In yet another embodiment, the carrier protein of the glycoconjugate is an enzymatically inactive fragment of SCP comprising or consisting of a polypeptide having at least 99.8% identity with SEQ ID NO: 113. In an embodiment, the carrier protein of the glycoconjugate is an enzymatically inactive fragment of SCP comprising or consisting of a polypeptide having at least 99.85% identity with SEQ ID NO: 113.

In one embodiment, the carrier protein of the glycoconjugate is an enzymatically inactive fragment of SCP comprising or consisting of a polypeptide having at least 90% identity with SEQ ID NO: 114. In another particular embodiment, the carrier protein of the glycoconjugate is an enzymatically inactive fragment of SCP comprising or consisting of a polypeptide having at least 95% identity with SEQ ID NO: 114. In still another embodiment, the carrier protein of the glycoconjugate is an enzymatically inactive fragment of SCP comprising or consisting of a polypeptide having at least 99% identity with SEQ ID NO: 114. In another embodiment, the carrier protein of the glycoconjugate is an enzymatically inactive fragment of SCP comprising or consisting of a polypeptide having at least 99.5% identity with SEQ ID NO: 114. In yet another embodiment, the carrier protein of the glycoconjugate is an enzymatically inactive fragment of SCP comprising or consisting of a polypeptide having at least 99.8% identity with SEQ ID NO: 114. In an embodiment, the carrier protein of the glycoconjugate is an enzymatically inactive fragment of SCP comprising or consisting of a polypeptide having at least 99.85% identity with SEQ ID NO: 114.

### Exemplary SCP Sequences are Shown Below:

SEQ ID NO: 113 sets forth an enzymatically inactive fragment of SCP that contains 950 amino acids:

MAKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ

SEQ ID NO: 114 sets forth an enzymatically inactive fragment of SCP that contains 949 amino acids:

AKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ

Saccharide Glycoconjugates Prepared using Click Chemistry

In particular embodiments, the glycoconjugates of the present disclosure are prepared using click chemistry. The present disclosure also relates to a method of making a glycoconjugate, as disclosed herein.

In some embodiments, the click chemistry reaction can comprise three steps,
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.

Following step (a) the saccharide is said to be activated and is referred to herein as “activated saccharide” or “activated saccharide with an azido linker”.

Following step (b) the carrier is said to be activated and is referred to as “activated carrier” or “activated SCP”.

In an embodiment, said carbonic acid derivative is selected from the group consisting of 1,1′-carbonyldiimidazole (CDI), 1,1′-carbonyl-di-(1,2,4-triazole) (CDT), disuccinimidyl carbonate (DSC), and N-hydroxysuccinimidyl chloroformate. In a particular embodiment, the carbonic acid derivative is CDI. In another particular embodiment, the carbonic acid derivative is CDI.

In some embodiments, said agent comprising an azide comprises the structure of Formula I below:

H₂N—X—N₃ (Formula I),

where X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ, and O(CH₂CH₂O)ₘCH₂CH₂, wherein n ranges from 1 to 10, and m ranges from 1 to 4. In some embodiments, said agent comprising an azide comprises the structure of Formula I and n is selected from the group consisting of 1, 2, 3, 4, 5, 6, 7, 8, 9, and 10. In some embodiments, said agent comprising an azide comprises the structure of Formula I and m is selected from the group consisting of 1, 2, 3, and 4.

In a particular embodiment, said agent comprising an azide is 3-azido-1-propylamine.

In an embodiment, said agent comprising an N-Hydroxysuccinimide (NHS) ester comprises the structure of Formula II below:

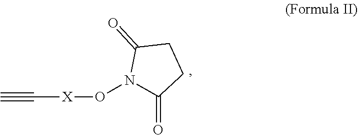

where X is selected from the group consisting of CH₂O(CH₂)ₙCH₂C═O and CH₂O(CH₂CH₂O)ₘ(CH₂)ₙCH₂C═O, and wherein n ranges from 0 to 10, and m ranges from 0 to 4. In some embodiments, said agent comprising an NHS ester comprises the structure of Formula II and n is selected from the group consisting of 1, 2, 3, 4, 5, 6, 7, 8, 9, and 10. In some embodiments, said agent comprising an NHS ester comprises the structure of Formula II and m is selected from the group consisting of 1, 2, 3, and 4.

In a particular embodiment, said agent comprising an N-Hydroxysuccinimide (NHS) ester is 3-propargyloxy-propanoic acid NHS ester.

In some embodiments, the cycloaddition reaction of step (3) is mediated by a metal. In some embodiments, the cycloaddition reaction of step (3) is mediated by Cooper. For example, in particular embodiments, the cycloaddition reaction of step (3) is mediated by Cu+1.

In an embodiment, at step a) the isolated saccharide is reacted with a carbonic acid derivative in an aprotic solvent.

In one embodiment the isolated saccharide is reacted with a carbonic acid derivative in a solution consisting essentially of dimethylsulphoxide (DMSO) or dimethylformamide (DMF). In an embodiment, the isolated saccharide is reacted with a carbonic acid derivative in a solution consisting essentially of N-methyl-2-pyrrolidone. In an embodiment, the isolated saccharide is reacted with a carbonic acid derivative in a solution consisting essentially of hexamethylphosphoramide (HMPA).

In a particular embodiment, the isolated saccharide is reacted with CDI or CDT in dimethylsulphoxide (DMSO). In an embodiment the isolated saccharide is reacted with CDI or CDT in anhydrous DMSO.

In some embodiments, reacting the isolated saccharide with CDI in an environment with a moisture level of about 0.1% to 1% (v/v) allows the avoidance of side reactions. Therefore, in one embodiment the isolated saccharide is reacted with CDI in an aprotic solvent (e.g., DMSO or DMF) comprising 0.1% to 1% (v/v) water. For example, in some embodiments, the isolated saccharide is reacted with CDI in an aprotic solvent comprising about 0.1%, about 0.2%, about 0.3%, about 0.4%, about 0.5%, about 0.6%, about 0.7%, about 0.8%, about 0.9%, or about 1% (v/v) water.

In one embodiment the free carbonic acid derivative is then quenched by the addition of water before the addition of the agent comprising an azide. Water can inactivate free CDI. Therefore, in an embodiment, carbonic acid derivative activation is followed by the addition of water. In an embodiment, water is added to bring the total water content in the mixture to between about 1% to about 10% (v/v). In an embodiment, water is added to bring the total water content in the mixture to between about 0.5% to about 5% (v/v). In some embodiments, water is added to bring the total water content in the mixture to about 1%, about 2%, about 3%, about 4%, or about 5%.

Once the saccharide has been reacted with carbonic acid derivative and following an eventual quenching of carbonic acid derivative with water, the carbonic acid derivative-activated saccharide is reacted with the agent comprising an azide.

In one embodiment the degree of activation (DoA) of the activated saccharide following step a) is between about 0.5 and 50%. The degree of activation of the saccharide being defined as the percentage of Repeating Unit linked to an azido linker. In some embodiments, the DoA of the activated saccharide is between about 1% and about 30%. For example, in some embodiments, the DoA of the activated saccharide is between about 5% and about 25%. In particular embodiments, the DoA of the activated saccharide is about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%.

In one embodiment the degree of activation (DoA) of the activated carrier following step b) is between about 1% and about 50%. The DoA of the activated carrier being defined as the number of lysine residues in the carrier protein that become linked to the agent comprising an N-Hydroxysuccinimide (NHS) ester.

In a particular embodiment, the carrier protein is SCP or a functional fragment thereof. In some embodiments the DoA of the activated SCP following step b) may be between about 1% and about 50%. In some embodiments, the DoA of the activated SCP following step b) is between about 10% and about 30%. For example, in some embodiments, the DoA of the activated SCP is about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%. In a particular embodiment, the DoA of the activated SCP following step b) is about 20%.

In an embodiment, the carrier protein is CRM₁₉₇, which contains 39 lysine residues. In said embodiment the DoA of the activated carrier following step b) may be between about 1% to about 50%. In some embodiments, the DoA of the activated CRM₁₉₇ is between about 1% and about 30%. For example, in some embodiments, the DoA of the CRM₁₉₇ carrier is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In another embodiment, the carrier protein is TT. In said embodiment the DoA of the activated carrier following step b) may be between about 1% to about 50%. In some embodiments, the DoA of the activated TT is between about 1% and about 30%. For example, in some embodiments, the DoA of the TT carrier is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In an embodiment, the conjugation reaction c) is carried out in aqueous buffer. In an embodiment, the conjugation reaction c) is carried out in aqueous buffer in the presence of copper as catalyst. In an embodiment, the conjugation reaction c) is carried out in aqueous buffer in the presence an oxidant and of copper as catalyst. In a particular embodiment, the conjugation reaction c) is carried out in aqueous buffer in the presence of copper as catalyst and ascorbate as oxidant. In an embodiment, THPTA (tris(3-hydroxypropyltriazolylmethyl)amine) and aminoguanidine may be further added to protect the protein from side reactions. Therefore, in a particular embodiment, the conjugation reaction c) is carried out in aqueous buffer in the presence of copper as catalyst and ascorbate as oxidant, wherein the reaction mixture further comprises THPTA (tris(3-hydroxypropyltriazolylmethyl)amine) and aminoguanidine.

Following the click conjugation reaction, there may remain unreacted azido groups in the conjugates, these may be capped using a suitable azido group capping agent. Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (V),

≡—X—OH (V)

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (VI),

N₃—X—OH (VI)

wherein X is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present disclosure comprises the step of purifying the glycoconjugate after it is produced.

In an aspect, the present disclosure provides a glycoconjugate produced according to any of the methods disclosed herein.

### Dosages of the Compositions

Dosage regimens may be adjusted to provide the optimum desired response. For example, a single dose of the glycoconjugate may be administered, several divided doses may be administered over time, or the dose may be proportionally reduced or increased as indicated by the exigencies of the situation. It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Determining appropriate dosages and regimens for administration of the therapeutic protein are well-known in the relevant art and would be understood to be encompassed by the skilled artisan once provided the teachings disclosed herein.

The amount of glycoconjugate(s) in each dose is selected as an amount which induces an immunoprotective response without significant, adverse side effects in typical vaccines. Such amount will vary depending upon which specific immunogen is employed and how it is presented.

The amount of a particular glycoconjugate in an immunogenic composition can be calculated based on total polysaccharide for that conjugate (conjugated and non-conjugated). For example, a glycoconjugate with 20% free polysaccharide will have about 80 μg of conjugated polysaccharide and about 20 μg of non-conjugated polysaccharide in a 100 μg polysaccharide dose. The amount of glycoconjugate can vary depending upon the E. coli serotype. The saccharide concentration can be determined by the uronic acid assay.

The “immunogenic amount” of the different polysaccharide components in the immunogenic composition, may diverge and each may comprise about 1.0 μg, about 2.0 μg, about 3.0 μg, about 4.0 μg, about 5.0 μg, about 6.0 μg, about 7.0 μg, about 8.0 μg, about 9.0 μg, about 10.0 μg, about 15.0 μg, about 20.0 μg, about 30.0 μg, about 40.0 μg, about 50.0 μg, about 60.0 μg, about 70.0 μg, about 80.0 μg, about 90.0 μg, or about 100.0 μg of any particular polysaccharide antigen. Generally, each dose will comprise between about 0.1 μg and about 100 μg of polysaccharide for a given serotype, particularly 0.1 μg to 10 μg, more particularly 0.1 μg to 5 μg, and even more particularly 0.2 μg to 2 μg. For example, in some embodiments, each dose will comprise about 0.2 μg, about 0.3 μg, about 0.4 μg, about 0.5 μg, about 0.6 μg, about 0.7 μg, about 0.8 μg, about 0.9 μg, about 1.0 μg, about 1.1 μg, about 1.2 μg, about 1.3 μg, about 1.4 μg, about 1.5 μg, about 1.6 μg, about 1.7 μg, about 1.8 μg, about 1.9 μg, or about 2.0 μg polysaccharide for a given serotype. In a particular embodiment, each dose will comprise about 0.2 μg of polysaccharide for a given serotype. In another embodiment, each dose will comprise about 2 μg of polysaccharide for a given serotype.

In some embodiments, each dose comprises between about 0.1 μg and about 50 μg of E. coli polysaccharide O25b. In some embodiments, each dose comprises between about 0.1 μg and about 10 μg of E. coli polysaccharide O25b. In some embodiments, each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b. In a preferred embodiment, each dose comprises about 4 μg of E. coli polysaccharide O25b. In another preferred embodiment, each dose comprises about 8 μg of E. coli polysaccharide O25b.

In some embodiments, each dose comprises between about 0.1 μg and about 50 μg of E. coli polysaccharide O1a. In some embodiments, each dose comprises between about 0.1 μg and about 10 μg of E. coli polysaccharide O1a. In some embodiments, each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O1a. In one embodiment, each dose comprises about 2 μg of E. coli polysaccharide O1a. In another embodiment, each dose comprises about 4 μg of E. coli polysaccharide O1a.

In some embodiments, each dose comprises between about 0.1 μg and about 50 μg of E. coli polysaccharide O2. In some embodiments, each dose comprises between about 0.1 μg and about 10 μg of E. coli polysaccharide O2. In some embodiments, each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O2. In one embodiment, each dose comprises about 2 μg of E. coli polysaccharide O2. In another embodiment, each dose comprises about 4 μg of E. coli polysaccharide O2.

In some embodiments, each dose comprises between about 0.1 μg and about 50 μg of E. coli polysaccharide O6. In some embodiments, each dose comprises between about 0.1 μg and about 10 μg of E. coli polysaccharide O6. In some embodiments, each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O6. In one embodiment, each dose comprises about 2 μg of E. coli polysaccharide O6. In another embodiment, each dose comprises about 4 μg of E. coli polysaccharide O6.

Generally, each dose will comprise 5 μg to 150 μg of carrier protein, particularly 10 μg to 100 μg of carrier protein, more particularly 15 μg to 100 μg of carrier protein, more particularly 25 μg to 75 μg of carrier protein, more particularly 30 μg to 70 μg of carrier protein, more particularly 30 μg to 60 μg of carrier protein, more particularly 30 μg to 50 μg of carrier protein and even more particularly 40 μg to 60 μg of carrier protein. In one embodiment, said carrier protein is SCP. In one embodiment, each dose will comprise about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, 68 μg, about 69 μg, about 70 μg, about 71 μg, about 72 μg, about 73 μg, about 74 μg or about 75 μg of carrier protein. In one embodiment, said carrier protein is SCP. In another embodiment, said carrier protein is CRM₁₉₇.

In some embodiments wherein the glycoconjugate is administered with a polypeptide derived from E. coli, the amount of the polypeptide derived from E. coli or fragment thereof in the composition, may range from about 10 μg to about 300 μg of each protein antigen. In some embodiments, the amount of the polypeptide derived from E. coli or fragment thereof in the composition may range from about 20 μg to about 200 μg of each protein antigen.

In some embodiments, the glycoconjugate is administered to a subject with a RNA molecule encoding a polypeptide derived from E. coli. In some embodiments, the glycoconjugate is administered to a subject with a RNA molecule encoding a polypeptide derived from E. coli FimH (FimH RNA). In some embodiments, the glycoconjugate is administered to a subject with FimH RNA, wherein each dose contains between about 1 μg and about 1,000 μg of FimH RNA. In some embodiments, the glycoconjugate is administered to a subject with FimH RNA, wherein each dose contains between about 1 μg and about 100 μg of FimH RNA. In some embodiments, the glycoconjugate is administered to a subject with FimH RNA, wherein each dose contains about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg of FimH RNA. In a preferred embodiment, the glycoconjugate is administered to a subject with FimH RNA, wherein each dose contains about 30 μg of FimH RNA. In another preferred embodiment, the glycoconjugate is administered to a subject with FimH RNA, wherein each dose contains about 60 μg of FimH RNA. In another preferred embodiment, the glycoconjugate is administered to a subject with FimH RNA, wherein each dose contains about 90 μg of FimH RNA.

In some embodiments, the glycoconjugate is administered to a subject with a RNA molecule encoding a polypeptide derived from E. coli FimH (FimH RNA), wherein the glycoconjugate comprises E. coli polysaccharide O25b (O25b glycoconjugate). In some embodiments, a O25b glycoconjugate is administered to a subject with FimH RNA, wherein each dose comprises between about 0.1 μg and about 50 μg of E. coli polysaccharide O25b and between about 1 μg and about 1,000 μg of FimH RNA. In some embodiments, a O25b glycoconjugate is administered to a subject with FimH RNA, wherein each dose comprises between about 0.1 μg and about 10 μg of E. coli polysaccharide O25b and between about 1 μg and about 100 μg of FimH RNA. In some embodiments, a O25b glycoconjugate is administered to a subject with FimH RNA, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg of FimH RNA. In a preferred embodiment, a O25b glycoconjugate is administered to a subject with FimH RNA, wherein each dose comprises about 4 μg of E. coli polysaccharide O25b and about 30 μg of FimH RNA. In another preferred embodiment, a O25b glycoconjugate is administered to a subject with FimH RNA, wherein each dose comprises about 8 μg of E. coli polysaccharide O25b and about 90 μg of FimH RNA.

In some embodiments, the glycoconjugate is administered to a subject with an adjuvant. In some embodiments, the glycoconjugate is administered to a subject with a liposomal adjuvant. In some embodiments, the glycoconjugate is administered to a subject with LiNA-2. In some embodiments, the glycoconjugate is administered to a subject with a liposomal adjuvant, wherein each dose contains between about 0.01 mg and about 10 mg of a saponin. In some embodiments, the glycoconjugate is administered to a subject with a liposomal adjuvant, wherein each dose contains between about 0.01 mg and about 10 mg of QS-21. In some embodiments, the glycoconjugate is administered to a subject with a liposomal adjuvant, wherein each dose contains between about 0.01 mg and about 1 mg of a saponin. In some embodiments, the glycoconjugate is administered to a subject with a liposomal adjuvant, wherein each dose contains between about 0.01 mg and about 1 mg of QS-21. In some embodiments, the glycoconjugate is administered to a subject with a liposomal adjuvant, wherein each dose contains between about 0.05 mg and about 0.15 mg of a saponin. In some embodiments, the glycoconjugate is administered to a subject with a liposomal adjuvant, wherein each dose contains between about 0.05 mg and about 0.15 mg of QS-21. In some embodiments, the glycoconjugate is administered to a subject with a liposomal adjuvant, wherein each dose contains about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, or about 0.15 mg of QS-21. In a preferred embodiment, the glycoconjugate is administered to a subject with a liposomal adjuvant, wherein each dose contains about 0.1 mg of QS-21.

In some embodiments, the glycoconjugate is administered to a subject with a liposomal adjuvant comprising QS-21, wherein the glycoconjugate comprises E. coli polysaccharide O25b (O25b glycoconjugate). In some embodiments, a O25b glycoconjugate is administered to a subject with a liposomal adjuvant comprising QS-21, wherein each dose comprises between about 0.1 μg and about 50 μg of E. coli polysaccharide O25b and between about 0.01 mg and about 10 mg of QS-21. In some embodiments, a O25b glycoconjugate is administered to a subject with a liposomal adjuvant comprising QS-21, wherein each dose comprises between about 0.1 μg and about 10 μg of E. coli polysaccharide O25b and between about 0.01 mg and about 1 mg of QS-21. In some embodiments, a O25b glycoconjugate is administered to a subject with a liposomal adjuvant comprising QS-21, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, or about 0.15 mg of QS-21. In a preferred embodiment, a O25b glycoconjugate is administered to a subject with a liposomal adjuvant comprising QS-21, wherein each dose comprises about 4 μg of E. coli polysaccharide O25b and about 0.1 mg of QS-21.

In some embodiments, the glycoconjugate is administered to a subject with a liposomal adjuvant, wherein each dose contains between about 1 mg and about 50 mg of 1,2-dimyristoyl-sn-glycero-3-phosphocholine (DMPC). In some embodiments, the glycoconjugate is administered to a subject with a liposomal adjuvant, wherein each dose contains between about 3.5 mg and about 10.5 mg of DMPC. In some embodiments, the glycoconjugate is administered to a subject with a liposomal adjuvant, wherein each dose contains about 5 mg, about 5.5 mg, about 6 mg, about 6.5 mg, about 7 mg, about 7.5 mg, about 8 mg, about 8.5 mg, about 9 mg, about 9.5 mg, or about 10 mg of DMPC. In a preferred embodiment, the glycoconjugate is administered to a subject with a liposomal adjuvant, wherein each dose contains about 7 mg of DMPC.

In some embodiments, the glycoconjugate is administered to a subject with a liposomal adjuvant comprising 1,2-dimyristoyl-sn-glycero-3-phosphocholine (DMPC), wherein the glycoconjugate comprises E. coli polysaccharide O25b (O25b glycoconjugate). In some embodiments, a O25b glycoconjugate is administered to a subject with a liposomal adjuvant comprising DMPC, wherein each dose comprises between about 0.1 μg and about 50 μg of E. coli polysaccharide O25b and between about 1 mg and about 50 mg of DMPC. In some embodiments, a O25b glycoconjugate is administered to a subject with a liposomal adjuvant comprising DMPC, wherein each dose comprises between about 0.1 μg and about 10 μg of E. coli polysaccharide O25b and between about 3.5 mg and about 10.5 mg of DMPC. In some embodiments, a O25b glycoconjugate is administered to a subject with a liposomal adjuvant comprising DMPC, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 5 mg, about 5.5 mg, about 6 mg, about 6.5 mg, about 7 mg, about 7.5 mg, about 8 mg, about 8.5 mg, about 9 mg, about 9.5 mg, or about 10 mg of DMPC. In a preferred embodiment, a O25b glycoconjugate is administered to a subject with a liposomal adjuvant comprising DMPC, wherein each dose comprises about 4 μg of E. coli polysaccharide O25b and about 7 mg of DMPC. In a further preferred embodiment, a O25b glycoconjugate is administered to a subject with a liposomal adjuvant comprising DMPC and QS-21, wherein each dose comprises about 4 μg of E. coli polysaccharide O25b, about 7 mg of DMPC, and about 0.1 mg of QS-21.

In some embodiments, the glycoconjugate is administered to a subject with a liposomal adjuvant, wherein each dose contains between about 0.1 mg and about 10 mg of 1,2-dimyristoyl-sn-glycero-3-phospho-(1′-rac-glycerol) (DMPG). In some embodiments, the glycoconjugate is administered to a subject with a liposomal adjuvant, wherein each dose contains between about 0.4 mg and about 1.2 mg of DMPG. In some embodiments, the glycoconjugate is administered to a subject with a liposomal adjuvant, wherein each dose contains about 0.5 mg, about 0.6 mg, about 0.7 mg, about 0.8 mg, about 0.9 mg, or about 1 mg of DMPG. In a preferred embodiment, the glycoconjugate is administered to a subject with a liposomal adjuvant, wherein each dose contains about 0.8 mg DMPG.

In some embodiments, the glycoconjugate is administered to a subject with a liposomal adjuvant comprising 1,2-dimyristoyl-sn-glycero-3-phospho-(1′-rac-glycerol) (DMPG), wherein the glycoconjugate comprises E. coli polysaccharide O25b (O25b glycoconjugate). In some embodiments, a O25b glycoconjugate is administered to a subject with a liposomal adjuvant comprising DMPG, wherein each dose comprises between about 0.1 μg and about 50 μg of E. coli polysaccharide O25b and between about 0.1 mg and about 10 mg of DMPG. In some embodiments, a O25b glycoconjugate is administered to a subject with a liposomal adjuvant comprising DMPG, wherein each dose comprises between about 0.1 μg and about 10 μg of E. coli polysaccharide O25b and between about 0.4 mg and about 1.2 mg of DMPG. In some embodiments, a O25b glycoconjugate is administered to a subject with a liposomal adjuvant comprising DMPG, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 0.5 mg, about 0.6 mg, about 0.7 mg, about 0.8 mg, about 0.9 mg, or about 1 mg of DMPG. In a preferred embodiment, a O25b glycoconjugate is administered to a subject with a liposomal adjuvant comprising DMPG, wherein each dose comprises about 4 μg of E. coli polysaccharide O25b and about 0.8 mg of DMPG. In a further preferred embodiment, a O25b glycoconjugate is administered to a subject with a liposomal adjuvant comprising DMPC, DMPG, and QS-21, wherein each dose comprises about 4 μg of E. coli polysaccharide O25b, about 7 mg of DMPC, about 0.8 mg of DMPG, and about 0.1 mg of QS-21.

In some embodiments, the glycoconjugate is administered to a subject with a liposomal adjuvant, wherein each dose contains between about 0.5 mg and about 50 mg of cholesterol. In some embodiments, the glycoconjugate is administered to a subject with a liposomal adjuvant, wherein each dose contains between about 2.5 and about 8.5 mg of cholesterol. In some embodiments, the glycoconjugate is administered to a subject with a liposomal adjuvant, wherein each dose contains about 5 mg, about 5.1 mg, about 5.2 mg, about 5.3 mg, about 5.4, about 5.5, about 5.6, about 5.7 mg, about 5.8 mg, about 5.9 mg, or about 6 mg of cholesterol. In a preferred embodiment, the glycoconjugate is administered to a subject with a liposomal adjuvant, wherein each dose contains about 5.5 mg of cholesterol.

In some embodiments, the glycoconjugate is administered to a subject with a liposomal adjuvant comprising cholesterol, wherein the glycoconjugate comprises E. coli polysaccharide O25b (O25b glycoconjugate). In some embodiments, a O25b glycoconjugate is administered to a subject with a liposomal adjuvant comprising cholesterol, wherein each dose comprises between about 0.1 μg and about 50 μg of E. coli polysaccharide O25b and between about 0.5 mg and about 50 mg of cholesterol. In some embodiments, a O25b glycoconjugate is administered to a subject with a liposomal adjuvant comprising cholesterol, wherein each dose comprises between about 0.1 μg and about 10 μg of E. coli polysaccharide O25b and between about 2.5 and about 8.5 mg of cholesterol. In some embodiments, a O25b glycoconjugate is administered to a subject with a liposomal adjuvant comprising cholesterol, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 5 mg, about 5.1 mg, about 5.2 mg, about 5.3 mg, about 5.4, about 5.5, about 5.6, about 5.7 mg, about 5.8 mg, about 5.9 mg, or about 6 mg of cholesterol. In a preferred embodiment, a O25b glycoconjugate is administered to a subject with a liposomal adjuvant comprising cholesterol, wherein each dose comprises about 4 μg of E. coli polysaccharide O25b and about 5.5 mg of cholesterol. In a further preferred embodiment, a O25b glycoconjugate is administered to a subject with a liposomal adjuvant comprising DMPC, DMPG, cholesterol, and QS-21, wherein each dose comprises about 4 μg of E. coli polysaccharide O25b, about 7 mg of DMPC, about 0.8 mg of DMPG, about 5.5 mg of cholesterol, and about 0.1 mg of QS-21.

In some embodiments, the glycoconjugate is administered to a subject with a liposomal adjuvant, wherein each dose contains between about 0.01 mg and about 10 mg of monophosphoryl 3-deacyl lipid A phosphorylated hexaacyl disaccharide (3D-PHAD®). In some embodiments, the glycoconjugate is administered to a subject with a liposomal adjuvant, wherein each dose contains between about 0.1 mg and about 0.3 mg of 3D-PHAD®. In some embodiments, the glycoconjugate is administered to a subject with a liposomal adjuvant, wherein each dose contains about 0.15 mg, about 0.16 mg, about 0.17 mg, about 0.18 mg, about 0.19 mg, about 0.2 mg, about 0.21 mg, about 0.22 mg, about 0.23 mg, about 0.24 mg, or about 0.25 mg of 3D-PHAD®. In a preferred embodiment, the glycoconjugate is administered to a subject with a liposomal adjuvant, wherein each dose contains about 0.2 mg of 3D-PHAD®.

In some embodiments, the glycoconjugate is administered to a subject with a liposomal adjuvant comprising 3D-PHAD®, wherein the glycoconjugate comprises E. coli polysaccharide O25b (O25b glycoconjugate). In some embodiments, a O25b glycoconjugate is administered to a subject with a liposomal adjuvant comprising 3D-PHAD®, wherein each dose comprises between about 0.1 μg and about 50 μg of E. coli polysaccharide O25b and between about 0.01 mg and about 10 mg of 3D-PHAD®. In some embodiments, a O25b glycoconjugate is administered to a subject with a liposomal adjuvant comprising 3D-PHAD©, wherein each dose comprises between about 0.1 μg and about 10 μg of E. coli polysaccharide O25b and between about 0.1 mg and about 0.3 mg of 3D-PHAD®. In some embodiments, a O25b glycoconjugate is administered to a subject with a liposomal adjuvant comprising 3D-PHAD®, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 0.15 mg, about 0.16 mg, about 0.17 mg, about 0.18 mg, about 0.19 mg, about 0.2 mg, about 0.21 mg, about 0.22 mg, about 0.23 mg, about 0.24 mg, or about 0.25 mg of 3D-PHAD®. In a preferred embodiment, a O25b glycoconjugate is administered to a subject with a liposomal adjuvant comprising 3D-PHAD®, wherein each dose comprises about 4 μg of E. coli polysaccharide O25b and about 0.2 mg of 3D-PHAD®. In a further preferred embodiment, a O25b glycoconjugate is administered to a subject with a liposomal adjuvant comprising DMPC, DMPG, cholesterol, 3D-PHAD®, and QS-21, wherein each dose comprises about 4 μg of E. coli polysaccharide O25b, about 7 mg of DMPC, about 0.8 mg of DMPG, about 5.5 mg of cholesterol, about 0.2 mg of 3D-PHAD®, and about 0.1 mg of QS-21.

In some embodiments, the glycoconjugate is administered to a subject with a nucleotide adjuvant. In some embodiments, the glycoconjugate is administered to a subject with a CpG oligodeoxynucleotide (CpG ODN). In some embodiments, the glycoconjugate is administered to a subject with a CpG ODN, wherein each dose contains between about 0.1 mg and about 100 mg of CpG ODN. In some embodiments, the glycoconjugate is administered to a subject with a CpG ODN, wherein each dose contains between about 1 mg and about 10 mg of CpG ODN. In some embodiments, the glycoconjugate is administered to a subject with a CpG ODN, wherein each dose contains between about 1.5 mg and about 2.5 mg of CpG ODN. In some embodiments, the glycoconjugate is administered to a subject with a CpG ODN, wherein each dose contains about 1.5 mg, about 1.6 mg, about 1.7 mg, about 1.8 mg, about 1.9 mg, or about 2 mg of CpG ODN. In a preferred embodiment, the glycoconjugate is administered to a subject with a CpG ODN, wherein each dose contains about 1.8 mg of CpG ODN.

In some embodiments, the glycoconjugate is administered to a subject with a CpG ODN, wherein the glycoconjugate comprises E. coli polysaccharide O25b (O25b glycoconjugate). In some embodiments, a O25b glycoconjugate is administered to a subject with a CpG ODN, wherein each dose comprises between about 0.1 μg and about 50 μg of E. coli polysaccharide O25b and between about 0.1 mg and about 100 mg of CpG ODN. In some embodiments, a O25b glycoconjugate is administered to a subject with a CpG ODN, wherein each dose comprises between about 0.1 μg and about 10 μg of E. coli polysaccharide O25b and between about 1 mg and about 10 mg of a CpG ODN. In some embodiments, a O25b glycoconjugate is administered to a subject with a CpG ODN, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 1.5 mg, about 1.6 mg, about 1.7 mg, about 1.8 mg, about 1.9 mg, or about 2 mg CpG ODN. In a preferred embodiment, a O25b glycoconjugate is administered to a subject with a CpG ODN, wherein each dose comprises about 4 μg of E. coli polysaccharide O25b and about 1.8 mg of CpG ODN.

In some embodiments, the glycoconjugate is administered to a subject with CpG 24555. In some embodiments, the glycoconjugate is administered to a subject with CpG 24555, wherein each dose contains between about 0.1 mg and about 100 mg of CpG 24555. In some embodiments, the glycoconjugate is administered to a subject with CpG 24555, wherein each dose contains between about 1 mg and about 10 mg of CpG 24555. In some embodiments, the glycoconjugate is administered to a subject with CpG 24555, wherein each dose contains between about 1.5 mg and about 2.5 mg of CpG 24555. In some embodiments, the glycoconjugate is administered to a subject with CpG 24555, wherein each dose contains about 1.5 mg, about 1.6 mg, about 1.7 mg, about 1.8 mg, about 1.9 mg, or about 2 mg of CpG 24555. In a preferred embodiment, the glycoconjugate is administered to a subject with CpG 24555, wherein each dose contains about 1.8 mg of CpG 24555.

In some embodiments, the glycoconjugate is administered to a subject with CpG 24555, wherein the glycoconjugate comprises E. coli polysaccharide O25b (O25b glycoconjugate). In some embodiments, a O25b glycoconjugate is administered to a subject with CpG 24555, wherein each dose comprises between about 0.1 μg and about 50 μg of E. coli polysaccharide O25b and between about 0.1 mg and about 100 mg of CpG 24555. In some embodiments, a O25b glycoconjugate is administered to a subject with CpG 24555, wherein each dose comprises between about 0.1 μg and about 10 μg of E. coli polysaccharide O25b and between about 1 mg and about 10 mg of CpG 24555. In some embodiments, a O25b glycoconjugate is administered to a subject with CpG 24555, wherein each dose comprises about 0.5 μg, about 1 μg, about 1.5 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg of E. coli polysaccharide O25b and about 1.5 mg, about 1.6 mg, about 1.7 mg, about 1.8 mg, about 1.9 mg, or about 2 mg of CpG 24555. In a preferred embodiment, a O25b glycoconjugate is administered to a subject with CpG 24555, wherein each dose comprises about 4 μg of E. coli polysaccharide O25b and about 1.8 mg of CpG 24555.

### Adjuvants

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein mayfurther comprise two adjuvants. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In one embodiment, the immunogenic compositions disclosed herein comprise aluminum phosphate or aluminum hydroxide as adjuvant. In an embodiment, the immunogenic compositions disclosed herein comprise from 0.1 mg/mL to 1 mg/mL or from 0.2 mg/mL to 0.3 mg/mL of elemental aluminum in the form of aluminum phosphate. In an embodiment, the immunogenic compositions disclosed herein comprise about 0.25 mg/mL of elemental aluminum in the form of aluminum phosphate.

Examples of known suitable immune modulatory type adjuvants that can be used in humans include, but are not limited to, saponin extracts from the bark of the Aquilla tree (QS21, Quil A), TLR4 agonists such as MPLA (Monophosphoryl Lipid A), 3DMPL (3-O-deacylated MPL) or GLA-AQ, LT/CT mutants, cytokines such as the various interleukins (e.g., IL-2, IL-12) or GM-CSF, AS01, and the like.

Examples of known suitable immune modulatory type adjuvants with both delivery and immune modulatory features that can be used in humans include, but are not limited to, ISCOMS (see, e.g., Sjölander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

For veterinary applications including but not limited to animal experimentation, one can use Complete Freund's Adjuvant (CFA), Freund's Incomplete Adjuvant (IFA), Emulsigen, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

Further exemplary adjuvants to enhance effectiveness of the immunogenic compositions disclosed herein include, but are not limited to (1) oil-in-water emulsion formulations (with or without other specific immunostimulating agents such as muramyl peptides (see below) or bacterial cell wall components), such as for example (a) SAF, containing 10% Squalane, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), for example MPL+CWS (DETOX™); (2) saponin adjuvants, such as QS21, STIMULON™ (Cambridge Bioscience, Worcester, Mass.), ABISCO® (Isconova, Sweden), or ISCOMATRIX® (Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB2220211, EP0689454) (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+1M2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

### Nucleotide Adjuvants

In some aspects, the immunogenic compositions as disclosed herein comprise nucleotides. In some embodiments, the immunogenic compositions as disclosed herein comprise DNA. In some embodiments, the immunogenic compositions as disclosed herein comprise DNA that is single-stranded. In some embodiments, the immunogenic compositions as disclosed herein comprise DNA that is double-stranded.

In an embodiment of the present disclosure, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068.

In an embodiment of the present disclosure, the immunogenic compositions as disclosed herein comprise any of the CpG Oligonucleotide described at page 3, line 22, to page 12, line 36, of WO 2010/125480.

CpG oligonucleotides may encompass various chemical modifications and substitutions, in comparison to natural RNA and DNA, involving a phosphodiester internucleoside bridge, a beta-D-ribose (deoxyhbose) unit and/or a natural nucleoside base (adenine, guanine, cytosine, thymine, uracil). Examples of chemical modifications are known to the skilled person and are described, for example in Uhlmann E. et al. (1990), Chem. Rev. 90:543; “Protocols for Oligonucleotides and Analogs”, Synthesis and Properties and Synthesis and Analytical Techniques, S. Agrawal, Ed., Humana Press, Totowa, USA 1993; Crooke, S T. et al. (1996) Annu. Rev. Pharmacol. Toxicol. 36:107-129; and Hunziker J. et al., (1995), Mod. Synth. Methods 7:331-417. Specifically, a CpG oligonucleotide can contain a modified cytosine. A modified cytosine is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluorocytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). For example, cytosines can include 5-methyl-cytosine, 5-fluoro-cytosine, 5-hydroxy-cytosine, 5-hydroxymethyl-cytosine, and N4-ethyl-cytosine.

A CpG oligonucleotide can also contain a modified guanine. A modified guanine is a naturally occurring or non-naturally occurring purine base analog of guanine which can replace this base without impairing the immunostimulatory activity of the oligonucleotide. Modified guanines include but are not limited to 7-deeazaguanine, 7-deaza-7-substituted guanine, hypoxanthine, N2-substituted guanines (e.g. N2-methyl-guanine), 5-amino-3-methyl-3H,6H-thiazolo[4,5-d]pyhmidine-2,7-dione, 2,6-diaminopuhne, 2-aminopuhne, purine, indole, adenine, substituted adenines (e.g. N6-methyl-adenine, 8-oxo-adenine), 8-substituted guanine (e.g. 8-hydroxyguanine and 8-bromoguanine), and 6-thioguanine. In some aspects of the disclosure, the guanine base is substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom.

In certain aspects, the CpG oligonucleotides include modified backbones. It has been demonstrated that modification of the nucleic acid backbone provides enhanced activity of nucleic acids when administered in vivo. Secondary structures, such as stem loops, can stabilize nucleic acids against degradation. Alternatively, nucleic acid stabilization can be accomplished via phosphate backbone modifications. In some embodiments, a stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made, e.g. as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A. (1990) Chem. Rev. 90:544; Goodchild, J. (1990) Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C. Constructs having phosphorothioate linkages provide maximal activity and protect the nucleic acid from degradation by intracellular exo- and endo-nucleases.

In an embodiment, all the internucleotide linkage of the CpG oligonucleotides disclosed herein are phosphodiester bonds (“soft” oligonucleotides, as described in WO 2007/026190). In another embodiment, CpG oligonucleotides of the disclosure are rendered resistant to degradation (e.g., are stabilized) and comprise phosphorothioate linkages.

The immunostimulatory oligonucleotides may have a chimeric backbone, which have combinations of phosphodiester and phosphorothioate linkages. For purposes of the instant disclosure, a chimeric backbone refers to a partially stabilized backbone, wherein at least one internucleotide linkage is phosphodiester, and wherein at least one other internucleotide linkage is a stabilized internucleotide linkage. When the phosphodiester linkage is located within the CpG motif such molecules are called “semi-soft” as described in WO 2007/026190.

In one aspect of the disclosure, the oligonucleotide includes at least one phosphodiester internucleotide linkage. In one aspect of the disclosure, the oligonucleotide includes at least one phosphorothioate internucleotide linkage. In a further aspect of the disclosure, the oligonucleotide includes a combination of phosphodiester internucleotide linkages and phosphorothioate internucleotide linkages. In another aspect all internucleotide linkages of the oligonucleotide are phosphodiester linkages. In another aspect all internucleotide linkages of the oligonucleotide are phosphorothioate linkages.

Other modified oligonucleotides include phosphodiester modified oligonucleotides, combinations of phosphodiester and phosphorothioate oligonucleotides, methylphosphonate, methyl phosphorothioate, phosphorordithioate, p-ethoxy, and combinations thereof. Each of these combinations and their particular effects on immune cells is discussed in more detail with respect to CpG nucleic acids in PCT Publication Nos. WO 96/02555 and WO 98/18810 and in U.S. Pat. Nos. 6,194,388 and 6,239,116.

Mixed backbone modified ODN may be synthesized as described in WO 2007/026190. In an aspect, the CpG oligonucleotides disclosed herein may comprise substitutions or modifications, such as in the bases and/or sugars as described in WO 2007/026190.

The CpG oligonucleotides may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′-3′-linkage may be a phosphodiester, phosphorothioate or any other modified internucleoside bridge. Methods for accomplishing such linkages are known in the art. For instance, such linkages have been described in Seliger, H. et al., Nucleosides and Nucleotides (1991), 10(1-3), 469-77 and Jiang, et al., Bioorganic and Medicinal Chemistry (1999), 7(12), 2727-2735.

Additionally, 3′-3′-linked oligonucleotides where the linkage between the 3′-terminal nucleosides is not a phosphodiester, phosphorothioate or other modified bridge, can be prepared using an additional spacer, such as tri- or tetra-ethyleneglycol phosphate moiety (Durand, M. et al., Biochemistry (1992), 31 (38), 9197-204, U.S. Pat. Nos. 5,658,738 and 5,668,265). Alternatively, the non-nucleotidic linker may be derived from ethanediol, propanediol, or from an abasic deoxyhbose (dSpacer) unit (Fontanel, Marie Laurence et al., Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two oligonucleotides to be linked.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR₁R₂-phosphoramidate, boranophosphate, a-hydroxybenzyl phosphonate, phosphate-(C₁-C₂₁)—O-alkyl ester, phosphate-[(C₆-C₂₁)aryl-(C₁-C₂₁)—O-alkyl]ester, (C₁-C₈)alkylphosphonate and/or (C₆-C₁₂)arylphosphonate bridges, (C₇-C₁₂)-a-hydroxymethyl-aryl (e.g. disclosed in PCT Publication No. WO 95/01363), wherein (C₆-C₁₂)aryl, (C₆-C₂₀)aryl and (C₆-C₁₄)aryl are optionally substituted by halogen, alkyl, alkoxy, nitro, cyano, and where R₁ and R₂ are, independently of each other, hydrogen, (C₁-C₁₈)-alkyl, (C₆-C₂₀)-aryl, (C₆-C₁₄)-aryl, (C₁-C₈)-alkyl, for example, hydrogen, (C₁-C₈)-alkyl, for example (C₁-C₄)-alkyl and/or methoxyethyl, or R₁ and R₂ form, together with the nitrogen atom carrying them, a 5 to 6-membered heterocyclic ring which can additionally contain a further heteroatom selected from the group O, S and N.

The replacement of a phosphodiester bridge located at the 3′ and/or the 5′ end of a nucleoside by a dephospho bridge (dephospho bridges are described, for example, in Uhlmann E. and Peyman A. in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

Different classes of CpG immunostimulatory oligonucleotides have been identified. These are referred to as A, B, C and P class, and are described in greater detail at page 3, line 22, to page 12, line 36, of WO 2010/125480. Methods of the disclosure embrace the use of these different classes of CpG immunostimulatory oligonucleotides.

In an aspect of the present disclosure, the adjuvant compounds as disclosed herein comprise an A class CpG ODN. In some aspects, the A class CpG oligonucleotide of the present disclosure comprises the nucleic acid sequence: 5′ GGGGACGACGTCGTGGGGGGG 3′ (SEQ ID NO: 115).

In any of the A class CpG oligonucleotide sequences, all of the linkages may be all phosphorothioate bonds. In another aspect, one or more of the linkages may be phosphodiester, for example between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In another aspect of the present disclosure, the adjuvant compounds as disclosed herein comprise a B class CpG ODN that activates B cells. In one aspect, the CpG oligonucleotide of the present disclosure is a B class CpG oligonucleotide represented by at least the formula: 5′ X₁X₂CGX₃X₄ 3′, wherein X1, X2, X3, and X4 are nucleotides. In one embodiment, X₂ is adenine, guanine, or thymine. In another embodiment, X₃ is cytosine, adenine, or thymine. The B class CpG oligonucleotide sequences of the present disclosure may include those described in WO 96/02555, WO 98/18810 and U.S. Pat. Nos. 6,194,388; 6,207,646; 6,214,806; 6,218,371; 6,239,116 and 6,339,068.

In some aspects, the B class CpG oligonucleotides of the present disclosure may include, but are not limited to, the following nucleic acid sequences:

(SEQ ID NO: 116, CpG 24555)
5′ TCGTCGTTTTTCGGTGCTTTT 3′,
(SEQ ID NO: 117, CpG 1018)
5′ TGACTGTGAACGTTCGAGATGA 3′;
(SEQ ID NO: 118, CpG 7909)
5′ TCGTCGTTTTGTCGTTTTGTCGTT 3′;
(SEQ ID NO: 119, CpG 10103)
5′ TCGTCGTTTTTCGGTCGTTTT 3′;
(SEQ ID NO: 120, CpG 1826)
5′ TCCATGACGTTCCTGACGTT 3′;
(SEQ ID NO: 121)
5′ TCGTCGTTTCGTCGTTTTGTCGTT 3′;
and
(SEQ ID NO: 22)
5′ TCGTCGTTTTGTCGTTTTTTTCGA 3′.

In some embodiments, the CpG oligonucleotide described herein contains palindromic repeats. In some embodiments, the CpG oligonucleotide described herein contains palindromic repeats following the formula 5′-purine-purine-CG-pyrimidine-pyrimidine-3′.

In any of the B class CpG oligonucleotide sequences, all of the linkages may be all phosphorothioate bonds. In another aspect, in any of these sequences, one or more of the linkages may be phosphodiester, for example between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In a particular aspect of the disclosure, the CpG ODN comprises the nucleic acid sequence 5′ T*C*G*T*C*G*T*T*T*T*T*C*G*G*T*G*C*T*T*T*T 3′ (SEQ ID NO: 123) wherein * indicates a phosphorothioate linkage. SEQ ID NO: 123 corresponds to the sequence of CpG 24555 wherein each of the internucleotide linkages are phosphorothioate linkages. CpG 24555 is a TLR9 agonist with potent Th1 cell activity that stimulates strong B-cell and NK-cell activation and is described in U.S. Pat. No. 8,552,165, incorporated by reference herein.

As used herein, “CpG 24555” refers to a sequence comprising or consisting of the sequence of either SEQ ID NO: 116 or SEQ ID NO: 123. CpG 24555 is described in U.S. Pat. No. 8,552,165, incorporated by reference herein in the entirety. In some embodiments, at least one CG dinucleotide within CpG 24555 comprises a cytosine that is unmethylated. In some embodiments, at least two or three CG dinucleotides within CpG 24555 comprises a cytosine that is unmethylated. In a particular embodiment, each CG dinucleotide within CpG 24555 comprises a cytosine that is unmethylated.

In an aspect of the present disclosure, the immunogenic compositions as disclosed herein comprise a C class CpG oligonucleotide. In some aspects, the C class CpG oligonucleotides of the present disclosure may include, but are not limited to, the following nucleic acid sequences:

(SEQ ID NO: 124)
5′ TCGCGTCGTTCGGCGCGCGCCG 3′;
(SEQ ID NO: 125)
5′ TCGTCGACGTTCGGCGCGCGCCG 3′;
(SEQ ID NO: 126)
5′ TCGGACGTTCGGCGCGCGCCG 3′;
(SEQ ID NO: 127)
5′ TCGGACGTTCGGCGCGCCG 3′;
(SEQ ID NO: 128)
5′ TCGCGTCGTTCGGCGCGCCG 3′;
(SEQ ID NO: 129)
5′ TCGACGTTCGGCGCGCGCCG 3′;
(SEQ ID NO: 130)
5′ TCGACGTTCGGCGCGCCG 3′;
(SEQ ID NO: 131)
5′ TCGCGTCGTTCGGCGCCG 3′;
(SEQ ID NO: 132)
5′ TCGCGACGTTCGGCGCGCGCCG 3′;
(SEQ ID NO: 133)
5′ TCGTCGTTTTCGGCGCGCGCCG 3′;
(SEQ ID NO: 134)
5′ TCGTCGTTTTCGGCGGCCGCCG 3′;
(SEQ ID NO: 135)
5′ TCGTCGTTTTACGGCGCCGTGCCG 3′;
and
(SEQ ID NO: 136)
5′ TCGTCGTTTTCGGCGCGCGCCGT 3′.

In any of the C class CpG oligonucleotide sequences, all of the linkages may be all phosphorothioate bonds. In another embodiment, in any of these sequences, one or more of the linkages may be phosphodiester, for example between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In an aspect of the present disclosure, the immunogenic compositions as disclosed herein comprise a P class CpG Oligonucleotide. In some aspects, the CpG oligonucleotides of the present disclosure may include a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In one aspect, the P class CpG oligonucleotide includes at least one unmethylated CpG dinucleotide. In another aspect, the TLR activation domain is TCG, TTCG, TTTCG, TYpR, TTYpR, TTTYpR, UCG, UUCG, UUUCG, TTT, or TTTT. In yet another aspect, the TLR activation domain is within the 5′ palindromic region. In another aspect, the TLR activation domain is immediately 5′ to the 5′ palindromic region. In some aspects, the P class CpG oligonucleotides of the disclosure comprise the nucleic acid sequence: 5′ seq 3′ (SEQ ID NO: 137).

In any of the P class CpG oligonucleotide sequences, all of the linkages may be all phosphorothioate bonds. In another aspect, one or more of the linkages may be phosphodiester, for example between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

In one aspect, the CpG ODN adjuvants described herein comprise between 15 and 30 nucleotides. For example, in some embodiments, the CpG ODN adjuvant comprises 15, 16, 17, 18, 19, 20, 21, 22, 23, 24, 25, 26, 27, 28, 29, or 30 nucleotides. In some embodiments, the CpG ODN adjuvant comprises 20, 21, 22, or 24 nucleotides. In a particular embodiment, the CpG ODN adjuvant comprises 21 nucleotides.

In another aspect, the CpG ODN adjuvants described herein comprise a CpG motif consisting of 6, 7, 8, 9, 10, 11, 12, 13, 14, 15, 16, 17, 18, 19, 20, 21, 22, 23, or 24 nucleotides. In a particular embodiment, the CpG ODN adjuvants comprises a CpG consisting of 16 nucleotides.

### Liposomal Adjuvants

In one embodiment, the adjuvant comprises liposomes. “Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6,126,966; 6,056,973; and 6,043,094. Liposomes can be made without hydrophilic polymers. Therefore, liposome adjuvants may or may not contain hydrophilic polymers. Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatidic acid, phosphatidylserine, phosphatidylglycerol, phosphatidylinositol, and sphingomyelin as disclosed in U.S. Pat. Nos. 6,056,973 and 5,874,104.

A liposomal adjuvant comprises liposomes. When a liposomal adjuvant is used in a vaccine formulation, water-soluble antigens, such as proteins, peptides, nucleic acids, or carbohydrates, are encapsulated in the internal aqueous volume of the liposomes (See Tretiakova et al. Liposomes as Adjuvants and Vaccine Delivery Systems. Biochem (Mosc) Suppl Ser A Membr Cell Biol. 2022; 16(1):1-20). Alternatively, when a liposomal adjuvant is combined with lipophilic/amphiphilic substances, such as lipopeptides and glycolipids, these agents are embedded in the lipid bilayer (ld.) Depending on the type of molecule that is combined with the liposomal adjuvant, additional interactions can include associating with the surface of liposomes by adsorption or covalent binding (Id.) Accordingly, in some embodiments, a liposomal adjuvant comprises water-soluble antigens and the antigens are encapsulated in the internal aqueous volume of the liposomes. In some embodiments, water-soluble antigens are proteins, peptides, nucleic acids, or carbohydrates. In some embodiments, a liposomal adjuvant is combined with lipophilic or amphiphilic molecules and these molecules are embedded in the lipid bilayer. In some embodiments, the lipophilic or amphiphilic molecules embedded in the lipid bilayer of the liposome comprise cholesterol, fatty acids, or lipids. In some embodiments, the lipophilic or amphiphilic molecules embedded in the lipid bilayer are lipidated.

Contemplated herein is the use of any liposomal adjuvant. In one embodiment, the liposomal adjuvant is AS01. AS01 comprises 3-O-deacylated monophosphoryl lipid A (3D-MPL) and QS21 in a “quenched form” with cholesterol (See U.S. Pat. No. 10,039,823). In AS01, the lipid bilayer is comprised of a neutral lipid that is “non-crystalline” at room temperature, such as dioleoyl phosphatidylcholine, cholesterol, MPLA, and QS-21 (See U.S. Pat. No. 10,039,823 and WO 1996/033739). During manufacture of AS01, small unilamellar liposomal vesicles (SUV) are first created and purified QS-21 is then added to the SUV. The QS-21 imparts unique properties in that it binds to the liposomal cholesterol where it causes perforations (holes) or other permanent structural changes in the liposomes (See, e.g., Paepenmuller et al., 2014, Int. J. Pharm., 475: 138-46). A reduced amount of free QS-21 presumably resulted in reduced local injection pain often caused by free QS-21 (See, e.g., Waite et al., 2001, Vaccine, 19: 3957-67; Mbawuike et al., 2007, Vaccine, 25: 3263-69). In some embodiments, AS01 contains cholesterol (sterol) at a mole percent concentration of between about 1 and about 50% (mol/mol), for example between about 20 and about 25% (mol/mol) (See U.S. Pat. No. 10,039,823). In some embodiments, AS01 (including for example, AS01A, AS01B, AS01C, AS01D, AS01E, and AS015) comprises dioleoyl phosphatidylcholine (DOPC), cholesterol, MPLA, for example 3D-MPL, and QS-21. In further embodiments, the liposomal adjuvant is selected from the group consisting of AS01A, AS01B, AS01C, AS01D, AS01E, and AS015. In one embodiment, the liposomal adjuvant is AS01A. In some embodiments, AS01A comprises 3D-MPL, toll-like receptor 4 agonist, and QS-21. In one embodiment, the liposomal adjuvant is AS01B. In some embodiments, AS01B comprises 1000 μg per dose DOPC, 250 μg per dose cholesterol, 50 μg per dose 3D-MPL, 50 μg per dose QS21, phosphate NaCl buffer, and water to a volume of 0.5 ml (See U.S. Pat. No. 10,039,823). In one embodiment, the liposomal adjuvant is AS01E. In some embodiments, AS01E comprises the same components as AS01B but at a lower concentration. In some embodiments, AS01E comprises 500 μg per dose dioleoyl phosphatidylcholine (DOPC), 125 μg per dose cholesterol, 25 μg per dose 3D-MPL, 25 μg per dose QS21, phosphate NaCl buffer, and water to a volume of 0.5 ml (See U.S. Pat. No. 10,039,823). In one embodiment, the liposomal adjuvant is AS015. In some embodiments, AS015 comprises dioleoyl phosphatidylcholine (DOPC), cholesterol, 3D-MPL, QS-21, and CpG.

In one embodiment, the liposomal adjuvant is LiNA-1. In some embodiments, LiNA-1 comprises MPLA and a saponin. In some embodiments, LiNA-1 comprises MPLA and QS-21. In other embodiments, LiNA-1 comprises phosphorylated hexaAcyl disaccharide (PHAD®) (i.e., monophosphoryl lipid A (synthetic) available from Avanti® polar lipids) and QS-21. In another particular embodiment, LiNA-1 comprises PHAD®, QS-21, cholesterol, and DOPC. In another particular embodiment, LiNA-1 comprises 3D-PHAD®, QS-21, cholesterol, and DOPC. In another particular embodiment, LiNA-1 comprises the following components per 0.5 mL dose: (i) 50 μg MPLA (i.e., 3D-PHAD®), (ii) 250 μg cholesterol, (iii) 50 μg QS-21, and (iv) 1000 μg DOPC. In another particular embodiment, LiNA-1 comprises the following components per 0.5 mL dose: (i) 50 μg MPLA (i.e., PHAD®), (ii) 250 μg cholesterol, (iii) 50 μg QS-21, and (iv) 1000 μg DOPC. In some embodiments, the LiNA-1 formulations may be LiNA-1 at 0.0625× concentration (0.0625×LiNA-1), LiNA-1 at 0.125× concentration (0.125×LiNA-1), LiNA-1 at 0.25× concentration (0.25×LiNA-1), LiNA-1 at 0.5× concentration (0.5×LiNA-1), LiNA-1 at 1× concentration (1×LiNA-1), LiNA-1 at 2× concentration (2×LiNA-1), LiNA-1 at 3× concentration (3×LiNA-1), or LiNA-1 at 4× concentration (4×LiNA-1).

In a particular embodiment, the liposomal adjuvant is ALFQ. In some embodiments, ALFQ comprises MPLA and saponin (See U.S. Pat. No. 10,434,167). In some embodiments, ALFQ comprises a lipid bilayer comprising phospholipids in which the hydrocarbon chains have a melting temperature in water of ≥23° C. In further embodiments, ALFQ comprises cholesterol at a mole percent concentration of greater than about 50% (mol/mol). In certain embodiments, ALFQ comprises between about 55% and about 71% (mol/mol) cholesterol. In particular embodiments, ALFQ comprises about 55% (mol/mol) cholesterol. In some embodiments, ALFQ comprises MPLA and QS-21. In other embodiments, ALFQ comprises monophosphoryl 3-deacyl lipid A phosphorylated hexaacyl disaccharide (3D-PHAD®) (i.e., monophosphoryl 3-Deacyl Lipid A (synthetic) available from Avanti*polar lipids) and a saponin. In another particular embodiment, ALFQ comprises 3D-PHAD®, QS-21, dimyristoyl phosphatidylcholine (DMPC), dimyristoyl phosphatidylglycerol (DMPG), and cholesterol. In another particular embodiment, ALFQ comprises (i) 7.0 mg/mL DMPC, (ii) 0.78 mg/ml DMPG, (iii) 5.4 mg/ml cholesterol, (iv) 0.2 mg/mL MPLA (3D-PHAD®), and (v) 0.1 mg/ml QS-21.

In a particular embodiment, the liposomal adjuvant is LiNA-2. LiNA-2 is described in WO2023/175454, incorporated by reference herein in the entirety. In some embodiments, LiNA-2 comprises MPLA and saponin. In some embodiments, LiNA-2 comprises a lipid bilayer comprising phospholipids in which the hydrocarbon chains have a melting temperature in water of ≥23° C. In further embodiments, LiNA-2 comprises cholesterol at a mole percent concentration of greater than about 50% (mol/mol). In certain embodiments, LiNA-2 comprises between about 55% to about 71% (mol/mol) cholesterol. In particular embodiments, LiNA-2 comprises about 55% (mol/mol) cholesterol. In some embodiments, LiNA-2 comprises MPLA and QS-21. In other embodiments, LiNA-2 comprises monophosphoryl 3-deacyl lipid A phosphorylated hexaacyl disaccharide (3D-PHAD®) and a saponin. In another particular embodiment, LiNA-2 comprises 3D-PHAD®, QS-21, dimyristoyl phosphatidylcholine (DMPC), dimyristoyl phosphatidylglycerol (DMPG) and cholesterol.

In some embodiments, the LiNA-2 adjuvant comprises a phosphate buffer. In some embodiments, the LiNA-2 adjuvant comprises a phosphate buffer at a concentration between about 1 mM and about 100 mM. In some embodiments, the LiNA-2 adjuvant comprises a phosphate buffer between about 1 mM and 10 mM. In some embodiments, the LiNA-2 adjuvant comprises a phosphate buffer of about 5 mM, about 10 mM, about 15 mM, about 20 mM, about 25 mM, about 30 mM, about 35 mM, about 40 mM, about 45 mM, or about 50 mM. In a particular embodiment, the LiNA-2 adjuvant comprises a phosphate buffer of about 10 mM. In another particular embodiment, the LiNA-2 adjuvant comprises 3D-PHAD®, QS-21, DMPC, DMPG, cholesterol, and a phosphate buffer. In a further particular embodiment, the LiNA-2 adjuvant comprises 3D-PHAD®, QS-21, DMPC, DMPG, cholesterol, and 10 mM phosphate buffer.

In some embodiments, the LiNA-2 adjuvant comprises sodium chloride. In some embodiments, the LiNA-2 adjuvant comprises between about 50 mM and about 500 mM sodium chloride. In other embodiments, the LiNA-2 adjuvant comprises about 25 mM, about 50 mM, about 75 mM, about 100 mM, about 125 mM, about 150 mM, about 175 mM, about 200 mM, about 225 mM, or about 250 mM sodium chloride. In a particular aspect, the LiNA-2 adjuvant comprises about 150 mM sodium chloride. In one embodiment, the LiNA-2 adjuvant comprises 3D-PHAD®, QS-21, DMPC, DMPG, cholesterol, sodium chloride, and a phosphate buffer. In a further particular embodiment, the LiNA-2 adjuvant comprises 3D-PHAD®, QS-21, DMPC, DMPG, cholesterol, 150 mM sodium chloride, and a 10 mM phosphate buffer.

In one embodiment, the adjuvant formulation is 0.5×LiNA-2 (also known as ALFQ), wherein the 0.5×LiNA-2 may be homogeneous or heterogeneous, comprising (i) 7.0 mg/mL DMPC, (ii) 0.78 mg/ml DMPG, (iii) 5.4 mg/ml cholesterol, (iv) 0.2 mg/mL MPLA (3D-PHAD®), and (v) 0.1 mg/ml QS-21. In another embodiment, the adjuvant formulation is 1×LiNA-2, wherein the 1×LiNA-2 may be homogeneous or heterogeneous, comprising (i) 14±7 mg/mL DMPC, (ii) 1.6±0.8 mg/ml DMPG, (iii) 11±6 mg/ml cholesterol, (iv) 0.40±0.20 mg/mL MPLA (3D-PHAD®), and (v) 0.20±0.10 mg/ml QS-21. In a further embodiment, the adjuvant formulation is 2×LiNA-2, wherein the 2×LiNA-2 may be homogeneous or heterogeneous, comprising (i) 28±14 mg/mL DMPC, (ii) 3.2±1.6 mg/ml DMPG, (iii) 22±11 mg/ml cholesterol, (iv) 0.80±0.40 mg/mL MPLA (3D-PHAD®), and (v) 0.40±0.20 mg/ml QS-21. In some embodiments, the LiNA-2 homogeneous or heterogeneous adjuvant formulations may be LiNA-2 at 0.0625× concentration (0.0625×LiNA-2), LiNA-2 at 0.125× concentration (0.125×LiNA-2), LiNA-2 at 0.25× concentration (0.25×LiNA-2), LiNA-2 at 0.5× concentration (0.5×LiNA-2), LiNA-2 at 1× concentration (1×LiNA-2), LiNA-2 at 2× concentration (2×LiNA-2), LiNA-2 at 3× concentration (3×LiNA-2), or LiNA-2 at 4× concentration (4×LiNA-2).

In some embodiments, the liposomal adjuvant is CAF09 (See Korsholm et al. Induction of CD8+ T-cell responses against subunit antigens by the novel cationic liposomal CAF09 adjuvant, Vaccine, Volume 32, Issue 31, 2014, Pages 3927-3935). In some embodiments, the liposomal adjuvant CAF09 comprises dimethyldioctadecylammonium (DDA), monomycoloyl glycerol (MMG)-1, and polyinosinic-polycytidylic acid (poly 1:C).

Phosphatidylcholine phospholipid (PC)/Phosphatidylqlycerol phospholipid (PG): In one embodiment wherein the adjuvant comprises liposomes, the liposomes comprise phosphatidylcholine phospholipid (PC). In some embodiments, the PC is selected from the group consisting of: dimyristoyl phosphatidylcholine (DMPC), dipalmitoyl phosphatidylcholine (DPPC), and distearyl phosphatidylcholine (DSPC). In one embodiment wherein the adjuvant comprises liposomes, the liposomes comprise phosphatidylglycerol phospholipid (PG). In some embodiments, the PG is selected from the group consisting of: dimyristoyl phosphatidylglycerol (DMPG), dipalmitoyl phosphatidylglycerol (DPPG), and distearyl phosphatidylglycerol (DSPG). In a further embodiment, the adjuvant comprises a combination of (i) a phosphatidylcholine phospholipid (PC) selected from the group consisting of: dimyristoyl phosphatidylcholine (DMPC), dipalmitoyl phosphatidylcholine (DPPC), and distearyl phosphatidylcholine (DSPC), and (ii) a phosphatidylglycerol phospholipid (PG) selected from the group consisting of: dimyristoyl phosphatidylglycerol (DMPG), dipalmitoyl phosphatidylglycerol (DPPG), and distearyl phosphatidylglycerol (DSPG). In some embodiments, the liposome composition of the adjuvant has a ratio of PC to PG (mol/mol) of about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In a particular embodiment, the liposome composition of the adjuvant comprises PC and PG, wherein the PC is dimyristoyl phosphatidylcholine (DMPC) and the PG is dimyristoyl phosphatidylglycerol (DMPG), having a mole ratio of PC to PG (mol/mol) of about 9:1.

Cholesterol: In some embodiments wherein the adjuvant comprises liposomes, the liposomes of the adjuvant comprise cholesterol. In one embodiment, the liposome composition of the adjuvant formulation comprises cholesterol at a mole percent concentration of over 50% (mol/mol), for example about 55% to about 71% (mol/mol). In a particular embodiment, the adjuvant comprises liposomes that comprise about 55% (mol/mol) cholesterol.

Cholesterol and Phospholipids: In some embodiments wherein the adjuvant comprises liposomes, the liposomes of the adjuvant comprise cholesterol and phospholipids. In some embodiments, the mole ratio of the cholesterol (b) to the phospholipids (a) is about 55:45 to about 71:29. In one embodiment, the mole ratio of the cholesterol (b) to the phospholipids (a) is about 55:50, about 55:45, about 55:40, about 55:35, or about 55:30. In a particular embodiment, the mole ratio of the cholesterol (b) to the phospholipids (a) is about 55:45.

Vesicle Species: In some embodiments wherein the adjuvant comprises liposomes, the liposomes comprise multi-lamellar vesicles (MLV) or small uni-lamellar vesicles (SUV), wherein small uni-lamellar vesicles are about 50 to about 100 nm in diameter, and wherein multi-lamellar vesicles are about 1 to about 4 μm in diameter.

MPLA: In another embodiment wherein the adjuvant comprises liposomes, the liposome composition comprises Lipid A. In another embodiment wherein the adjuvant comprises liposomes, the liposome composition comprises monophosphoryl lipid A (MPLA). In one embodiment, the liposome composition comprises pentaacylated MPLA (P-MPLA). In another embodiment, the liposome composition comprises monophosphoryl lipid A phosphorylated hexaAcyl disaccharide (PHAD®). In a particular embodiment, the MPLA is monophosphoryl 3-deacyl lipid A phosphorylated hexaacyl disaccharide (3D-PHAD®). In one embodiment, the liposome composition comprises about 5 mg or less, about 4 mg or less, about 3 mg or less, about 2 mg or less, about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less of MPLA, PHAD®, or 3D-PHAD®, etc. (total weight per m1 liposome suspension).

MPLA and Phospholipids: In one embodiment, wherein the adjuvant comprises liposomes, the liposomes comprise MPLA and phospholipids. In another embodiment, wherein the adjuvant comprises liposomes, the liposomes comprise PHAD® or 3D-PHAD® and phospholipids. In one embodiment, the liposome composition of the adjuvant has a MPLA:phospholipid mole ratio of about 1:5.6 to about 1:880, or about 1:88 to about 1:220. In one embodiment, the liposome composition of the adjuvant comprises a PC and a PG, wherein the PC is dimyristoyl phosphatidylcholine (DMPC) and the PG is dimyristoyl phosphatidylglycerol (DMPG), having a MPLA:phospholipid mole ratio of about 1:220, about 1:88 or about 1:5.6, in particular 1:88. In one embodiment, the liposome composition of the adjuvant comprises DMPC, DMPG, and 3D-PHAD® and has a 3D-PHAD®:phospholipid mole ratio between about 1:5 and about 1:6, for example 1:5.6. In one embodiment, the liposome composition of the adjuvant comprises DMPC, DMPG, and 3D-PHAD® and has a 3D-PHAD®:phospholipid mole ratio between about 1:200 and about 1:240, for example 1:220. In another embodiment, the liposome composition of the adjuvant comprises DMPC, DMPG, and 3D-PHAD® and has a 3D-PHAD®:phospholipid mole ratio between about 1:80 and about 1:95. In another particular embodiment, the liposome composition of the adjuvant formulation comprises DMPC, DMPG, and 3D-PHAD® and has a 3D-PHAD®:phospholipid mole ratio of about 1:88.

Saponin: In another embodiment, the adjuvant comprises liposomes that comprise a saponin. In some embodiments, the saponin is Quil A, its derivatives thereof, or any purified component thereof (for example, QS-7, QS-18, QS-21, or a mixture thereof). In a particular embodiment, the adjuvant comprises liposomes which comprise QS-21. In some embodiments, the adjuvant formulation has a content of saponin (total weight per m1 liposome suspension) of about 1 mg or less, about 0.9 mg or less, about 0.8 mg or less, about 0.7 mg or less, about 0.6 mg or less, about 0.5 mg or less, about 0.4 mg or less, about 0.3 mg or less, about 0.2 mg or less, about 0.1 mg or less, about 0.09 mg or less, about 0.08 mg or less, about 0.07 mg or less, about 0.06 mg or less, about 0.05 mg or less, about 0.04 mg or less, about 0.03 mg or less, about 0.02 mg or less, or about 0.01 mg or less. In a particular embodiment, the adjuvant formulation comprises a content of saponin of about 0.15 to 0.4 mg/ml.

MPLA and Saponin: In another embodiment wherein the adjuvant comprises liposomes, the adjuvant comprises a MPLA-containing liposome composition and at least one saponin (e.g., QS-21). In another embodiment, the adjuvant comprises a monophosphoryl lipid A (MPLA)-containing liposome composition and at least one saponin, wherein the liposome composition comprises i) a lipid bilayer comprising phospholipids and ii) cholesterol at a mole percent concentration of the liposome composition of greater than about 50% (mol/mol). The saponin may be QS-7, QS-18, QS-21, or a mixture thereof. In a particular embodiment, the saponin is QS-21. In another embodiment, the adjuvant comprises a MPLA-containing liposome that comprises (1) a lipid bilayer comprising phospholipids in which the hydrocarbon chains have a melting temperature in water of >23° C., usually dimyristoyl phosphatidylcholine (DMPC, e.g. 1,2-dimyristoyl-sn-glycero-3-phosphocholine) and dimyristoyl phosphatidylglycerol (DMPG, e.g. 1,2-dimyristoyl-sn-glycero-3-phospho-(1′-rac-glycerol)); (2) cholesterol (Chol) as a stabilizer; and (3) monophosphoryl lipid A (MPLA) as an immunostimulator.

Homogenous Liposomes: In another embodiment, the adjuvant comprises homogenous liposomes. In one embodiment, the adjuvant comprises homogenous liposomes that range in size from between about 1 nm and about 500 nm. In some embodiments, the homogenous liposomes within the adjuvant range in size from between about 10 nm, about 20 nm, about 30 nm, about 40 nm, or about 50 nm and about 400 nm. In other embodiments, the homogenous liposomes within the adjuvant range in size from between about 10 nm, about 20 nm, about 30 nm, about 40 nm, or about 50 nm and about 300 nm. In other embodiments, the homogenous liposomes within the adjuvant range in size from between about 10 nm, about 20 nm, about 30 nm, about 40 nm, or about 50 nm and about 200 nm. In some embodiments, the homogenous liposomes within the adjuvant have a size of less than about 300 nm, about 250 nm, about 200 nm, about 150 nm, or about 100 nm. In a particular embodiment, the homogenous liposomes within the adjuvant have a size of less than about 200 nm. In one embodiment, the homogeneous liposomes have a polydispersity index (PDI) between about 0.05, about 0.1, about 0.015, or about 0.2 and about 0.3, about 0.35, about 0.4, about 0.45, or about 0.5. In some embodiments, the homogenous liposomes that have a PDI less than about 0.3, about 0.35, about 0.4, about 0.45, or about 0.5. In a particular embodiment, the homogenous liposomes within the adjuvant have a PDI of less than about 0.3.

Heterogenous Liposomes: In another embodiment, the adjuvant comprises heterogenous liposomes. In one embodiment, the heterogeneous liposomes range in size from between about 1 nm and about 10 μM. In some embodiments, the heterogenous liposomes range in size from between about 30 nm and about 4 μM. In other embodiments, the heterogenous liposomes range in size from between about 30 nm and about 1400 nm. In still other embodiments, the heterogenous liposomes range in size from between about 30 nm and about 1000 nm. In some embodiments, the heterogenous liposomes range in size from between about 100 nm, about 200 nm, about 300 nm, about 400 nm, or about 500 nm and about 1000 nm. In a particular embodiment, the heterogenous liposomes within the adjuvant range in size from between about 300 nm and about 1000 nm. In other embodiments, the heterogenous liposomes within the adjuvant have a size of greater than about 500 nm, about 400 nm, about 300 nm, about 200 nm, or about 100 nm. In a particular embodiment, the heterogenous liposomes within the adjuvant have a size of greater than 300 nm. In another embodiment, the heterogeneous liposomes have a polydispersity index (PDI) between about 0.4 and about 1. In some embodiments, the heterogeneous liposomes have a PDI about 0.4, about 0.5, about 0.6, about 0.7, about 0.8, about 0.9, about 1, or more. In a particular embodiment, the heterogeneous liposomes of the adjuvant have a PDI of more than about 0.4. In another particular embodiment, the heterogeneous liposomes of the adjuvant have a PDI of more than about 0.5.

In one embodiment wherein the adjuvant comprises liposomes, the adjuvant is ALFQ comprising homogenous liposomes. In another embodiment wherein the adjuvant comprises liposomes, the adjuvant is ALFQ comprising heterogenous liposomes. In another particular embodiment wherein the adjuvant comprises liposomes, the adjuvant is LiNA-2 comprising homogenous liposomes (as referred to as LiNA-2A). In another particular embodiment wherein the adjuvant comprises liposomes, the adjuvant is LiNA-2 comprising heterogenous liposomes (as referred to as LiNA-2B).

In an embodiment, the formulation includes 1, 2, 3, or more adjuvants. In one embodiment, the formulation comprises one adjuvant, wherein the adjuvant comprises liposomes. In another embodiment, the formulation comprises at least two adjuvants, one of which comprises liposomes. In one embodiment, the formulation comprises one adjuvant, wherein the adjuvant comprises MPLA and a saponin. In another embodiment, the formulation comprises at least two adjuvants, one of which comprises MPLA and a saponin. In one embodiment, the formulation comprises one adjuvant, wherein the adjuvant comprises LiNA-2. In another embodiment, the formulation comprises at least two adjuvants, one of which is LiNA-2. In another particular embodiment, the formulation comprises only a LiNA-2 adjuvant.

### Further Components of the Composition

Immunogenic compositions of the disclosure comprise conjugated saccharide antigen(s) (glycoconjugate(s)). They may also include further antigen(s) from E. coli or other pathogens. In some embodiments, the immunogenic compositions disclosed herein may comprise at least 1, 2, 3, 4, 5, or more antigens. In a particular embodiment, the immunogenic composition further comprises polypeptides or nucleic acid specific for E. coli fimbrial adhesin (FimH). In another particular embodiment, immunogenic composition further comprises antigens specific for K. pneumoniae. In some embodiments, the immunogenic composition comprises saccharides specific for K. pneumoniae.

Klebsiella pneumoniae

Klebsiella pneumoniae (K. pneumoniae) is a Gram-negative pathogen, known to cause urinary tract infections, bacteremia, and sepsis. Multidrug-resistant K. pneumoniae infections are an increasing cause of mortality in vulnerable populations at risk. The O-antigen serotypes are highly prevalent among strains causing invasive disease globally and derived O-antigen glycoconjugates are attractive as vaccine antigens.

In one aspect, any of the compositions disclosed herein may further comprise at least one saccharide that is, or is derived from, at least one K. pneumoniae serotype selected from O1 (and d-Gal-III variants), O2 (and d-Gal-III variants), O2ac, O3, O4, O5, O7, O8, and O12. In a particular embodiment, any of the compositions disclosed herein may further comprise a polypeptide derived from K. pneumoniae selected from a polypeptide derived from K. pneumoniae Type I fimbrial protein or an immunogenic fragment thereof; or a polypeptide derived from K. pneumoniae Type III fimbrial protein or an immunogenic fragment thereof; or a combination thereof.

As is known in the art, K. pneumoniae O1 and O2 O-antigens and their corresponding v1 and v2 subtypes are polymeric galactans that differ in the structures of their repeat units. K. pneumoniae O1 and O2 antigens contain homopolymer galactose units (or galactans). K. pneumoniae O1 and O2 antigens each contain D-galactan I units (sometimes referred to as the O2a repeat unit), but O1 antigens differ in that O1 antigens have a D-galactan II cap structure. D-galactan III (d-Gal-III) is a variant of D-galactan I. Structures of the base galactans I and III that define the two distinct serotype O2 subtypes, O2v1 and O2v2; and structures of the derived chimeras resulting from capping by galactan II which yields subtypes O1v1 and O1v2, are shown in Kelly S D, et al. J Biol Chem 2019; 294:10863-76; and Clarke B R, et al. J Biol Chem 2018; 293:4666-79.

In some embodiments, the saccharide derived from K. pneumoniae O1 includes a repeat unit of [→3)-β-D-Galf-(1→3)-α-D-Galp-(1→]. In some embodiments, the saccharide derived from K. pneumoniae O1 includes a repeat unit of [→3)-α-D-Galp-(1→3)-β-D-Galp-(1→]. In some embodiments, the saccharide derived from K. pneumoniae O1 includes a repeat unit of [→3)-β-D-Galf-(1→3)-α-D-Galp-(1→], and a repeat unit of [→3)-α-D-Galp-(1→3)-β-D-Galp-(1→]. In some embodiments, the saccharide derived from K. pneumoniae O1 includes a repeat unit of →3)-β-D-Galf-(1→3)-[α-D-Galp-(1→4)]-α-D-Galp-(1→] (referred to as the D-Gal-III repeat unit). (Kol O., et al. (1992) Carbohydr. Res. 236, 339-344; Whitfield C., et al. (1991) J. Bacteriol. 173, 1420-1431).

In some embodiments, the saccharide derived from K. pneumoniae O2 includes a repeat unit of [→3)-α-D-Galp-(1→3)-β-D-Galf-(1→] (which may be an element of K. pneumoniae serotype O2a antigen). In some embodiments, the saccharide derived from K. pneumoniae O2 includes a repeat unit of [→3)-β-D-GlcpNAc-(1→5)-β-D-Galf-(1→] (which may be an element of K. pneumoniae serotype O2c antigen). In some embodiments, the saccharide derived from K. pneumoniae O2 includes a modification of the O2a repeat unit by side chain addition of (1→4)-linked Galp residues (which may be an element of the K. pneumoniae O2afg antigen). In some embodiments, the saccharide derived from K. pneumoniae O2 includes a modification of the O2a repeat unit by side chain addition of (1→2)-linked Galp residues (which may be an element of the K. pneumoniae O2aeh antigen). (Whitfield C., et al. (1992) J. Bacteriol. 174, 4913-4919).

Without being bound by mechanism or theory, O-antigen polysaccharide structure of K. pneumoniae serotypes 03 and 05 are disclosed in the art to be identical to those of E. coli serotypes 09a (Formula O9a) and 08 (Formula O8), respectively.

In some embodiments, the saccharide derived from K. pneumoniae O4 includes a repeat unit of [→4)-α-D-Galp-(1→2)-β-D-Ribf-(1→)]. In some embodiments, the saccharide derived from K. pneumoniae O7 includes a repeat unit of [→2-a-L-Rhap-(1→2)-β-D-Ribf-(1→3)-α-L-Rhap-(1→3)-α-L-Rhap-(1→]. In some embodiments, the saccharide derived from K. pneumoniae O8 serotype includes the same repeat-unit structure as K. pneumoniae O2a, but is nonstoichiometrically O-acetylated. In some embodiments, the saccharide derived from K. pneumoniae O12 serotype includes a repeat unit of [α-Rhap-(1→3)-β-GlcpNAc] disaccharide repeat unit.

In one aspect, the disclosure includes a composition including a polypeptide derived from E. coli FimH or a fragment thereof; and at least one saccharide that is, or derived from, at least one K. pneumoniae serotype selected from O1 (and d-Gal-III variants), O2 (and d-Gal-III variants), O2ac, O3, O4, O5, O7, O8, and O12. In some embodiments, the composition includes saccharides from or derived from one or more of serotypes O1, O2, O3, and 05, or a combination thereof. In some embodiments, the composition includes saccharides from or derived from each of serotypes O1, O2, O3, and O5.

In some embodiments, the composition includes a saccharide from or derived from one or more of K. pneumoniae serotypes O1, O2, O3 and O5, or a combination thereof. In some embodiments, the composition includes a saccharide from or derived from each of K. pneumoniae serotypes O1, O2, O3 and O5. In some embodiments, the composition includes a saccharide derived from an E. coli O-antigen having Formula O9 and does not include a saccharide derived from K. pneumoniae serotype O3. In some embodiments, the composition includes a saccharide derived from an E. coli O-antigen having Formula O8 and does not include a saccharide derived from K. pneumoniae serotype O5.

In some embodiments, the composition includes at least one saccharide derived from K. pneumoniae type O1. In one aspect of this embodiment, the K. pneumoniae O-antigen is selected from subtype v1 (O1v) or subtype v2 (O1v2). In one aspect of this embodiment, the K. pneumoniae O-antigen is selected from subtype v1 (O1v) and subtype v2 (O1v2). In some embodiments, the composition includes at least one saccharide derived from K. pneumoniae type O2. In one aspect of this embodiment, the K. pneumoniae O-antigen is selected from subtype v1 (O2v1) or subtype v2 (O2v2). In one aspect of this embodiment, the K. pneumoniae O-antigen is selected from subtype v1 (O2v1) and subtype v2 (O2v2). In another aspect, the K. pneumoniae O-antigen is selected from the group consisting of: a) serotype O1 subtype v1 (O1v1), b) serotype O1 subtype v2 (O1v2), c) serotype O2 subtype v1 (O2v1), and d) serotype O2 subtype v2 (O2v2). In one aspect of this embodiment, the K. pneumoniae O-antigen is subtype v1 (O1v1). In one aspect of this embodiment, the K. pneumoniae O-antigen is subtype v2 (O1v2). In one aspect of this embodiment, the K. pneumoniae O-antigen is subtype v1 (O2v1). In one aspect of this embodiment, the K. pneumoniae O-antigen is subtype v2 (O2v2).

In another aspect of this embodiment, the composition comprises one, two, three or four K. pneumoniae O-antigen selected from the group consisting of: a) serotype O1 subtype v1 (O1v1), b) serotype O1 subtype v2 (O1v2), c) serotype O2 subtype v1 (O2v1), and d) serotype O2 subtype v2 (O2v2).In some embodiments, the composition includes a combination of saccharides derived from K. pneumoniae, wherein a first saccharide is derived from any one of K. pneumoniae types selected from the group consisting of O1, O2, O3, and 05; and a second saccharide is derived from a saccharide is derived from any one of K. pneumoniae types selected from the group consisting of O1 (and d-Gal-III variants), 02 (and d-Gal-III variants), O2ac, O3, O4, O5, O7, O8, and O12. For example, in some embodiments, the composition includes at least one saccharide derived from K. pneumoniae type O1 and at least one saccharide derived from K. pneumoniae type O2. In a particular embodiment, the saccharide derived from K. pneumoniae is conjugated to a carrier protein; and the saccharide derived from E. coli is conjugated to a carrier protein.

In another aspect, the disclosure includes a composition including a polypeptide derived from E. coli FimH or a fragment thereof; and at least one saccharide derived from any one K. pneumoniae type selected from the group consisting of O1, O2, O3, and 05.

In some embodiments, the composition includes at least one saccharide derived from K. pneumoniae type O1; and at least one saccharide derived from E. coli having a structure selected from the group consisting of Formula O8 and Formula O9. In another embodiment, the composition includes at least one saccharide derived from K. pneumoniae type O2; and at least one saccharide derived from E. coli having a structure selected from the group consisting of Formula O8 and Formula O9. In another embodiment, the composition includes at least one saccharide derived from K. pneumoniae type O1; at least one saccharide derived from K. pneumoniae type O2; and at least one saccharide derived from E. coli having a structure selected from the group consisting of Formula O8 and Formula O9.

In one embodiment, the disclosure provides a method of inducing an immune response to K. pneumoniae in a subject comprising administering to the subject an immunologically effective amount of an immunogenic composition comprising at least one glycoconjugate from E. coli serotype O8 or O9, wherein said immunogenic composition does not comprise glycoconjugates from K. pneumoniae serotype O5 or O3. In one aspect, the composition includes a saccharide derived from an E. coli O-antigen having Formula O8 and does not include a saccharide derived from K. pneumoniae serotype O5. In another aspect, the composition includes a saccharide derived from an E. coli O-antigen having Formula O9 and does not include a saccharide derived from K. pneumoniae serotype O3.

In another embodiment, the disclosure provides a method of inducing an immune response to E. coli in a subject comprising administering to the subject an immunologically effective amount of an immunogenic composition comprising at least one glycoconjugate from K. pneumoniae serotype O5 or O3, or a variant thereof, wherein said immunogenic composition does not comprise glycoconjugates from E. coli serotype O8 or O9. In one aspect, the composition includes a saccharide derived from K. pneumoniae serotype O5 and does not include a saccharide derived from an E. coli O-antigen having Formula O8. In another aspect, the composition includes a saccharide derived from K. pneumoniae serotype O3 and does not include a saccharide derived from an E. coli O-antigen having Formula O9.

In some embodiments, the composition includes at least one saccharide that is, or is derived from, at least one K. pneumoniae serotype selected from O1 (and d-Gal-III variants), O2 (and d-Gal-III variants), O2ac, O3, O4, O5, O7, O8, and O12; at least one saccharide derived from E. coli having a structure selected from the group consisting of Formula O8 and Formula O9. In some embodiments, the composition includes at least one saccharide that is, or derived from, at least one K. pneumoniae serotype selected from O1 (and d-Gal-III variants), O2 (and d-Gal-III variants), O2ac, O3, O4, O5, O7, O8, and O12; at least one saccharide derived from E. coli having a structure selected from the group consisting of Formula O1A, Formula O1B, Formula O2, Formula O6, and Formula O25B.

In some embodiments, the composition further includes a polypeptide derived from K. pneumoniae selected from a polypeptide derived from K. pneumoniae Type I fimbrial protein or an immunogenic fragment thereof; or a polypeptide derived from K. pneumoniae Type III fimbrial protein or an immunogenic fragment thereof, or a combination thereof. The sequences of said polypeptides are known in the art.

### FimH

Fimbrial adhesins, including type 1 fimbriae, bind to mannosylated glycoproteins in the epithelial layer or are secreted into the urine. Type 1 fimbriae are highly conserved among clinical UPEC isolates and are encoded by a cluster of genes called fim, which encode accessory proteins (FimC, FimD), various structural subunits (FimE, FimF, FimG) and an adhesin called FimH. FimH is composed of two domains, the lectin binding domain (FimH_{LD}) responsible for binding to mannosylated glycoproteins, and the pilin domain. The pilin domain serves to link FimH to other structural subunits of the pilus such as FimG, via a mechanism called donor strand exchange. The FimH pilin domain forms an incomplete immunoglobulin fold, resulting in a groove that provides a binding site for the N-terminal β-strand of FimG, forming a strong intermolecular linkage between FimH and FimG. While FimH_{LD} can be expressed in a soluble, stable form, full length FimH is unstable alone unless in a complex with the chaperone FimC or complemented with the donor strand peptide of FimG in peptide form or as a fusion protein. Accordingly, the expression of a full length FimH molecule that is stable is possible by linking the FimG donor peptide to the C-terminus of full length FimH via a glycine-serine linker, and is designated FimH-DSG.

As used herein, the term “FimH polypeptide” refers to any domain of the full-length wild type E. coli FimH polypeptide, any combination of domains of the full-length wild type E. coli FimH polypeptide, or to the full-length E. coli FimH polypeptide, or any fragment thereof. For example, in one embodiment the present disclosure provides a mutated FimH polypeptide that is a mutated FimH_{LD} polypeptide, or a FimH-DSG polypeptide.

In some embodiments, the composition comprising a glycoconjugate further comprises a polypeptide derived from fimbrial adhesin (FimH). Embodiments of polypeptides derived from FimH, and nucleic acids encoding the same, are provided in WO2023/111907 and WO2024/256962, incorporated by reference herein in the entirety. In some embodiments, the composition comprising a glycoconjugate further comprises a nucleic acid encoding a polypeptide derived from FimH. In a particular embodiment, the composition comprising a glycoconjugate further comprises RNA encoding a polypeptide derived from fimbrial adhesin (FimH). In some embodiments, the polypeptide derived from FimH is selected from the group consisting of SEQ ID Nos: 1-64, 75, 77, 79, 81, 83, 85, 87, or 89.

In some embodiments, the composition comprising a glycoconjugate further comprises a polypeptide, or a nucleic acid encoding a polypeptide, derived from FimH comprising a wild-type amino acid sequence. In some embodiments, the composition comprising a glycoconjugate further comprises a polypeptide, or a nucleic acid encoding a polypeptide, derived from FimH comprising the sequence of SEQ ID NO: 59.

In some embodiments, the composition comprising a glycoconjugate further comprises a polypeptide, or a nucleic acid encoding a polypeptide, derived from FimH comprising a mutated amino acid sequence. For example, in one embodiment, the mutated amino acid sequence of the FimH polypeptide comprises a mutation at the position selected from the group consisting of F1, P12, G14, G15, G16, A18, P26, V27, V28, Q32, N33, L34, V35, R60, S62, Y64, G65, L68, F71, T86, L107, Y108, L109, V112, S113, A115, G116, V118, A119, A127, L129, Q133, F144, V154, V155, V156, P157, T158, V163, and V185, wherein the amino acid positions are numbered according to SEQ ID NO: 59.

In another embodiment, the mutated amino acid sequence of the FimH polypeptide comprises a mutation selected from the group consisting of: F11; F1L; F1V; F1M; F1Y; F1W; P12C; G14C; G15A; G15P; G16A; G16P; A18C; P26C; V27A; V27C; V28C; Q32C; N33C; L34C; L34N; L34S; L34T; L34D; L34E; L34K; L34R; V35C; R60P; S62C; Y64C; G65A; L68C; F71C; T86C; L107C; Y108C; L109C; V112C; S113C; A115V; G116C; V118C; A119C; A119N; A119S; A119T; A119D; A119E; A119K; A119R; A127C; L129C; Q133K; F144C; V154C; V156C; P157C; T158C; V1631; and V1851, wherein the amino acid positions are numbered according to SEQ ID NO: 59.

In particular embodiments, the composition comprising a glycoconjugate further comprises a polypeptide, or a nucleic acid encoding a polypeptide, derived from FimH comprising the mutation G15A. In another particular embodiment, the composition comprising a glycoconjugate further comprises a polypeptide, or a nucleic acid encoding a polypeptide, derived from FimH comprising the mutation G16A. In still another particular embodiment, the composition comprising a glycoconjugate further comprises a polypeptide, or a nucleic acid encoding a polypeptide, derived from FimH comprising the mutation V27A. In one exemplary embodiment, the composition comprising a glycoconjugate further comprises a polypeptide, or a nucleic acid encoding a polypeptide, derived from FimH comprising each of the mutations of G15A, G16A, V27A. In another exemplary embodiment, the composition comprising a glycoconjugate further comprises a polypeptide, or a nucleic acid encoding a polypeptide, derived from FimH comprising the sequence of SEQ ID NO: 62.

In some embodiments, a composition disclosed herein comprising a polypeptide, or a nucleic acid encoding a polypeptide, derived from FimH further comprises an adjuvant. In some embodiments, a composition disclosed herein comprising a polypeptide, or a nucleic acid encoding a polypeptide, derived from FimH further comprises a liposomal adjuvant. In some embodiments, a composition disclosed herein comprising a polypeptide, or a nucleic acid encoding a polypeptide, derived from FimH further comprises LiNA-2. In a preferred embodiment, the composition comprises RNA encoding a polypeptide derived from FimH and LiNA-2. In another preferred embodiment, the composition comprises modRNA encoding a polypeptide derived from FimH and LiNA-2.

In some embodiments, the composition comprises a polypeptide, or a nucleic acid encoding a polypeptide, derived from FimH, an adjuvant, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In some embodiments, the composition comprises a polypeptide, or a nucleic acid encoding a polypeptide, derived from FimH, a liposomal adjuvant, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In some embodiments, the composition comprises a polypeptide, or a nucleic acid encoding a polypeptide, derived from FimH, LiNA-2, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In a preferred embodiment, the composition comprises RNA encoding a polypeptide derived from FimH, LiNA-2, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In another preferred embodiment, the composition comprises modRNA encoding a polypeptide derived from FimH, LiNA-2, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b.

In some embodiments, a composition disclosed herein comprising a polypeptide, or a nucleic acid encoding a polypeptide, derived from FimH further comprises an adjuvant comprising a CpG ODN. In some embodiments, a composition disclosed herein comprising a polypeptide, or a nucleic acid encoding a polypeptide, derived from FimH further comprises an adjuvant comprising CpG 24555. In a preferred embodiment, the composition comprises RNA encoding a polypeptide derived from FimH and CpG 24555. In another preferred embodiment, the composition comprises modRNA encoding a polypeptide derived from FimH and CpG 24555.

In some embodiments, the composition comprises a polypeptide, or a nucleic acid encoding a polypeptide, derived from FimH, an adjuvant comprising a CpG ODN, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In some embodiments, the composition comprises a polypeptide, or a nucleic acid encoding a polypeptide, derived from FimH, an adjuvant comprising CpG 24555, a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In a preferred embodiment, the composition comprises RNA encoding a polypeptide derived from FimH, CpG 24555, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In another preferred embodiment, the composition comprises modRNA encoding a polypeptide derived from FimH, CpG 24555, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b.

### RNA

In some embodiments, the composition comprising a glycoconjugate further comprises a nucleic acid encoding a polypeptide. In some embodiments, the nucleic acid is DNA. In other embodiments, the nucleic acid is RNA. In some embodiments, the composition comprising a glycoconjugate further comprises a nucleic acid encoding a polypeptide derived from FimH. In particular embodiments, the composition comprising a glycoconjugate further comprises a RNA molecule encoding a polypeptide derived from FimH. In another particular embodiment, the composition comprises a RNA molecule encoding a polypeptide derived from FimH and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In another particular embodiment, the composition comprises a RNA molecule encoding a polypeptide derived from FimH and more than one glycoconjugate. In another particular embodiment, the composition comprises a RNA molecule encoding a polypeptide derived from FimH and glycoconjugates comprising E. coli saccharides comprising the structures of each of O1a, O2, O6, and O25b. In another particular embodiment, the composition comprises a RNA molecule encoding a polypeptide derived from FimH and glycoconjugates comprising E. coli saccharides comprising the structures of each of O1a, O2, O6, and O25b, wherein the glycoconjugate comprising O25b comprises a streptococcal C5a peptidase (SCP) carrier protein, or a functional fragment thereof, covalently bound.

In some embodiments, a composition is provided wherein a glycoconjugate is combined with RNA and a lipid nanoparticle (LNP), wherein the RNA and LNP function as an adjuvant. In some embodiments, a composition is provided wherein more than one glycoconjugate is combined with RNA and a LNP, wherein the RNA and LNP function as an adjuvant (i.e., a multivalent vaccine). In a particular embodiment, the composition comprises a RNA molecule encoding a polypeptide derived from FimH, a LNP, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In a particular embodiment, the composition comprises a RNA molecule encoding a polypeptide derived from FimH, a LNP, and glycoconjugates comprising E. coli saccharides comprising the structures of each of O1a, O2, O6, and O25b, wherein the glycoconjugate comprising O25b comprises a streptococcal C5a peptidase (SCP) carrier protein, or a functional fragment thereof, covalently bound to the E. coli saccharide.

In some embodiments, a composition is provided wherein a glycoconjugate is combined with modRNA and a LNP, wherein the modRNA and LNP function as an adjuvant. In some embodiments, a composition is provided wherein more than one glycoconjugate is combined with modRNA and a LNP, wherein the modRNA and LNP function as an adjuvant (i.e., a multivalent vaccine). In a particular embodiment, the composition comprises a modRNA molecule encoding a polypeptide derived from FimH, a LNP, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b. In a particular embodiment, the composition comprises a modRNA molecule encoding a polypeptide derived from FimH, a LNP, and glycoconjugates comprising E. coli saccharides comprising the structures of each of O1a, O2, O6, and O25b, wherein the glycoconjugate comprising O25b comprises a streptococcal C5a peptidase (SCP) carrier protein, or a functional fragment thereof, covalently bound to the E. coli saccharide.

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) OFRs, which may be a sequence of codons that is translatable into a polypeptide or protein of interest.

A number of mRNA vaccine platforms are available in the prior art. The basic structure of in vitro transcribed (IVT) mRNA closely resembles “mature” eukaryotic mRNA and consists of (i) a protein-encoding open reading frame (ORF), flanked by (ii) 5′ and 3′ untranslated regions (UTRs), and at the end sides (iii) a 5′ cap structure and (iv) a 3′ poly(A) tail. The non-coding structural features play important roles in the pharmacology of mRNA and can be individually optimized to modulate the mRNA stability, translation efficiency, and immunogenicity.

By incorporating modified nucleosides, mRNA transcripts referred to as “nucleoside-modified mRNA” or “modRNA” can be produced with reduced immunostimulatory activity, and therefore an improved safety profile can be obtained. In addition, modified nucleosides allow the design of mRNA vaccines with strongly enhanced stability and translation capacity, as they can avoid the direct antibacterial pathways that are induced by type IFNs and are programmed to degrade and inhibit invading mRNA. For instance, the replacement of uridine with pseudouridine in in vitro transcribed (IVT) mRNA reduces the activity of 2′-5′-oligoadenylate synthetase, which regulates the mRNA cleavage by RNase L. In addition, lower activities are measured for protein kinase R, an enzyme that is associated with the inhibition of the mRNA translation process.

Besides the incorporation of modified nucleotides, other approaches have been validated to increase the translation capacity and stability of mRNA. One example is the development of “sequence-engineered mRNA”. Here, mRNA expression can be strongly increased by sequence optimizations in the ORF and UTRs of mRNA, for instance by enriching the GC content, or by selecting the UTRs of natural long-lived mRNA molecules.

Also, several modifications have been implemented at the end structures of mRNA. Anti-reverse cap (ARCA) modifications can ensure the correct cap orientation at the 5′ end, which yields almost complete fractions of mRNA that can efficiently bind the ribosomes. Other cap modifications, such as phosphorothioate cap analogs, can further improve the affinity towards the eukaryotic translation initiation factor 4E, and increase the resistance against the RNA decapping complex.

Conversely, by modifying its structure, the potency of mRNA to trigger innate immune responses can be further improved, but to the detriment of translation capacity. By stabilizing the mRNA with either a phosphorothioate backbone, or by its precipitation with the cationic protein protamine, antigen expression can be diminished, but stronger immune-stimulating capacities can be obtained.

In one aspect the invention relates to an immunogenic composition comprising an mRNA molecule that encodes one or more polypeptides or fragments thereof of E. coli FimH as an antigen. In some embodiments, the mRNA molecule comprises a nucleoside-modified mRNA.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence. In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA.

In some aspects, the RNA molecule includes from about 20 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides.

mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR), at least one 5′-cap region, and a 3′-stabilizing region. In some embodiments, the mRNA of the invention further includes a poly-A region or a Kozak sequence (e.g., in the 5′-UTR). In some cases, mRNA of the invention may contain one or more intronic nucleotide sequences capable of being excised from the polynucleotide. In some embodiments, mRNA of the invention may include a 5′ cap structure, a chain terminating nucleotide, a stem loop, a poly A sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl-pseudouridine), and/or a 5-substituted cytidine (e.g., 5-methyl-cytidine). In a preferred aspect, the modified nucleoside is 1-methyl-pseudouridine.

In some embodiments, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a payload (e.g., an E. coli FimH protein); a 3′ untranslated region (3′ UTR); and a Poly-A sequence.

In some embodiments, a LNP includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, 25 lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 6.0:1, about 6.5:1, or about 7.0:1.

Modified Nucleobases: In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

mRNA of the invention may include one or more naturally occurring components, including any of the canonical nucleotides A (adenosine), G (guanosine), C (cytosine), U (uridine), or T (thymidine). In one embodiment, all or substantially all of the nucleotides comprising (a) the 5′-UTR, (b) the open reading frame (ORF), (c) the 3′-UTR, (d) the poly A tail, and any combination of (a, b, c, or d above) comprise naturally occurring canonical nucleotides A (adenosine), G (guanosine), C (cytosine), U (uridine), or T (thymidine).

mRNA of the invention may include one or more alternative components, as described herein, which impart useful properties including increased stability and/or the lack of a substantial induction of the innate immune response of a cell into which the polynucleotide is introduced. For example, a modRNA may exhibit reduced degradation in a cell into which the modRNA is introduced, relative to a corresponding unaltered mRNA. These alternative species may enhance the efficiency of protein production, intracellular retention of the polynucleotides, and/or viability of contacted cells, as well as possess reduced immunogenicity.

mRNA of the invention may include one or more modified (e.g., altered or alternative) nucleobases, nucleosides, nucleotides, or combinations thereof. The mRNA useful in a LNP can include any useful modification or alteration, such as to the nucleobase, the sugar, or the internucleoside linkage (e.g., to a linking phosphate/to a phosphodiester linkage/to the phosphodiester backbone). In certain embodiments, alterations (e.g., one or more alterations) are present in each of the nucleobase, the sugar, and the internucleoside linkage. Alterations according to the present disclosure may be alterations of ribonucleic acids (RNAs), e.g., the substitution of the 2′-OH of the ribofuranosyl ring to 2′-H, threose nucleic acids (TNAs), glycol nucleic acids (GNAs), peptide nucleic acids (PNAs), locked nucleic acids (LNAs), or hybrids thereof.

mRNA of the invention may or may not be uniformly altered along the entire length of the molecule. For example, one or more or all types of nucleotide (e.g., purine or pyrimidine, or any one or more or all of A, G, U, C) may or may not be uniformly altered in a mRNA, or in a given predetermined sequence region thereof. In some instances, all nucleotides X in a mRNA (or in a given sequence region thereof) are altered, wherein X may be any one of nucleotides A, G, U, C, or any one of the combinations A+G, A+U, A+C, G+U, G+C, U+C, A+G+U, A+G+C, G+U+C or A+G+C.

Different sugar alterations and/or internucleoside linkages (e.g., backbone structures) may exist at various positions in a polynucleotide. One of ordinary skill in the art will appreciate that the nucleotide analogs or other alteration(s) may be located at any position(s) of a polynucleotide such that the function of the polynucleotide is not substantially decreased. An alteration may also be a 5′- or 3′-terminal alteration. In some embodiments, the polynucleotide includes an alteration at the 3′-terminus. The polynucleotide may contain from about 1% to about 100% alternative nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of a canonical nucleotide (e.g., A, G, U, or C).

Polynucleotides may contain at a minimum zero and at maximum 100% alternative nucleotides, or any intervening percentage, such as at least 5% alternative nucleotides, at least 10% alternative nucleotides, at least 25% alternative nucleotides, at least 50% alternative nucleotides, at least 80% alternative nucleotides, or at least 90% alternative nucleotides. For example, polynucleotides may contain an alternative pyrimidine such as an alternative uracil or cytosine. In some embodiments, at least 5%, at least 10%, at least 25%, at least 50%, at least 80%, at least 90% or 100% of the uracil in a polynucleotide is replaced with an alternative uracil (e.g., a 5-substituted uracil). The alternative uracil can be replaced by a compound having a single unique structure or can be replaced by a plurality of compounds having different structures (e.g., 2, 3, 4 or more unique structures). In some instances, at least 5%, at least 10%, at least 25%, at least 50%, at least 80%, at least 90% or 100% of the cytosine in the polynucleotide is replaced with an alternative cytosine (e.g., a 5-substituted cytosine). The alternative cytosine can be replaced by a compound having a single unique structure or can be replaced by a plurality of compounds having different structures (e.g., 2, 3, 4 or more unique structures).

In some instances, nucleic acids do not substantially induce an innate immune response of a cell into which the polynucleotide (e.g., mRNA) is introduced. Features of an induced innate immune response include 1) increased expression of pro-inflammatory cytokines, 2) activation of intracellular PRRs (RIG-I, MDA5, etc., and/or 3) termination or reduction in protein translation.

In some embodiments, the mRNA comprises one or more alternative nucleoside or nucleotides. The alternative nucleosides and nucleotides can include an alternative nucleobase. A nucleobase of a nucleic acid is an organic base such as a purine or pyrimidine or a derivative thereof. A nucleobase may be a canonical base (e.g., adenine, guanine, uracil, thymine, and cytosine). These nucleobases can be altered or wholly replaced to provide polynucleotide molecules having enhanced properties, e.g., increased stability such as resistance to nucleases. Non-canonical or modified bases may include, for example, one or more substitutions or modifications including but not limited to alkyl, aryl, halo, oxo, hydroxyl, alkyloxy, and/or thio substitutions; one or more fused or open rings; oxidation; and/or reduction.

In some embodiments, the nucleobase is an alternative uracil. Exemplary nucleobases and nucleosides having an alternative uracil include pseudouridine (LP), pyridin-4-one ribonucleoside, 5-aza-uracil, 6-aza-uracil, 2-thio-5-aza-uracil, 2-thio-uracil (s²U), 4-thio-uracil (s⁴U), 4-thiopseudouridine (s4ψ), 2-thiopseudouridine (s2ψ), 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s2U), 5-aminomethyl-2-thio-uracil (nmVu), 5-methylaminomethyl-uracil (mnm⁵U), 5-methylaminomethyl-2-thio-uracil (mnmVu), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnmVu), 5-propynyl-uracil, 1-propynyl-pseudouracil, 5-taurinomethyl-uracil (xm⁵U), 1-taurinomethyl-pseudouridine, 5-taurinomethyl-2-thio-uracil(xm⁵s²U), 1-taurinomethyl-4-thio-pseudouridine, 5-methyl-uracil (m⁵U, i.e., having the nucleobase deoxythymine), 1-methyl-pseudouridine (mV), 5-methyl-2-thio-uracil (m⁵s2U), 1-methyl-4-thio-pseudouridine (ms4ψ), 4-thio-1-methyl-pseudouridine, 3-methyl-pseudouridine (m Ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m5D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, NI-methyl-pseudouridine, 3-(3-amino-3-carboxypropyl)uracil (acpU), I-methyl-3-(3-amino-3-carboxypropyl)pseudouridine (acp ψ), 5-(isopentenylaminomethyl)uracil (inm5U), 5-(isopentenylaminomethyl)-2-thio-uracil (inm5s2U), 5,2′-0-dimethyl-uridine (m5Um), 2-thio-2′-0-methyl-uridine (s2Um), 5-methoxycarbonylmethyl-2′-0-methyl-uridine (mem Um), 5-carbamoylmethyl-2′-0-methyl-uridine (ncm5Um), 5-carboxymethylaminomethyl-2′-0-methyl-uridine (cmnm5Um), 3,2′-0-dimethyl-uridine (mUm), and 5-(isopentenylaminomethyl)-2′-0-methyl-uridine (inm5Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(I-E-propenylamino)]uracil. Pseudouridine” is one example of a modified nucleoside that is an isomer of uridine, where the uracil is attached to the pentose ring via a carbon-carbon bond instead of a nitrogen-carbon glycosidic bond.

In some embodiments, the nucleobase is an alternative cytosine. Exemplary nucleobases and nucleosides having an alternative cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocy tidine, 4-thio-1-methy 1-pseudoisocy tidine, 4-thio-1-methyl-1-deaza-pseudoisocytidine, 1-methyl-1-deaza-pseudoisocyti dine, zebularine, 5-aza-zebularine, 5-methy 1-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-1-methyl-pseudoisocytidine, lysidine (k2C), 5,2′-0-dimethyl-cytidine (m5Cm), N4-acetyl-2′-0-methyl-cytidine (ac4Cm), N4,2′-0-dimethyl-cytidine (m4Cm), 5-formyl-2′-0-methyl-cytidine (f5Cm), N4,N4,2′-0-trimethyl-cytidine (m42Cm), 1-thio-cytosine, 5-hydroxy-cytosine, 5-(3-azidopropyl)-cytosine, and 5-(2-azidoethyl)-cytosine.

In some embodiments, the nucleobase is an alternative adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methy 1-adenine (ml A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-0-trimethyl-adenosine (m62Am), I,2′-0-dimethyl-adenosine (ml Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In some embodiments, the nucleobase is an alternative guanine. Exemplary nucleobases and nucleosides having an alternative guanine include inosine (I), 1-methyl-inosine (mil), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQl), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (mIG), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-0-methyl-guanosine (m2Gm), N2,N2-dimethyl-2′-0-methyl-guanosine (m22Gm), 1-methyl-2′-0-methyl-guanosine (mlGm), N2,7-dimethyl-2′-0-methyl-guanosine (m2,7Gm), 2′-0-methyl-inosine (Im), I,2′-0-dimethyl-inosine (mllm), 1-thio-guanine, and O-6-methyl-guanine.

The alternative nucleobase of a nucleotide can be independently a purine, a pyrimidine, a purine or pyrimidine analog. For example, the nucleobase can be an alternative to adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

In some aspects, the RNA molecule comprises a nucleic acid sequence having at least one uridine replaced by pseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by 1-methyl-pseudouridine. In some aspects, the RNA molecule comprises a nucleic acid sequence having all uridines replaced by 1-methyl-pseudouridine.

5′ CAP: The mRNA may include a 5′-cap structure. The 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The cap further assists the removal of 5′-proximal introns removal during mRNA splicing.

Endogenous polynucleotide molecules may be 5′-end capped generating a 5′-ppp-5′-triphosphate linkage between a terminal guanosine cap residue and the 5-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5-decapping through hydrolysis and cleavage of the guanylate cap structure may target a polynucleotide molecule, such as an mRNA molecule, for degradation.

Alterations to polynucleotides may generate a non-hydrolyzable cap structure preventing decapping and thus increasing polynucleotide half-life. Because cap structure hydrolysis requires cleavage of 5′-ppp-5′ phosphorodiester linkages, alternative nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional alternative guanosine nucleotides may be used such as a-methyl-phosphonate and seleno-phosphate nucleotides. Additional alterations include, but are not limited to, 2′-0-methylation of the ribose sugars of 5-terminal and/or 5-anteterminal nucleotides of the polynucleotide (as mentioned above) on the 2′-hydroxy group of the sugar. Multiple distinct 5′-cap structures can be used to generate the 5′-cap of an mRNA molecule.

Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, while retaining cap function. Cap analogs may be chemically (i.e., non-enzymatically) or enzymatically synthesized and/linked to a polynucleotide. For example, the Anti-Reverse Cap Analog (ARCA) cap contains two guanosines linked by a 5′-5′-triphosphate group, wherein one guanosine contains an N7-methyl group as well as a 3′-0-methyl group (i.e., N7,2′-0-dimethyl-guanosine-5′-triphosphate-5′-guanosine, m7G-3′mppp-G, which may equivalently be designated 3′ 0-Me-m7G(5′)ppp(5′)G). The 3′-0 atom of the other, unaltered, guanosine becomes linked to the 5′-terminal nucleotide of the capped polynucleotide (e.g., an mRNA). The N7- and 3′-0-methylated guanosine provides the terminal moiety of the capped polynucleotide (e.g., mRNA). Another exemplary cap is mCAP, which is similar to ARCA but has a 2′-0-methyl group on guanosine (i.e., N7,2′-0-dimethyl-guanosine-5′-triphosphate-5′-guanosine, m7Gm-ppp-G).

A cap may be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the cap structures of which are herein incorporated by reference.

Alternatively, a cap analog may be a N7-(4-chlorophenoxy ethyl) substituted dinucleotide cap analog known in the art and/or described herein. Non-limiting examples of N7-(4-chlorophenoxy ethyl) substituted dinucleotide cap analogs include a N7-(4-chlorophenoxyethyl)-G(5)ppp(5′)G and a N7-(4-chlorophenoxyethyl)-m3′-OG(5)ppp(5′)G cap analog (see, e.g., the various cap analogs and the methods of synthesizing cap analogs described in Kore et al. Bioorganic & Medicinal Chemistry 2013 21:4570-4574; the cap structures of which are herein incorporated by reference). In other instances, a cap analog useful in the polynucleotides of the present disclosure is a 4-chloro/bromophenoxy ethyl analog.

While cap analogs allow for the concomitant capping of a polynucleotide in an in vitro transcription reaction, up to 20% of transcripts remain uncapped. This, as well as the structural differences of a cap analog from endogenous 5′-cap structures of polynucleotides produced by the endogenous, cellular transcription machinery, may lead to reduced translational competency and reduced cellular stability.

Alternative polynucleotides may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in the polynucleotides of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-0-methyltransferase enzyme can create a canonical 5′-5′-triphosphate linkage between the 5-terminal nucleotide of a polynucleotide and a guanosine cap nucleotide wherein the cap guanosine contains an N7-methylation and the 5-terminal nucleotide of the polynucleotide contains a 2′-0-methyl. Such a structure is termed the CapI structure. This cap results in a higher translational-competency, cellular stability, and a reduced activation of cellular pro-inflammatory cytokines, as compared, e.g., to other 5′ cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

A further cap structure includes N¹-methylpseudouridine-5′-triphosphate (also known as N¹-methylpseudouridine-5′-triphosphate, NimeΨTP, m¹ΨTP, 1-methyl-pseudouridine phosphoramidite or N¹-methyl-pseudouridine-5′-triphosphate; (TriLink Biotechnologies) having the structure set forth below:

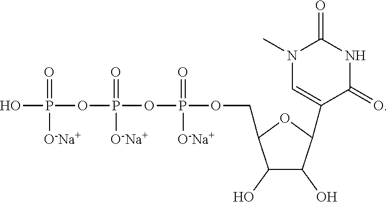

Because the alternative polynucleotides may be capped post-transcriptionally, and because this process is more efficient, nearly 100% of the mRNA may be capped. This is in contrast to −80% when a cap analog is linked to a polynucleotide in the course of an in vitro transcription reaction.

5′-terminal caps may include endogenous caps or cap analogs. A 5′-terminal cap may include a guanosine analog. Useful guanosine analogs include inosine, NI-methyl-guanosine, 2′-fluoro-guanosine, 7-deaza-guanosine, 8-oxo-guanosine, 2-amino-guanosine, LNA-guanosine, and 2-azido-guanosine. In some cases, a polynucleotide contains a modified 5′-cap. A modification on the 5′-cap may increase the stability of polynucleotide, increase the half-life of the polynucleotide, and could increase the polynucleotide translational efficiency. The modified 5-cap may include, but is not limited to, one or more of the following modifications: modification at the 2′- and/or 3′-position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety.

Untranslated Reqions (UTRs): The 5′ UTR is a regulatory region situated at the 5′ end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

A 5′-UTR may be provided as a flanking region to the mRNA. A 5′-UTR may be homologous or heterologous to the coding region found in a polynucleotide. Multiple 5′-UTRs may be included in the flanking region and may be the same or of different sequences. Any portion of the flanking regions, including none, may be codon optimized and any may independently contain one or more different structural or chemical alterations, before and/or after codon optimization.

To alter one or more properties of an mRNA, 5′UTRs which are heterologous to the coding region of an mRNA may be engineered. The mRNA may then be administered to cells, tissue or organisms and outcomes such as protein level, localization, and/or half-life may be measured to evaluate the beneficial effects the heterologous 5′ UTR may have on the mRNA.

Variants of the 5′UTRs may be utilized wherein one or more nucleotides are added or removed to the termini, including A, T, C or G. 5′UTRs may also be codon-optimized, or altered in any manner described herein.

In some aspects, the RNA molecule includes a 5′ untranslated region (5′-UTR). In some aspects, the 5′ UTR comprises a sequence selected from any of SEQ ID NO: 93 to SEQ ID NO: 99. In some aspects, the 5′ UTR comprises a sequence having at least 90%, 91%, 92%, 93%, 94%, 95%, 96%, 97%, 98% or 99% or higher identity to any of SEQ ID NO: 93 to SEQ ID NO: 99. In some aspects, the 5′ UTR comprises a sequence selected from any of SEQ ID NO: 93 to SEQ ID NO: 99. In some aspects, the 5′ UTR comprises a sequence consisting of any of SEQ ID NO: 93 to SEQ ID NO: 99.

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a Xhol site.

In some aspects, the RNA molecules and RNA-LNPs include a 3′ untranslated region (3′-UTR). In some aspects, the 3′ UTR comprises a sequence selected from any of SEQ ID NO: 100 to SEQ ID NO: 102. In some aspects, the 3′ UTR comprises a sequence having at least 90%, 91%, 92%, 93%, 94%, 95%, 96%, 97%, 98% or 99% or higher identity to any of SEQ ID NO: 100 to SEQ ID NO: 102. In some aspects, the 3′ UTR comprises a sequence selected from any of SEQ ID NO: 100 to SEQ ID NO: 102. In some aspects, the 3′ UTR comprises a sequence consisting of any of SEQ ID NO: 100 to SEQ ID NO: 102.

mRNAs may include a stem loop such as, but not limited to, a histone stem loop. The stem loop may be a nucleotide sequence that is about 25 or about 26 nucleotides in length. The histone stem loop may be located 3′-relative to the coding region (e.g., at the 3′-terminus of the coding region). As a non-limiting example, the stem loop may be located at the 3′-end of a polynucleotide described herein. In some cases, an mRNA includes more than one stem loop (e.g., two stem loops). A stem loop may be located in a second terminal region of a polynucleotide. As a non-limiting example, the stem loop may be located within an untranslated region (e.g., 3′-UTR) in a second terminal region. In some cases, a mRNA which includes the histone stem loop may be stabilized by the addition of a 3′-stabilizing region (e.g., a 3′-stabilizing region including at least one chain terminating nucleoside). Not wishing to be bound by theory, the addition of at least one chain terminating nucleoside may slow the degradation of a polynucleotide and thus can increase the half-life of the polynucleotide. In other cases, a mRNA, which includes the histone stem loop may be stabilized by an alteration to the 3′-region of the polynucleotide that can prevent and/or inhibit the addition of oligio(U). In yet other cases, a mRNA, which includes the histone stem loop may be stabilized by the addition of an oligonucleotide that terminates in a 3′-deoxynucleoside, 2′,3′-dideoxynucleoside 3′-0-methylnucleosides, 3-0-ethylnucleosides, 3′-arabinosides, and other alternative nucleosides known in the art and/or described herein. In some instances, the mRNA of the present disclosure may include a histone stem loop, a poly-A region, and/or a 5′-cap structure. The histone stem loop may be before and/or after the poly-A region. The polynucleotides including the histone stem loop and a poly-A region sequence may include a chain terminating nucleoside described herein. In other instances, the polynucleotides of the present disclosure may include a histone stem loop and a 5′-cap structure. The 5′-cap structure may include, but is not limited to, those described herein and/or known in the art. In some cases, the conserved stem loop region may include a miR sequence described herein. As a non-limiting example, the stem loop region may include the seed sequence of a miR sequence described herein. In another non-limiting example, the stem loop region may include a miR-122 seed sequence.

mRNA may include at least one histone stem-loop and a poly-A region or polyadenylation signal. In certain cases, the polynucleotide encoding for a histone stem loop and a poly-A region or a polyadenylation signal may code for a pathogen antigen or fragment thereof. In other cases, the polynucleotide encoding for a histone stem loop and a poly-A region or a polyadenylation signal may code for a therapeutic protein. In some cases, the polynucleotide encoding for a histone stem loop and a poly-A region or a polyadenylation signal may code for a tumor antigen or fragment thereof. In other cases, the polynucleotide encoding for a histone stem loop and a poly-A region or a polyadenylation signal may code for an allergenic antigen or an autoimmune self-antigen.

Open Reading Frame (ORF): The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, which are known in the art. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

The present disclosure provides for an RNA molecule comprising at least one open reading frame encoding an E. coli FimH polypeptide as described herein. In some aspects, an RNA molecule comprising at least one open reading frame encoding an E. coli FimH protein as described herein.

Genes of Interest: The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. The sequence for a particular gene of interest is readily identified by one of skill in the art using public and private databases, e.g., GENBANK®.

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

The term “antigen” may refer to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g. an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from E. coli fimbrial antigen (FimH).

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

In some aspects, the RNA molecule encodes a FimH protein comprising the sequence of any one of SEQ ID NOs: 1 to 64, 75, 77, 79, 81, 83, 85, 87 or 89, or a fragment or variant thereof.

In some aspects, the RNA molecule encodes an E. coli FimH protein synthesized from the nucleic acid sequence comprising any one of SEQ ID NOs: SEQ ID NO: 74, SEQ ID NO: 76, SEQ ID NO: 78, SEQ ID NO: 66 to SEQ ID NO: 73, SEQ ID NO: 80, SEQ ID NO: 82, SEQ ID NO: 84, SEQ ID NO: 86 or SEQ ID NO: 88, or fragment or variant thereof.

Poly-A Tail: In some aspects, RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

An mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of a nucleic acid. During RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Unique poly-A region lengths may provide certain advantages to the alternative polynucleotides of the present disclosure. Generally, the length of a poly-A region of the present disclosure is at least 30 nucleotides in length. In another embodiment, the poly-A region is at least 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 70 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1700 nucleotides. In another embodiment, the length is at least 1800 nucleotides. In another embodiment, the length is at least 1900 nucleotides. In another embodiment, the length is at least 2000 nucleotides. In another embodiment, the length is at least 2500 nucleotides. In another embodiment, the length is at least 3000 nucleotides. In some instances, the poly-A region may be 80 nucleotides, 120 nucleotides, 160 nucleotides in length on an alternative polynucleotide molecule described herein. In other instances, the poly-A region may be 20, 30, 40, 80, 100, 120, 140 or 160 nucleotides in length on an alternative polynucleotide molecule described herein. In some cases, the poly-A region is designed relative to the length of the overall alternative polynucleotide. This design may be based on the length of the coding region of the alternative polynucleotide, the length of a particular feature or region of the alternative polynucleotide (such as mRNA) or based on the length of the ultimate product expressed from the alternative polynucleotide. When relative to any feature of the alternative polynucleotide (e.g., other than the mRNA portion which includes the poly-A region) the poly-A region may be 10%, 20%, 30%, 40%, 50%, 60%, 70%, 80%, 90% or 100% greater in length than the additional feature. The poly-A region may also be designed as a fraction of the alternative polynucleotide to which it belongs. In this context, the poly-A region may be 10%, 20%, 30%, 40%, 50%, 60%, 70%, 80%, or 90% or more of the total length of the construct or the total length of the construct minus the poly-A region.

In certain cases, engineered binding sites and/or the conjugation of mRNA for poly-A binding protein may be used to enhance expression. The engineered binding sites may be sensor sequences which can operate as binding sites for ligands of the local microenvironment of the mRNA. As a non-limiting example, the mRNA may include at least one engineered binding site to alter the binding affinity of poly-A binding protein (PABP) and analogs thereof. The incorporation of at least one engineered binding site may increase the binding affinity of the PABP and analogs thereof.

Additionally, multiple distinct mRNA may be linked together to the PABP (poly-A binding protein) through the 3′-end using alternative nucleotides at the 3′-terminus of the poly-A region. Transfection experiments can be conducted in relevant cell lines at and protein production can be assayed by ELISA at 12 hours, 24 hours, 48 hours, 72 hours, and day 7 post-transfection. As a non-limiting example, the transfection experiments may be used to evaluate the effect on PABP or analogs thereof binding affinity as a result of the addition of at least one engineered binding site. In certain cases, a poly-A region may be used to modulate translation initiation. While not wishing to be bound by theory, the poly-A region recruits PABP which in turn can interact with translation initiation complex and thus may be essential for protein synthesis. In some cases, a poly-A region may also be used in the present disclosure to protect against 3′-5′-exonuclease digestion. In some instances, an mRNA may include a polyA-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant mRNA may be assayed for stability, protein production and other parameters including half-life at various time points. It has been discovered that the polyA-G quartet results in protein production equivalent to at least 75% of that seen using a poly-A region of 120 nucleotides alone. In some cases, mRNA may include a poly-A region and may be stabilized by the addition of a 3′-stabilizing region. The mRNA with a poly-A region may further include a 5′-cap structure. In other cases, mRNA may include a poly-A-G Quartet. The mRNA with a poly-A-G Quartet may further include a 5′-cap structure. In some cases, the 3′-stabilizing region which may be used to stabilize mRNA includes a poly-A region or poly-A-G Quartet. In other cases, the 3′-stabilizing region which may be used with the present disclosure include a chain termination nucleoside such as 3′-deoxyadenosine (cordycepin), 3′-deoxyuridine, 3′-deoxycytosine, 3′-deoxyguanosine, 3′-deoxy thymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside. In other cases, mRNA which includes a polyA region or a poly-A-G Quartet may be stabilized by an alteration to the 3′-region of the polynucleotide that can prevent and/or inhibit the addition of oligo(U). In yet other instances, mRNA which includes a poly-A region or a poly-A-G Quartet may be stabilized by the addition of an oligonucleotide that terminates in a 3′-deoxynucleoside, 2′,3′-dideoxynucleoside 3-O-methylnucleosides, 3′-O-ethylnucleosides, 3′-arabinosides, and other alternative nucleosides known in the art and/or described herein.

In one aspect, an RNA disclosed herein comprises a poly-A tail comprising a sequence having at least, at most, exactly, or between any two of 99%, 98%, 97%, 96%, 95%, 90%, 85%, or 80% identity to SEQ ID NO: 90. In one aspects, the poly-A tail comprises a sequence of SEQ ID NO: 90.

RNA Transcription: In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include, e.g., but are not limited to mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, RNase inhibitor, pyrophosphatase, and/or T7 RNA polymerase. In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor may contain at least, at most, exactly, or between any two of 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration at least, at most, exactly, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg2+, Na+, Ca2+), RNA fragments, residual nucleotides, free phosphates, or any combinations thereof.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 pm. In some aspects, ultrafiltration membranes are typically classified by molecular weight cutoff (MWCO) rather than pore size. For example, the MWCO may be at least, at most, exactly, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for the ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to at least, at most, exactly, or between any two of 30 kDa, 40 kDa, 50 kDa, 60 kDa, 70 kDa, 80 kDa, 90 kDa, or more. In some aspects, a TFF membrane may have a MWCO equal to at least, at most, exactly, or between any two of 100 kDa, 150 kDa, 200 kDa, 250 kDa, 300 kDa, 350 kDa, 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to at least, at most, exactly, or between any two of 2, 3, 4, 5, 6, 7, 8, 9, 10, or more. In some aspects, the second diafiltration step is conducted with diavolumes equal to at least, at most, exactly, or between any two of 5, 6, 7, 8, 9, 10, 11, 12, 13, 14, 15, 16, 17, 18, 19, 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to at least, at most, exactly, or between any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size of at least, at most, exactly, or between any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

RNA Encapsulation: The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof.

Lipid nanoparticles may include a lipid component and one or more additional components, such as a therapeutic and/or prophylactic. A LNP may be designed for one or more specific applications or targets. The elements of a LNP may be selected based on a particular application or target, and/or based on the efficacy, toxicity, expense, ease of use, availability, or other feature of one or more elements. Similarly, the particular formulation of a LNP may be selected for a particular application or target according to, for example, the efficacy and toxicity of particular combinations of elements. The efficacy and tolerability of a LNP formulation may be affected by the stability of the formulation.

Lipid nanoparticles may be designed for one or more specific applications or targets. For example, a LNP may be designed to deliver a therapeutic and/or prophylactic such as an RNA to a particular cell, tissue, organ, or system or group thereof in a mammal's body.

Physiochemical properties of lipid nanoparticles may be altered in order to increase selectivity for particular bodily targets. For instance, particle sizes may be adjusted based on the fenestration sizes of different organs. The therapeutic and/or prophylactic included in a LNP may also be selected based on the desired delivery target or targets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a LNP may include an mRNA encoding a polypeptide of interest capable of being translated within a cell to produce the polypeptide of interest. Such a composition may be designed to be specifically delivered to a particular organ. In some embodiments, a composition may be designed to be specifically delivered to a mammalian liver. In some embodiments, a composition may be designed to be specifically delivered to a lymph node. In some embodiments, a composition may be designed to be specifically delivered to a mammalian spleen.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs may be designed to protect RNA molecules (e.g., mRNA) from extracellular RNases and/or may be engineered for systemic delivery of the RNA to target cells. In some aspects, such LNPs may be particularly useful to deliver RNA molecules (e.g., mRNA, modRNA) when RNA molecules are intravenously administered to a subject in need thereof. In some aspects, such LNPs may be particularly useful to deliver RNA molecules (e.g., mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., an E. coli FimH protein) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that may be used to deliver an active agent or therapeutic agent, such as a nucleic acid (e.g., mRNA, modRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA, modRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA, modRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, provided RNA molecules (e.g., mRNA, modRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” “size” or “mean size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

Lipid nanoparticles may be characterized by a variety of methods. For example, microscopy (e.g., transmission electron microscopy or scanning electron microscopy) may be used to examine the morphology and size distribution of a LNP. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a LNP, such as particle size, polydispersity index, and zeta potential.

The mean size of a LNP may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a LNP may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a LNP may be from about 70 nm to about 100 nm. In a particular embodiment, the mean size may be about 80 nm. In other embodiments, the mean size may be about 100 nm.

A LNP may be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. For example, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have, have at least, or have at least, at most, exactly, or between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein may be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than it is achieved in the absence of interactions between nucleic acids and at least one of the lipid components.

The efficiency of encapsulation of a therapeutic and/or prophylactic describes the amount of therapeutic and/or prophylactic that is encapsulated or otherwise associated with a LNP after preparation, relative to the initial amount provided. The encapsulation efficiency is desirably high (e.g., close to 100%). The encapsulation efficiency may be measured, for example, by comparing the amount of therapeutic and/or prophylactic in a solution containing the lipid nanoparticle before and after breaking up the lipid nanoparticle with one or more organic solvents or detergents. Fluorescence may be used to measure the amount of free therapeutic and/or prophylactic (e.g., RNA) in a solution. For the lipid nanoparticles described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A LNP may optionally comprise one or more coatings. For example, a LNP may be formulated in a capsule, film, or tablet having a coating. A capsule, film, or tablet including a composition described herein may have any useful size, tensile strength, hardness, or density.

Formulations comprising amphiphilic polymers and lipid nanoparticles may be formulated in whole or in part as pharmaceutical compositions. Pharmaceutical compositions may include one or more amphiphilic polymers and one or more lipid nanoparticles. For example, a pharmaceutical composition may include one or more amphiphilic polymers and one or more lipid nanoparticles including one or more different therapeutics and/or prophylactics. Pharmaceutical compositions may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, MD, 2006. Conventional excipients and accessory ingredients may be used in any pharmaceutical composition, except insofar as any conventional excipient or accessory ingredient may be incompatible with one or more components of a LNP or the one or more amphiphilic polymers in the formulation of the disclosure. An excipient or accessory ingredient may be incompatible with a component of a LNP or the amphiphilic polymer of the formulation if its combination with the component or amphiphilic polymer may result in any undesirable biological effect or otherwise deleterious effect.

In some embodiments, one or more excipients or accessory ingredients may make up greater than 50% of the total mass or volume of a pharmaceutical composition including a LNP. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia. Relative amounts of the one or more amphiphilic polymers, the one or more lipid nanoparticles, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more lipid nanoparticles. As another example, a pharmaceutical composition may comprise between 0.1% and 15% (wt/vol) of one or more amphiphilic polymers (e.g., 0.5%, 1%, 2.5%, 5%, 10%, or 12.5% w/v).

In certain embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more amphiphilic polymers and one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles by adding an effective amount of an amphiphilic polymer and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

The chemical properties of the LNP, LNP suspension, lyophilized LNP composition, or LNP formulation of the present disclosure may be characterized by a variety of methods. In some embodiments, electrophoresis (e.g., capillary electrophoresis) or chromatography (e.g., reverse phase liquid chromatography) may be used to examine the mRNA integrity.

In some embodiments, the LNP integrity of the LNP, LNP suspension, lyophilized LNP composition, or LNP formulation of the present disclosure is about 20% or higher, about 25% or higher, about 30% or higher, about 35% or higher, about 40% or higher, about 45% or higher, about 50% or higher, about 55% or higher, about 60% or higher, about 65% or higher, about 70% or higher, about 75% or higher, about 80% or higher, about 85% or higher, about 90% or higher, about 95% or higher, about 96% or higher, about 97% or higher, about 98% or higher, or about 99% or higher.

In some embodiments, the LNP integrity of the LNP, LNP suspension, lyophilized LNP composition, or LNP formulation of the present disclosure is higher than the LNP integrity of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more, about 10 folds or more, about 20 folds or more, about 30 folds or more, about 40 folds or more, about 50 folds or more, about 100 folds or more, about 200 folds or more, about 300 folds or more, about 400 folds or more, about 500 folds or more, about 1000 folds or more, about 2000 folds or more, about 3000 folds or more, about 4000 folds or more, about 5000 folds or more, or about 10000 folds or more.

In some embodiments, the Txo % of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the Txo % of the LNP, LNP suspension, lyophilized LNP composition, or LNP formulation of the present disclosure is longer than the Txo % of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more.

In some embodiments, the T1/2 of the LNP, LNP suspension, lyophilized LNP composition, or LNP formulation of the present disclosure is about 12 months or longer, about 15 months or longer, about 18 months or longer, about 21 months or longer, about 24 months or longer, about 27 months or longer, about 30 months or longer, about 33 months or longer, about 36 months or longer, about 48 months or longer, about 60 months or longer, about 72 months or longer, about 84 months or longer, about 96 months or longer, about 108 months or longer, about 120 months or longer.

In some embodiments, the T1/2 of the LNP, LNP suspension, lyophilized LNP composition, or LNP formulation of the present disclosure is longer than the T1/2 of the LNP, LNP suspension, lyophilized LNP composition, or LNP formulation produced by a comparable method by about 5% or higher, about 10% or more, about 15% or more, about 20% or more, about 30% or more, about 40% or more, about 50% or more, about 60% or more, about 70% or more, about 80% or more, about 90% or more, about 1 folds or more, about 2 folds or more, about 3 folds or more, about 4 folds or more, about 5 folds or more

As used herein, “Tx” refers to the amount of time lasted for the nucleic acid integrity (e.g., mRNA integrity) of a LNP, LNP suspension, lyophilized LNP composition, or LNP formulation to degrade to about X of the initial integrity of the nucleic acid (e.g., mRNA) used for the preparation of the LNP, LNP suspension, lyophilized LNP composition, or LNP formulation. For example, “T80%” refers to the amount of time lasted for the nucleic acid integrity (e.g., mRNA integrity) of a LNP, LNP suspension, lyophilized LNP composition, or LNP formulation to degrade to about 80% of the initial integrity of the nucleic acid (e.g., mRNA) used for the preparation of the LNP, LNP suspension, lyophilized LNP composition, or LNP formulation. For another example, “T1/2” refers to the amount of time lasted for the nucleic acid integrity (e.g., mRNA integrity) of a LNP, LNP suspension, lyophilized LNP composition, or LNP formulation to degrade to about ½ of the initial integrity of the nucleic acid (e.g., mRNA) used for the preparation of the LNP, LNP suspension, lyophilized LNP composition, or LNP formulation.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

The lipid component of a LNP may include, for example, a cationic lipid, a phospholipid (such as an unsaturated lipid, e.g., DOPE or DSPC), a polymer lipid (e.g. PEG lipid), and a structural lipid. The elements of the lipid component may be provided in specific fractions.

In some embodiments, the LNP further comprises a phospholipid, a PEG lipid, a structural lipid, or any combination thereof. Suitable phospholipids, PEG lipids, and structural lipids for the methods of the present disclosure are further disclosed herein.

In some embodiments, the lipid component of a LNP includes a cationic lipid, a phospholipid, a PEG lipid, and a structural lipid. In certain embodiments, the lipid component of the lipid nanoparticle includes about 30 mol % to about 60 mol % cationic lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the lipid nanoparticle includes about 35 mol % to about 55 mol % compound of cationic lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % said cationic lipid, about 10 mol % phospholipid, about 38.5 mol % structural lipid, and about 1.5 mol % of PEG lipid. In another particular embodiment, the lipid component includes about 40 mol % said cationic lipid, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

The amount of a therapeutic and/or prophylactic in a LNP may depend on the size, composition, desired target and/or application, or other properties of the lipid nanoparticle as well as on the properties of the therapeutic and/or prophylactic. For example, the amount of an RNA useful in a LNP may depend on the size, sequence, and other characteristics of the RNA. The relative amounts of a therapeutic and/or prophylactic (i.e. pharmaceutical substance) and other elements (e.g., lipids) in a LNP may also vary. In some embodiments, the wt/wt ratio of the lipid component to a therapeutic and/or prophylactic in a LNP may be from about 5:1 to about 60:1, such as5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a LNP may, for example, be measured using absorption spectroscopy (e.g., ultraviolet-visible spectroscopy).

Cationic Polymeric Materials: Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence orfragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

Lipids and Lipid-Like Materials: The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, the LNPs encompass, or encapsulate, the nucleic acid molecules.

Cationic Lipids: Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may be at least, at most, exactly, or between any two of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DM A), N-(2-Hydroxyethyl)-N,N-dimethyl-2,3-bis(tetradecyloxy)-1-propanaminium bromide (DMRIE), (±)—N-(3-aminopropyl)-N,N-dimethyl-2,3-bis(cis-9-tetradecenyloxy)-1-propanaminium bromide (GAP-DMORIE), (±)—N-(3-aminopropyl)-N,N-dimethyl-2,3-bis(dodecyloxy)-1-propanaminium bromide (GAP-DLRIE), (±)—N-(3-aminopropyl)-N,N-dimethyl-2,3-bis(tetradecyloxy)-I-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy)propan-1-aminium bromide (DMORIE), di((Z)-non-2-en-1-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-1-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid O2-200); or heptadecan-9-yl 8-((2-hydroxyethyl) (6-oxo-6-(undecyloxy)hexyl) amino) octanoate (SM-102).

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

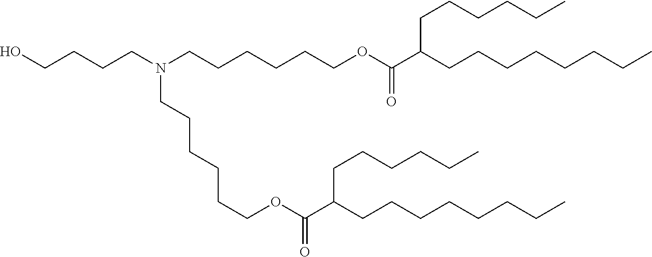 [SMILES: CCCCCCCCC(CCCCCC)C(=O)OCCCCCCN(CCCCO)CCCCCCOC(=O)C(CCCCCC)CCCCCCCC]

Cationic lipids are disclosed in, e.g., U.S. Pat. No. 10,166,298, the full disclosures of which are herein incorporated by reference in their entirety for all purposes. Representative cationic lipids include:

| No. | Structure |
|---|---|
| 1 | 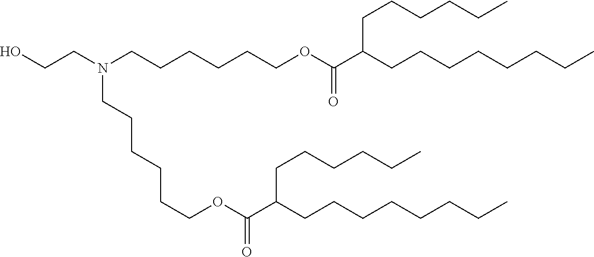 [SMILES: CCCCCCCCC(CCCCCC)C(=O)OCCCCCCN(CCO)CCCCCCOC(=O)C(CCCCCC)CCCCCCCC] |
| 2 | 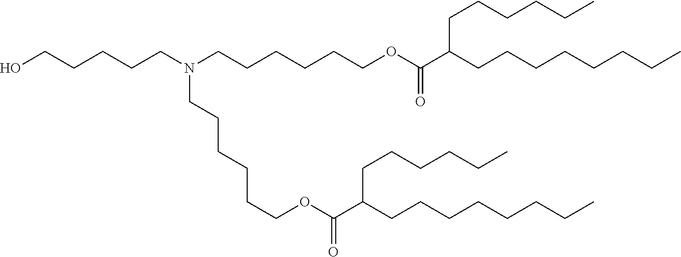 [SMILES: CCCCCCCCC(CCCCCC)C(=O)OCCCCCCN(CCCCCO)CCCCCCOC(=O)C(CCCCCC)CCCCCCCC] |
| 3 | 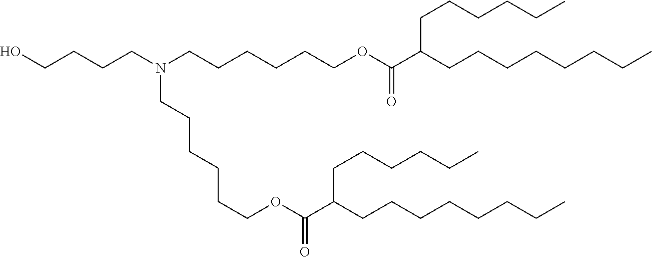 [SMILES: CCCCCCCCC(CCCCCC)C(=O)OCCCCCCN(CCCCO)CCCCCCOC(=O)C(CCCCCC)CCCCCCCC] |
| 4 | 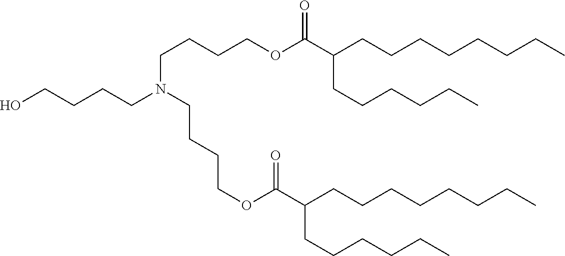 [SMILES: CCCCCCCCC(CCCCCC)C(=O)OCCCCN(CCCCO)CCCCOC(=O)C(CCCCCC)CCCCCCCC] |
| 5 | 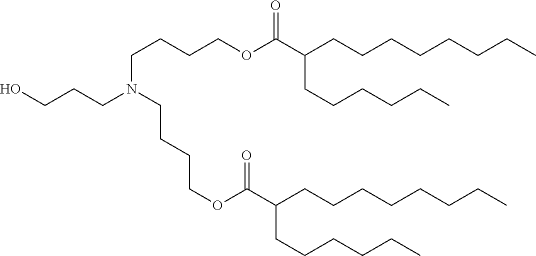 [SMILES: CCCCCCCCC(CCCCCC)C(=O)OCCCCN(CCCO)CCCCOC(=O)C(CCCCCC)CCCCCCCC] |
| 6 | 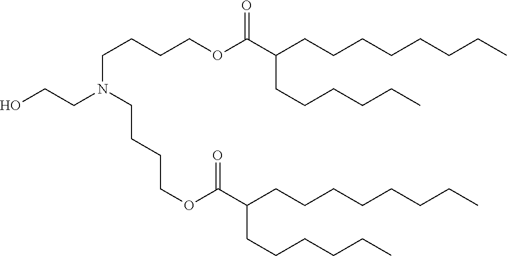 [SMILES: CCCCCCCCC(CCCCCC)C(=O)OCCCCN(CCO)CCCCOC(=O)C(CCCCCC)CCCCCCCC] |
| 7 | 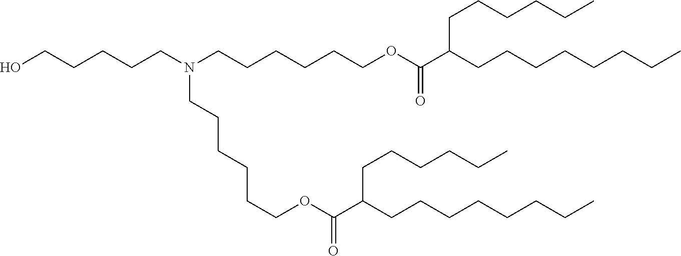 [SMILES: CCCCCCCCC(CCCCCC)C(=O)OCCCCCCN(CCCCCO)CCCCCCOC(=O)C(CCCCCC)CCCCCCCC] |
| 8 | 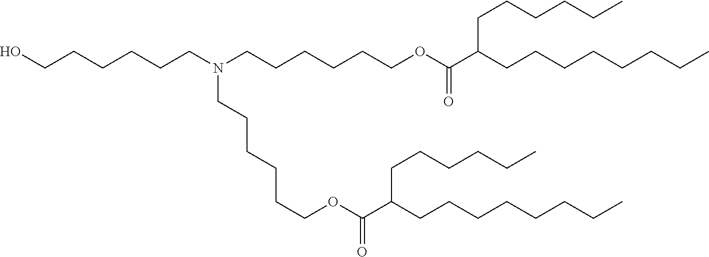 [SMILES: CCCCCCCCC(CCCCCC)C(=O)OCCCCCCN(CCCCCCO)CCCCCCOC(=O)C(CCCCCC)CCCCCCCC] |
| 9 | 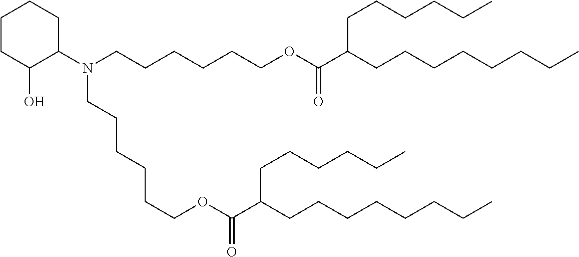 [SMILES: CCCCCCCCC(CCCCCC)C(=O)OCCCCCCN(CCCCCCOC(=O)C(CCCCCC)CCCCCCCC)C1CCCCC1O] |
| 10 | 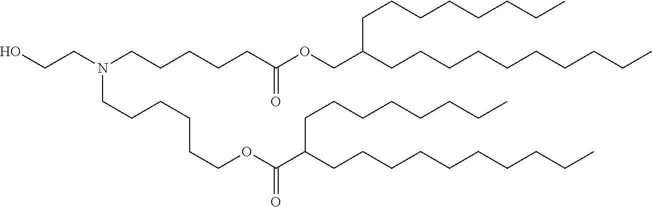 [SMILES: CCCCCCCCCCC(CCCCCCCC)COC(=O)CCCCCN(CCO)CCCCCCOC(=O)C(CCCCCCCC)CCCCCCCCCC] |
| 11 | 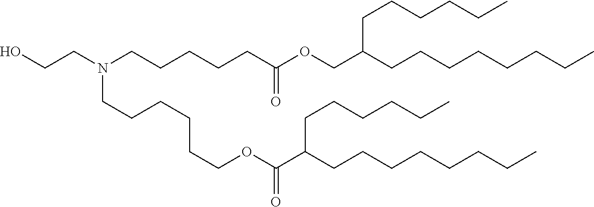 [SMILES: CCCCCCCCC(CCCCCC)COC(=O)CCCCCN(CCO)CCCCCCOC(=O)C(CCCCCC)CCCCCCCC] |
| 12 | 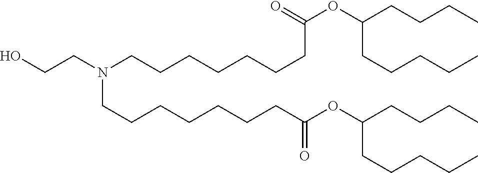 [SMILES: CCCCCC(CCCC)OC(=O)CCCCCCCN(CCO)CCCCCCCC(=O)OC(CCCC)CCCCC] |
| 13 | 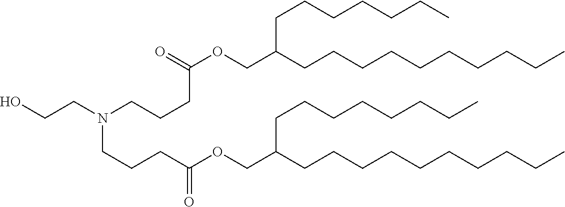 [SMILES: CCCCCCCCCCC(CCCCCCC)COC(=O)CCCN(CCO)CCCC(=O)OCC(CCCCCCCC)CCCCCCCCCC] |
| 14 | 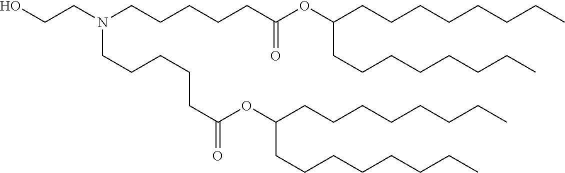 [SMILES: CCCCCCCCC(CCCCCCCC)OC(=O)CCCCCN(CCO)CCCCCC(=O)OC(CCCCCCCC)CCCCCCCC] |
| 15 | 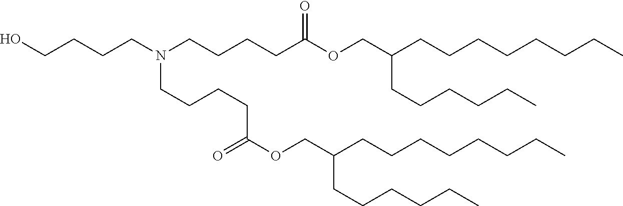 [SMILES: CCCCCCCCC(CCCCCC)COC(=O)CCCCN(CCCCO)CCCCC(=O)OCC(CCCCCC)CCCCCCCC] |
| 16 | 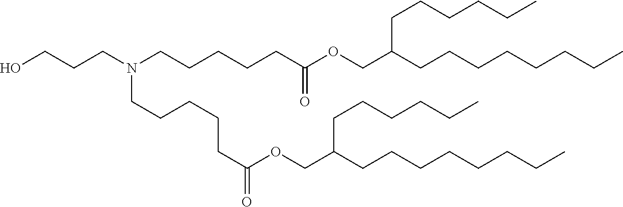 [SMILES: CCCCCCCCC(CCCCCC)COC(=O)CCCCCN(CCCO)CCCCCC(=O)OCC(CCCCCC)CCCCCCCC] |
| 17 | 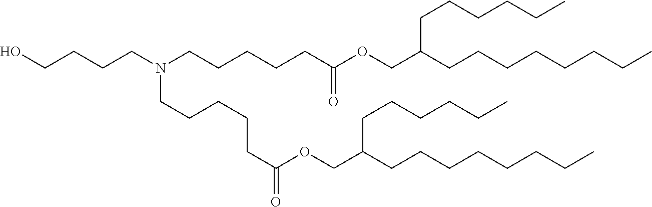 [SMILES: CCCCCCCCC(CCCCCC)COC(=O)CCCCCN(CCCCO)CCCCCC(=O)OCC(CCCCCC)CCCCCCCC] |
| 18 | 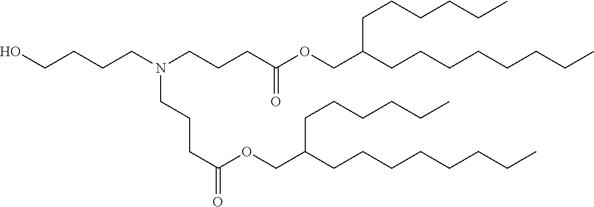 [SMILES: CCCCCCCCC(CCCCCC)COC(=O)CCCN(CCCCO)CCCC(=O)OCC(CCCCCC)CCCCCCCC] |
| 19 | 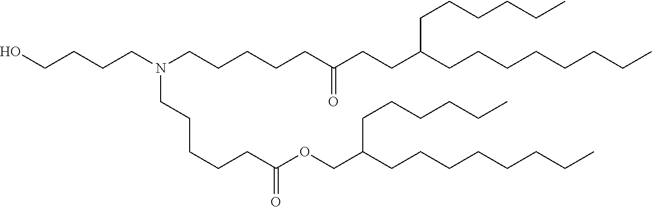 [SMILES: CCCCCCCCC(CCCCCC)CCC(=O)CCCCCN(CCCCO)CCCCCC(=O)OCC(CCCCCC)CCCCCCCC] |
| 20 | 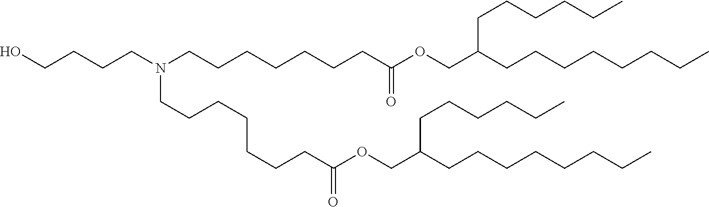 [SMILES: CCCCCCCCC(CCCCCC)COC(=O)CCCCCCCN(CCCCO)CCCCCCCC(=O)OCC(CCCCCC)CCCCCCCC] |
| 21 | 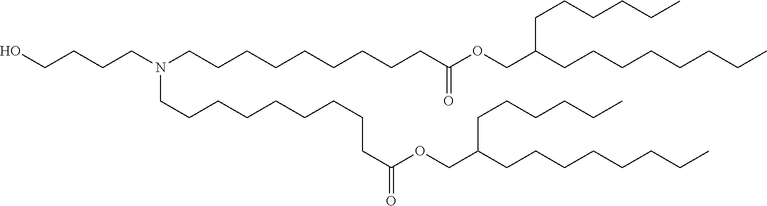 [SMILES: CCCCCCCCC(CCCCCC)COC(=O)CCCCCCCCCN(CCCCO)CCCCCCCCCC(=O)OCC(CCCCCC)CCCCCCCC] |
| 22 | 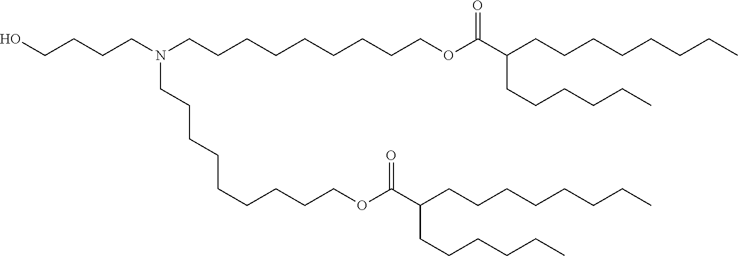 [SMILES: CCCCCCCCC(CCCCCC)C(=O)OCCCCCCCCCN(CCCCO)CCCCCCCCCOC(=O)C(CCCCCC)CCCCCCCC] |
| 23 | 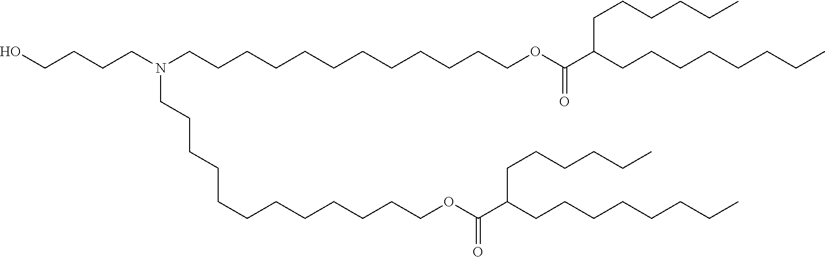 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 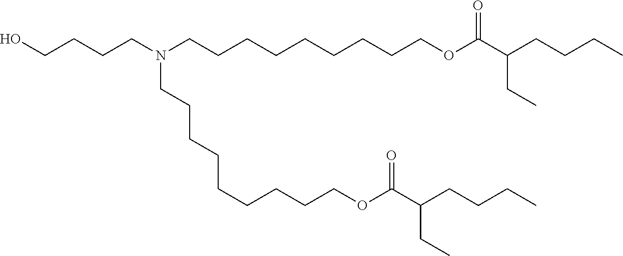 [SMILES: CCCCC(CC)C(=O)OCCCCCCCCCN(CCCCO)CCCCCCCCCOC(=O)C(CC)CCCC] |
| 25 | 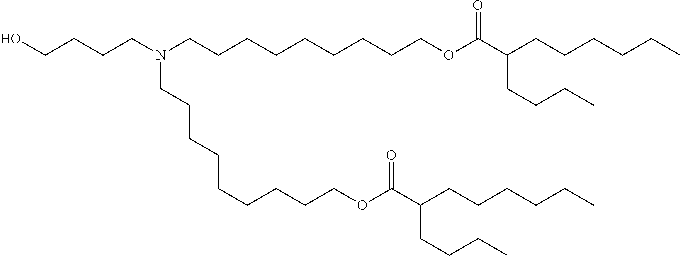 [SMILES: CCCCCCC(CCCC)C(=O)OCCCCCCCCCN(CCCCO)CCCCCCCCCOC(=O)C(CCCC)CCCCCC] |
| 26 | 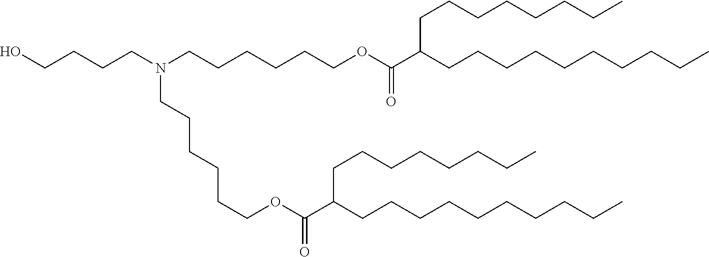 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 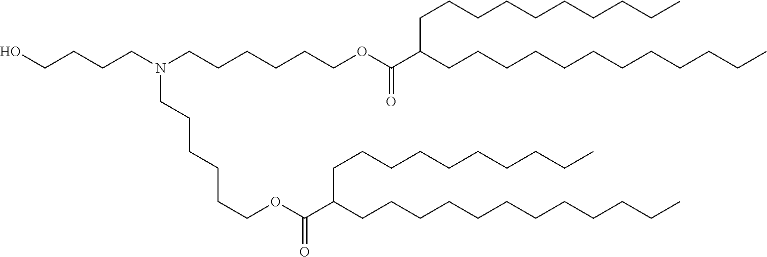 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| 28 | 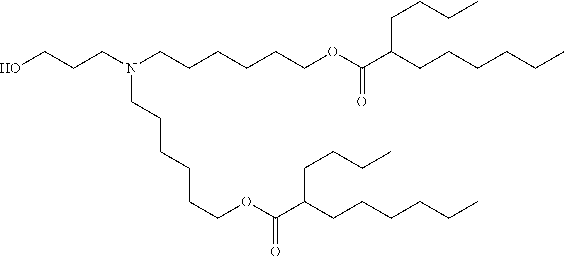 [SMILES: CCCCCCC(CCCC)C(=O)OCCCCCCN(CCCO)CCCCCCOC(=O)C(CCCC)CCCCCC] |
| 29 | 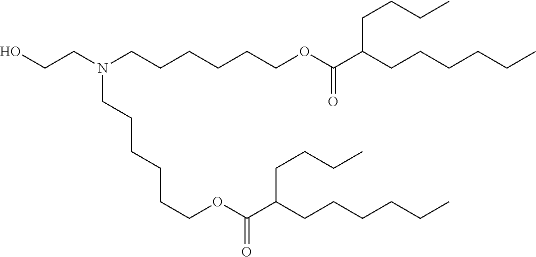 [SMILES: CCCCCCC(CCCC)C(=O)OCCCCCCN(CCO)CCCCCCOC(=O)C(CCCC)CCCCCC] |
| 30 | 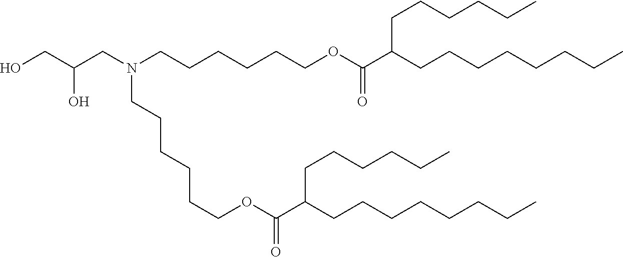 [SMILES: CCCCCCCCC(CCCCCC)C(=O)OCCCCCCN(CCCCCCOC(=O)C(CCCCCC)CCCCCCCC)CC(O)CO] |
| 31 | 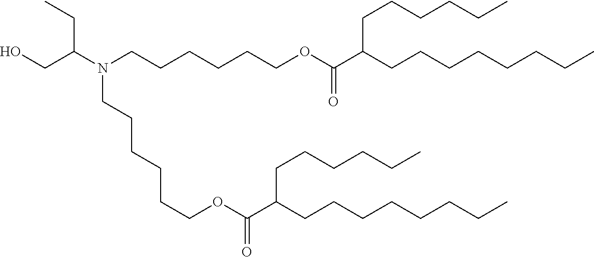 [SMILES: CCCCCCCCC(CCCCCC)C(=O)OCCCCCCN(CCCCCCOC(=O)C(CCCCCC)CCCCCCCC)C(CC)CO] |
| 32 | 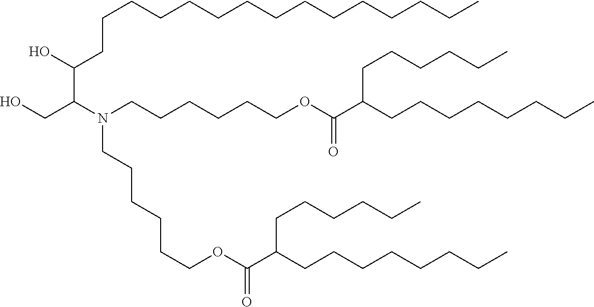 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| 33 | 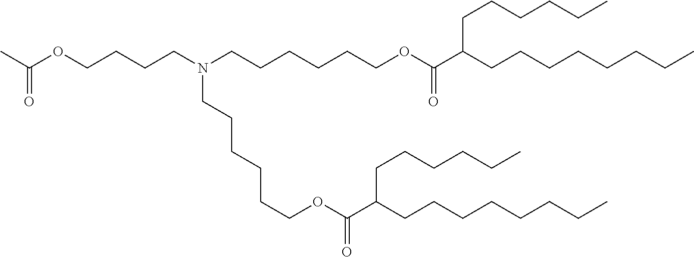 [SMILES: CCCCCCCCC(CCCCCC)C(=O)OCCCCCCN(CCCCCCOC(=O)C(CCCCCC)CCCCCCCC)CCCCOC(C)=O] |
| 34 | 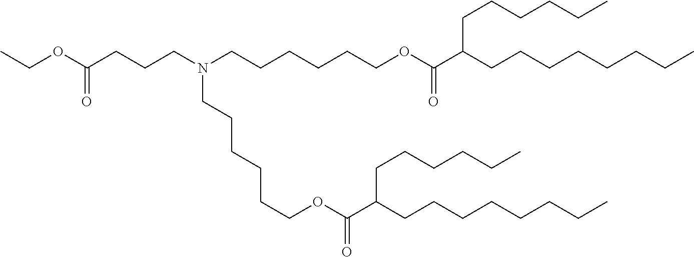 [SMILES: CCCCCCCCC(CCCCCC)C(=O)OCCCCCCN(CCCCCCOC(=O)C(CCCCCC)CCCCCCCC)CCCC(=O)OCC] |
| 35 | 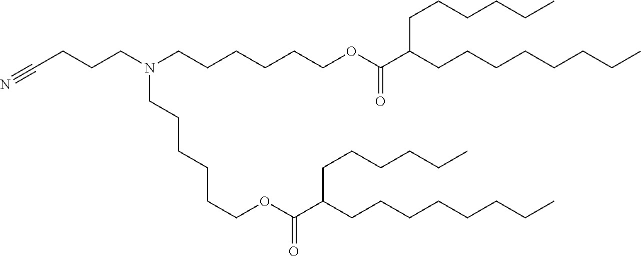 [SMILES: CCCCCCCCC(CCCCCC)C(=O)OCCCCCCN(CCCC#N)CCCCCCOC(=O)C(CCCCCC)CCCCCCCC] |
| 36 | 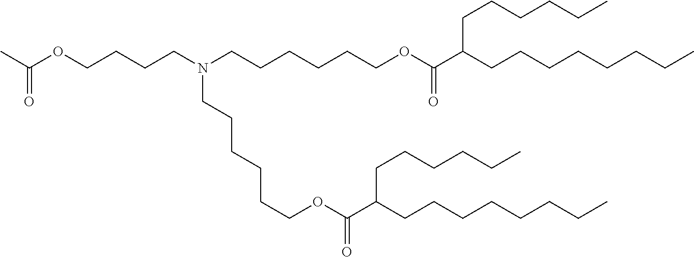 [SMILES: CCCCCCCCC(CCCCCC)C(=O)OCCCCCCN(CCCCCCOC(=O)C(CCCCCC)CCCCCCCC)CCCCOC(C)=O] |

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between any two of about 40, 41, 42, 43, 44, 45, 46, 47, 48, 49, 50, 51, 52, 53, 54, 55, 56, 57, 58, 59 or 60 mole percent, respectively.

In some aspects of the disclosure the LNP comprises a combination or mixture of any the lipids described above.

Polymer Conjugated Lipid: In some aspects, the LNPs comprise a polymer conjugated lipid. The term “polymer conjugated lipid” or “polymer lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]—N,N-ditetradecylacetamide, and the like.

In certain aspects, the LNP comprises an additional, stabilizing-lipid which is a polyethylene glycol-lipid (pegylated lipid). A polymer conjugated lipid (e.g. PEG-lipid) refers to a molecule comprising both a lipid portion and a polymer portion. An example of a polymer conjugated lipid is a PEG-lipid. A PEG-lipid refers to a molecule comprising both a lipid portion and a polyethylene glycol portion. PEG-lipids include, but are not limited to, PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one aspect, the polyethylene glycol-lipid is N-[(methoxy polyethylene glycol)2000)carbamyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. PEG-lipids are disclosed in, e.g., U.S. Pat. No. 9,737,619, the full disclosures of which are herein incorporated by reference in their entirety for all purposes.

In some aspects, the lipid nanoparticles comprise a polymer conjugated lipid. In one aspect, the lipid nanoparticle comprises 2-[(polyethylene glycol)-2000]—N,N-ditetradecylacetamide (ALC-O159), having the formula:

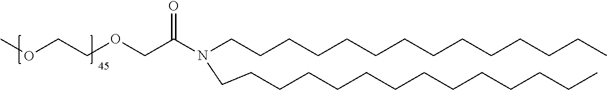

In another aspect, a polymer may be selected from, but is not limited to, polyamines, polyethers, polyamides, polyesters, poly carbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(gly colic acid) (PGA), poly(lactic acid-co-gly colic acid) (PLGA), poly(L-lactic acid-co-gly colic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

In various aspects, the molar ratio of the cationic lipid to the pegylated lipid or polymer lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid or polymer lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle.

Additional Lipids: In certain aspects, the LNP comprises one or more additional lipids or lipid-like materials that stabilize the formation of particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DpyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC), having the formula:

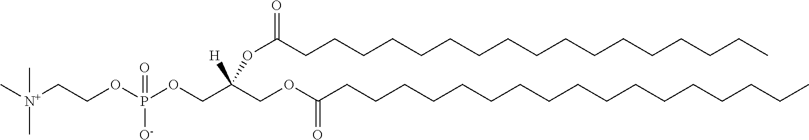 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

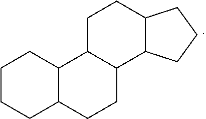 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In one aspect, the cholesterol has the formula:

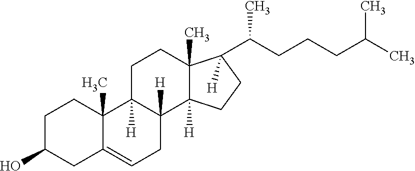

Without being bound by any theory, the amount ofthe at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from about 2:1 to about 8:1, or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle. In some aspects, the non-cationic lipid, e.g., neutral lipid (e.g., one or more phospholipids and/or cholesterol), may be at least, at most, exactly, or between any two of 0 mol %, 10 mol %, 20 mol %, 30 mol %, 40 mol %, 50 mol %, 60 mol %, 70 mol %, 80 mol %, or 90 mol % of the total lipid present in the particle.

Other Materials: Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A LNP may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, surface active agents, buffering agents, preservatives, and other species.

Surface active agents and/or emulsifiers may include, but are not limited to, natural emulsifiers (e.g., acacia, alginic acid, sodium alginate, cholesterol, and lecithin), sorbitan fatty acid esters (e.g., polyoxy ethylene sorbitan monolaurate [TWEEN®20], polyoxy ethylene sorbitan [TWEEN®60], polyoxy ethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER®188, cetrimonium bromide, cetylpyridinium chloride, benzalkonium chloride, docusate sodium, and/or combinations thereof.

Examples of preservatives may include, but are not limited to, antioxidants, chelating agents, free radical scavengers, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxy toluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxy toluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN® II, NEOLONE™, KATHON™, and/or EUXYL®. An exemplary free radical scavenger includes butylated hydroxytoluene (BHT or butylhydroxytoluene) or deferoxamine.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In some embodiments, the formulation including a LNP may further include a salt, such as a chloride salt. In some embodiments, the formulation including a LNP may further includes a sugar such as a disaccharide. In some embodiments, the formulation further includes a sugar but not a salt, such as a chloride salt. In some embodiments, a LNP may further include one or more small hydrophobic molecules such as a vitamin (e.g., vitamin A or vitamin E) or a sterol. Carbohydrates may include simple sugars (e.g., glucose) and polysaccharides (e.g., glycogen and derivatives and analogs thereof).

The characteristics of a LNP may depend on the components thereof. For example, a LNP including cholesterol as a structural lipid may have different characteristics than a LNP that includes a different structural lipid. As used herein, the term “structural lipid” refers to sterols and also to lipids containing sterol moieties. As defined herein, “sterols” are a subgroup of steroids consisting of steroid alcohols. In some embodiments, the structural lipid is a steroid. In some embodiments, the structural lipid is cholesterol. In some embodiments, the structural lipid is an analog of cholesterol. In some embodiments, the structural lipid is alpha-tocopherol.

In some embodiments, the characteristics of a LNP may depend on the absolute or relative amounts of its components. For instance, a LNP including a higher molar fraction of a phospholipid may have different characteristics than a LNP including a lower molar fraction of a phospholipid. Characteristics may also vary depending on the method and conditions of preparation of the lipid nanoparticle. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Particular phospholipids can facilitate fusion to a membrane. In some embodiments, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue. Non-natural phospholipid species including natural species with modifications and substitutions including branching, oxidation, cyclization, and alkynes are also contemplated. In some embodiments, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye). Phospholipids include, but are not limited to, glycerophospholipids such as phosphatidylcholines, phosphatidyl-ethanolamines, phosphatidylserines, phosphatidylinositols, phosphatidy glycerols, and phosphatidic acids. Phospholipids also include phosphosphingolipid, such as sphingomyelin. In some embodiments, a phospholipid useful or potentially useful in the present invention is an analog or variant of DSPC.

Characterization and Analysis of RNA Molecule: The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a 5′ cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest (e.g., an antigen) is at least 2-fold to at least 10-fold. In some aspects, elevated expression of a gene of interest (e.g., an antigen) is at least 2-fold. In some aspects, elevated expression of a gene of interest (e.g., an antigen) is at least 3-fold. In some aspects, elevated expression of a gene of interest (e.g., an antigen) is at least 4-fold. In some aspects, elevated expression of a gene of interest (e.g., an antigen) is at least 6-fold. In some aspects, elevated expression of a gene of interest (e.g., an antigen) is at least 8-fold. In some aspects, elevated expression of a gene of interest (e.g., an antigen) is at least 10-fold.

In some aspects, elevated expression of a gene of interest (e.g., an antigen) is about 2-fold to about 50-fold. In some aspects, elevated expression of a gene of interest (e.g., an antigen) is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest (e.g., an antigen) is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest (e.g., an antigen) persists for at least, at most, exactly, or between any two of 24 hours, 36 hours, 48 hours, 60 hours, 72 hours, 84 hours, 96 hours, 108 hours, or 120 hours, or any range or value derivable therein.

### Uses of the Compositions

In one aspect, the disclosure provides compositions comprising glycoconjugates for use in eliciting an immune response against E. coli or K. pneumoniae in a subject. In another aspect, the disclosure provides compositions comprising glycoconjugates for preventing E. coli or K. pneumoniae infection in a subject. In other embodiments, the disclosure provides compositions comprising glycoconjugates for use in the manufacture of a medicament for eliciting an immune response against E. coli or K. pneumoniae, or for preventing E. coli or K. pneumoniae infection in a subject.

In another aspect, the disclosure provides compositions comprising nucleic acids for use in eliciting an immune response against E. coli or K. pneumoniae in a subject. In some embodiments, the disclosure provides compositions comprising RNA for use in eliciting an immune response against E. coli or K. pneumoniae in a subject. In another aspect, the disclosure provides compositions comprising nucleic acids for preventing E. coli or K. pneumoniae infection in a subject. In some embodiments, the disclosure provides compositions comprising RNA for preventing E. coli or K. pneumoniae infection in a subject. In other embodiments, the disclosure provides compositions comprising nucleic acids for use in the manufacture of a medicament for eliciting an immune response against E. coli or K. pneumoniae, or for preventing E. coli or K. pneumoniae infection in a subject. In some embodiments, the disclosure provides compositions comprising RNA for use in the manufacture of a medicament for eliciting an immune response against E. coli or K. pneumoniae, or for preventing E. coli or K. pneumoniae infection in a subject.

In other aspects, the present disclosure provides a method of eliciting an immune response against E. coli or K. pneumoniae in a subject, such as a human, comprising administering to the subject an effective amount of a composition comprising a glycoconjugate described herein. The present disclosure also provides a method of preventing E. coli or K. pneumoniae infection in a subject, comprising administering to the subject an effective amount of a pharmaceutical composition, such as a vaccine, comprising a glycoconjugate described herein.

In another aspect, the present disclosure provides a method of eliciting an immune response against E. coli or K. pneumoniae in a subject, such as a human, comprising administering to the subject an effective amount of a composition comprising a nucleic acid described herein. In some embodiments, the present disclosure provides a method of eliciting an immune response against E. coli or K. pneumoniae in a subject, such as a human, comprising administering to the subject an effective amount of a composition comprising an RNA molecule described herein. The present disclosure also provides a method of preventing E. coli or K. pneumoniae infection in a subject, comprising administering to the subject an effective amount of a pharmaceutical composition, such as a vaccine, comprising a nucleic acid described herein. The present disclosure also provides a method of preventing E. coli or K. pneumoniae infection in a subject, comprising administering to the subject an effective amount of a pharmaceutical composition, such as a vaccine, comprising a RNA molecule described herein.

As used herein, “subject” means a mammal, in particular a human. In some particular embodiments, the human is a child, such as an infant. In some other particular embodiments, the human is a woman. The compositions of the disclosure may be administered to the subject with or without administration of an adjuvant. The effective amount administered to the subject is an amount that is sufficient to elicit an immune response against an E. coli or K. pneumoniae antigen in the subject. Subjects that can be selected for treatment include those that are at risk for developing an E. coli or K. pneumoniae infection because of exposure or the possibility of exposure to E. coli or K. pneumoniae. Because humans may be infected with E. coli or K. pneumoniae by the age of 2, the entire birth cohort is included as a relevant population for immunization. This could be done, for example, by beginning an immunization regimen anytime from birth to 6 months of age, from 6 months of age to 5 years of age, in pregnant women (or women of child-bearing age) to protect their infants by passive transfer of antibody, infants still in utero, and subjects greater than 50 years of age.

In some embodiments, a subject that is administered a composition of the disclosure is an adult. In some embodiments, a subject that is administered a composition of the disclosure is at least 16, 17, 18, 19, 20, 21, 22, 23, 24, or 25 years of age. In a preferred embodiment, the subject is at least 18 years of age. In some embodiments, the subject is less than 80, 75, 70, 65, 60, 55, or 50 years of age. In some embodiments, the subject is less than 70, 69, 68, 67, 66, 65, 64, 63, 62, 61, or 60 years of age. In a preferred embodiment, the subject is less than 65 years of age. In some embodiments, the subject is between 16, 17, 18, 19, 20, 21, 22, 23, 24, or 25 years of age and 70, 69, 68, 67, 66, 65, 64, 63, 62, 61, or 60 years of age. In a preferred embodiment, the subject is between 18 and 64 years of age.

In some embodiments, a subject that is administered compositions of the disclosure has a history of infections related to E. coli or K. pneumoniae. In a preferred embodiment, the subject that is administered a composition of the disclosure has a history of frequent urinary tract infections (UTIs).

Administration of the compositions provided by the present disclosure, such as pharmaceutical compositions, can be carried out using standard routes of administration. Non-limiting embodiments include parenteral administration, such as intradermal, intramuscular, subcutaneous, transcutaneous, mucosal, or oral administration.

The total dose of the composition provided to a subject during one administration can be varied as is known to the skilled practitioner.

It is also possible to provide one or more booster administrations of one or more of the vaccine compositions. If a boosting vaccination is performed, typically, such a boosting vaccination will be administered to the same subject at a moment between one week and 10 years, for example between two weeks and six months, after administering the composition to the subject for the first time (which is in such cases referred to as “priming vaccination”).

In certain embodiments, the administration comprises a priming administration and at least one booster administration. In certain other embodiments, the administration is provided annually. In still other embodiments, the administration is provided annually together with an influenza vaccine.

In some embodiments, the subject will receive multiple doses of the composition comprising a glycoconjugate described herein. In a particular embodiment, the subject is provided 2 doses of the composition comprising a glycoconjugate. In another particular embodiment, the subject is provided 3 doses of the composition comprising a glycoconjugate. In some embodiments, the second dose is administered between about two weeks and about 2 years after the first dose. For example, the second dose can be administered about 3 months, about 6 months, about 1 year, or about 2 years after the first dose. In some embodiments, the third dose is administered between about two weeks and about 2 years after the second dose. For example, the third dose can be administered about 3 months, about 6 months, about 1 year, or about 2 years after the second dose.

The vaccines provided by the present disclosure may be used together with one or more other vaccines. For example, in adults they may be used together with an influenza vaccine, Prevnar, tetanus vaccine, diphtheria vaccine, and/or pertussis vaccine. For pediatric use, vaccines provided by the present disclosure may be used with any other vaccine indicated for pediatric patients.

### EXAMPLES

In order that this disclosure may be better understood, the following examples are set forth. These examples are for purposes of illustration only and are not to be construed as limiting the scope of the disclosure in any manner. The following Examples illustrate some embodiments of the disclosure.

### Example 1: Synthesis of E. Coli O25B Polysaccharide-SCP Conjugate Using Click Chemistry

The following three steps are used to synthesize the E. coli O25b polysaccharide-SCP conjugate using click chemistry.

Step 1: Activation of E. coli O25b Polysaccharide with Azido Linker

To begin the process of activation of E. coli O25b polysaccharide with azido linker, E. coli. O25b polysaccharide was mixed with imidazole and then frozen and lyophilized. After lyophilization, the lyophilized polysaccharide was reconstituted with anhydrous DMSO. The reaction mixture was then warmed to 40° C., and 1,1′-carbonyldiimidazole (CDI) was added. The reaction mixture was stirred at 40° C. for 3 hrs. After the reaction mixture was cooled to 23° C., WFI (2% v/v) was added to quench free CDI and then the reaction mixture was stirred further for 30 min at 23° C. 3-azido-1-propylamine was added to the reaction mixture. After 20 hrs at 40° C., the reaction mixture was diluted using chilled 10 mM sodium phosphate buffer in saline (pH 7) (5×, v/v) at a temperature of 5° C. The diluted reaction mixture was then purified by ultrafiltration/diafiltration (UF/DF) using a 5K molecular weight cut off (MWCO) regenerated cellulose (RC) membrane against 10 mM sodium phosphate buffer in saline (pH 7) (30× diavolume).

Step 2: Activation of SCP to Alkyne-SCP with Alkyne NHS Ester

To begin the process of activation of SCP to alkyne-SCP with alkyne NHS ester, WFI and 0.5 M sodium phosphate buffer (pH 8.3) were added to the SCP solution to result in a 4 mg/mL concentration with a buffer strength of 0.1 M. After cooling the reaction mixture to 8° C., 3-propargyloxy-propanoic acid NHS ester was added dropwise while maintaining the reaction temperature at 8±3° C. Next, the reaction mixture was stirred for 30 min at 8° C., purified by UF/DF using a 10K MWCO polyethersulfone (PES) membrane (Millipore® Pellicon® 2 Mini) against 100 mM sodium phosphate buffer in saline (pH 7.0) (30× diavolume).

### Step 3: Click Conjugation: Activated Azido Poly and Alkyne SCP are Conjugated by a Cu+1 Mediated Azide-Alkyne Cycloaddition Reaction

In a process referred to as the “click reaction” (illustrated in FIG. 1), activated azido poly and alkyne SCP were conjugated by a Cu+1 mediated azide-alkyne cycloaddition reaction. To being the process, a mixture of 5 mM copper sulfate (CuSO₄) and 25 mM Tris(3hydroxypropyltriazolylmethyl)amine (THPTA) (1:1, v/v) was added to the mixture of E. coli O25b polysaccharide activated with azido linker (see step 1 above) and alkyne-SCP (see step 2 above) (in 100 mM Sodium Phosphate Buffer (SPB) in saline, pH 7.0) at 23° C. and followed by the addition of 100 mM aminoguanidine (at the same volume as the CuSO4-THPTA mixture) and 100 mM sodium ascorbate (at the same volume as the CuSO4-THPTA mixture). Next, the reaction mixture was stirred for 3 hours at 23° C., before the first quenching reaction, half of the amount of the CuSO4-THPTA, aminoguanidine, and sodium ascorbate solutions were added. Subsequently, the unreacted azido group was capped by propargyl alcohol for 1 hours at 23° C. After the first capping, the unreacted alkyne group was capped by 3-azido-1-propanol for 1 hour at 23° C. Next, the reaction mixture was purified by UF/DF using 100K MWCO PES membrane against 10 mM EDTA+10 mM SPB in saline (pH 7.0) (30× diavolume), followed by 5 mM succinate in saline (pH 6.0) (30× diavolume).

### Example 2: In Vivo Immune Response Comparison of O25B-Scp (Click Chemistry) and O25B-CRM (RAC/DMSO)

In this Example, the in vivo immune response to a vaccine comprising E. coli O25b conjugated to SCP using click chemistry was compared to a vaccine comprising E. coli O25b conjugated to CRM₁₉₇ using RAC/DMSO. The click-SCP conjugate vaccine was synthesized by a click conjugation method that conjugated polysaccharide antigens to a carrier protein (SCP) in a highly controlled and site-specific manner which reduced conjugation heterogeneity. The Click conjugate was cross-linked through linker (or spacer). In contrast, a RAC/DMSO conjugate was cross-linked directly. Furthermore, the click-SCP conjugation method required optimization of the degree of activation (DoA) parameters for the polysaccharide (with 3-Azido-1-propylamine (APA) linker), as well as the SCP carrier protein (with NHS ester linker). DoA was controlled by the milliequivalent (MEq) amount of CDI or linker added.

The specifications for the E. coli O25b-SCP conjugates are presented in Table 3 below. The specifications for the E. coli O25b-CRM conjugates are presented in Table 4 below.

TABLE 3
O25b Click-SCP Conjugates

| Conjugate Variant | Click-SCP | Click-SCP |
|---|---|---|
| Azido poly yield (%) | 63 | 61 |
| Azido poly Mw (kDa) | 63 | 160 |
| DOA of azido poly | ~6-10% | 20-24% |
| DOA of alkyne-SCP | 21 | 21 |
| Input ratio | 1 | 1 |

Conjugate Data

| | | |
|---|---|---|
| Conjugation Scale | 100 mg | 100 mg |
| Conjugate Yield (%) | 66 | 80 |
| SPRatio | 0.82 | 0.83 |
| Free Sacc (%) | <5% | <5% |
| Conjugate MW (kDa) | 843 | 1245 |
| Endotoxin (EU/ug) | 0.007 | 0.005 |
| Poly Conc (mg/mL) | 0.63 | 0.77 |
| Protein Conc (mg/mL) | 0.77 | 0.93 |

TABLE 4
O25b RAC-CRM Conjugates

| Conjugate Variant | RAC-CRM |
|---|---|
| Poly MW (kDa) | 49.8 |
| DO | 15.8 |
| Act poly Mw (kDa) | 49.9 |

Conjugate Data

| | |
|---|---|
| Yield (%) | 56 |
| SPRatio | 0.96 |
| Free Sacc (%) | 6.6 |
| Conjugate MW (kDa) | 5888 |
| Sacc Conc (mg/mL) | 0.48 |
| Protein Conc (mg/mL) | 0.5 |
| Endotoxin (EU/ug) | <0.004 |
| Conjugate (DS) Matrix | 5 mM Succ/Saline, pH 6.0 |

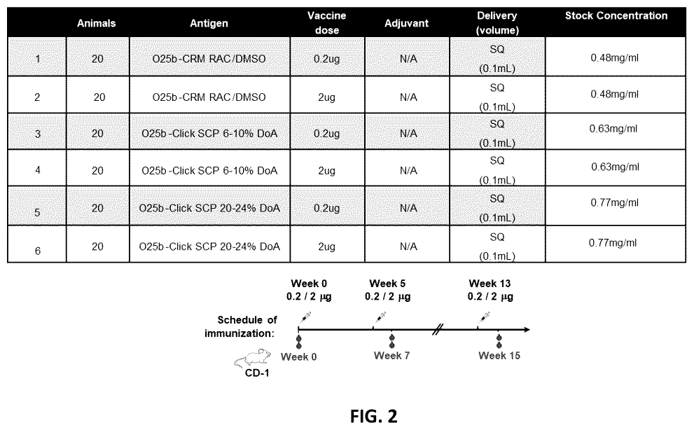

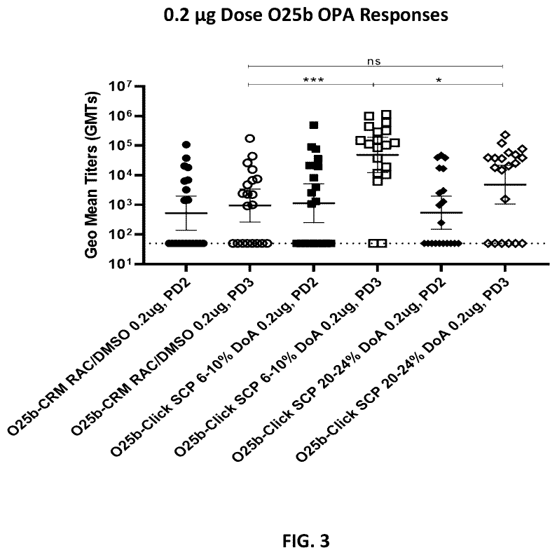

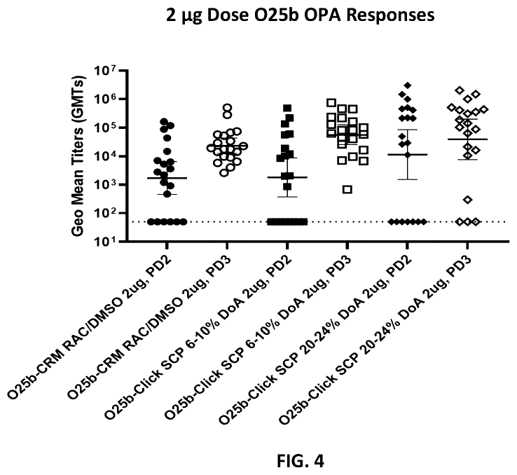

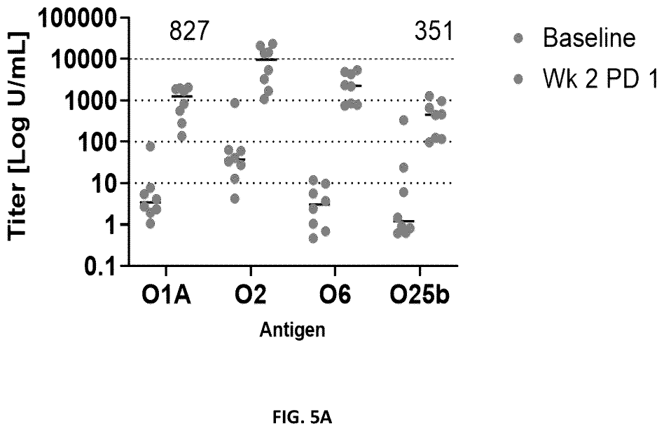

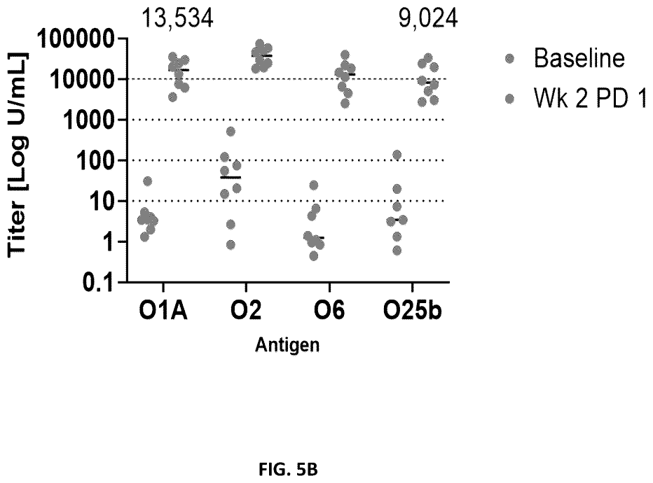

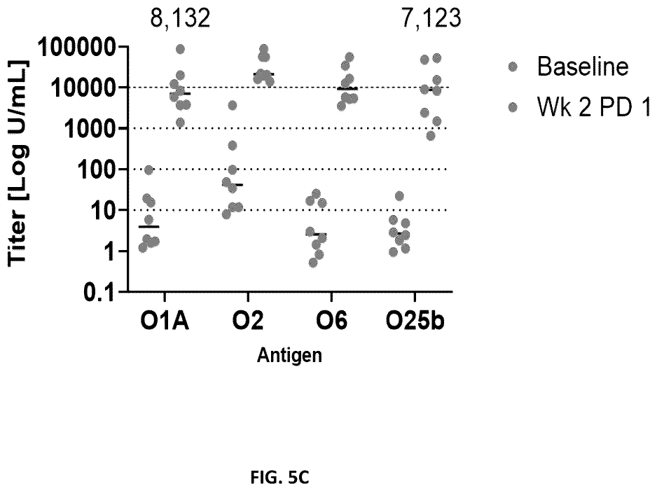

An opsonophagocytic assay (OPA) of mice subcutaneously administered vaccine compositions was completed to assess the O25b-SCP and O25b-CRM₁₉₇ lattice platform chemistries. FIG. 2 presents a schematic of the schedule of immunization for the mouse study (VAC-2023-PRL-EC-246). As shown in FIG. 2, CD-1 mice were provided subcutaneous (SQ) injections of the O25b-containing immunogenic compositions at week 0, week 5, and week 13. Each test group included 20 mice. The test groups included 0.2 μg O25b-CRM RAC/DMSO, 2 μg O25b-CRM RAC/DMSO, 0.2 μg O25b-SCP click 6-10% DoA, 2 μg O25b-SCP click 6-10% DoA, 0.2 μg O25b-SCP click 20-24% DoA, and 2 μg O25b-SCP click 20-24% DoA. FIG. 3 graphically depicts OPA titers at post-dose 2 (PD2) and post-dose 3 (PD3) for mice treated with the respective 0.2 μg dose immunogenic compositions. FIG. 4 graphically depicts OPA titers at PD2 and PD3 for mice treated with the respective 2 μg dose immunogenic compositions. The OPA geo mean titer (GMT) data is also presented in Table 5 below.

TABLE 5
OPA GMT Summary Data

| | | Pre | PD2, | PD2, | PD3, | PD3, |
|---|---|---|---|---|---|---|
| Group | N | Pool | GMT | RR(%) | GMT | RR(%) |
| O25b-CRM | 20 | 50 | 521 | 45.0 | 946 | 60.0 |
| RAC/DMSO 0.2 μg | | | | | | |
| O25b-CRM | 20 | 50 | 1714 | 70.0 | 23293 | 100.0 |
| RAC/DMSO 2.0 μg | | | | | | |
| O25b-SCP Click 6- | 20 | 50 | 1131 | 55.0 | 48519 | 89.5 |
| 10% DoA 0.2 μg | | | | | | |
| O25b-SCP Click 6- | 20 | 50 | 1807 | 60.0 | 56725 | 100.0 |
| 10% DoA 2.0 μg | | | | | | |
| O25b-SCP Click 20- | 20 | 50 | 542 | 50.0 | 4805 | 70.0 |
| 24% DoA 0.2 μg | | | | | | |
| O25b-SCP Click 20- | 20 | 50 | 11421 | 65.0 | 38979 | 85.0 |
| 24% DoA 2.0 μg | | | | | | |

RR(%) = responder rate percentage

At PD2 with 2 μg of antigen, the highest OPA titers were observed in the mice treated with O25b-SCP with 20-24% DoA (click chemistry).

At PD2 and with 2 μg of antigen, the O25b-SCP with 20-24% DoA (click chemistry) generated higher OPA titers than O25b-SCP with 6-10% DoA (click chemistry) or the benchmark O25b-CRM₁₉₇ (RAC/DMSO) conjugate.

At PD3 and with 0.2 μg of antigen, the O25b conjugated to SCP using click chemistry (at either DoA) were more immunogenic than the benchmark O25b-CRM RAC/DMSO conjugate. Furthermore, it was observed that between the 6-10% DoA and 20-24% DoA O25b-SCP conjugates, the OPA titer for 6-10% DoA was significantly higher.

Thus, it was concluded that at PD2 using 2 μg of antigen E. coli O25b conjugated to SCP 20-24% DoA (click chemistry) was more immunogenic compared to E. coli O25b conjugated to CRM₁₉₇ (RAC/DMSO). Additionally, it was concluded that at PD3 using 0.2 μg of antigen E. coli O25b conjugated to SCP using click chemistry (at either DoA) was more immunogenic compared to E. coli O25b conjugated to CRM₁₉₇ (RAC/DMSO). Accordingly, it was concluded that under these conditions, the O25b-SCP conjugate generated an increased immune response as compared to O25b-CRM₁₉₇.

### Example 3: In Vivo Immune Response Comparison of 4 SCP O-Antigen Conjugates (Click Chemistry) and 4 CRM O-Antigen Conjugates (RAC/DMSO)

In this study, the use of CRM₁₉₇ as a carrier for E. coli O-antigens O1A, O2, O6, and O25b was compared to the use of SCP as a carrier. The effects on immunogenicity were tested in a mouse model. The study also evaluated the effect of adding a QS21/MPLA liposomal adjuvant.

The synthesis of the respective CRM₁₉₇ and SP O-antigen conjugates was carried out as described in Examples 1 and 2, above. Summary data for the SOP conjugates for each O-antigen serotype is provided in Table 6, below. Summary data for the CRM₁₉₇ conjugates for each O-antigen serotype is provided in Table 7, below.

TABLE 6
Click-SCP Conjugation - Conjugate
Data for 4 Valent SCP O-Antigens

| Serotype | O1a | O2 | O6 | O25b |
|---|---|---|---|---|
| Poly Mw (kDa) | 46 | 48 | 46 | 53 |
| Act Poly DoA | 20 | 20 | 21 | 20 |
| Act poly Mw (kDa) | 42 | 49 | 51 | 56 |
| alkyne-SCP (A-SCP) DoA | 20 | 20 | 20 | 20 |
| A-SCP Mw (kDa) | 101 | 101 | 101 | 101 |

Conjugate Data

| | | | | |
|---|---|---|---|---|
| Yield (%) | 66 | 70 | 68 | 54 |
| SP Ratio | 0.77 | 0.65 | 0.73 | 0.66 |
| Free Saccharide (%) | 6 | 7 | 6 | <5 |
| Conjugate Mw (kDa) | 1160 | 628 | 1166 | 1033 |
| Saccharide Conc (mg/mL) | 0.73 | 0.56 | 0.8 | 0.44 |
| Protein Conc (mg/mL) | 0.95 | 0.86 | 1.1 | 0.67 |
| Endotoxin (EU/ug) | 0.001 | 0.006 | <0.001 | 0.004 |

| | |
|---|---|
| Conjugate Matrix | 10 mM Tris Buffer, pH 7.4 |

TABLE 7
RAC/DMSO CRM₁₉₇ Conjugation - Conjugate
Data for 4 Valent CRM₁₉₇ O-Antigens

| Serotype | O1a | O2 | O6 | O25b |
|---|---|---|---|---|
| Poly MW (kDa) | 44 | 46 | 42 | 48 |
| DOA | 16 | 16 | 14 | 16 |
| Act poly Mw (kDa) | 44 | 45 | 38 | 48 |

Conjugate Data

| | | | | |
|---|---|---|---|---|
| Yield (%) | 75 | 76 | 70 | 56 |
| SP Ratio | 0.87 | 0.98 | 0.97 | 1.0 |
| Free Saccharide (%) | 5 | 7 | <5 | <5 |
| Conjugate MW (kDa) | 2928 | 3043 | 1688 | 3411 |
| Saccharide Conc | 0.52 | 0.46 | 0.57 | 0.42 |
| (mg/mL) | | | | |
| Protein Conc (mg/mL) | 0.60 | 0.47 | 0.59 | 0.42 |
| Endotoxin (EU/ug) | <0.0009 | <0.001 | 0.003 | <0.002 |

| | |
|---|---|
| Conjugate Matrix | 10 mM Tris Buffer, pH 7.4 |

The study design is shown in Table 8, below. The study compared the immunogenicity of 4-valent SOP versus 4-valent CRM₁₉₇ formulated with and without liposomal adjuvant LUNA-2.

TABLE 8
Study Design 4-Valent O-Antigen Conjμgate Chemistry Study

| | | | | | | Dose | | |
|---|---|---|---|---|---|---|---|---|
| Group | | | | | Adjuvant | Volume/ | Vax | Bleed |
| # | Animals | Antigen | Dose | Adjuvant | Dose | Route | (Week) | (Week) |
| 1 | 20 | 4 Valent O-Antigen | 0.2 μg | NA | NA | SC | 0, 5, 13 | 0, 3, 7, 15 |
| | | (O-Ag) RAC-CRM | | | | (0.1 mL) | | |
| | | (O1/O2/O6/O25b) | | | | | | |
| 2 | 20 | 4 Valent O-Ag Click | 0.2 μg | NA | NA | SC | 0, 5, 13 | 0, 3, 7, 15 |
| | | SCP | | | | (0.1 mL) | | |
| | | (O1/O2/O6/O25b) | | | | | | |
| 3 | 20 | 4 Valent O-Ag RAC- | 0.2 μg | LiNA-2 | 10 μg | SC | 0, 5, 13 | 0, 3, 7, 15 |
| | | CRM | | | PHAD/ | (0.1 mL) | | |
| | | (O1/O2/O6/O25b) | | | 5 μg | | | |
| | | | | | QS-21 | | | |
| 4 | 20 | 4 Valent O-Ag Click | 0.2 μg | LiNA-2 | 10 μg | SC | 0, 5, 13 | 0, 3, 7, 15 |
| | | SCP | | | PHAD/ | (0.1 mL) | | |
| | | (O1/O2/O6/O25b) | | | 5 μg | | | |
| | | | | | QS-21 | | | |

The IgG GMT titers for the 4-valent O-antigens of this study are shown in Table 9, below. The 4-plex O-antigen (O—Ag) IgG dLIA serology assay was completed as follows: Long chain E. coli O—Ag polysaccharides of serotypes O25b, O1a, O2 and O6 were conjugated to poly-L-lysine and then to MagPlex*—C microspheres (Luminex) with EDC/NHS. Use of beads with distinct spectral addresses for each O-antigen enabled four-fold multiplexing. Beads were incubated with individual mouse sera or control mAbs serially diluted in blocking buffer with shaking at 2-8° C. for 18 h. After washing, bound serotype-specific IgG was detected with a Phycoerythrin (PE)-conjugated goat anti-mouse IgG mouse (F(ab′)2 fragment specific) secondary antibody (90 min RT incubation, with shaking). Microplates were read on a FlexMap 3D instrument (Biorad®). Serotype specific mouse IgG mAbs with similar binding properties (generated in-house) were used as internal standards to quantify IgG levels. Plots of standard curves for each mAb yielded overlapping linear slope profiles across 103 serum dilutions (log luminescence vs log serum dilution). The magnitude of the fluorescent PE signal was directly proportional to the amount of anti-O-Antigen IgG bound to the polysaccharide coupled microspheres. The data was analyzed using a custom SAS application, which uses a log/log linear regression model of the standard curves to interpolate antigen-specific antibody concentrations (μg/mL) from median fluorescent intensity. A lower limit of quantitation (LLOQ) for the 4-plex IgG dLIA of 0.153 ug/mL was calculated from standard curve bias, which was the same for each of the four antigens.

The OPA GMT titers for the 4-valent O-antigens of this study are shown in Table 10, below. Each table provides the responder rate percentage (% RR). O1a, O2, O6 and O25b OPAs used to evaluate immune sera were completed as follows: OPA assay protocols for K-antigen encapsulated serotype O1a and O25b test strains are described in previous publications (Chorro et al Infect Immun 2022 Vol. 90 Issue 4 Pages e0002222; Chorro et al Microbiology Spectrum 2024 Vol. 12 Issue 6 Pages eO4213-23). Analogous assays for serotype K-antigen encapsulated O2 and O6 OPA strains were similarly developed and optimized to identify appropriate concentrations of baby rabbit complement and HL60 effector cells particular to each strain.

Cell banks for all four OPAs were prepared by growing bacteria to an OD600 of between 0.5 to 1.0 in Todd-Hewitt broth with 0.5% Yeast extract (THY) media, adding glycerol to a final concentration of 20% prior to freezing. These exploratory assays used a 384-well microplate format. Briefly, pre-titered thawed bacteria were diluted to 1×10⁴ CFU/mL in OPA buffer [Hanks balanced salt solution (Life Technologies), 0.1% gelatin], and 20 μL (10³ CFU) of the bacterial suspension was combined with 10 μL of serially diluted serum incubated shaken at 2,000 rpm for 30 min at 37° C. in a 384-well tissue culture microplate. Subsequently, 10 μL of complement (baby rabbit serum Pel-Freez) and 10 μL of HL60 cells were added to each well, and the mixture was shaken at 2,000 rpm for 45 min at 37° C. in a 5% CO2 incubator. After the incubation, 10 μL of each 50 μL reaction mixture was transferred into the corresponding wells of a prewetted 384-well Millipore Sigma® MultiScreen HTS HV filter plate containing 50 μL DMEM/well. The liquid was removed by vacuum filtration and the plate incubated overnight at 37° C. in a sealed bag. The next day, the colonies were enumerated after staining with Coomassie dye using an ImmunoSpot® analyzer and Immunocapture software (Cellular Technology, Ltd.™).

The OPAs include control reactions without HL60 cells or complement, to demonstrate the dependence of any observed killing on these components. Serotype-specific rabbit immune serum standards were included on each assay plate to confirm reproducibility. Individual OPA titers were calculated from 11-point serum serial dilution titrations using variable-slope curve fitting (Excel®). Combined EC₅₀ OPA titer data were plotted using GraphPad Prism® to generate GMTs and associated P values for statistical significance (Welch's unpaired t-test with log-transformed data).

TABLE 9
4-Valent O-Antigen Conjμgate PD2 and PD3 IgG Titer Summary

| | | | O1a | | O2 | | O6 | | O25b | |
|---|---|---|---|---|---|---|---|---|---|---|
| Group | Vaccine | Timepoint | GMT % | RR | GMT | % RR | GMT | % RR | GMT | % RR |
| 1 | 4v O-Ag | PD2 | 1.701 | 65 | 4.633 | 80 | 2.648 | 65 | 0.168 | 0 |
| | RAC- | PD3 | 6.889 | 95 | 25.43 | 100 | 18.931 | 100 | 0.341 | 20 |
| | DMSO | | | | | | | | | |
| | 0.2 μg | | | | | | | | | |
| 2 | 4v O-Ag | PD2 | 6.249 | 95 | 17.81 | 95 | 4.012 | 80 | 0.215 | 10 |
| | Click | PD3 | 15.044 | 95 | 60.31 | 100 | 27.508 | 100 | 0.992 | 45 |
| | SCP | | | | | | | | | |
| | 0.2 μg | | | | | | | | | |
| 3 | 4v O-Ag | PD2 | 6.785 | 90 | 6.993 | 80 | 7.567 | 90 | 0.193 | 0 |
| | RAC- | PD3 | 5.518 | 90 | 8.348 | 90 | 28.850 | 90 | 3.777 | 85 |
| | DMSO 0.2 μg | | | | | | | | | |
| | LiNA-2 | | | | | | | | | |
| 4 | 4v O-Ag | PD2 | 9.735 | 100 | 22.33 | 100 | 7.077 | 90 | 0.205 | 5 |
| | Click | PD3 | 15.050 | 100 | 27.91 | 100 | 44.198 | 100 | 3.298 | 70 |
| | SCP | | | | | | | | | |
| | 0.2 μg | | | | | | | | | |
| | LiNA-2 | | | | | | | | | |

TABLE 10
4-Valent O-Antigen Conjμgate PD2 and PD3 OPA Titer Summary

| | | | O1a | | O2 | | O6 | | O25b | |
|---|---|---|---|---|---|---|---|---|---|---|
| Group # | Group | PD | GMT | % RR | GMT | % RR | GMT | % RR | GMT | % RR |
| 1 | 4 valent O-Ag | PD2 | 462 | 45% | 19699 | 100% | 1065 | 90% | 145 | 25% |
| | RAC-CRM | PD3 | 4535 | 80% | 30036 | 100% | 735 | 90% | 1009 | 65% |
| | 0.2 μg | Δ | 9.81 | | 1.52 | | 0.69 | | 6.96 | |
| 2 | 4 valent O-Ag | PD2 | 2962 | 80% | 13145 | 90% | 600 | 75% | 242 | 35% |
| | Click SCP | PD3 | 28046 | 100% | 46619 | 100% | 3225 | 95% | 1437 | 70% |
| | 0.2 μg | Δ | 9.47 | | 3.55 | | 5.37 | | 5.94 | |
| 3 | 4 valent O-Ag | PD2 | 11234 | 90% | 41434 | 100% | 4026 | 95% | 325 | 60% |
| | RAC-CRM | PD3 | 8429 | 90% | 16708 | 100% | 3039 | 85% | 5639 | 95% |
| | 0.2 μg + | Δ | 0.75 | | 0.40 | | 0.75 | | 17.37 | |
| | LiNA-2 | | | | | | | | | |
| 4 | 4 valent O-Ag | PD2 | 7242 | 95% | 68644 | 100% | 2597 | 90% | 229 | 45% |
| | Click SCP | PD3 | 31311 | 100% | 56886 | 100% | 7888 | 100% | 4618 | 90% |
| | 0.2 μg + LiNA- | Δ | 4.32 | | 0.83 | | 3.04 | | 20.21 | |
| | 2 | | | | | | | | | |

### Results and Discussion

The following observations were made with regard to the serotype O1a IgG and OPA responses to the tetravalent SCP and CRM₁₉₇ conjugates: At PD2: O1a IgG response to the SCP conjugates was significantly greater than the O1a IgG response to the CRM₁₉₇ conjugates 10 (>3.5-fold). Formulation of the CRM₁₉₇ conjugates with LiNA-2 similarly enhanced the response (>3.5-fold). The O1a OPA response to the SCP conjugates was substantially greater than the O1a response to the CRM₁₉₇ conjugates (6-fold higher). Formulation of the CRM₁₉₇ conjugates with LiNA-2 enhanced the OPA response (22-fold higher). At PD3: the O1a IgG response to the SCP conjugates was significantly greater than the O1a IgG response to the CRM₁₉₇ conjugates 15 (about 2.2-fold). Even so, formulation of either conjugate mixture with LiNA-2 didn't improve responses relative to unadjuvanted. The O1a OPA response to the SCP conjugates was significantly greater than the O1a OPA response to the CRM₁₉₇ conjugates (about 6.2 fold higher). Formulation of either conjugate type with LiNA-2 generated similar responses compared to the unadjuvanted conjugates.

The following observations were made with regard to the serotype O2 IgG and OPA responses to the tetravalent SCP and CRM₁₉₇ conjugates: At PD2: the O2 IgG response to the SCP conjugates was significantly greater than the O2 IgG response to the CRM₁₉₇ conjugates (>3.5-fold). Formulation with LiNA-2 did not significantly improve responses to either conjugate. The O2 OPA response to the SCP conjugates was comparable to the CRM₁₉₇ conjugates. Even so, formulation of the SCP conjugates with LiNA-2 significantly enhanced responses relative to unadjuvanted (about 5-fold). At PD3, the O2 IgG response to the SCP conjugates was significantly greater than the response to the CRM₁₉₇ conjugates (>2.4-fold). Formulation of either conjugate type with LiNA-2 did not improve the IgG responses. The O2 OPA response to the SCP conjugates and the CRM₁₉₇ conjugates was comparable.

The following observations were made with regard to the serotype O6 IgG and OPA responses to the tetravalent SCP and CRM₁₉₇ conjugates: At PD2: the O6 IgG response to the SCP conjugates was marginally better than to the CRM₁₉₇ conjugates. For both conjugate types, formulation with LiNA-2 resulted in a modest boost in titers. The O6 OPA response to the SCP conjugates was comparable to the CRM₁₉₇ conjugates. For both conjugate types, formulation with LiNA-2 significantly enhanced the responses (>3.5-fold). At PD3, O6 IgG responses to the SCP conjugates were similar to the CRM₁₉₇ conjugates. Furthermore, formulation of either conjugate type with LiNA-2 failed to show a significant enhancement. In contrast, the O6 OPA response to the SCP conjugates was significantly greater than the OPA response to the CRM₁₉₇ conjugates (4.6-fold higher). Furthermore, formulation of the CRM₁₉₇ conjugates with LiNA-2 significantly enhanced the response relative to unadjuvanted (4.3-fold higher).

The following observations were made with regard to the serotype O25b IgG and OPA responses to the tetravalent SCP and CRM₁₉₇ conjugates: At PD2: both the O25b IgG and OPA responses to the SCP and CRM₁₉₇ conjugates were low. Furthermore, at PD2, LiNA-2 did not significantly improve the responses. At PD3, O25b IgG response to the SCP conjugates was marginally better than to the CRM₁₉₇ conjugates. Formulation of either conjugate with LiNA-2 significantly improved the responses. O25b OPA response to the SCP conjugates was slightly higher than the CRM₁₉₇ conjugates. For both conjugation chemistries, formulation with LiNA-2 significantly enhanced the O25b OPA response.

### Conclusions

In conclusion, in analyzing the IgG and OPA responses of E. coli O-antigens O1a, O2, 06, and O25b, numerous data points indicate enhanced immunogenicity with the selection of the SCP carrier over the CRM₁₉₇ carrier. Additionally, numerous data points indicate enhanced immunogenicity from formulation of both SCP and CRM₁₉₇ conjugates with LiNA-2 adjuvant.

### Example 4: Immunogenicity of Lina-2 Adjuvanted 4 Valent O-Antigen Scp Glycoconjugates in Rhesus Macaques

In this study, the impact of two different adjuvants on increasing the immune response of rhesus macaque non-human primates (NHPs) to 4 different E. coli O-antigen glycoconjugates was assessed. Specifically, the O-antigen glycoconjugates were serotype O1A, O2, O6, and O25b each conjugated to a SCP carrier, as described in the examples above. The adjuvants evaluated were LiNA-2 at a 2× concentration (2×LiNA-2), as described herein, and gpi-anchored FimH-DSG modRNA (SEQ ID NO: 88) combined with LNP formulations described in this Example.

The RNAs were formulated into LNPs comprising the following lipids: cationic lipid (ALC-0315), cholesterol, DSPC (1,2distearoyl-sn-glycero-3-phosphocholine), and PEG-lipid (ALC-O159) which were solubilized in ethanol at a molar ratio of about 46.3:42.7:9.4:1.6. The physicochemical properties and the structures of the 4 lipids are shown in the Table hereinbelow. Lipid nanoparticles were prepared and tested according to the general procedures described in U.S. Pat. No. 9,737,619 (PCT Pub. No. WO2015/199952) and U.S. patent Ser. No. 10/166,298 (WO 2017/075531) and WO2020/146805, each of which is hereby incorporated by reference in its entirety.

40 male rhesus macaques were selected for this study. The study design is shown in Table 11, below. Five groups of eight male Rhesus macaques were vaccinated with 4 valent O-antigen SCP glycoconjugates. The study included two groups vaccinated with 4 valent O-antigen SCP glycoconjugates and LiNA-2, one group at a high dose of glycoconjugates, and one group at a lower dose of glycoconjugates. Likewise, the study included two groups vaccinated with 4 valent O-antigen SCP and FimH modRNA, one group at a high dose of glycoconjugates, and one group at a lower dose of glycoconjugates. The dosing of the higher dose groups was 2 μg of each of serotypes O1a, O2, and O6, as well as 4 μg of O25b. The dosing of the lower dose groups was 0.5 μg of each of serotypes O1a, O2, and O6, as well as 1 μg of O25b. Vaccination was administered at months 0, 2, and 6. Animals were bled prior to vaccination and at weeks 1, 2, 4, 6, 9, 10, 12, and 25. 1.0 mL of vaccine formulation was injected intramuscularly into the left leg for the priming dose and into the right leg for the subsequent booster doses. Geometric mean titers (GMTs) of IgG antibodies specific for each of the respective O-antigen serotypes O1a, O2, O6, and O25b were quantified using a Luminex 4-plex dLIA assay. Opsonophagocytic assay (OPA) assays were also completed and OPA titers were determined for each of O-antigen serotypes O1a, O2, O6, and O25b.

TABLE 11
Study Design

| Group | | | Adjuvant/ | Dose | Month Vaccine |
|---|---|---|---|---|---|
| # | Antigen | Vaccine Dose | Dose | Volume | Administered |
| 1 | O1A, O2, O6, | 2/2/2/4 μg | None | 0.5 mL | 0, 2, 6 |
| | and O25b with | O-antigen | | | |
| | SCP carrier | | | | |
| 2 | O1A, O2, O6, | 2/2/2/4 μg | LiNA-2 | 0.5 mL | 0, 2, 6 |
| | and O25b with | O-antigen | 200 μg | | |
| | SCP carrier | | | | |
| 3 | O1A, O2, O6, | 0.5/0.5/0.5/ | LiNA-2 | 0.5 mL | 0, 2, 6 |
| | and O25b with | 1 μg O- | 200 μg | | |
| | SCP carrier | antigen | | | |
| 4 | O1A, O2, O6, | 2/2/2/4 μg | None | 0.5 mL | 0, 2, 6 |
| | and O25b with | O-antigen + | | | |
| | SCP carrier + | 30 μg | | | |
| | FimH modRNA | modRNA/LNP | | | |
| 5 | O1A, O2, O6, | 0.5/0.5/0.5/ | None | 0.5 mL | 0, 2, 6 |
| | and O25b with | 1 μg O- | | | |
| | SCP carrier + | antigen + | | | |
| | FimH modRNA | 30 μg | | | |
| | | modRNA/LNP | | | |

### Results and Discussion

The 4-plex O-antigen IgG dLIA serology assay was completed as follows: The primate serology assay utilized the same O-antigen MagPlex®-C microspheres (Luminex) as the mouse serology assay described in Example 3, above. Microspheres were incubated with serially diluted NHP serum samples, controls, and human polyclonal serum standards overnight at 2-8° C. while shaking. In this case, bound serotype-specific IgG was detected with PE-conjugated goat anti-human IgG. Fcγ fragment-specific secondary antibody (Jackson Laboratories) and fluorescence was measured on the FlexMap 3D instrument (Biorad®). Human serum containing IgG to all four O-antigens was used as an internal standard for interpolating NHP serum IgG concentrations. An arbitrary value of 100 U/mL was assigned for each antigen-specific IgG of the standard, which yielded comparable slope profiles and signal output. Lower limit of quantification (LLOQ) was defined as 2 U/mL.

As demonstrated by the serology data shown in Tables 12-15 below, trends in immunogenicity were similar for all four serotypes. Robust uniform geometric mean titers (GMTs) were achieved two weeks after a single unadjuvanted dose at the base dose of2/2/2/4 μg O-antigen glycoconjugate. LiNA-2 had a major impact on further enhancement of the responses after one, two, or three doses, with the greatest impact observed with the serotype O1a and O25b O-antigen glycoconjugates, for which greater than ten-fold increases in GMTs were observed. When formulated with LiNA-2, IgG responses to the four-fold lower dose level of the glycoconjugates (0.5/0.5/0.5/1 μg) were comparable to the base dose level (2/2/2/4 μg), highlighting the dose-sparing properties of the adjuvant. Compared with LiNA-2, the FimH modRNA LNP consistently conferred a smaller adjuvanting effect on O-antigen IgG GMTs.

TABLE 12
Serotype 01A IgG GMT Titers

| | | | 2 weeks | 4 weeks | 4 weeks | 4 weeks |
|---|---|---|---|---|---|---|
| Group | | | post-dose | post-dose | post-dose | post-dose |
| # | | Week 0 | 1 | 1 | 2 | 3 |
| 1 | 4 valent O- | 0.59 | 48.49 | 54.86 | 93.55 | 73.99 |
| | antigen SCP | | | | | |
| | 2/2/2/4 μg | | | | | |
| 2 | 4 valent O- | 0.54 | 726.67 | 295.29 | 1,023.50 | 2,796.47 |
| | antigen SCP | | | | | |
| | 2/2/2/4 μg | | | | | |
| | + | | | | | |
| | LiNA-2 | | | | | |
| | Fold rise vs. | | 15 | 5 | 11 | 38 |
| | unadjuvanted | | | | | |
| 3 | 4 valent O- | 0.51 | 1,151.30 | 356.76 | 991.63 | 1,609.23 |
| | antigen SCP | | | | | |
| | 0.5/0.5/0.5/1 μg | | | | | |
| | + | | | | | |
| | LiNA-2 | | | | | |
| | Fold rise vs. | | 24 | 7 | 11 | 22 |
| | unadjuvanted | | | | | |
| 4 | 4 valent O- | 0.63 | 573.14 | 203.77 | 428.45 | 450.20 |
| | antigen SCP | | | | | |
| | 2/2/2/4 μg | | | | | |
| | + | | | | | |
| | FimH modRNA/ | | | | | |
| | LNP | | | | | |
| | Fold rise vs. | | 12 | 4 | 5 | 6 |
| | unadjuvanted | | | | | |
| 5 | 4 valent O- | 0.41 | 266.69 | 94.47 | 332.23 | 308.02 |
| | antigen SCP | | | | | |
| | 0.5/0.5/0.5/1 μg | | | | | |
| | + | | | | | |
| | FimH modRNA/ | | | | | |
| | LNP | | | | | |
| | Fold rise vs. | | 5 | 2 | 4 | 4 |
| | unadjuvanted | | | | | |

TABLE 13
Serotype O2 IgG GMT Titers

| | | | 2 weeks | 4 weeks | 4 weeks | 4 weeks |
|---|---|---|---|---|---|---|
| Group | | Week | post-dose | post-dose | post-dose | post-dose |
| # | | 0 | 1 | 1 | 2 | 3 |
| 1 | 4 valent O- | 2.89 | 336.38 | 199.59 | 238.50 | 195.50 |
| | antigen SCP | | | | | |
| | 2/2/2/4 μg | | | | | |
| 2 | 4 valent O- | 2.44 | 1,786.69 | 674.40 | 1,655.62 | 2,574.73 |
| | antigen SCP | | | | | |
| | 2/2/2/4 μg | | | | | |
| | + | | | | | |
| | LiNA-2 | | | | | |
| | Fold rise vs. | | 5 | 3 | 7 | 13 |
| | unadjuvanted | | | | | |
| 3 | 4 valent O- | 2.26 | 2,009.21 | 603.29 | 1,833.46 | 2,337.06 |
| | antigen SCP | | | | | |
| | 0.5/0.5/0.5/1 μg | | | | | |
| | + | | | | | |
| | LiNA-2 | | | | | |
| | Fold rise vs. | | 6 | 3 | 8 | 12 |
| | unadjuvanted | | | | | |
| 4 | 4 valent O- | 4.65 | 1,851.04 | 574.24 | 662.68 | 590.01 |
| | antigen SCP | | | | | |
| | 2/2/2/4 μg | | | | | |
| | + | | | | | |
| | FimH modRNA/ | | | | | |
| | LNP | | | | | |
| | Fold rise vs. | | 6 | 3 | 3 | 3 |
| | unadjuvanted | | | | | |
| 5 | 4 valent O- | 1.66 | 1,020.73 | 296.97 | 755.59 | 768.22 |
| | antigen SCP | | | | | |
| | 0.5/0.5/0.5/1 μg | | | | | |
| | + | | | | | |
| | FimH modRNA/ | | | | | |
| | LNP | | | | | |
| | Fold rise vs. | | 3 | 1 | 3 | 4 |
| | unadjuvanted | | | | | |

TABLE 14
Serotype O6 IgG GMT Titers

| | | | 2 weeks | 4 weeks | 4 weeks | 4 weeks |
|---|---|---|---|---|---|---|
| Group | | Week | post-dose | post-dose | post-dose | post-dose |
| # | | 0 | 1 | 1 | 2 | 3 |
| 1 | 4 valent O- | 0.25 | 127.41 | 58.34 | 117.36 | 77.74 |
| | antigen SCP | | | | | |
| | 2/2/2/4 μg | | | | | |
| 2 | 4 valent O- | 0.26 | 526.09 | 248.50 | 1,375.88 | 2,954.74 |
| | antigen SCP | | | | | |
| | 2/2/2/4 μg | | | | | |
| | + | | | | | |
| | LiNA-2 | | | | | |
| | Fold rise vs. | | 4 | 4 | 12 | 38 |
| | unadjuvanted | | | | | |
| 3 | 4 valent O- | 0.20 | 1,115.13 | 374.10 | 1,882.54 | 1,637.68 |
| | antigen SCP | | | | | |
| | 0.5/0.5/0.5/1 μg | | | | | |
| | + | | | | | |
| | LiNA-2 | | | | | |
| | Fold rise vs. | | 9 | 6 | 16 | 21 |
| | unadjuvanted | | | | | |
| 4 | 4 valent O- | 0.42 | 872.86 | 294.59 | 600.02 | 652.68 |
| | antigen SCP | | | | | |
| | 2/2/2/4 μg | | | | | |
| | + | | | | | |
| | FimH modRNA/ | | | | | |
| | LNP | | | | | |
| | Fold rise vs. | | 7 | 5 | 5 | 8 |
| | unadjuvanted | | | | | |
| 5 | 4 valent O- | 0.87 | 391.94 | 121.39 | 535.34 | 482.69 |
| | antigen SCP | | | | | |
| | 0.5/0.5/0.5/1 μg | | | | | |
| | + | | | | | |
| | FimH modRNA/ | | | | | |
| | LNP | | | | | |
| | Fold rise vs. | | 3 | 2 | 5 | 6 |
| | unadjuvanted | | | | | |

TABLE 15
Serotype O25b IgG GMT Titers

| | | | 2 weeks | 4 weeks | 4 weeks | 4 weeks |
|---|---|---|---|---|---|---|
| Group | | Week | post-dose | post-dose | post-dose | post-dose |
| # | | 0 | 1 | 1 | 2 | 3 |
| 1 | 4 valent O- | 0.40 | 24.07 | 18.85 | 60.72 | 87.36 |
| | antigen SCP | | | | | |
| | 2/2/2/4 μg | | | | | |
| 2 | 4 valent O- | 0.62 | 407.52 | 149.41 | 1,264.55 | 4,881.03 |
| | antigen SCP | | | | | |
| | 2/2/2/4 μg | | | | | |
| | + | | | | | |
| | LiNA-2 | | | | | |
| | Fold rise vs. | | 17 | 8 | 21 | 56 |
| | unadjuvanted | | | | | |
| 3 | 4 valent O- | 0.34 | 766.55 | 266.00 | 3,207.40 | 4,939.67 |
| | antigen SCP | | | | | |
| | 0.5/0.5/0.5/1 μg | | | | | |
| | + | | | | | |
| | LiNA-2 | | | | | |
| | Fold rise vs. | | 32 | 14 | 53 | 57 |
| | unadjuvanted | | | | | |
| 4 | 4 valent O- | 0.45 | 428.93 | 197.31 | 1,103.02 | 1,024.21 |
| | antigen SCP | | | | | |
| | 2/2/2/4 μg | | | | | |
| | + | | | | | |
| | FimH modRNA/ | | | | | |
| | LNP | | | | | |
| | Fold rise vs. | | 18 | 10 | 18 | 12 |
| | unadjuvanted | | | | | |
| 5 | 4 valent O- | 0.31 | 221.27 | 127.73 | 766.62 | 853.40 |
| | antigen SCP | | | | | |
| | 0.5/0.5/0.5/1 μg | | | | | |
| | + | | | | | |
| | FimH modRNA/ | | | | | |
| | LNP | | | | | |
| | Fold rise vs. | | 9 | 7 | 13 | 10 |
| | unadjuvanted | | | | | |

The OPA response data is shown in Tables 16-19, below. The O1a, O2, O6, and O25b OPA assays used to evaluate the immune sera were completed as described in Example 3, above. Due to the presence of substantial non-specific bactericidal activity detected in pre-immune sera with serotype O2, 06 and O25b O-antigen OPA titers, pooled instead of individual NHP sera was evaluated following absorption with O-antigen knockout bacteria. For the serotype O6 OPA response assay, total affinity-purified IgG was evaluated.

Overall, the trends in serotype-specific OPA functional immunogenicity paralleled the IgG data. LiNA-2 had a stronger adjuvanting effect than the modRNA/LNP.

TABLE 16
Immunogenicity of Pooled Sera with O1a OPA Strain

| Group | | | 2 weeks | 2 weeks | 2 weeks |
|---|---|---|---|---|---|
| # | | Week 0 | post-dose 1 | post-dose 2 | post-dose 3 |
| 1 | 4 valent O- | 50 | 133 | 14015 | 11124 |
| | antigen SCP | | | | |
| | 2/2/2/4 μg | | | | |
| 2 | 4 valent O- | 50 | 4715 | 137162 | 763292 |
| | antigen SCP | | | | |
| | 2/2/2/4 μg + | | | | |
| | LiNA-2 | | | | |
| | Fold rise vs. | | 35 | 10 | 69 |
| | unadjuvanted | | | | |
| 3 | 4 valent O- | 50 | 7681 | 82755 | 287918 |
| | antigen SCP | | | | |
| | 0.5/0.5/0.5/ | | | | |
| | 1 μg + | | | | |
| | LiNA-2 | | | | |
| | Fold rise vs. | | 58 | 6 | 26 |
| | unadjuvanted | | | | |
| 4 | 4 valent O- | 50 | 15705 | 34379 | 45329 |
| | antigen SCP | | | | |
| | 2/2/2/4 μg + | | | | |
| | FimH modRNA/ | | | | |
| | LNP | | | | |
| | Fold rise vs. | | 118 | 2 | 4 |
| | unadjuvanted | | | | |
| 5 | 4 valent O- | 50 | 6629 | 37808 | 93592 |
| | antigen SCP | | | | |
| | 0.5/0.5/0.5/ | | | | |
| | 1 μg + | | | | |
| | FimH modRNA/ | | | | |
| | LNP | | | | |
| | Fold rise vs. | | 50 | 3 | 8 |
| | unadjuvanted | | | | |

TABLE 17
Immunogenicity of Pooled Sera with O2 OPA Strain

| Group | | | 2 weeks | 2 weeks | 2 weeks |
|---|---|---|---|---|---|
| # | | Week 0 | post-dose 1 | post-dose 2 | post-dose 3 |
| 1 | 4 valent O- | 50 | 1102 | 2898 | 1569 |
| | antigen SCP | | | | |
| | 2/2/2/4 μg | | | | |
| 2 | 4 valent O- | 50 | 37255 | 107791 | 154670 |
| | antigen SCP | | | | |
| | 2/2/2/4 μg + | | | | |
| | LiNA-2 | | | | |
| | Fold rise vs. | | 34 | 37 | 99 |
| | unadjuvanted | | | | |
| 3 | 4 valent O- | 50 | 49465 | 29400 | 34187 |
| | antigen SCP | | | | |
| | 0.5/0.5/0.5/ | | | | |
| | 1 μg + | | | | |
| | LiNA-2 | | | | |
| | Fold rise vs. | | 45 | 10 | 22 |
| | unadjuvanted | | | | |
| 4 | 4 valent O- | 50 | 77546 | 6604 | 3824 |
| | antigen SCP | | | | |
| | 2/2/2/4 μg + | | | | |
| | FimH modRNA/ | | | | |
| | LNP | | | | |
| | Fold rise vs. | | 70 | 2 | 2 |
| | unadjuvanted | | | | |
| 5 | 4 valent O- | 50 | 6624 | 3126 | 1458 |
| | antigen SCP | | | | |
| | 0.5/0.5/0.5/ | | | | |
| | 1 μg + | | | | |
| | FimH modRNA/ | | | | |
| | LNP | | | | |
| | Fold rise vs. | | 6 | 1 | 1 |
| | unadjuvanted | | | | |

TABLE 18
Immunogenicity of Pooled Sera with O6 OPA Strain (IgG-purified)

| Group | | | 2 weeks | 2 weeks | 2 weeks |
|---|---|---|---|---|---|
| # | | Week 0 | post-dose 1 | post-dose 2 | post-dose 3 |
| 1 | 4 valent O- | 0.0001 | 0.0110 | 0.0198 | 0.0320 |
| | antigen SCP | | | | |
| | 2/2/2/4 μg | | | | |
| 2 | 4 valent O- | 0.0001 | 0.1598 | 0.5345 | 0.9804 |
| | antigen SCP | | | | |
| | 2/2/2/4 μg + | | | | |
| | LiNA-2 | | | | |
| | Fold rise vs. | | 15 | 27 | 31 |
| | unadjuvanted | | | | |
| 3 | 4 valent O- | 0.0001 | 0.9804 | 0.5082 | 0.7634 |
| | antigen SCP | | | | |
| | 0.5/0.5/0.5/ | | | | |
| | 1 μg + | | | | |
| | LiNA-2 | | | | |
| | Fold rise vs. | | 89 | 26 | 24 |
| | unadjuvanted | | | | |
| 4 | 4 valent O- | 0.0001 | 0.3857 | 0.2552 | 0.2938 |
| | antigen SCP | | | | |
| | 2/2/2/4 μg + | | | | |
| | FimH modRNA/ | | | | |
| | LNP | | | | |
| | Fold rise vs. | | 35 | 13 | 9 |
| | unadjuvanted | | | | |
| 5 | 4 valent O- | 0.0001 | 0.0402 | 0.0768 | 0.1679 |
| | antigen SCP | | | | |
| | 0.5/0.5/0.5/ | | | | |
| | 1 μg + | | | | |
| | FimH modRNA/ | | | | |
| | LNP | | | | |
| | Fold rise vs. | | 4 | 4 | 5 |
| | unadjuvanted | | | | |

TABLE 19
Immunogenicity of Pooled Sera with O25b OPA Strain

| Group | | | 2 weeks | 2 weeks | 2 weeks |
|---|---|---|---|---|---|
| # | | Week 0 | post-dose 1 | post-dose 2 | post-dose 3 |
| 1 | 4 valent O- | 50 | 3784 | 59730 | 33980 |
| | antigen SCP | | | | |
| | 2/2/2/4 μg | | | | |
| 2 | 4 valent O- | 50 | 40946 | 1323850 | 574485 |
| | antigen SCP | | | | |
| | 2/2/2/4 μg + | | | | |
| | LiNA-2 | | | | |
| | Fold rise vs. | | 11 | 22 | 17 |
| | unadjuvanted | | | | |
| 3 | 4 valent O- | 50 | 95774 | 545595 | 654410 |
| | antigen SCP | | | | |
| | 0.5/0.5/0.5/ | | | | |
| | 1 μg + | | | | |
| | LiNA-2 | | | | |
| | Fold rise vs. | | 25 | 9 | 19 |
| | unadjuvanted | | | | |
| 4 | 4 valent O- | 554 | 22896 | 232790 | 69090 |
| | antigen SCP | | | | |
| | 2/2/2/4 μg + | | | | |
| | FimH modRNA/ | | | | |
| | LNP | | | | |
| | Fold rise vs. | | 6 | 4 | 2 |
| | unadjuvanted | | | | |
| 5 | 4 valent O- | 50 | 8315 | 460916 | 144350 |
| | antigen SCP | | | | |
| | 0.5/0.5/0.5/ | | | | |
| | 1 μg + | | | | |
| | FimH modRNA/ | | | | |
| | LNP | | | | |
| | Fold rise vs. | | 2 | 8 | 4 |
| | unadjuvanted | | | | |

### Conclusions

In this study, for each of the four O-antigen serotypes, an adjuvanting effect was demonstrated by the addition of LiNA-2 or FimH modRNA LNPs as determined by serotype IgG responses and OPA responses in rhesus NHPs. At the four-fold lower vaccine dose level, immune responses in the presence of LiNA-2 or modRNA LNP were consistently higher than to the unadjuvanted glycoconjugate, confirming the dose sparing impact of either adjuvant formulation.

modRNA LNP Production:

DNA plasmids encoding E. coli FimH proteins were prepared and utilized for in vitro transcription reactions to generate RNA. In vitro transcription of RNA is known in the art and is described herein. DNA templates were cloned into a modRNA cloning entry vector with backbone sequence elements (T7 promoter, 5′ UTR, 3′ UTR, and 3′ poly-A tail) with improved RNA stability and translational efficiency. The DNA was purified, spectrophotometrically quantified and in vitro-transcribed by T7 RNA polymerase in the presence of a trinucleotide cap1 analogue ((m₂^{7,3′-O})Gppp(m^{2′-O})ApG) known as CleanCap® AG; TriLink Biotechnologies) and N¹-methylpseudouridine-5′-triphosphate (also known as N¹-methylpseudouridine-5′-triphosphate, N¹meΨTP, m¹ΨTP, 1-methyl-pseudouridine phosphoramidite or N¹-methyl-pseudouridine-5′-triphosphate; TriLink Biotechnologies) in order to replace the uridine residues and thereby form the modified RNA (modRNA).

The FimH RNA was generated from codon-optimized (CO) DNA for stabilization and superior protein expression. DNA constructs of the present disclosure, and corresponding RNA sequences, comprising a 5′ UTR, an open reading frame encoding a FimH polypeptide, a 3′ UTR and a 3′ poly-A tail are described herein.

Lipids in the LNP Formulation

| Lipid | Molecular | Molecular Formula | |
|---|---|---|---|
| (CAS | Weight | Physical state and | |
| number) | [Da] | Storage condition | Chemical name and structure |
| Cationic Lipid ALC-0315 (Not applicable) | 766 | C₄₈H₉₅NO₅ Liquid (oil) −20° C. | 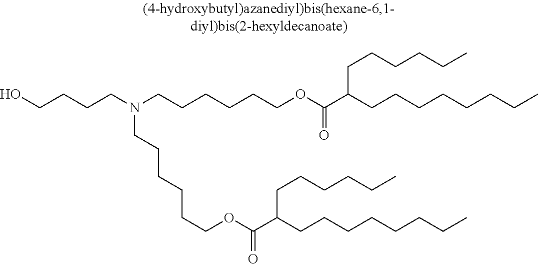 [SMILES: CCCCCCCCC(CCCCCC)C(=O)OCCCCCCN(CCCCO)CCCCCCOC(=O)C(CCCCCC)CCCCCCCC] |
| PEG-Lipid ALC-0159 (1849616- 42-7) | ~2400- 2600 | C₃₀H₆₀NO(C₂H₄O)ₙOCH₃ n = 45-50 Solid −20° C. | 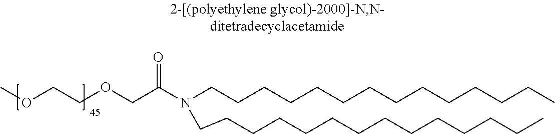 |
| DSPC (816-94-4) | 790 | C₃₀H₈₈NO₈P Solid −20° C. | 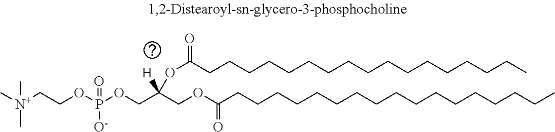 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol (57-88-5) | 387 | C₂₇H₄₆O Solid −20° C. | 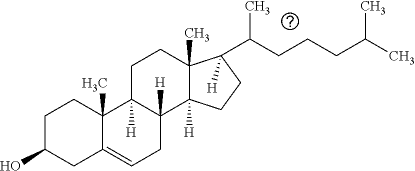 |

CAS = Chemical Abstract Service; DSPC = 1,2-disteroyl-sn-glycero-3-phosphocholine

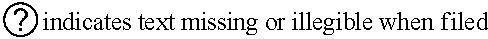

### Example 5: Immunogenicity of CPG Adjuvanted 4 Valent O-Antigen Scp Glycoconjugates in Rhesus Macaques

In this study, the adjuvanting effect of nucleotide adjuvant CpG 24555 on immunogenicity of E. coli O-antigen SCP glycoconjugates of serotypes O1a, O2, O6, and O25b was assessed in a NHP (Rhesus Macaques) model.

20 male rhesus macaques were selected for this study. The study design is shown in Table 20, below. Three groups of Rhesus macaques were vaccinated with the 4 valent O-antigen SCP glycoconjugates including one group without CpG adjuvant, one group with CpG adjuvant and a lower dose of the 4 valent O-antigen SCP glycoconjugates, and one group with CpG adjuvant and a higher dose of 4 valent O-antigen SCP glycoconjugates. The no adjuvant control group, as well as the group with CpG adjuvant and the higher dose of glycoconjugates included 2 μg of serotypes O1a, O2, and O6, as well as 4 μg of serotype O25b. The group with CpG adjuvant and the lower dose of glycoconjugates included 0.5 μg of serotypes O1a, O2, and O6, as well as 1 μg of serotype O25b. Vaccination was administered at months 0, 2, and 6. Animals were bled prior to vaccination and at weeks 1, 2, 4, 6, 9, 10, 12, 25, and 26. 0.5 mL of vaccine formulation was injected intramuscularly (IM).

TABLE 20
Study Design

| Group | | | | | Adjuvant | Dose Vol/ | Vax |
|---|---|---|---|---|---|---|---|
| # | NHPs | Antigen | Vaccine Dose | Adjuvant | Dose | Route | (month) |
| 1 | 4 | 4 Valent O- | 2/2/2/4 μg (O-Ag) | — | — | 0.5 mL/IM | 0, 2, 6 |
| | | antigen (O- | | | | | |
| | | Ag) SCP | | | | | |
| | | (O1/O2/O6/O25b) | | | | | |
| 2 | 8 | 4 Valent O-Ag | 2/2/2/4 μg (O-Ag) | CpG | 1800 μg | 0.5 mL/IM | 0, 2, 6 |
| | | SCP | | | | | |
| | | (O1/O2/O6/ | | | | | |
| | | O25b) | | | | | |
| 3 | 8 | 4 Valent O-Ag | 0.5/0.5/0.5/1 μg | CpG | 1800 μg | 0.5 mL/IM | 0, 2, 6 |
| | | SCP | (O-Ag) | | | | |
| | | (O1/O2/O6/ | | | | | |
| | | O25b) | | | | | |

### Results and Discussion

Geometric mean titers (GMTs) of IgG antibodies specific for each of the respective 0-antigen serotypes O1a, O2, O6, and O25b were quantified using a Luminex 4-plex dLIA assay. The 4-plex O-antigen IgG dLIA serology assay was completed as described in Example 4, above. Table 21 and 22, below, present data for IgG GMT titers for each of serotypes O1a, O2, O6, and O25b. Table 21 provides data from IgG dLIA assays with polyclonal serum (pAb) standards.

Table 22 provides data from IgG dLIA assays with mAb standards. The method was the same as for the IgG dLIA with pAb standards, described above, except that humanized mAbs (instead of human sera) were used as standards for interpolating IgG titers. In this case, the mouse IgG Fc region of mAbs used in the mouse 4-plex serology assay was replaced with the human IgG1 backbone by recloning of the variable light and heavy chain regions of the O1a, O2, O6 and O25b mAb standards into the pTT5 vector (Li D, et al. 2022. Protocol for high-throughput cloning, expression, purification, and evaluation of bispecific antibodies. STAR Protocols 3:101428.).

Results from the two IgG dLIA assays showed similar trends. Peak IgG responses after a single dose were observed for all serotypes at 2 weeks after vaccination, declining by week six. A second dose at week 8 boosted the responses to higher levels.

CpG adjuvant had the greatest impact on serotype O25b IgG titers, with 20-fold to 40-fold increases achieved after each vaccination relative to unadjuvanted titers. 10-fold to 20-fold increases were observed for serotype O1a and increases of less than 10-fold were observed for serotypes O2 and O6.

TABLE 21
O-Antigen Serotype IgG GMT Titers (IgG dLIA with
pAb Standards)

| | | Pre- | 1 wk | 2 wk | 6 wk | 2 wk |
|---|---|---|---|---|---|---|
| | | Vx | PD1 | PD1 | PD1 | PD2 |
| Group | O1A | wk-2 | wk1 | wk2 | wk6 | wk10 |
| 1 | O1/O2/O6/O25b | 0.29 | 2 | 11.01 | 12.32 | 57.72 |
| | 2/2/2/4 μg | | | | | |
| 2 | O1/O2/O6/O25b 2/2/2/4 | 0.58 | 14.4 | 213.17 | 98.48 | 753.19 |
| | μg + CpG | | | | | |
| | Fold rise vs | | 7 | 19 | 8 | 13 |
| | unadjuvanted: | | | | | |
| 3 | O1/O2/O6/O25b | 0.37 | 6.5 | 105.13 | 31.07 | 362.55 |
| | 0.5/0.5/0.5/1 μg + CpG | | | | | |
| | Fold rise vs | | 3 | 10 | 3 | 6 |
| | unadjuvanted: | | | | | |
| | O2 | | | | | |
| 1 | O1/O2/O6/O25b | 2.61 | 99.3 | 228.58 | 99.81 | 250.58 |
| | 2/2/2/4 μg | | | | | |
| 2 | O1/O2/O6/O25b | 3.85 | 142.9 | 853.27 | 406.52 | 1377.13 |
| | 2/2/2/4 μg + CpG | | | | | |
| | Fold rise vs | | 1 | 4 | 4 | 5 |
| | unadjuvanted: | | | | | |
| 3 | O1/O2/O6/25b | 1.99 | 49.6 | 517.83 | 186.52 | 1245.45 |
| | 0.5/0.5/0.5/1 μg + CpG | | | | | |
| | Fold rise vs | | 0 | 2 | 2 | 5 |
| | unadjuvanted: | | | | | |
| | O6 | | | | | |
| 1 | O1/O2/O6/O25b | 0.25 | 15.5 | 56.09 | 31.1 | 137.86 |
| | 2/2/2/4 μg | | | | | |
| 2 | O1/O2/O6/O25b | 0.21 | 20.5 | 255.77 | 130.25 | 830.71 |
| | 2/2/2/4 μg + CpG | | | | | |
| | Fold rise vs | | 1 | 5 | 4 | 6 |
| | unadjuvanted: | | | | | |
| 3 | O1/O2/O6/O25b | 0.15 | 6.5 | 187.52 | 72.19 | 948.85 |
| | 0.5/0.5/0.5/1 μg + CpG | | | | | |
| | Fold rise vs | | 0 | 3 | 2 | 7 |
| | unadjuvanted: | | | | | |
| | O25B | | | | | |
| 1 | O1/O2/O6/O25b | 0.06 | 0.5 | 3.39 | 4.63 | 41.95 |
| | 2/2/2/4 μg | | | | | |
| 2 | O1/O2/O6/O25b | 0.3 | 9.7 | 125.6 | 77.01 | 1696.09 |
| | 2/2/2/4 μg | | | | | |
| | + CpG | | | | | |
| | Fold rise vs | | 19 | 37 | 17 | 40 |
| | unadjuvanted: | | | | | |
| 3 | O1/O2/O6/O25b | 0.69 | 8.3 | 187.89 | 44.7 | 702.46 |
| | 0.5/0.5/0.5/1 | | | | | |
| | μg + CpG | | | | | |
| | Fold rise vs | | 17 | 55 | 10 | 17 |
| | unadjuvanted: | | | | | |

TABLE 22
O-Antigen Serotype IgG GMT Titers (IgG dLIA with mAb Standards)
GMT of IgG Levels (μg/ml)

| Bleed Time | Vaccine Group | O1a | O2 | O6 | O25b |
|---|---|---|---|---|---|
| Week (−2) | 4 V O—Ag SCP | 0.15 | 0.46 | 0.10 | 0.02 |
| | 2/2/2/4 μg | | | | |
| (Pre) | 4 V O—Ag SCP | 0.12 | 0.63 | 0.04 | 0.05 |
| | 2/2/2/4 μg + CPG | | | | |
| | Fold rise vs. | 1 | 1 | 0 | 3 |
| | unadjuvanted | | | | |
| | 4 V O—Ag SCP | 0.15 | 0.48 | 0.05 | 0.10 |
| | 0.5/0.5/0.5/1 μg + | | | | |
| | CPG | | | | |
| | Fold rise vs. | 1 | 1 | 1 | 5 |
| | unadjuvanted | | | | |
| Week 2 | 4 V O—Ag SCP | 3.73 | 34.35 | 24.81 | 0.66 |
| | 2/2/2/4 μg | | | | |
| (2 wk PD1) | 4 V O—Ag SCP | 108.60 | 402.79 | 146.05 | 46.05 |
| | 2/2/2/4 μg + CPG | | | | |
| | Fold rise vs. | 29 | 12 | 6 | 70 |
| | unadjuvanted | | | | |
| | 4 V O—Ag SCP | 55.80 | 204.89 | 102.26 | 58.17 |
| | 0.5/0.5/0.5/1 μg + | | | | |
| | CPG | | | | |
| | Fold rise vs. | 15 | 6 | 4 | 88 |
| | unadjuvanted | | | | |
| Week 10 | 4 V O—Ag SCP | 12.56 | 43.11 | 46.96 | 5.47 |
| | 2/2/2/4 μg | | | | |
| (2 wk PD2) | 4 V O—Ag SCP | 279.21 | 417.81 | 324.27 | 372.74 |
| | 2/2/2/4 μg + CPG | | | | |
| | Fold rise vs. | 22 | 10 | 7 | 68 |
| | unadjuvanted | | | | |
| | 4 V O—Ag SCP | 108.57 | 348.78 | 273.53 | 131.87 |
| | 0.5/0.5/0.5/1 μg + | | | | |
| | CPG | | | | |
| | Fold rise vs. | 9 | 8 | 6 | 24 |
| | unadjuvanted | | | | |
| Week 26 | 4 V O—Ag SCP | 13.96 | 34.42 | 39.09 | 10.59 |
| | 2/2/2/4 μg | | | | |
| (2 wk PD3) | 4 V O—Ag SCP | 316.64 | 409.22 | 281.74 | 366.72 |
| | 2/2/2/4 μg + CPG | | | | |
| | Fold rise vs. | 23 | 12 | 7 | 35 |
| | unadjuvanted | | | | |
| | 4 V O—Ag SCP | 198.54 | 382.46 | 300.79 | 317.16 |
| | 0.5/0.5/0.5/1 μg + | | | | |
| | CPG | | | | |
| | Fold rise vs. | 14 | 11 | 8 | 30 |
| | unadjuvanted | | | | |

Opsonophagocytic assay (OPA) assays were also completed and OPA titers were determined for each of O-antigen serotypes O1a, O2, O6, and O25b. The Ola, O2, O6, and O25b OPA assays used to evaluate the immune sera were completed as described in Example 3, above. Table 23, below, presents data for OPA GMT titers for each of serotypes O1a, O2, O6, and O25b. For OPA testing, IgG was affinity-purified from serum pools using protein G magnetic beads. Affinity purification of IgG from the serum pools resulted in undetectable baseline pre-immune titers, suggesting that serum matrix effects, rather than non-specific E. coli antibodies, might be responsible for the high non-specific activity observed in the preimmune serum from these animals.

Functional OPA responses to the CpG adjuvanted formulations generally tracked with the IgG titers. For the less immunogenic O1a and O25b serotypes, no OPA activity was detected in IgGs purified from unadjuvanted group pooled sera at PD1 or PD2, while bactericidal activity was readily detected in IgG purified from the CpG adjuvanted NHP pools. In contrast, serotype O2 and O6 OPA activity was readily detected in IgG antibodies purified from unadjuvanted group serum pools sampled at PD1 or PD2 timepoints. After the third vaccine dose, O1a and O25b OPA titers increased further, while the analogous 02 and 06 titers remained relatively unchanged, similar to the PD2 titers.

Significantly, when formulated with CpG, the low-dose 4V-SCP O-antigen glycoconjugate (0.5/0.5/0.5/1 μg) elicited a similar level of serotype-specific functional activity as the four-fold higher base glycoconjugate dose (2/2/2/4 μg), clearly demonstrating the dose-sparing property of the adjuvant.

TABLE 23
OPA Evaluation of Affinity-Purified IgG from NHP Serum Pools
Mean OPA IC₅₀ Titers (Serum Pool IgG)

| Bleed Time | Vaccine Group | O1a | O2 | O6 | O25b |
|---|---|---|---|---|---|
| Week (−2) | 4 V O—Ag SCP | 0.00005 | 0.00005 | 0.00005 | 0.00005 |
| | 2/2/2/4 μg | | | | |
| (Pre) | 4 V O—Ag SCP | 0.00005 | 0.00005 | 0.00005 | 0.00005 |
| | 2/2/2/4 μg + CPG | | | | |
| | Fold rise vs. | | | | |
| | unadjuvanted | | | | |
| | 4 V O—Ag SCP | 0.00005 | 0.00005 | 0.00005 | 0.00005 |
| | 0.5/0.5/0.5/ | | | | |
| | 1 μg + CPG | | | | |
| | Fold rise vs. | | | | |
| | unadjuvanted | | | | |
| Week 2 | 4 V O—Ag SCP | 0.00005 | 0.1553 | 0.0288 | 0.0001 |
| | 2/2/2/4 μg | | | | |
| (2 wk PD1) | 4 V O—Ag SCP | 0.00005 | 0.6669 | 0.1731 | 0.0142 |
| | 2/2/2/4 μg + CPG | | | | |
| | Fold rise vs. | 1 | 4 | 6 | 284 |
| | unadjuvanted | | | | |
| | 4 V O—Ag SCP | 0.0003 | 0.2580 | 0.0711 | 0.0052 |
| | 0.5/0.5/0.5/ | | | | |
| | 1 μg + CPG | | | | |
| | Fold rise vs. | 6 | 2 | 2 | 104 |
| | unadjuvanted | | | | |
| Week 10 | 4 V O—Ag SCP | 0.00005 | 0.0339 | 0.0129 | 0.0001 |
| | 2/2/2/4 μg | | | | |
| (2 wk PD2) | 4 V O—Ag SCP | 0.0047 | 0.5115 | 0.2895 | 0.1158 |
| | 2/2/2/4 μg + CPG | | | | |
| | Fold rise vs. | 94 | 15 | 22 | 2316 |
| | unadjuvanted | | | | |
| | 4 V O—Ag SCP | 0.0062 | 0.8327 | 0.3494 | 0.0605 |
| | 0.5/0.5/0.5/ | | | | |
| | 1 μg + CPG | | | | |
| | Fold rise vs. | 124 | 25 | 27 | 1210 |
| | unadjuvanted | | | | |
| Week 26 | 4 V O—Ag SCP | 0.00005 | 0.1027 | 0.0383 | 0.0026 |
| | 2/2/2/4 μg | | | | |
| (2 wk PD3) | 4 V O—Ag SCP | 0.0511 | 0.9804 | 0.3831 | 0.2753 |
| | 2/2/2/4 μg + CPG | | | | |
| | Fold rise vs. | 1022 | 10 | 10 | 106 |
| | unadjuvanted | | | | |
| | 4 V O—Ag SCP | 0.0228 | 0.9804 | 0.7711 | 0.2989 |
| | 0.5/0.5/0.5/ | | | | |
| | 1 μg + CPG | | | | |
| | Fold rise vs. | 456 | 10 | 20 | 115 |
| | unadjuvanted | | | | |

### Conclusions

In conclusion, the study results demonstrate robust adjuvanting of O-antigen serotype specific IgG and OPA titers by a CpG nucleotide adjuvant in a NHP model. Formulation with CpG resulted in substantial gains in IgG titers independent of O-antigen dose level, with up to ten-fold increases observed for serotypes 02 and O6, and greater than twenty-fold increases observed for the serotype O25b and O1a O-antigens. The overall impact of the CpG adjuvant was to increase the magnitude of the IgG responses to all O-antigen serotypes to similar order of magnitude levels after one or two vaccine doses.

### Additional Sequences

TABLE 24
FimH wild type and mutant sequences

SEQ ID NO: 1 >FimHLD_WT
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 2 >FimHLD_G65A_V27A
FACKTASGTAIPIGGGSANVYVNLAPAVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 3 >FimHLD_F11
IACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 4 >FimHLD_F1L
LACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 5 >FimHLD_F1V
VACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 6 >FimHLD_F1M
MACKTASGTAIPIGGGSANVYVNLAPVVNVGQNLVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 7 >FimHLD_F1Y
YACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 8 >FimHLD_F1W
WACKTASGTAIPIGGGSANVYVNLAPVVNVGQNLVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 9 >FimHLD_Q133K
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 10 >FimHLD_G15A
FACKTASGTAIPIGAGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 11 >FimHLD_G15P
FACKTASGTAIPIGPGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 12 >FimHLD_G16A
FACKTASGTAIPIGGASANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 13 >FimHLD_G16P
FACKTASGTAIPIGGPSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 14 >FimHLD_G15A_G16A
FACKTASGTAIPIGAASANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 15 >FimHLD_R60P
FACKTASGTAIPIGGGSANVYVNLAPVVNVGQNLVVDLSTQIFCHNDYPETITDYVTLQPGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 16 >FimHLD_G65A
FACKTASGTAIPIGGGSANVYVNLAPVVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 17 >FimHLD_P12C_A18C
FACKTASGTAICIGGGSCNVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 18 >FimHLD_G14C_F144C
FACKTASGTAIPICGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQCVWNIYANNDVVVPTGG
SEQ ID NO: 19 >FimHLD_P26C_V35C
FACKTASGTAIPIGGGSANVYVNLACVVNVGQNLCVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 20 >FimHLD_P26C_V154C
FACKTASGTAIPIGGGSANVYVNLACVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDCVVPTGG
SEQ ID NO: 21 >FimHLD_P26C_V156C
FACKTASGTAIPIGGGSANVYVNLACVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVCPTGG
SEQ ID NO: 22 >FimHLD_V27C_L34C
FACKTASGTAIPIGGGSANVYVNLAPCVNVGQNCVVDLSTQIFCHNDYPETITDYVTLQRGSAYGGVL
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 23 >FimHLD_V28C_N33C
FACKTASGTAIPIGGGSANVYVNLAPVCNVGQCLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 24 >FimHLD_V28C_P157C
FACKTASGTAIPIGGGSANVYVNLAPVCNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVCTGG
SEQ ID NO: 25 >FimHLD_Q32C_Y108C
FACKTASGTAIPIGGGSANVYVNLAPVVNVGCNLVVDLSTQIFCHNDYPETITDYVTLQRGSAYGGVLS
SFSGTVKYSGSSYPFPTTSETPRVVYNSRTDKPWPVALCLTPVSSAGGVAIKAGSLIAVLILRQTNNYN
SDDFQFVWNIYANNDVVVPTGG
SEQ ID NO: 26 >FimHLD_N33C_L109C
FACKTASGTAIPIGGGSANVYVNLAPVVNVGQCLVVDLSTQIFCHNDYPETITDYVTLQRGSAYGGVLS
SFSGTVKYSGSSYPFPTTSETPRVVYNSRTDKPWPVALYCTPVSSAGGVAIKAGSLIAVLILRQTNNYN
SDDFQFVWNIYANNDVVVPTGG
SEQ ID NO: 27 >FimHLD_N33C_P157C
FACKTASGTAIPIGGGSANVYVNLAPVVNVGQCLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVCTGG
SEQ ID NO: 28 >FimHLD_V35C_L107C
FACKTASGTAIPIGGGSANVYVNLAPVVNVGQNLCVDLSTQIFCHNDYPETITDYVTLQRGSAYGGVLS
SFSGTVKYSGSSYPFPTTSETPRVVYNSRTDKPWPVACYLTPVSSAGGVAIKAGSLIAVLILRQTNNYN
SDDFQFVWNIYANNDVVVPTGG
SEQ ID NO: 29 >FimHLD_V35C_L109C
FACKTASGTAIPIGGGSANVYVNLAPVVNVGQNLCVDLSTQIFCHNDYPETITDYVTLQRGSAYGGVLS
SFSGTVKYSGSSYPFPTTSETPRVVYNSRTDKPWPVALYCTPVSSAGGVAIKAGSLIAVLILRQTNNYN
SDDFQFVWNIYANNDVVVPTGG
SEQ ID NO: 30 >FimHLD_S62C_T86C
FACKTASGTAIPIGGGSANVYVNLAPVVNVGQNLVVDLSTQIFCHNDYPETITDYVTLQRGCAYGGVLS
SFSGTVKYSGSSYPFPCTSETPRVVYNSRTDKPWPVALYLTPVSSAGGVAIKAGSLIAVLILRQTNNYN
SDDFQFVWNIYANNDVVVPTGG
SEQ ID NO: 31 >FimHLD_S62C_L129C
FACKTASGTAIPIGGGSANVYVNLAPVVNVGQNLVVDLSTQIFCHNDYPETITDYVTLQRGCAYGGVLS
SFSGTVKYSGSSYPFPTTSETPRVVYNSRTDKPWPVALYLTPVSSAGGVAIKAGSLIAVCILRQTNNYN
SDDFQFVWNIYANNDVVVPTGG
SEQ ID NO: 32 >FimHLD_Y64C_L68C
FACKTASGTAIPIGGGSANVYVNLAPVVNVGQNLVVDLSTQIFCHNDYPETITDYVTLQRGSACGGVC
SSFSGTVKYSGSSYPFPTTSETPRVVYNSRTDKPWPVALYLTPVSSAGGVAIKAGSLIAVLILRQTNNY
NSDDFQFVWNIYANNDVVVPTGG
SEQ ID NO: 33 >FimHLD_Y64C_A127C
FACKTASGTAIPIGGGSANVYVNLAPVVNVGQNLVVDLSTQIFCHNDYPETITDYVTLQRGSACGGVLS
SFSGTVKYSGSSYPFPTTSETPRVVYNSRTDKPWPVALYLTPVSSAGGVAIKAGSLICVLILRQTNNYN
SDDFQFVWNIYANNDVVVPTGG
SEQ ID NO: 34 >FimHLD_L68C_F71C
FACKTASGTAIPIGGGSANVYVNLAPVVNVGQNLVVDLSTQIFCHNDYPETITDYVTLQRGSAYGGVCS
SCSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 35 >FimHLD_V112C_T158C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPCSSAGGVAIKAGSLIAVLILRQTNNYN
SDDFQFVWNIYANNDVVVPCGG
SEQ ID NO: 36 >FimHLD_S113C_G116C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVCSACGVAIKAGSLIAVLILRQTNNYN
SDDFQFVWNIYANNDVVVPTGG
SEQ ID NO: 37 >FimHLD_S113C_T158C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVCSAGGVAIKAGSLIAVLILRQTNNYN
SDDFQFVWNIYANNDVVVPCGG
SEQ ID NO: 38 >FimHLD_V118C_V156C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGCAIKAGSLIAVLILRQTNNYN
SDDFQFVWNIYANNDVVCPTGG
SEQ ID NO: 39 >FimHLD_A119C_V155C
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVCIKAGSLIAVLILRQTNNYN
SDDFQFVWNIYANNDVCVPTGG
SEQ ID NO: 40 >FimHLD_L34N_V27A
FACKTASGTAIPIGGGSANVYVNLAPAVNVGQNNVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 41 >FimHLD_L34S_V27A
FACKTASGTAIPIGGGSANVYVNLAPAVNVGQNSVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 42 >FimHLD_L34T_V27A
FACKTASGTAIPIGGGSANVYVNLAPAVNVGQNTVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 43 >FimHLD_A119N_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVNIKAGSLIAVLILRQTNNYN
SDDFQFVWNIYANNDVVVPTGG
SEQ ID NO: 44 >FimHLD_A119S_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVSIKAGSLIAVLILRQTNNYN
SDDFQFVWNIYANNDVVVPTGG
SEQ ID NO: 45 >FimHLD_A119T_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVTIKAGSLIAVLILRQTNNYN
SDDFQFVWNIYANNDVVVPTGG
SEQ ID NO: 46 >FimH-DSG_A115V
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSVGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 47 >FimH-DSG_V1631
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDISARDVTVTLPDYPGSVPIPLTVYCAKSQNLGYYLSGTTADAGN
SIFTNTASFSPAQGVGVQLTRQGTIIPANNTVSLGAVGTSAVSLGLTANYARTGGQVTAGNVQSIIGVT
FVYQGGSSGGGADVTITVNGKVVAK
SEQ ID NO: 48 >FimH-DSG_V1851
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTIYCAKSQNLGYYLSGTTADAGN
SIFTNTASFSPAQGVGVQLTRQGTIIPANNTVSLGAVGTSAVSLGLTANYARTGGQVTAGNVQSIIGVT
FVYQGGSSGGGADVTITVNGKVVAK
SEQ ID NO: 49 >FimH-DSG_DSG_V31
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADITITVNGKVVAK
SEQ ID NO: 50 >FimHLD_G15A_V27A
FACKTASGTAIPIGAGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 51 >FimHLD_G16A_V27A
FACKTASGTAIPIGGASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 52 >FimHLD_G15P_V27A
FACKTASGTAIPIGPGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 53 >FimHLD_G16P_V27A
FACKTASGTAIPIGGPSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 54 >FimHLD_G15A_G16A_V27A
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 55 >FimHLD_V27A_R60P
FACKTASGTAIPIGGGSANVYVNLAPAVNVGQNLVVDLSTQIFCHNDYPETITDYVTLQPGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 56 >FimHLD_G65A_V27A
FACKTASGTAIPIGGGSANVYVNLAPAVNVGQNLVVDLSTQIFCHNDYPETITDYVTLQRGSAYAGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGG
SEQ ID NO: 57 >FimHLD_V27A_Q133K
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 58 >FimHLD_G15A_G16A_V27A_Q133K
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGG
SEQ ID NO: 59 >FimH-DSG_WT
FACKTASGTAIPIGGGSANVYVNLAPVVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 60 >FimH-DSG_V27A
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO:61 >FimH-DSG_G15A_V27A
FACKTASGTAIPIGAGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 62 >FimH-DSG_G15A_G16A_V27A
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRQTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 63 >FimH-DSG_V27A_Q133K
FACKTASGTAIPIGGGSANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 64 >FimH-DSG_G15A_G16A_V27A_Q133K
FACKTASGTAIPIGAASANVYVNLAPAVNVGQNLVVDLSTQIFCHNDYPETITDYVTLQRGSAYGGVLS
SFSGTVKYSGSSYPFPTTSETPRVVYNSRTDKPWPVALYLTPVSSAGGVAIKAGSLIAVLILRKTNNYN
SDDFQFVWNIYANNDVVVPTGGCDVSARDVTVTLPDYPGSVPIPLTVYCAKSQNLGYYLSGTTADAG
NSIFTNTASFSPAQGVGVQLTRQGTIIPANNTVSLGAVGTSAVSLGLTANYARTGGQVTAGNVQSIIGV
TFVYQGGSSGGGADVTITVNGKVVAK
SEQ ID NO: 65 >mouse Ig Kappa signal peptide
ETDTLLLWVLLLWVPGSTG

TABLE 25
Additional Sequences

| Description | Sequence | |
|---|---|---|
| 80A polyA | AAAAAAAAAAAAAAAAAAAAAAAAAAAAAAAAAAAAAAAAAAAAAAAAAAAAAA | SEQ ID NO: 90 |
| tail | AAAAAAAAAAAAAAAAAAAAAAAAAAAA | |
| “30L70” | AAAAAAAAAAAAAAAAAAAAAAAAAAAAAAGCAUAUGACUAAAAAAAAAAAAA | SEQ ID NO: 91 |
| polyA tail | AAAAAAAAAAAAAAAAAAAAAAAAAAAAAAAAAAAAAAAAAAAAAAAAAAAAAA | |
| | AA | |
| Glycine- | GSSGSGSS | SEQ ID NO: 92 |
| Serine linker | | |
| substitution | | |
| in the DAF | | |
| GPI anchor | | |
| 5′ | AGGAAAUAAGAGAGGAUAAGACGACUAAGGAGACAUACAGAAUAAGAGGCA | SEQ ID NO: 93 |
| UTR_BMD2 | GCCACC | |
| 5′ | AGAAGAGAACCUCGUCGAGUCCUGGUAGUAGUAAUCCUAGAGGAGCCACC | SEQ ID NO: 94 |
| UTR_BMD70 | | |
| 5′ | AGGAGGGUAAUUCGCUUAGCGAUAGUACUAUCGAAGCGUACAGAGCCACC | SEQ ID NO: 95 |
| UTR_BMD91 | | |
| 5′UTR_BMD | AGGAGGACUGCGCGAACCUGCAUAGUGAUCAUAAGGUCAUGAUAGCCACC | SEQ ID NO: 96 |
| 105 | | |
| 5′UTR_BMD | AGGAAAUAAGAGAAAGAGGAUAAGACGACUAAGGAGACAUACAGAAUAAGA | SEQ ID NO: 97 |
| 562 | GGCAGCCACC | |
| 5′UTR BMD | AGGAAAUAAGAAAGAAGACAGAAGAAGACAGAAGAAGAACCAGAGAAGGAC | SEQ ID NO: 98 |
| 3 | AAGCCACC | |
| 5′ | AGGAGGACUGGUCGAACCUGCAUAGUGAUCAUAAGGUCAGCAUAGCCACC | SEQ ID NO: 99 |
| UTR_BMD5 | | |
| 76 | | |
| 3′ | GCUCGCUUUCUUGCUGUCCAAUUUCUAUUAAAGGUUCCUUUGUUCCCUAA | SEQ ID NO: 100 |
| UTR_hHBB | GUCCAACUACUAAACUGGGGGAUAUUAUGAAGGGCCUUGAGCAUCUGGAU | |
| | UCUGCCUAAUAAAAAACAUUUAUUUUCAUUGC | |
| 3′ | GUGUGUGGAGGACACCCUGAACCCCCCGCUUUCAAACAAGUUUUCAAAUU | SEQ ID NO: 101 |
| UTR_CYP2 | GUUUGAGGUCAGGAUUUCUCAAACUGAUUCCUUUCUUUGCAUAUGAGUAU | |
| E1 | UUGAAAAUAAAUAUUUUCCC | |
| 3′ | GCUCGCUUUCUUGCUGUCCAAUUUCUAUUAAAGGUUCCUUUGUUCCCUAA | SEQ ID NO: 102 |
| UTR_hHBB- | GUCCAACUACUAAACUGGGGGAUAUUAUGAAGGGCCUUGAGCAUCUGGAU | |
| AES | UCUGCCUAAUAAAAAACAUUUAUUUUCAUUGCAACCCUCGACUGGUACUGC | |
| | AUGCACGCAAUGCUAGCUGCCCCUUUCCCGUCCUGGGUACCCCGAGUCUC | |
| | CCCCGACCUCGGGUCCCAGGUAUGCUCCCACCUCCACCUGCCCCACUCAC | |
| | CACCUCUGCUAGUUCCAGACACCUCC | |

### FimH Transcript Nucleotide Sequences

RNA transcript sequences of ten constructs are listed below and include 5′UTR, FimH gene, 3′UTR and 3′polyA sequences. The provided alias describes the constructs in terms of 5′UTR/FimH gene variant/3′UTR_polyA type. Sequence annotations are as follows: AUG, first methionine of the gene of interest (bold); UGAUAG or UGAUGA, stop codons after gene of interest (bold and italics); 5′UTRs are underlined; 5′UTR #15 is also known as BMD2; 5′UTR #16 is also known as BMD3; 3′UTR #2: from human hemoglobin beta (hHBB); 3′UTR #7: from CYP2E1 (aka C31PO); 3′UTR #62: dual 3′ UTR comprised of 132 nt of hHBB and 136 nt of AES (human amino-terminal enhancer of split) mRNA sequences. PolyA tract is either 80nt (SEQ ID NO: 90) or the split polyA, which is referred to as the “301L70” polyA (SEQ ID NO: 91) (italics).

>BMD2/FimH_{DSG}-GPI/hHBB-80pA
(SEQ ID NO: 66)
AGGAAAUAAGAGAGGAUAAGACGACUAAGGAGACAUACAGAAUAAGAGGCAGCCACCAU
GGAGACCGACACACUGCUGCUGUGGGUGCUGCUGCUGUGGGUGCCCGGCUCCACCGG
CUUCGCUUGCAAGACAGCCAGCGGAACCGCCAUCCCUAUCGGCGCAGCAAGCGCCAAC
GUCUACGUGAAUCUGGCUCCCGCAGUGAACGUGGGACAGAAUCUGGUGGUGGAUCUGA
GCACCCAGAUCUUCUGCCACAAUGACUACCCCGAGACCAUCACAGACUACGUGACACUG
CAGAGAGGAAGCGCCUACGGCGGCGUGCUGAGCAGCUUCAGCGGAACCGUGAAAUACA
GCGGCUCCAGCUACCCCUUCCCCACAACCAGCGAGACCCCUAGAGUGGUCUAUAACUC
UAGAACAGACAAGCCUUGGCCCGUGGCUCUGUAUCUGACCCCCGUGUCCAGCGCUGGA
GGAGUGGCCAUCAAGGCCGGCAGCCUCAUCGCCGUCCUCAUUCUGAGGCAGACCAACA
ACUACAACAGCGACGACUUCCAGUUCGUGUGGAACAUCUACGCCAACAACGACGUGGU
GGUCCCCACCGGCGGAUGUGACGUGUCCGCCAGAGAUGUGACCGUGACACUGCCCGAU
UACCCCGGAAGCGUCCCUAUCCCUCUGACAGUGUACUGCGCCAAGAGCCAAAAUCUGG
GCUACUAUCUGUCCGGAACCACAGCCGACGCCGGAAACUCCAUCUUCACCAACACCGCC
AGCUUUUCCCCCGCCCAAGGAGUGGGAGUCCAGCUGACAAGAAGCGGCACCAUCAUCC
CCGCCAGCAACACAGUGUCUCUGGGCGCUGUGGGCACAUCCGCUGUGUCUCUGGGAC
UGACAGCUAAUUAUGCCAGAACCGGAGGCCAAGUGACCGCUGGAAAUGUGCAGAGCAU
UAUUGGGGUGACCUUCGUGUACCAGGGCGGAAGUAGCGGAGGCGGUGCCGACGUGAC
CAUCACCGUGAACGGCAAGGUGGUGGCCAAGAGUUCUGGUGGCGGUCCCAACAAAGGG
AGUGGAACAACAUCCGGGACUACGCGAUUGCUUUCCGGCCAUACUUGCUUUACACUGA
CUGGCCUGUUGGGGACGCUCGUUACGAUGGGUCUGCUCACCUGAUAGGCUCGCUUUC
UUGCUGUCCAAUUUCUAUUAAAGGUUCCUUUGUUCCCUAAGUCCAACUACUAAACUGGG
GGAUAUUAUGAAGGGCCUUGAGCAUCUGGAUUCUGCCUAAUAAAAAACAUUUAUUUUCA
UUGCAAAAAAAAAAAAAAAAAAAAAAAAAAAAAAAAAAAAAAAAAAAAAAAAAAAAAAAAAAA
AAAAAAAAAAAAAAAAAAAAAAA
>BMD70/FimH_{DSG}-GPI/hHBB_80pA
(SEQ ID NO: 67)
AGAAGAGAACCUCGUCGAGUCCUGGUAGUAGUAAUCCUAGAGGAGCCACCAUGGAGAC
CGACACACUGCUGCUGUGGGUGCUGCUGCUGUGGGUGCCCGGCUCCACCGGCUUCGC
UUGCAAGACAGCCAGCGGAACCGCCAUCCCUAUCGGCGCAGCAAGCGCCAACGUCUAC
GUGAAUCUGGCUCCCGCAGUGAACGUGGGACAGAAUCUGGUGGUGGAUCUGAGCACCC
AGAUCUUCUGCCACAAUGACUACCCCGAGACCAUCACAGACUACGUGACACUGCAGAGA
GGAAGCGCCUACGGCGGCGUGCUGAGCAGCUUCAGCGGAACCGUGAAAUACAGCGGCU
CCAGCUACCCCUUCCCCACAACCAGCGAGACCCCUAGAGUGGUCUAUAACUCUAGAACA
GACAAGCCUUGGCCCGUGGCUCUGUAUCUGACCCCCGUGUCCAGCGCUGGAGGAGUG
GCCAUCAAGGCCGGCAGCCUCAUCGCCGUCCUCAUUCUGAGGCAGACCAACAACUACAA
CAGCGACGACUUCCAGUUCGUGUGGAACAUCUACGCCAACAACGACGUGGUGGUCCCC
ACCGGCGGAUGUGACGUGUCCGCCAGAGAUGUGACCGUGACACUGCCCGAUUACCCCG
GAAGCGUCCCUAUCCCUCUGACAGUGUACUGCGCCAAGAGCCAAAAUCUGGGCUACUA
UCUGUCCGGAACCACAGCCGACGCCGGAAACUCCAUCUUCACCAACACCGCCAGCUUUU
CCCCCGCCCAAGGAGUGGGAGUCCAGCUGACAAGAAGCGGCACCAUCAUCCCCGCCAG
CAACACAGUGUCUCUGGGCGCUGUGGGCACAUCCGCUGUGUCUCUGGGACUGACAGCU
AAUUAUGCCAGAACCGGAGGCCAAGUGACCGCUGGAAAUGUGCAGAGCAUUAUUGGGG
UGACCUUCGUGUACCAGGGCGGAAGUAGCGGAGGCGGUGCCGACGUGACCAUCACCG
UGAACGGCAAGGUGGUGGCCAAGAGUUCUGGUGGCGGUCCCAACAAAGGGAGUGGAAC
AACAUCCGGGACUACGCGAUUGCUUUCCGGCCAUACUUGCUUUACACUGACUGGCCUG
UUGGGGACGCUCGUUACGAUGGGUCUGCUCACCUGAUAGGCUCGCUUUCUUGCUGUC
CAAUUUCUAUUAAAGGUUCCUUUGUUCCCUAAGUCCAACUACUAAACUGGGGGAUAUUA
UGAAGGGCCUUGAGCAUCUGGAUUCUGCCUAAUAAAAAACAUUUAUUUUCAUUGCAAAA
AAAAAAAAAAAAAAAAAAAAAAAAAAAAAAAAAAAAAAAAAAAAAAAAAAAAAAAAAAAAAAA
AAAAAAAAAAAAAAA
>BMD91/FimH_{DSG}-GPI/CYP2E1_80pA
(SEQ ID NO: 68)
AGGAGGGUAAUUCGCUUAGCGAUAGUACUAUCGAAGCGUACAGAGCCACCAUGGAGAC
CGACACACUGCUGCUGUGGGUGCUGCUGCUGUGGGUGCCCGGCUCCACCGGCUUCGC
UUGCAAGACAGCCAGCGGAACCGCCAUCCCUAUCGGCGCAGCAAGCGCCAACGUCUAC
GUGAAUCUGGCUCCCGCAGUGAACGUGGGACAGAAUCUGGUGGUGGAUCUGAGCACCC
AGAUCUUCUGCCACAAUGACUACCCCGAGACCAUCACAGACUACGUGACACUGCAGAGA
GGAAGCGCCUACGGCGGCGUGCUGAGCAGCUUCAGCGGAACCGUGAAAUACAGCGGCU
CCAGCUACCCCUUCCCCACAACCAGCGAGACCCCUAGAGUGGUCUAUAACUCUAGAACA
GACAAGCCUUGGCCCGUGGCUCUGUAUCUGACCCCCGUGUCCAGCGCUGGAGGAGUG
GCCAUCAAGGCCGGCAGCCUCAUCGCCGUCCUCAUUCUGAGGCAGACCAACAACUACAA
CAGCGACGACUUCCAGUUCGUGUGGAACAUCUACGCCAACAACGACGUGGUGGUCCCC
ACCGGCGGAUGUGACGUGUCCGCCAGAGAUGUGACCGUGACACUGCCCGAUUACCCCG
GAAGCGUCCCUAUCCCUCUGACAGUGUACUGCGCCAAGAGCCAAAAUCUGGGCUACUA
UCUGUCCGGAACCACAGCCGACGCCGGAAACUCCAUCUUCACCAACACCGCCAGCUUUU
CCCCCGCCCAAGGAGUGGGAGUCCAGCUGACAAGAAGCGGCACCAUCAUCCCCGCCAG
CAACACAGUGUCUCUGGGCGCUGUGGGCACAUCCGCUGUGUCUCUGGGACUGACAGCU
AAUUAUGCCAGAACCGGAGGCCAAGUGACCGCUGGAAAUGUGCAGAGCAUUAUUGGGG
UGACCUUCGUGUACCAGGGCGGAAGUAGCGGAGGCGGUGCCGACGUGACCAUCACCG
UGAACGGCAAGGUGGUGGCCAAGAGUUCUGGUGGCGGUCCCAACAAAGGGAGUGGAAC
AACAUCCGGGACUACGCGAUUGCUUUCCGGCCAUACUUGCUUUACACUGACUGGCCUG
UUGGGGACGCUCGUUACGAUGGGUCUGCUCACCUGAUAGGUGUGUGGAGGACACCCU
GAACCCCCCGCUUUCAAACAAGUUUUCAAAUUGUUUGAGGUCAGGAUUUCUCAAACUGA
UUCCUUUCUUUGCAUAUGAGUAUUUGAAAAUAAAUAUUUUCCCAAAAAAAAAAAAAAAAA
AAAAAAAAAAAAAAAAAAAAAAAAAAAAAAAAAAAAAAAAAAAAAAAAAAAAAAAAAAAAAAAA
>BMD105/FimH_{DSG}-GPI/hHBB_80pA
(SEQ ID NO: 69)
AGGAGGACUGCGCGAACCUGCAUAGUGAUCAUAAGGUCAUGAUAGCCACCAUGGAGAC
CGACACACUGCUGCUGUGGGUGCUGCUGCUGUGGGUGCCCGGCUCCACCGGCUUCGC
UUGCAAGACAGCCAGCGGAACCGCCAUCCCUAUCGGCGCAGCAAGCGCCAACGUCUAC
GUGAAUCUGGCUCCCGCAGUGAACGUGGGACAGAAUCUGGUGGUGGAUCUGAGCACCC
AGAUCUUCUGCCACAAUGACUACCCCGAGACCAUCACAGACUACGUGACACUGCAGAGA
GGAAGCGCCUACGGCGGCGUGCUGAGCAGCUUCAGCGGAACCGUGAAAUACAGCGGCU
CCAGCUACCCCUUCCCCACAACCAGCGAGACCCCUAGAGUGGUCUAUAACUCUAGAACA
GACAAGCCUUGGCCCGUGGCUCUGUAUCUGACCCCCGUGUCCAGCGCUGGAGGAGUG
GCCAUCAAGGCCGGCAGCCUCAUCGCCGUCCUCAUUCUGAGGCAGACCAACAACUACAA
CAGCGACGACUUCCAGUUCGUGUGGAACAUCUACGCCAACAACGACGUGGUGGUCCCC
ACCGGCGGAUGUGACGUGUCCGCCAGAGAUGUGACCGUGACACUGCCCGAUUACCCCG
GAAGCGUCCCUAUCCCUCUGACAGUGUACUGCGCCAAGAGCCAAAAUCUGGGCUACUA
UCUGUCCGGAACCACAGCCGACGCCGGAAACUCCAUCUUCACCAACACCGCCAGCUUUU
CCCCCGCCCAAGGAGUGGGAGUCCAGCUGACAAGAAGCGGCACCAUCAUCCCCGCCAG
CAACACAGUGUCUCUGGGCGCUGUGGGCACAUCCGCUGUGUCUCUGGGACUGACAGCU
AAUUAUGCCAGAACCGGAGGCCAAGUGACCGCUGGAAAUGUGCAGAGCAUUAUUGGGG
UGACCUUCGUGUACCAGGGCGGAAGUAGCGGAGGCGGUGCCGACGUGACCAUCACCG
UGAACGGCAAGGUGGUGGCCAAGAGUUCUGGUGGCGGUCCCAACAAAGGGAGUGGAAC
AACAUCCGGGACUACGCGAUUGCUUUCCGGCCAUACUUGCUUUACACUGACUGGCCUG
UUGGGGACGCUCGUUACGAUGGGUCUGCUCACCUGAUAGGCUCGCUUUCUUGCUGUC
CAAUUUCUAUUAAAGGUUCCUUUGUUCCCUAAGUCCAACUACUAAACUGGGGGAUAUUA
UGAAGGGCCUUGAGCAUCUGGAUUCUGCCUAAUAAAAAACAUUUAUUUUCAUUGCAAAA
AAAAAAAAAAAAAAAAAAAAAAAAAAAAAAAAAAAAAAAAAAAAAAAAAAAAAAAAAAAAAAA
AAAAAAAAAAAAAAAA
>BMD562/FimH_{DSG}-GPI/hHBB_80pA
(SEQ ID NO: 70)
AGGAAAUAAGAGAAAGAGGAUAAGACGACUAAGGAGACAUACAGAAUAAGAGGCAGCCA
CCAUGGAGACCGACACACUGCUGCUGUGGGUGCUGCUGCUGUGGGUGCCCGGCUCCA
CCGGCUUCGCUUGCAAGACAGCCAGCGGAACCGCCAUCCCUAUCGGCGCAGCAAGCGC
CAACGUCUACGUGAAUCUGGCUCCCGCAGUGAACGUGGGACAGAAUCUGGUGGUGGAU
CUGAGCACCCAGAUCUUCUGCCACAAUGACUACCCCGAGACCAUCACAGACUACGUGAC
ACUGCAGAGAGGAAGCGCCUACGGCGGCGUGCUGAGCAGCUUCAGCGGAACCGUGAAA
UACAGCGGCUCCAGCUACCCCUUCCCCACAACCAGCGAGACCCCUAGAGUGGUCUAUAA
CUCUAGAACAGACAAGCCUUGGCCCGUGGCUCUGUAUCUGACCCCCGUGUCCAGCGCU
GGAGGAGUGGCCAUCAAGGCCGGCAGCCUCAUCGCCGUCCUCAUUCUGAGGCAGACCA
ACAACUACAACAGCGACGACUUCCAGUUCGUGUGGAACAUCUACGCCAACAACGACGUG
GUGGUCCCCACCGGCGGAUGUGACGUGUCCGCCAGAGAUGUGACCGUGACACUGCCC
GAUUACCCCGGAAGCGUCCCUAUCCCUCUGACAGUGUACUGCGCCAAGAGCCAAAAUC
UGGGCUACUAUCUGUCCGGAACCACAGCCGACGCCGGAAACUCCAUCUUCACCAACACC
GCCAGCUUUUCCCCCGCCCAAGGAGUGGGAGUCCAGCUGACAAGAAGCGGCACCAUCA
UCCCCGCCAGCAACACAGUGUCUCUGGGCGCUGUGGGCACAUCCGCUGUGUCUCUGG
GACUGACAGCUAAUUAUGCCAGAACCGGAGGCCAAGUGACCGCUGGAAAUGUGCAGAG
CAUUAUUGGGGUGACCUUCGUGUACCAGGGCGGAAGUAGCGGAGGCGGUGCCGACGU
GACCAUCACCGUGAACGGCAAGGUGGUGGCCAAGAGUUCUGGUGGCGGUCCCAACAAA
GGGAGUGGAACAACAUCCGGGACUACGCGAUUGCUUUCCGGCCAUACUUGCUUUACAC
UGACUGGCCUGUUGGGGACGCUCGUUACGAUGGGUCUGCUCACCUGAUAGGCUCGCU
UUCUUGCUGUCCAAUUUCUAUUAAAGGUUCCUUUGUUCCCUAAGUCCAACUACUAAACU
GGGGGAUAUUAUGAAGGGCCUUGAGCAUCUGGAUUCUGCCUAAUAAAAAACAUUUAUUU
UCAUUGCAAAAAAAAAAAAAAAAAAAAAAAAAAAAAAAAAAAAAAAAAAAAAAAAAAAAAAA
AAAAAAAAAAAAAAAAAAAAAAAAAAAA
>BMD3/FimH_{DSG}-GPI/hHBB-AES_80pA
(SEQ ID NO: 71)
AGGAAAUAAGAAAGAAGACAGAAGAAGACAGAAGAAGAACCAGAGAAGGACAAGCCACCA
UGGAGACCGACACACUGCUGCUGUGGGUGCUGCUGCUGUGGGUGCCCGGCUCCACCG
GCUUCGCUUGCAAGACAGCCAGCGGAACCGCCAUCCCUAUCGGCGCAGCAAGCGCCAA
CGUCUACGUGAAUCUGGCUCCCGCAGUGAACGUGGGACAGAAUCUGGUGGUGGAUCUG
AGCACCCAGAUCUUCUGCCACAAUGACUACCCCGAGACCAUCACAGACUACGUGACACU
GCAGAGAGGAAGCGCCUACGGCGGCGUGCUGAGCAGCUUCAGCGGAACCGUGAAAUAC
AGCGGCUCCAGCUACCCCUUCCCCACAACCAGCGAGACCCCUAGAGUGGUCUAUAACU
CUAGAACAGACAAGCCUUGGCCCGUGGCUCUGUAUCUGACCCCCGUGUCCAGCGCUGG
AGGAGUGGCCAUCAAGGCCGGCAGCCUCAUCGCCGUCCUCAUUCUGAGGCAGACCAAC
AACUACAACAGCGACGACUUCCAGUUCGUGUGGAACAUCUACGCCAACAACGACGUGGU
GGUCCCCACCGGCGGAUGUGACGUGUCCGCCAGAGAUGUGACCGUGACACUGCCCGAU
UACCCCGGAAGCGUCCCUAUCCCUCUGACAGUGUACUGCGCCAAGAGCCAAAAUCUGG
GCUACUAUCUGUCCGGAACCACAGCCGACGCCGGAAACUCCAUCUUCACCAACACCGCC
AGCUUUUCCCCCGCCCAAGGAGUGGGAGUCCAGCUGACAAGAAGCGGCACCAUCAUCC
CCGCCAGCAACACAGUGUCUCUGGGCGCUGUGGGCACAUCCGCUGUGUCUCUGGGAC
UGACAGCUAAUUAUGCCAGAACCGGAGGCCAAGUGACCGCUGGAAAUGUGCAGAGCAU
UAUUGGGGUGACCUUCGUGUACCAGGGCGGAAGUAGCGGAGGCGGUGCCGACGUGAC
CAUCACCGUGAACGGCAAGGUGGUGGCCAAGAGUUCUGGUGGCGGUCCCAACAAAGGG
AGUGGAACAACAUCCGGGACUACGCGAUUGCUUUCCGGCCAUACUUGCUUUACACUGA
CUGGCCUGUUGGGGACGCUCGUUACGAUGGGUCUGCUCACCUGAUAGGCUCGCUUUC
UUGCUGUCCAAUUUCUAUUAAAGGUUCCUUUGUUCCCUAAGUCCAACUACUAAACUGGG
GGAUAUUAUGAAGGGCCUUGAGCAUCUGGAUUCUGCCUAAUAAAAAACAUUUAUUUUCA
UUGCAACCCUCGACUGGUACUGCAUGCACGCAAUGCUAGCUGCCCCUUUCCCGUCCUG
GGUACCCCGAGUCUCCCCCGACCUCGGGUCCCAGGUAUGCUCCCACCUCCACCUGCCC
CACUCACCACCUCUGCUAGUUCCAGACACCUCCAAAAAAAAAAAAAAAAAAAAAAAAAAAA
AAAAAAAAAAAAAAAAAAAAAAAAAAAAAAAAAAAAAAAAAAAAAAAAAAAAA
>BMD2/FimH_{LD}-GPI/hHBB_80pA
(SEQ ID NO: 72)
AGGAAAUAAGAGAGGAUAAGACGACUAAGGAGACAUACAGAAUAAGAGGCAGCCACCAU
GGAGACCGACACACUGCUGCUGUGGGUGCUUUUGUUGUGGGUGCCCGGUUCUACCGG
AUUUGCGUGUAAGACCGCCUCUGGGACUGCGAUACCCAUCGGUGCUGCAUCCGCUAAC
GUGUAUGUGAAUCUCGCCCCAGCCGUCAACGUAGGUCAAAACUUGGUUGUUGACUUGU
CUACGCAGAUAUUUUGUCACAAUGAUUACCCAGAAACGAUUACCGACUAUGUUACACUC
CAACGGGGCAGCGCCUAUGGCGGUGUACUCAGCAGUUUCAGUGGUACAGUGAAAUAUU
CUGGCAGCAGUUAUCCAUUUCCCACAACUAGCGAAACCCCUAGAGUUGUAUAUAACUCA
CGAACGGACAAGCCUUGGCCGGUGGCGCUCUAUCUGACCCCGGUUAGCUCAGCAGGG
GGAGUGGCAAUUAAGGCGGGGAGUUUGAUCGCCGUGCUUAUACUGCGCCAAACCAACA
AUUACAAUAGUGACGAUUUUCAAUUUGUCUGGAACAUAUACGCCAAUAACGACGUCGUU
GUGCCAACUGGAGGUAGUUCUGGUGGCGGUCCCAACAAAGGGAGUGGAACAACAUCCG
GGACUACGCGAUUGCUUUCCGGCCAUACUUGCUUUACACUGACUGGCCUGUUGGGGAC
GCUCGUUACGAUGGGUCUGCUCACCUGAUGAGCUCGCUUUCUUGCUGUCCAAUUUCUA
UUAAAGGUUCCUUUGUUCCCUAAGUCCAACUACUAAACUGGGGGAUAUUAUGAAGGGC
CUUGAGCAUCUGGAUUCUGCCUAAUAAAAAACAUUUAUUUUCAUUGCAAAAAAAAAAAAA
AAAAAAAAAAAAAAAAAAAAAAAAAAAAAAAAAAAAAAAAAAAAAAAAAAAAAAAAAAAAAAA
AAAAAA
>BMD2/FimH_{DSG}-Sec/hHBB_80pA
(SEQ ID NO: 73)
AGGAAAUAAGAGAGGAUAAGACGACUAAGGAGACAUACAGAAUAAGAGGCAGCCACCAU
GGAGACCGACACACUGCUGCUGUGGGUGCUGCUGCUGUGGGUGCCCGGCUCCACCGG
CUUCGCUUGCAAGACAGCCAGCGGAACCGCCAUCCCUAUCGGCGCAGCAAGCGCCAAC
GUCUACGUGAAUCUGGCUCCCGCAGUGAACGUGGGACAGAAUCUGGUGGUGGAUCUGA
GCACCCAGAUCUUCUGCCACAAUGACUACCCCGAGACCAUCACAGACUACGUGACACUG
CAGAGAGGAAGCGCCUACGGCGGCGUGCUGAGCAGCUUCAGCGGAACCGUGAAAUACA
GCGGCUCCAGCUACCCCUUCCCCACAACCAGCGAGACCCCUAGAGUGGUCUAUAACUC
UAGAACAGACAAGCCUUGGCCCGUGGCUCUGUAUCUGACCCCCGUGUCCAGCGCUGGA
GGAGUGGCCAUCAAGGCCGGCAGCCUCAUCGCCGUCCUCAUUCUGAGGCAGACCAACA
ACUACAACAGCGACGACUUCCAGUUCGUGUGGAACAUCUACGCCAACAACGACGUGGU
GGUCCCCACCGGCGGAUGUGACGUGUCCGCCAGAGACGUGACCGUGACACUGCCCGAU
UACCCCGGAAGCGUCCCUAUCCCUCUGACAGUGUACUGCGCCAAGAGCCAAAAUCUGG
GCUACUAUCUGUCCGGAACCACAGCCGACGCCGGAAACUCCAUCUUCACCAACACCGCC
AGCUUUUCCCCCGCCCAAGGAGUGGGAGUCCAGCUGACAAGAAGCGGCACCAUCAUCC
CCGCCAGCAACACAGUGUCUCUGGGCGCUGUGGGCACAUCCGCUGUGUCUCUGGGAC
UGACAGCUAAUUAUGCCAGAACCGGAGGCCAAGUGACCGCUGGAAAUGUGCAGAGCAU
UAUUGGGGUGACCUUCGUGUACCAGGGCGGAAGUAGCGGAGGCGGUGCCGACGUGAC
CAUCACCGUGAACGGCAAGGUGGUGGCCAAGUGAUGAGCUCGCUUUCUUGCUGUCCAA
UUUCUAUUAAAGGUUCCUUUGUUCCCUAAGUCCAACUACUAAACUGGGGGAUAUUAUGA
AGGGCCUUGAGCAUCUGGAUUCUGCCUAAUAAAAAACAUUUAUUUUCAUUGCAAAAAAA
AAAAAAAAAAAAAAAAAAAAAAAAAAAAAAAAAAAAAAAAAAAAAAAAAAAAAAAAAAAAAAA
AAAAAAAAAAAA

### FimH Antigen Amino Acid Sequences

Underlined are Gly-Ser linkers separating full length FimH from the stabilizing donor strand G-peptide (DSG) and the C-terminal glycosylphosphatidylinositol (GPI) membrane anchoring signal of the human DAF protein. Asterisks represent stop codons. The FimH_{DSG} contains alleles N7S, G15A, G16A, V27A, N70S, N228S and N235S (numbering based on processed polypeptide starting with the proximal phenylalanine residue).

>FimH_{LD}-CtDAFGPI:
(SEQ ID NO: 74)
ATGGAGACCGACACACTGCTGCTGTGGGTGCTTTTGTTGTGGGTGCCCGGTTCTACCGGA
TTTGCGTGTAAGACCGCCTCTGGGACTGCGATACCCATCGGTGCTGCATCCGCTAACGTG
TATGTGAATCTCGCCCCAGCCGTCAACGTAGGTCAAAACTTGGTTGTTGACTTGTCTACGC
AGATATTTTGTCACAATGATTACCCAGAAACGATTACCGACTATGTTACACTCCAACGGGG
CAGCGCCTATGGCGGTGTACTCAGCAGTTTCAGTGGTACAGTGAAATATTCTGGCAGCAG
TTATCCATTTCCCACAACTAGCGAAACCCCTAGAGTTGTATATAACTCACGAACGGACAAG
CCTTGGCCGGTGGCGCTCTATCTGACCCCGGTTAGCTCAGCAGGGGGAGTGGCAATTAA
GGCGGGGAGTTTGATCGCCGTGCTTATACTGCGCCAAACCAACAATTACAATAGTGACGA
TTTTCAATTTGTCTGGAACATATACGCCAATAACGACGTCGTTGTGCCAACTGGAGGTAGT
TCTGGTGGCGGTCCCAACAAAGGGAGTGGAACAACATCCGGGACTACGCGATTGCTTTCC
GGCCATACTTGCTTTACACTGACTGGCCTGTTGGGGACGCTCGTTACGATGGGTCTGCTC
ACCTGATGA
Translation:
(SEQ ID NO: 75)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGSSGGGPNKGS
GTTSGTTRLLSGHTCFTLTGLLGTLVTMGLLT**
>Secreted FimH_{DSG}:
(SEQ ID NO: 76)
ATGGAGACCGACACACTGCTGCTGTGGGTGCTGCTGCTGTGGGTGCCCGGCTCCACCGG
CTTCGCTTGCAAGACAGCCAGCGGAACCGCCATCCCTATCGGCGCAGCAAGCGCCAACG
TCTACGTGAATCTGGCTCCCGCAGTGAACGTGGGACAGAATCTGGTGGTGGATCTGAGCA
CCCAGATCTTCTGCCACAATGACTACCCCGAGACCATCACAGACTACGTGACACTGCAGA
GAGGAAGCGCCTACGGCGGCGTGCTGAGCAGCTTCAGCGGAACCGTGAAATACAGCGGC
TCCAGCTACCCCTTCCCCACAACCAGCGAGACCCCTAGAGTGGTCTATAACTCTAGAACA
GACAAGCCTTGGCCCGTGGCTCTGTATCTGACCCCCGTGTCCAGCGCTGGAGGAGTGGC
CATCAAGGCCGGCAGCCTCATCGCCGTCCTCATTCTGAGGCAGACCAACAACTACAACAG
CGACGACTTCCAGTTCGTGTGGAACATCTACGCCAACAACGACGTGGTGGTCCCCACCGG
CGGATGTGACGTGTCCGCCAGAGACGTGACCGTGACACTGCCCGATTACCCCGGAAGCG
TCCCTATCCCTCTGACAGTGTACTGCGCCAAGAGCCAAAATCTGGGCTACTATCTGTCCGG
AACCACAGCCGACGCCGGAAACTCCATCTTCACCAACACCGCCAGCTTTTCCCCCGCCCA
AGGAGTGGGAGTCCAGCTGACAAGAAGCGGCACCATCATCCCCGCCAGCAACACAGTGT
CTCTGGGCGCTGTGGGCACATCCGCTGTGTCTCTGGGACTGACAGCTAATTATGCCAGAA
CCGGAGGCCAAGTGACCGCTGGAAATGTGCAGAGCATTATTGGGGTGACCTTCGTGTACC
AGGGCGGAAGTAGCGGAGGCGGTGCCGACGTGACCATCACCGTGAACGGCAAGGTGGT
GGCCAAGTGATGA
Translation:
(SEQ ID NO: 77)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQGGSSGGGADVTITVNGKVVA
K**
>FimH_{DSG}-CtDAFGPI:
(SEQ ID NO: 78)
ATGGAGACCGACACACTGCTGCTGTGGGTGCTGCTGCTGTGGGTGCCCGGCTCCACCGG
CTTCGCTTGCAAGACAGCCAGCGGAACCGCCATCCCTATCGGCGCAGCAAGCGCCAACG
TCTACGTGAATCTGGCTCCCGCAGTGAACGTGGGACAGAATCTGGTGGTGGATCTGAGCA
CCCAGATCTTCTGCCACAATGACTACCCCGAGACCATCACAGACTACGTGACACTGCAGA
GAGGAAGCGCCTACGGCGGCGTGCTGAGCAGCTTCAGCGGAACCGTGAAATACAGCGGC
TCCAGCTACCCCTTCCCCACAACCAGCGAGACCCCTAGAGTGGTCTATAACTCTAGAACA
GACAAGCCTTGGCCCGTGGCTCTGTATCTGACCCCCGTGTCCAGCGCTGGAGGAGTGGC
CATCAAGGCCGGCAGCCTCATCGCCGTCCTCATTCTGAGGCAGACCAACAACTACAACAG
CGACGACTTCCAGTTCGTGTGGAACATCTACGCCAACAACGACGTGGTGGTCCCCACCGG
CGGATGTGACGTGTCCGCCAGAGACGTGACCGTGACACTGCCCGATTACCCCGGAAGCG
TCCCTATCCCTCTGACAGTGTACTGCGCCAAGAGCCAAAATCTGGGCTACTATCTGTCCGG
AACCACAGCCGACGCCGGAAACTCCATCTTCACCAACACCGCCAGCTTTTCCCCCGCCCA
AGGAGTGGGAGTCCAGCTGACAAGAAGCGGCACCATCATCCCCGCCAGCAACACAGTGT
CTCTGGGCGCTGTGGGCACATCCGCTGTGTCTCTGGGACTGACAGCTAATTATGCCAGAA
CCGGAGGCCAAGTGACCGCTGGAAATGTGCAGAGCATTATTGGGGTGACCTTCGTGTACC
AGGGCGGAAGTAGCGGAGGCGGTGCCGACGTGACCATCACCGTGAACGGCAAGGTGGT
GGCCAAGAGTTCTGGTGGCGGTCCCAACAAAGGGAGTGGAACAACATCCGGGACTACGC
GATTGCTTTCCGGCCATACTTGCTTTACACTGACTGGCCTGTTGGGGACGCTCGTTACGAT
GGGTCTGCTCACCTGATGA
Translation:
(SEQ ID NO: 79)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQGGSSGGGADVTITVNGKVVA
KSSGGGPNKGSGTTSGTTRLLSGHTCFTLTGLLGTLVTMGLLT**

### FimH Transcript and Antigen Sequences

RNA transcript sequences of five constructs are listed below and include 5′UTR, FimH gene variant, 3′UTR and 3′polyA sequences. Sequence annotations are as follows: AUG, first methionine of the gene of interest (bold); UGAUAG or UGAUGA, stop codons after gene of interest (bold and italics); 5′UTRs are underlined; polyA, 80nt tract (italics); GSSGSGSS (SEQ ID NO:92), eight amino acid Glycine-Serine linker substitution in the DAF GPI anchor (underlined and italics). The bridging or reference constructs include BMD2/FimH_{DSG-}Sec/hHBB_80 pA, and BMD2/FimH_{DSG-}GPI/hHBB_80 pA, which contains the unmodified native GPI anchor.

>BMD2/FimH_{DSG}-SerGlyGPI/hHBB_80pA:
(SEQ ID NO: 80)
AGGAAAUAAGAGAGGAUAAGACGACUAAGGAGACAUACAGAAUAAGAGGCAGCCACCAU
GGAGACCGACACACUGCUGCUGUGGGUGCUGCUGCUGUGGGUGCCCGGCUCCACCGG
CUUCGCUUGCAAGACAGCCAGCGGAACCGCCAUCCCUAUCGGCGCAGCAAGCGCCAAC
GUCUACGUGAAUCUGGCUCCCGCAGUGAACGUGGGACAGAAUCUGGUGGUGGAUCUGA
GCACCCAGAUCUUCUGCCACAAUGACUACCCCGAGACCAUCACAGACUACGUGACACUG
CAGAGAGGAAGCGCCUACGGCGGCGUGCUGAGCAGCUUCAGCGGAACCGUGAAAUACA
GCGGCUCCAGCUACCCCUUCCCCACAACCAGCGAGACCCCUAGAGUGGUCUAUAACUC
UAGAACAGACAAGCCUUGGCCCGUGGCUCUGUAUCUGACCCCCGUGUCCAGCGCUGGA
GGAGUGGCCAUCAAGGCCGGCAGCCUCAUCGCCGUCCUCAUUCUGAGGCAGACCAACA
ACUACAACAGCGACGACUUCCAGUUCGUGUGGAACAUCUACGCCAACAACGACGUGGU
GGUCCCCACCGGCGGAUGUGACGUGUCCGCCAGAGACGUGACCGUGACACUGCCCGAU
UACCCCGGAAGCGUCCCUAUCCCUCUGACAGUGUACUGCGCCAAGAGCCAAAAUCUGG
GCUACUAUCUGUCCGGAACCACAGCCGACGCCGGAAACUCCAUCUUCACCAACACCGCC
AGCUUUUCCCCCGCCCAAGGAGUGGGAGUCCAGCUGACAAGAAGCGGCACCAUCAUCC
CCGCCAGCAACACAGUGUCUCUGGGCGCUGUGGGCACAUCCGCUGUGUCUCUGGGAC
UGACAGCUAAUUAUGCCAGAACCGGAGGCCAAGUGACCGCUGGAAAUGUGCAGAGCAU
UAUUGGGGUGACCUUCGUGUACCAGGGCGGAAGUAGCGGAGGCGGUGCCGACGUGAC
CAUCACCGUGAACGGCAAGGUGGUGGCCAAGAGUUCUGGUGGCGGUGGUUCAAGUGG
UAGUGGCAGUUCAAGUGGGACAACACGACUGUUGAGCGGGCAUACGUGUUUUACGCUG
ACAGGUCUUCUGGGCACGCUGGUUACUAUGGGCUUGCUUACGUGAUGAGCUCGCUUU
CUUGCUGUCCAAUUUCUAUUAAAGGUUCCUUUGUUCCCUAAGUCCAACUACUAAACUGG
GGGAUAUUAUGAAGGGCCUUGAGCAUCUGGAUUCUGCCUAAUAAAAAACAUUUAUUUUC
AUUGCAAAAAAAAAAAAAAAAAAAAAAAAAAAAAAAAAAAAAAAAAAAAAAAAAAAAAAAAAA
AAAAAAAAAAAAAAAAAAAAAAAA
FimH_{DSG}-SerGlyGPI Translation:
(SEQ ID NO: 81)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQADVTITVNGKVVAKGSSGSG
SSSGTTRLLSGHTCFTLTGLLGTLVTMGLLT**
>BMD562/FimH_{DSG}-Sec/hHBB_80pA:
(SEQ ID NO: 82)
AGGAAAUAAGAGAAAGAGGAUAAGACGACUAAGGAGACAUACAGAAUAAGAGGCAGCCA
CCAUGGAGACCGACACACUGCUGCUGUGGGUGCUGCUGCUGUGGGUGCCCGGCUCCA
CCGGCUUCGCUUGCAAGACAGCCAGCGGAACCGCCAUCCCUAUCGGCGCAGCAAGCGC
CAACGUCUACGUGAAUCUGGCUCCCGCAGUGAACGUGGGACAGAAUCUGGUGGUGGAU
CUGAGCACCCAGAUCUUCUGCCACAAUGACUACCCCGAGACCAUCACAGACUACGUGAC
ACUGCAGAGAGGAAGCGCCUACGGCGGCGUGCUGAGCAGCUUCAGCGGAACCGUGAAA
UACAGCGGCUCCAGCUACCCCUUCCCCACAACCAGCGAGACCCCUAGAGUGGUCUAUAA
CUCUAGAACAGACAAGCCUUGGCCCGUGGCUCUGUAUCUGACCCCCGUGUCCAGCGCU
GGAGGAGUGGCCAUCAAGGCCGGCAGCCUCAUCGCCGUCCUCAUUCUGAGGCAGACCA
ACAACUACAACAGCGACGACUUCCAGUUCGUGUGGAACAUCUACGCCAACAACGACGUG
GUGGUCCCCACCGGCGGAUGUGACGUGUCCGCCAGAGACGUGACCGUGACACUGCCC
GAUUACCCCGGAAGCGUCCCUAUCCCUCUGACAGUGUACUGCGCCAAGAGCCAAAAUC
UGGGCUACUAUCUGUCCGGAACCACAGCCGACGCCGGAAACUCCAUCUUCACCAACACC
GCCAGCUUUUCCCCCGCCCAAGGAGUGGGAGUCCAGCUGACAAGAAGCGGCACCAUCA
UCCCCGCCAGCAACACAGUGUCUCUGGGCGCUGUGGGCACAUCCGCUGUGUCUCUGG
GACUGACAGCUAAUUAUGCCAGAACCGGAGGCCAAGUGACCGCUGGAAAUGUGCAGAG
CAUUAUUGGGGUGACCUUCGUGUACCAGGGCGGAAGUAGCGGAGGCGGUGCCGACGU
GACCAUCACCGUGAACGGCAAGGUGGUGGCCAAGUGAUGAGCUCGCUUUCUUGCUGUC
CAAUUUCUAUUAAAGGUUCCUUUGUUCCCUAAGUCCAACUACUAAACUGGGGGAUAUUA
UGAAGGGCCUUGAGCAUCUGGAUUCUGCCUAAUAAAAAACAUUUAUUUUCAUUGCAAAA
AAAAAAAAAAAAAAAAAAAAAAAAAAAAAAAAAAAAAAAAAAAAAAAAAAAAAAAAAAAAAAA
AAAAAAAAAAAAAAA
FimH_{DSG}-Sec Translation:
(SEQ ID NO: 83)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQGGSSGGGADVTITVNGKVVA
K**
>BMD562/FimH_{DSG}-SerGlyGPI/hHBB_80pA
(SEQ ID NO: 84)
AGGAAAUAAGAGAAAGAGGAUAAGACGACUAAGGAGACAUACAGAAUAAGAGGCAGCCA
CCAUGGAGACCGACACACUGCUGCUGUGGGUGCUGCUGCUGUGGGUGCCCGGCUCCA
CCGGCUUCGCUUGCAAGACAGCCAGCGGAACCGCCAUCCCUAUCGGCGCAGCAAGCGC
CAACGUCUACGUGAAUCUGGCUCCCGCAGUGAACGUGGGACAGAAUCUGGUGGUGGAU
CUGAGCACCCAGAUCUUCUGCCACAAUGACUACCCCGAGACCAUCACAGACUACGUGAC
ACUGCAGAGAGGAAGCGCCUACGGCGGCGUGCUGAGCAGCUUCAGCGGAACCGUGAAA
UACAGCGGCUCCAGCUACCCCUUCCCCACAACCAGCGAGACCCCUAGAGUGGUCUAUAA
CUCUAGAACAGACAAGCCUUGGCCCGUGGCUCUGUAUCUGACCCCCGUGUCCAGCGCU
GGAGGAGUGGCCAUCAAGGCCGGCAGCCUCAUCGCCGUCCUCAUUCUGAGGCAGACCA
ACAACUACAACAGCGACGACUUCCAGUUCGUGUGGAACAUCUACGCCAACAACGACGUG
GUGGUCCCCACCGGCGGAUGUGACGUGUCCGCCAGAGACGUGACCGUGACACUGCCC
GAUUACCCCGGAAGCGUCCCUAUCCCUCUGACAGUGUACUGCGCCAAGAGCCAAAAUC
UGGGCUACUAUCUGUCCGGAACCACAGCCGACGCCGGAAACUCCAUCUUCACCAACACC
GCCAGCUUUUCCCCCGCCCAAGGAGUGGGAGUCCAGCUGACAAGAAGCGGCACCAUCA
UCCCCGCCAGCAACACAGUGUCUCUGGGCGCUGUGGGCACAUCCGCUGUGUCUCUGG
GACUGACAGCUAAUUAUGCCAGAACCGGAGGCCAAGUGACCGCUGGAAAUGUGCAGAG
CAUUAUUGGGGUGACCUUCGUGUACCAGGGCGGAAGUAGCGGAGGCGGUGCCGACGU
GACCAUCACCGUGAACGGCAAGGUGGUGGCCAAGAGUUCUGGUGGCGGUGGUUCAAG
UGGUAGUGGCAGUUCAAGUGGGACAACACGACUGUUGAGCGGGCAUACGUGUUUUACG
CUGACAGGUCUUCUGGGCACGCUGGUUACUAUGGGCUUGCUUACGUGAUGAGCUCGC
UUUCUUGCUGUCCAAUUUCUAUUAAAGGUUCCUUUGUUCCCUAAGUCCAACUACUAAAC
UGGGGGAUAUUAUGAAGGGCCUUGAGCAUCUGGAUUCUGCCUAAUAAAAAACAUUUAUU
UUCAUUGCAAAAAAAAAAAAAAAAAAAAAAAAAAAAAAAAAAAAAAAAAAAAAAAAAAAAAA
AAAAAAAAAAAAAAAAAAAAAAAAAAAA
FimH_{DSG}-SerGly Translation:
(SEQ ID NO: 85)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQGGSSGGGADVTITVNGKVVA
KSSGGGGSSGSGSSSGTTRLLSGHTCFTLTGLLGTLVTMGLLT**
>BMD576/FimH_{DSG}-Sec/hHBB_80pA
(SEQ ID NO: 86)
AGGAGGACUGGUCGAACCUGCAUAGUGAUCAUAAGGUCAGCAUAGCCACCAUGGAGAC
CGACACACUGCUGCUGUGGGUGCUGCUGCUGUGGGUGCCCGGCUCCACCGGCUUCGC
UUGCAAGACAGCCAGCGGAACCGCCAUCCCUAUCGGCGCAGCAAGCGCCAACGUCUAC
GUGAAUCUGGCUCCCGCAGUGAACGUGGGACAGAAUCUGGUGGUGGAUCUGAGCACCC
AGAUCUUCUGCCACAAUGACUACCCCGAGACCAUCACAGACUACGUGACACUGCAGAGA
GGAAGCGCCUACGGCGGCGUGCUGAGCAGCUUCAGCGGAACCGUGAAAUACAGCGGCU
CCAGCUACCCCUUCCCCACAACCAGCGAGACCCCUAGAGUGGUCUAUAACUCUAGAACA
GACAAGCCUUGGCCCGUGGCUCUGUAUCUGACCCCCGUGUCCAGCGCUGGAGGAGUG
GCCAUCAAGGCCGGCAGCCUCAUCGCCGUCCUCAUUCUGAGGCAGACCAACAACUACAA
CAGCGACGACUUCCAGUUCGUGUGGAACAUCUACGCCAACAACGACGUGGUGGUCCCC
ACCGGCGGAUGUGACGUGUCCGCCAGAGACGUGACCGUGACACUGCCCGAUUACCCCG
GAAGCGUCCCUAUCCCUCUGACAGUGUACUGCGCCAAGAGCCAAAAUCUGGGCUACUA
UCUGUCCGGAACCACAGCCGACGCCGGAAACUCCAUCUUCACCAACACCGCCAGCUUUU
CCCCCGCCCAAGGAGUGGGAGUCCAGCUGACAAGAAGCGGCACCAUCAUCCCCGCCAG
CAACACAGUGUCUCUGGGCGCUGUGGGCACAUCCGCUGUGUCUCUGGGACUGACAGCU
AAUUAUGCCAGAACCGGAGGCCAAGUGACCGCUGGAAAUGUGCAGAGCAUUAUUGGGG
UGACCUUCGUGUACCAGGGCGGAAGUAGCGGAGGCGGUGCCGACGUGACCAUCACCG
UGAACGGCAAGGUGGUGGCCAAGUGAUGAGCUCGCUUUCUUGCUGUCCAAUUUCUAUU
AAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAAAAAAAAAAAAAAAAAAAAAAAAAAAAAAAAAAAAAAAAAAAAAAA
AAAA
FimHDSG-Sec Translation:
(SEQ ID NO: 87)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQADVTITVNGKVVAK**
>BMD576/FimH_{DSG}-SerGlyGPI/hHBB_80pA
(SEQ ID NO: 88)
AGGAGGACUGGUCGAACCUGCAUAGUGAUCAUAAGGUCAGCAUAGCCACCAUGGAGAC
CGACACACUGCUGCUGUGGGUGCUGCUGCUGUGGGUGCCCGGCUCCACCGGCUUCGC
UUGCAAGACAGCCAGCGGAACCGCCAUCCCUAUCGGCGCAGCAAGCGCCAACGUCUAC
GUGAAUCUGGCUCCCGCAGUGAACGUGGGACAGAAUCUGGUGGUGGAUCUGAGCACCC
AGAUCUUCUGCCACAAUGACUACCCCGAGACCAUCACAGACUACGUGACACUGCAGAGA
GGAAGCGCCUACGGCGGCGUGCUGAGCAGCUUCAGCGGAACCGUGAAAUACAGCGGCU
CCAGCUACCCCUUCCCCACAACCAGCGAGACCCCUAGAGUGGUCUAUAACUCUAGAACA
GACAAGCCUUGGCCCGUGGCUCUGUAUCUGACCCCCGUGUCCAGCGCUGGAGGAGUG
GCCAUCAAGGCCGGCAGCCUCAUCGCCGUCCUCAUUCUGAGGCAGACCAACAACUACAA
CAGCGACGACUUCCAGUUCGUGUGGAACAUCUACGCCAACAACGACGUGGUGGUCCCC
ACCGGCGGAUGUGACGUGUCCGCCAGAGACGUGACCGUGACACUGCCCGAUUACCCCG
GAAGCGUCCCUAUCCCUCUGACAGUGUACUGCGCCAAGAGCCAAAAUCUGGGCUACUA
UCUGUCCGGAACCACAGCCGACGCCGGAAACUCCAUCUUCACCAACACCGCCAGCUUUU
CCCCCGCCCAAGGAGUGGGAGUCCAGCUGACAAGAAGCGGCACCAUCAUCCCCGCCAG
CAACACAGUGUCUCUGGGCGCUGUGGGCACAUCCGCUGUGUCUCUGGGACUGACAGCU
AAUUAUGCCAGAACCGGAGGCCAAGUGACCGCUGGAAAUGUGCAGAGCAUUAUUGGGG
UGACCUUCGUGUACCAGGGCGGAAGUAGCGGAGGCGGUGCCGACGUGACCAUCACCG
UGAACGGCAAGGUGGUGGCCAAGAGUUCUGGUGGCGGUGGUUCAAGUGGUAGUGGCA
GUUCAAGUGGGACAACACGACUGUUGAGCGGGCAUACGUGUUUUACGCUGACAGGUCU
UCUGGGCACGCUGGUUACUAUGGGCUUGCUUACGUGAUGAGCUCGCUUUCUUGCUGU
CCAAUUUCUAUUAAAGGUUCCUUUGUUCCCUAAGUCCAACUACUAAACUGGGGGAUAUU
AUGAAGGGCCUUGAGCAUCUGGAUUCUGCCUAAUAAAAAACAUUUAUUUUCAUUGCAAA
AAAAAAAAAAAAAAAAAAAAAAAAAAAAAAAAAAAAAAAAAAAAAAAAAAAAAAAAAAAAAAA
AAAAAAAAAAAAAAAA
FimH_{DSG}-SerGlyGPI Translation:
(SEQ ID NO: 89)
METDTLLLWVLLLWVPGSTGFACKTASGTAIPIGAASANVYVNLAPAVNVGQNLVVDLSTQIFC
HNDYPETITDYVTLQRGSAYGGVLSSFSGTVKYSGSSYPFPTTSETPRVVYNSRTDKPWPVAL
YLTPVSSAGGVAIKAGSLIAVLILRQTNNYNSDDFQFVWNIYANNDVVVPTGGCDVSARDVTVT
LPDYPGSVPIPLTVYCAKSQNLGYYLSGTTADAGNSIFTNTASFSPAQGVGVQLTRSGTIIPASN
TVSLGAVGTSAVSLGLTANYARTGGQVTAGNVQSIIGVTFVYQGGSSGGGADVTITVNGKVVA
KSSGGGGSSGSGSSSGTTRLLSGHTCFTLTGLLGTLVTMGLLT**

The following clauses describe additional embodiments of the disclosure:
C1. A glycoconjugate comprising a streptococcal C5a peptidase (SCP) carrier protein, or a functional fragment thereof, bound to a saccharide, the saccharide comprising a structure selected from the group consisting of: Formula O1, Formula O1A, Formula O1A1, Formula O1B, Formula O1C, Formula O2, Formula O3, Formula O4, Formula O4:K52, Formula O4:K6, Formula O5, Formula O5ab, Formula O5ac, Formula O6, Formula O6:K2, Formula O6:K13, Formula O6:K15, Formula O6:K54, Formula O7, Formula O8, Formula O9, Formula O9a, Formula O10, Formula O11, Formula O12, Formula O13, Formula O15, Formula O16, Formula O17, Formula O18, Formula O18A, Formula O18ac, Formula O18A1, Formula O18B, Formula O18B1, Formula O19, Formula O19ab, Formula O20, Formula O20ab, Formula O20ac, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O35, Formula O36, Formula O37, Formula O38, Formula O39, Formula O40, Formula O41, Formula O42, Formula O43, Formula O44, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D₁, Formula O63, Formula O64, Formula O65, Formula O66, Formula O68, Formula O69, Formula O70, Formula O71, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O112ab, Formula O112ac, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O174ab, Formula O174ac, Formula O175, Formula O176, Formula O177, Formula O178, Formula O179, Formula O180, Formula O181, Formula O182, Formula O183, Formula O184, Formula O185, Formula O186, Formula O187, and Formula O188.
C2. The glycoconjugate of C1, wherein the saccharide comprises a structure selected from the group consisting of O1A, O2, O6, and O25b.
C3. The glycoconjugate of C2, wherein the saccharide comprises the structure of O25b.
C4. The glycoconjugate of any one of C1-C3, wherein the saccharide is an Escherichia coli (E. coli) saccharide.
C5. A pharmaceutical composition comprising a glycoconjugate, wherein the glycoconjugate comprises a streptococcal C5a peptidase (SCP) carrier protein, or a functional fragment thereof, bound to a saccharide, the saccharide comprising a structure selected from the group consisting of: Formula O1, Formula O1A, Formula O1A1, Formula O1B, Formula O1C, Formula O2, Formula O3, Formula O4, Formula O4:K52, Formula O4:K6, Formula O5, Formula O5ab, Formula O5ac, Formula O6, Formula O6:K2, Formula O6:K13, Formula O6:K15, Formula O6:K54, Formula O7, Formula O8, Formula O9, Formula O9a, Formula O10, Formula O11, Formula O12, Formula O13, Formula O15, Formula O16, Formula O17, Formula O18, Formula O18A, Formula O18ac, Formula O18A1, Formula O18B, Formula O18B1, Formula O19, Formula O19ab, Formula O20, Formula O20ab, Formula O20ac, Formula O21, Formula O22, Formula O23, Formula O23A, Formula O24, Formula O25, Formula O25a, Formula O25b, Formula O26, Formula O27, Formula O28, Formula O29, Formula O30, Formula O32, Formula O33, Formula O35, Formula O36, Formula O37, Formula O38, Formula O39, Formula O40, Formula O41, Formula O42, Formula O43, Formula O44, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D₁, Formula O63, Formula O64, Formula O65, Formula O66, Formula O68, Formula O69, Formula O70, Formula O71, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O112ab, Formula O112ac, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O174ab, Formula O174ac, Formula O175, Formula O176, Formula O177, Formula O178, Formula O179, Formula O180, Formula O181, Formula O182, Formula O183, Formula O184, Formula O185, Formula O186, Formula O187, and Formula O188.
C6. A pharmaceutical composition comprising a) a glycoconjugate comprising an E. coli saccharide and b) a RNA molecule encoding a polypeptide derived from FimH, or a functional fragment thereof.
C7. The pharmaceutical composition of C6, wherein the glycoconjugate comprises a streptococcal C5a peptidase (SCP) carrier protein, or a functional fragment thereof, bound to the saccharide.
C8. The composition of any one of C5-C7, wherein the saccharide comprises a structure selected from the group consisting of O1A, O2, O6, and O25b.
C9. The composition of C8, wherein the saccharide comprises the structure of O25b.
C10. The composition of any one of C5 or C₈-C₉, wherein the saccharide is an Escherichia coli (E. coli) saccharide.
C11. The composition of any one of C5-C10, wherein the composition comprises a glycoconjugate for each of the saccharide structures of O1A, O2, O6, and O25b.
C12. The composition of C11, wherein the composition comprises a glycoconjugate comprising SCP, or a functional fragment thereof, for each of the saccharide structures of O1A, O2, O6, and O25b.
C13. The composition of C9, wherein the composition further comprises a glycoconjugate for each of the saccharide structures of O1A, O2, and O6, wherein at least one of saccharide structures of O1A, O2, or O6 is conjugated to CRM₁₉₇.
C14. The composition of C9, wherein the composition further comprises a glycoconjugate for each of the saccharide structures of O1A, O2, and O6, wherein at least one of saccharide structures of O1A, O2, or O6 is conjugated to a carrier selected from the group consisting of CRM₁₉₇, diphtheria toxin fragment B (DTFB), DTFB C8, Diphtheria toxoid (DT), tetanus toxoid (TT), fragment C of TT, pertussis toxoid, cholera toxoid, exotoxin A from Pseudomonas aeruginosa (P. aeruginosa), detoxified Exotoxin A of P. aeruginosa (EPA), maltose binding protein (MBP), detoxified hemolysin A of Staphylococcus aureus (S. aureus), clumping factor A, clumping factor B, Cholera toxin B subunit (CTB), Streptococcus pneumoniae Pneumolysin or detoxified variants thereof, Campylobacter jejuni (C. jejuni) AcrA, and C. jejuni natural glycoproteins.
C15. The composition of C9, wherein the composition further comprises a glycoconjugate for each of the saccharide structures of O1A, O2, and O6, wherein each of the saccharide structures of O1A, O2, and O6 is conjugated to CRM₁₉₇.
C16. The composition of any one of C5-C15, further comprising at least one additional glycoconjugate, wherein the additional glycoconjugate comprises a saccharide structure selected from the group consisting of Formula O1, Formula O1A1, Formula O1B, Formula O1C, Formula O3, Formula O4, Formula O4:K52, Formula O4:K6, Formula O5, Formula O5ab, Formula O5ac, Formula O7, Formula O8, Formula O9, Formula O9a, Formula O10, Formula O11, Formula O12, Formula O13, Formula O15, Formula O16, Formula O17, Formula O18, Formula O18A, Formula O18ac, Formula O18A1, Formula O18B, Formula O18B1, Formula O19, Formula O19ab, Formula O20, Formula O20ab, Formula O20ac, Formula O21, Formula O22, Formula O23, Formula O23A, Formula O24, Formula O25, Formula O25a, Formula O26, Formula O27, Formula O28, Formula O29, Formula O30, Formula O32, Formula O33, Formula O35, Formula O36, Formula O37, Formula O38, Formula O39, Formula O40, Formula O41, Formula O42, Formula O43, Formula O44, Formula O45, Formula O45rel, Formula O46, Formula O48, Formula O49, Formula O50, Formula O51, Formula O52, Formula O53, Formula O54, Formula O55, Formula O56, Formula O57, Formula O58, Formula O59, Formula O60, Formula O61, Formula O62, Formula 62D1, Formula O63, Formula O64, Formula O65, Formula O66, Formula O68, Formula O69, Formula O70, Formula O71, Formula O73, Formula O74, Formula O75, Formula O76, Formula O77, Formula O78, Formula O79, Formula O80, Formula O81, Formula O82, Formula O83, Formula O84, Formula O85, Formula O86, Formula O87, Formula O88, Formula O90, Formula O91, Formula O92, Formula O93, Formula O95, Formula O96, Formula O97, Formula O98, Formula O99, Formula O100, Formula O101, Formula O102, Formula O103, Formula O104, Formula O105, Formula O106, Formula O107, Formula O108, Formula O109, Formula O110, Formula O111, Formula O112, Formula O112ab, Formula O112ac, Formula O113, Formula O114, Formula O115, Formula O116, Formula O117, Formula O118, Formula O119, Formula O120, Formula O121, Formula O123, Formula O124, Formula O125, Formula O126, Formula O127, Formula O128, Formula O129, Formula O130, Formula O131, Formula O132, Formula O133, Formula O134, Formula O135, Formula O136, Formula O137, Formula O138, Formula O139, Formula O140, Formula O141, Formula O142, Formula O143, Formula O145, Formula O146, Formula O147, Formula O148, Formula O149, Formula O150, Formula O151, Formula O152, Formula O153, Formula O154, Formula O155, Formula O156, Formula O157, Formula O158, Formula O159, Formula O160, Formula O161, Formula O162, Formula O163, Formula O164, Formula O165, Formula O166, Formula O167, Formula O168, Formula O169, Formula O170, Formula O171, Formula O172, Formula O173, Formula O174, Formula O174ab, Formula O174ac, Formula O175, Formula O176, Formula O177, Formula O178, Formula O179, Formula O180, Formula O181, Formula O182, Formula O183, Formula O184, Formula O185, Formula O186, Formula O187, and Formula O188.
C17. The composition of any one of C₅-C₁₅, further comprising at least one additional glycoconjugate, wherein the additional glycoconjugate comprises a saccharide structure selected from the group consisting of Formula O4, Formula O4:K52, Formula O4:K6, Formula O8, Formula O9, O9a, Formula O11, Formula O13, Formula O15, Formula O16, Formula O17, Formula O18, Formula O18A, Formula O18ac, Formula O18A1, Formula O18B, Formula O18B1, Formula O21, Formula O75, and Formula O86.
C18. The composition of any one of C₅-C₁₅, further comprising at least 2, 3, 4, 5, or 6 glycoconjugates, wherein the additional glycoconjugates comprise a saccharide structure selected from the group consisting of Formula O4, Formula O4:K52, Formula O4:K6, Formula O8, Formula O9, O9a, Formula O11, Formula O13, Formula O15, Formula O16, Formula O17, Formula O18, Formula O18A, Formula O18ac, Formula O18A1, Formula O18B, Formula O18B1, Formula O21, Formula O75, and Formula O86.
C19. The composition of any one of C₅-C₁₅, further comprising 6 additional glycoconjugates, wherein the additional glycoconjugates comprise a saccharide structure selected from the group consisting of Formula O4, Formula O4:K52, Formula O4:K6, Formula O8, Formula O9, O9a, Formula O11, Formula O13, Formula O15, Formula O16, Formula O17, Formula O18, Formula O18A, Formula O18ac, Formula O18A1, Formula O18B, Formula O18B1, Formula O21, Formula O75, and Formula O86.
C20. The composition of any one of C₁₄-C₁₉, wherein at least one of the additional glycoconjugates comprises a carrier selected from the group consisting of CRM₁₉₇, diphtheria toxin fragment B (DTFB), DTFB C8, Diphtheria toxoid (DT), tetanus toxoid (TT), fragment C of TT, pertussis toxoid, cholera toxoid, exotoxin A from Pseudomonas aeruginosa (P. aeruginosa), detoxified Exotoxin A of P. aeruginosa (EPA), maltose binding protein (MBP), detoxified hemolysin A of Staphylococcus aureus (S. aureus), clumping factor A, clumping factor B, Cholera toxin B subunit (CTB), Streptococcus pneumoniae Pneumolysin or detoxified variants thereof, Campylobacter jejuni (C. jejuni) AcrA, and C. jejuni natural glycoproteins.
C21. The composition of any one of C14-C20, wherein at least one of the additional glycoconjugates comprises SCP, or a functional fragment thereof.
C22. The composition of anyone of C14-C21, wherein each of the additional glycoconjugates comprises SCP, or a functional fragment thereof.
C23. The composition of any one of C14-C21, wherein at least one of the additional glycoconjugates comprises CRM₁₉₇.
C24. The glycoconjugate or composition of any one of C1-C23, wherein at least one of the glycoconjugate(s) comprise a saccharide covalently bound to a carrier protein, or a functional fragment thereof.
C25. The glycoconjugate or composition of any one of C1-C24, wherein each of the glycoconjugate(s) comprise a saccharide covalently bound to a carrier protein, or a functional fragment thereof.
C26. The glycoconjugate or composition of any one of C1-C23, wherein at least one of the glycoconjugate(s) comprise a saccharide noncovalently bound to a carrier protein, or a functional fragment thereof.
C27. The glycoconjugate or composition of any one of C1-C23 or C26, wherein each of the glycoconjugate(s) comprise a saccharide noncovalently bound to a carrier protein, or a functional fragment thereof.
C28. The glycoconjugate or composition of C26 or C27, wherein said saccharide is bound to said carrier protein, or a functional fragment thereof, via a noncovalent interaction between biotin and streptavidin.
C29. The glycoconjugate or composition of any one of C1-C28, wherein n is an integer consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more in the Formula for each saccharide molecule according to Table 1.
C30. The glycoconjugate or composition of any one of C1-C28, wherein n is an integer consisting of 1 to 100 in the Formula for each saccharide molecule according to Table 1.
C31. The glycoconjugate or composition of any one of C1-C28, wherein n in the Formula for each saccharide molecule according to Table 1 is an integer greater than or equal to 20, 21, 22, 23, 24, 25, 26, 27, 28, 29, 30, 31, 32, 33, 34, 35, 36, 37, 38, 39, or 40.
C32. The glycoconjugate or composition of any one of C1-C28, wherein n is an integer consisting of 31 to 90 in the Formula for each saccharide molecule according to Table 1.
C33. The glycoconjugate or composition of any one of C1-C5 or C7-C32, wherein the SCP, or functional fragment thereof, is an enzymatically inactive SCP.
C34. The glycoconjugate or composition of any one of C1-C5 or C7-C33, wherein the SCP, or functional fragment thereof, is present in at least one Group B streptococcus (SCPB) bacterial strain.
C35. The glycoconjugate or composition of any one of C1-C5 or C7-C34, wherein the SCP, or functional fragment thereof, is an enzymatically inactive SCP fragment.
C36. The glycoconjugate or composition of any one of C1-C35, wherein the carrier protein is a functional fragment of SCP.
C37. The glycoconjugate or composition of C36, wherein the SCP fragment comprises the protease domain, the protease-associated domain (PA domain) and the three fibronectin type III (Fn) domains but does not comprise the export signal presequence, the pro-sequence, or the cell wall anchor domain.
C38. The glycoconjugate or composition of C36, wherein the SCP fragment is an enzymatically inactive SCP and comprises the protease domain, the protease-associated domain (PA domain) and the three fibronectin type III (Fn) domains but does not comprise the export signal presequence, the pro-sequence, or the cell wall anchor domain.
C39. The glycoconjugate or composition of C36, wherein the SCP fragment is an enzymatically inactive SCP and comprises the protease domain, the protease-associated domain (PA domain) and the three fibronectin type III (Fn) domains but does not comprise the export signal presequence, the pro-sequence, or the cell wall anchor domain, where said inactivation is accomplished by replacing at least one amino acid of the wild type sequence, and wherein said replacement is selected from the group consisting of D130A, H193A, N295A and S512A.
C40. The glycoconjugate or composition of C36, wherein the SCP fragment is an enzymatically inactive SCP and comprises the protease domain, the protease-associated domain (PA domain) and the three fibronectin type III (Fn) domains but does not comprise the export signal presequence, the pro-sequence, and the cell wall anchor domain, where said inactivation is accomplished by replacing at least two amino acids of the wild type sequence, wherein said at least two amino acids replacements are D130A and S512A.
C41. The glycoconjugate or composition of C36, wherein the SCP fragment is an enzymatically inactive SCP and comprises a polypeptide with at least 95%, 96%, 97%, 98%, or 99% sequence identity with SEQ ID NO: 113.
C42. The glycoconjugate or composition of C36, wherein the SCP fragment is an enzymatically inactive SCP and consists of a polypeptide with at least 95%, 96%, 97%, 98%, or 99% sequence identity with SEQ ID NO: 113.
C43. The glycoconjugate or composition of C36, wherein the SCP fragment is an enzymatically inactive SCP and comprises the sequence of SEQ ID NO: 113.
C44. The glycoconjugate or composition of C36, wherein the SCP fragment is an enzymatically inactive SCP and consists of the sequence of SEQ ID NO: 113.
C45. The glycoconjugate or composition of C36, wherein the SCP fragment is an enzymatically inactive SCP and comprises a polypeptide with at least 95%, 96%, 97%, 98%, or 99% sequence identity with SEQ ID NO: 114.
C46. The glycoconjugate or composition of C36, wherein the SCP fragment is an enzymatically inactive SCP and consists of a polypeptide with at least 95%, 96%, 97%, 98%, or 99% sequence identity with SEQ ID NO: 114.
C47. The glycoconjugate or composition of C36, wherein the SCP fragment is an enzymatically inactive SCP and comprises the sequence of SEQ ID NO: 114.
C48. The glycoconjugate or composition of C36, wherein the SCP fragment is an enzymatically inactive SCP and consists of the sequence of SEQ ID NO: 114.
C49. The composition of any one of C5 or C8-C48, further comprising a polypeptide derived from FimH or a functional fragment thereof.
C50. The composition of any one of C5 or C8-C48, further comprising a nucleic acid encoding a polypeptide derived from FimH, or a functional fragment thereof.
C51. A composition comprising a nucleic acid encoding a polypeptide derived from FimH, or a functional fragment thereof, and a CpG oligonucleotide adjuvant.
C52. A composition comprising a nucleic acid encoding a polypeptide derived from FimH, or a functional fragment thereof, a CpG oligonucleotide adjuvant, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b.
C53. A composition comprising a nucleic acid encoding a polypeptide derived from FimH, or a functional fragment thereof, and a liposomal adjuvant.
C54. A composition comprising a nucleic acid encoding a polypeptide derived from FimH, or a functional fragment thereof, a liposomal adjuvant, and a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b.
C55. The composition of C6, C7, or C49-C54, wherein the polypeptide comprises a mutation selected from the group consisting of G15A, G16A, and V27A, wherein the amino acid positions are numbered according to SEQ ID NO: 59.
C56. The composition of C6, C7, or C49-C55, wherein the polypeptide comprises each of the mutations G15A, G16A, and V27A, wherein the amino acid positions are numbered according to SEQ ID NO: 59.
C57. The composition of C6, C7, or C49-C56, wherein the polypeptide comprises the amino acid sequence of SEQ ID NO: 62.
C58. The composition of C6, C7, or C49-C57, wherein the polypeptide consists of the amino acid sequence of SEQ ID NO: 62.
C59. The composition of any one of C50-C58, wherein the nucleic acid is DNA.
C60. The composition of any one of C50-C58, wherein the nucleic acid is RNA.
C61. The composition of any one of C50-C58, wherein the nucleic acid is modRNA.
C62. The composition of C6, C7, C60, or C61, wherein the RNA is encapsulated in LNP.
C63. The composition of any one of C5-C62, further comprising an adjuvant.
C64. The composition of C63, wherein the adjuvant is a CpG oligonucleotide.
C65. A pharmaceutical composition comprising a glycoconjugate and a CpG oligonucleotide, wherein the glycoconjugate comprises a streptococcal C5a peptidase (SCP) carrier protein, or a functional fragment thereof, covalently bound to an E. coli saccharide, and wherein the saccharide comprises the structure of Formula O25b.
C66. The composition of C64 or 65, wherein the CpG oligonucleotide consists of 20, 21, 22, 23, or 24 nucleotides.
C67. The composition of any one of 64-C66, wherein the CpG oligonucleotide comprises the sequence of any one of SEQ ID Nos: 115-137.
C68. The composition of any one of C64-C67, wherein the CpG oligonucleotide consists of the sequence of any one of SEQ ID Nos: 115-137.
C69. The composition of any one of C64-C68, wherein the CpG oligonucleotide comprises the sequence of SEQ ID NO: 116 or 123.
C70. The composition of any one of C64-C69, wherein the CpG oligonucleotide consists of the sequence of SEQ ID NO: 116 or 123.
C71. The composition of C63, wherein the adjuvant is liposomal.
C72. The composition of C71, wherein the adjuvant comprises liposomes that range in mean diameter size from between about 30 nm and about 400 nm.
C73. The composition of C71 or C72, wherein the adjuvant comprises liposomes and wherein the liposomes have a mean diameter size of about 200 nm or less.
C74. The composition of any one of C71-C73, wherein the adjuvant comprises liposomes and wherein the liposomes have a polydispersity index (PDI) between about 0.05 and about 0.3.
C75. The composition of any one of C71-C74, wherein the adjuvant comprises liposomes and wherein the liposomes have a polydispersity index (PDI) of less than about 0.3.
C76. The composition of any one of C71-C75, wherein the liposomal adjuvant comprises MPLA and a saponin.
C77. The composition of any one of C71-C76, wherein the liposomal adjuvant comprises monophosphoryl 3-deacyl lipid A phosphorylated hexaacyl disaccharide (3D-PHAD®) and a saponin.
C78. The composition of any one of C71-C77, wherein the adjuvant comprises 3D-PHAD®, QS-21, 1,2-dimyristoyl-sn-glycero-3-phosphocholine (DMPC), 1,2-dimyristoyl-sn-glycero-3-phospho-(1′-rac-glycerol) (DMPG), and cholesterol.
C79. The composition of any one of C71-C78, wherein the liposomal adjuvant is a LiNA-2 adjuvant.
C80. The composition of any one of C5-C79, further comprising at least one saccharide derived from Klebsiella pneumoniae.
C81. The composition of C80, wherein the saccharide derived from Klebsiella pneumoniae is selected from the group consisting of type O1, O2, O3, and O5.
C82. The composition of any one of C5-C79, further comprising a saccharide derived from Klebsiella pneumoniae type O1 and O2.
C83. The glycoconjugate or composition of any one of C1-C5 or C7-C82, wherein the glycoconjugate(s) comprising SCP, or functional fragment thereof, are produced by a click chemistry reaction.
C84. The glycoconjugate or composition of C83, wherein said glycoconjugates are produced by an azide-alkyne cycloaddition reaction.
C85. The glycoconjugate or composition of C83 or C84, wherein the reaction is mediated by copper.
C86. A method of producing a glycoconjugate with a SCP carrier protein, or a functional fragment thereof, comprising the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C87. A method of producing a glycoconjugate with a SCP carrier protein, or a functional fragment thereof, consisting essentially of the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C88. A method of producing a glycoconjugate with a SCP carrier protein, or a functional fragment thereof, consisting of the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C89. The glycoconjugate or composition of any one of C1-C85, wherein the glycoconjugate(s) comprising SCP is produced by a method comprising the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C90. The glycoconjugate or composition of any one of C1-C85, wherein the glycoconjugate(s) comprising SCP is produced by a method consisting essentially of the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C91. The glycoconjugate or composition of any one of C1-C85, wherein the glycoconjugate(s) comprising SCP is produced by a method consisting of the steps of:
(a) reacting an isolated saccharide with a carbonic acid derivative and an agent comprising an azide to produce an activated saccharide with an azido linker,
(b) reacting SCP with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate.
C92. The method, glycoconjugate, or composition of any one of C86-C91, wherein in step a) the isolated saccharide is reacted with a carbonic acid derivative in an aprotic solvent.
C93. The method, glycoconjugate, or composition of C92, wherein the aprotic solvent is DMSO.
C94. The method, glycoconjugate, or composition of any one of C86-C93, wherein the carbonic acid derivative is selected from the group consisting of 1,1′-carbonyldiimidazole (CDI), 1,1′-carbonyl-di-(1,2,4-triazole) (CDT), disuccinimidyl carbonate (DSC), and N-hydroxysuccinimidyl chloroformate.
C95. The method, glycoconjugate, or composition ofany one of C86-C94, wherein the carbonic acid derivative is 1,1′-carbonyldiimidazole (CDI).
C96. The method, glycoconjugate, or composition ofany one of C86-C94, wherein the carbonic acid derivative is 1,1′-Carbonyl-di-(1,2,4-triazole) (CDT).
C97. The method, glycoconjugate, or composition of any one of C86-C96, wherein the agent comprising an azide comprises the structure of Formula I,

H₂N—X—N₃ (Formula I),
wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ, and O(CH₂CH₂O)ₘCH₂CH₂, and wherein n ranges from 1 to 10, and m ranges from 1 to 4.
C98. The method, glycoconjugate, or composition of C97, wherein n is selected from the group consisting of 1, 2, 3, 4, 5, 6, 7, 8, 9, and 10.
C99. The method, glycoconjugate, or composition of C97 or C98, wherein m is selected from the group consisting of 1, 2, 3, and 4.
C100. The method, glycoconjugate, or composition of any one of C86-C99, wherein the agent comprising an azide comprises 3-azido-1-propylamine.
C101. The method, glycoconjugate, or composition of any one of C86-C100, wherein the agent comprising an N-Hydroxysuccinimide (NHS) ester comprises the structure of Formula II,

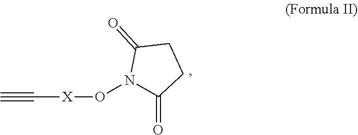

wherein X is selected from the group consisting of CH₂O(CH₂)ₙCH₂C═O and CH₂O(CH₂CH₂O)ₘ(CH₂)ₙCH₂C═O, and wherein n ranges rom 0 to 10, and m ranges from 0 to 4.
C102. The method, glycoconjugate, or composition of C101, wherein n is selected from the group consisting of 1, 2, 3, 4, 5, 6, 7, 8, 9, and 10.
C103. The method, glycoconjugate, or composition of C101 or C102, wherein m is selected from the group consisting of 1, 2, 3, and 4.
C104. The method, glycoconjugate, or composition of any one of C86-C103, wherein the agent comprising an N-Hydroxysuccinimide (NHS) ester comprises 3-propargyloxy-propanoic acid NHS ester.
C105. The method, glycoconjugate, or composition of anyone of C86-C104, wherein the method further comprises a step of capping the unreacted azido groups retained in the conjugate with an azido group capping agent.
C106. The method, glycoconjugate, or composition of C105, wherein the azido group capping agent is propargyl alcohol.
C107. The method, glycoconjugate, or composition of anyone of C86-C106, wherein the method further comprises a step of capping the unreacted alkyne groups retained in the conjugate with an alkyne group capping agent.
C108. The method, glycoconjugate, or composition of C107, wherein the alkyne group capping agent is 3-azido-1-propanol.
C109. The method, glycoconjugate, or composition of any one of C86-C108, wherein the cycloaddition reaction is mediated by Cu+1.
C110. The method, glycoconjugate, or composition of any one of C86-C109, wherein the degree of activation (DoA) of the activated saccharide is between about 5% and about 25%.
C111. The method, glycoconjugate, or composition of C110, wherein the DoA of the activated saccharide is between about 5% and about 10%.
C112. The method, glycoconjugate, or composition of C110, wherein the DoA of the activated saccharide is between about 20% and about 25%.
C113. The method, glycoconjugate, or composition of any one of C86-C112, wherein the degree of activation (DoA) of the activated SCP, or functional fragment thereof, is between about 15% and about 25%.
C114. The method, glycoconjugate, or composition of C113, wherein the degree of activation (DoA) of the activated SCP, or functional fragment thereof, is about 20%.
C115. A method of eliciting an immune response against an O25b expressing E. coli in a subject, comprising administering to the subject an effective amount of the glycoconjugate or composition of any one of C1-C85 or C89-C114.
C116. A method of eliciting an immune response against an O25b, O1a, O2, and/or O6 expressing E. coli in a subject, comprising administering to the subject an effective amount of the glycoconjugate or composition of any one of C1-C85 or C89-C114.
C117. Use of the glycoconjugate or composition of any one of C1-C85 or C89-C114, for inducing an immune response against an O25b expressing E. coli in a subject.
C118. Use of the conjugate or composition of any one of C1-C85 or C89-C114, for inducing an immune response against an O25b, O1a, O2, and/or O6 expressing E. coli in a subject.
C119. Use of the glycoconjugate or composition of any one of C1-C85 or C89-C114, in the manufacture of a medicament for inducing an immune response against an O25b expressing E. coli in a subject.
C120. Use of the glycoconjugate or composition of any one of C1-C85 or C89-C114, in the manufacture of a medicament for inducing an immune response against an O25b, O1a, O2, and/or O6 expressing E. coli in a subject.
C121. The method or use of any one of C115-C120, wherein the immune response elicits antibodies that have opsonophagocytic activity against an O25b expressing E. coli in a human subject.
C122. The method or use of any one of C115-C121, wherein the immune response elicits antibodies that have opsonophagocytic activity against an O25b O1a, O2, and/or O6 expressing E. coli in a human subject.
C123. The method or use of any one of C115-C122, wherein the immune response protects the subject from an E. coli infection.
C124. The method or use of C123, wherein the glycoconjugate(s) conjugated to SCP, or functional fragment thereof, induce a higher OPA titer versus a glycoconjugate conjugated to another carrier selected from the group consisting of CRM₁₉₇, diphtheria toxin fragment B (DTFB), DTFB C8, Diphtheria toxoid (DT), tetanus toxoid (TT), fragment C of TT, pertussis toxoid, cholera toxoid, exotoxin A from Pseudomonas aeruginosa (P. aeruginosa), detoxified Exotoxin A of P. aeruginosa (EPA), maltose binding protein (MBP), detoxified hemolysin A of Staphylococcus aureus (S. aureus), clumping factor A, clumping factor B, Cholera toxin B subunit (CTB), Streptococcus pneumoniae Pneumolysin or detoxified variants thereof, Campylobacter jejuni (C. jejuni) AcrA, and C. jejuni natural glycoproteins.
C125. The method or use of C124, wherein the glycoconjugate(s) conjugated to SCP, or a functional fragment thereof, induce a higher OPA titer versus a glycoconjugate conjugated CRM₁₉₇.
C126. The method or use of any one of C115-C125, wherein the subject has received one dose of the glycoconjugate(s) or composition.
C127. The method or use of any one of C115-C125, wherein the subject has received two doses of the glycoconjugate(s) or composition sequentially.
C128. The method or use of any one of C1115-C125, wherein the subject has received three doses of the glycoconjugate(s) or composition sequentially.
C129. The method or use of any one of C126-C128, wherein each dose comprises between about 0.1 μg and about 10 μg of E. coli polysaccharide O25b.
C130. The method or use of any one of C126-C129, wherein each dose comprises about 4 μg of E. coli polysaccharide O25b.
C131. The method or use of any one of C126-C129, wherein each dose comprises about 8 μg of E. coli polysaccharide O25b.
C132. The method or use of any one of C126-C131, wherein each dose comprises between about 0.1 μg and about 10 μg of E. coli polysaccharide O1a.
C133. The method or use of any one of C126-C132, wherein each dose comprises about 2 μg of E. coli polysaccharide O1a.
C134. The method or use of any one of C126-C132, wherein each dose comprises about 4 μg of E. coli polysaccharide O1a.
C135. The method or use of any one of C126-C134, wherein each dose comprises between about 0.1 μg and about 10 μg of E. coli polysaccharide O2.
C136. The method or use of any one of C126-C135, wherein each dose comprises about 2 μg of E. coli polysaccharide O2.
C137. The method or use of any one of C126-C135, wherein each dose comprises about 4 μg of E. coli polysaccharide O2.
C138. The method or use of any one of C126-C137, wherein each dose comprises between about 0.1 μg and about 10 μg of E. coli polysaccharide O6.
C139. The method or use of any one of C126-C138, wherein each dose comprises about 2 μg of E. coli polysaccharide O6.
C140. The method or use of any one of C126-C138, wherein each dose comprises about 4 μg of E. coli polysaccharide O6.
C141. The method or use of any one of C126-C140, wherein each dose comprises between about 10 μg and about 100 μg of FimH RNA.
C142. The method or use of any one of C126-C141, wherein each dose comprises about 30 μg of FimH RNA.
C143. The method or use of any one of C126-C141, wherein each dose comprises about 60 μg of FimH RNA.
C144. The method or use of any one of C126-C141, wherein each dose comprises about 90 μg of FimH RNA.
C145. The method or use of any one of C126-C144, wherein each dose comprises between about 0.05 mg and about 0.15 mg of QS-21.
C146. The method or use of any one of C126-C145, wherein each dose comprises about 0.1 mg of QS-21.
C147. The method or use of any one of C126-C146, wherein each dose comprises between about 3.5 mg and about 10.5 mg of 1,2-dimyristoyl-sn-glycero-3-phosphocholine (DMPC).
C148. The method or use of any one of C126-C147, wherein each dose comprises about 7 mg of DMPC.
C149. The method or use of any one of C126-C148, wherein each dose comprises between about 0.4 mg and about 1.2 mg 1,2-dimyristoyl-sn-glycero-3-phospho-(1′-rac-glycerol) (DMPG).
C150. The method or use of any one of C126-C149, wherein each dose comprises about 0.8 mg of DMPG.
C151. The method or use of any one of C126-C150, wherein each dose comprises between about 2.5 mg and about 8.5 mg of cholesterol.
C152. The method or use of any one of C126-C151, wherein each dose comprises about 5.5 mg of cholesterol.
C153. The method or use of any one of C126-C152, wherein each dose comprises between about 0.1 mg and about 0.3 mg of monophosphoryl 3-deacyl lipid A phosphorylated hexaacyl disaccharide (3D-PHAD®).
C154. The method or use of any one of C126-C153, wherein each dose comprises about 0.2 mg of 3D-PHAD®
C155. The method or use of any one of C126-C144, wherein each dose comprises between about 1 mg and about 10 mg of a CpG oligonucleotide.
C156. The method or use of any one of C126-C144 or C155, wherein each dose comprises between about 1 mg and about 10 mg of the CpG oligonucleotide of SEQ ID NO: 116 or 123.
C157. The method or use of any one of C126-C144 or C155-C156, wherein each dose comprises between about 1.5 mg and about 2.5 mg of a CpG oligonucleotide.
C158. The method or use of any one of C126-C144 or C155-C157, wherein each dose comprises between about 1.5 mg and about 2.5 mg of the CpG oligonucleotide of SEQ ID NO: 116 or 123.
C159. The method or use of any one of C126-C144 or C155-C158, wherein each dose comprises about 1.8 mg of a CpG oligonucleotide.
C160. The method or use of any one of C126-C144 or C155-C159, wherein each dose comprises about 1.8 mg of the CpG oligonucleotide of SEQ ID NO: 116 or 123.
C161. The method or use of any one of C126-C154, wherein each dose comprises about 4 μg of E. coli polysaccharide O25b and about 0.1 mg of a saponin.
C162. The method or use of any one of C126-C154 or C161, wherein each dose comprises about 4 μg of E. coli polysaccharide O25b and about 0.1 mg of QS-21.
C163. The method or use of any one of C126-C154 or C161-C162, wherein each dose comprises about 4 μg of E. coli polysaccharide O25b and about 7 mg of DMPC.
C164. The method or use of any one of C126-C154 or C161-C163, wherein each dose comprises about 4 μg of E. coli polysaccharide O25b and about 0.8 mg of DMPG.
C165. The method or use of any one of C126-C154 or C161-C164, wherein each dose comprises about 4 μg of E. coli polysaccharide O25b and about 5.5 mg of cholesterol.
C166. The method or use of any one of C126-C154 or C161-C165, wherein each dose comprises about 4 μg of E. coli polysaccharide O25b and about 0.2 mg of 3D-PHAD®.
C167. The method or use of any one of C126-C154 or C161-C166, wherein each dose comprises about 4 μg of E. coli polysaccharide O25b, about 7 mg of DMPC, about 0.8 mg of DMPG, about 5.5 mg of cholesterol, about 0.2 mg of 3D-PHAD®, and about 0.1 mg of QS-21.
C168. The method or use of any one of C126-C144 or C155-C160, wherein each dose comprises about 4 μg of E. coli polysaccharide O25b and about 1.8 mg of a CpG oligonucleotide.
C169. The method or use of any one of C126-C144, C155-C160, or C168, wherein each dose comprises about 4 μg of E. coli polysaccharide O25b and about 1.8 mg of the CpG oligonucleotide of SEQ ID NO: 116 or 123.
C170. The method or use of any one of C126-C169, wherein each dose comprises about 4 μg of E. coli polysaccharide O25b and about 30 μg of FimH RNA.
C171. The method or use of any one of C126-C169, wherein each dose comprises about 4 μg of E. coli polysaccharide O25b and about 60 μg of FimH RNA.
C172. The method or use of any one of C126-C169, wherein each dose comprises about 8 μg of E. coli polysaccharide O25b and about 90 μg of FimH RNA.
C173. The method or use of any one of C126-C172, wherein the subject is at least 18 years of age.
C174. The method or use of any one of C126-C173, wherein the subject is less than 70, 69, 68, 67, 66, 65, 64, 63, 62, 61, or 60 years of age.
C175. The method or use of any one of C126-C174, wherein the subject is between 18 and 64 years of age.
C176. The method or use of any one of C126-C175, wherein the subject has a history of frequent urinary tract infections (UTIs).
C177. A method of eliciting an immune response against Klebsiella pneumoniae in a subject, comprising administering to the mammal an effective amount of the composition of any one of C80-C85 or C89-C114.
C178. Use of the composition of any one of C80-C85 or C89-C114, for inducing an immune response against Klebsiella pneumoniae in a subject.
C179. Use of the composition of any one of C80-C85 or C89-C114, in the manufacture of a medicament for inducing an immune response against Klebsiella pneumoniae in a subject.
C180. The method or use of any one of C177-C179, wherein the immune response comprises opsonophagocytic antibodies against Klebsiella pneumoniae in a subject.
C181. The method or use of any one of C177-C180, wherein the immune response protects the subject from a Klebsiella pneumoniae infection.

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The contents of all cited references (including literature references, issued patents, published patent applications, and GENBANK® Accession numbers as cited throughout this application) recited in the application, to the extent that they provide exemplary procedural or other details supplementary to those set forth herein, are hereby specifically and expressly incorporated by reference. When definitions of terms in documents that are incorporated by reference herein conflict with those used herein, the definitions used herein govern.

1. A pharmaceutical composition comprising a glycoconjugate, wherein the glycoconjugate comprises a streptococcal C5a peptidase (SCP) carrier protein, or a functional fragment thereof, covalently bound to an E. coli saccharide, and wherein the saccharide comprises the structure of Formula O25b.

2. A pharmaceutical composition comprising a glycoconjugate comprising an E. coli saccharide and a RNA molecule encoding a polypeptide derived from FimH, or a functional fragment thereof.

3-4. (canceled)

5. The composition of claim 1, further comprising at least one additional glycoconjugate, wherein the additional glycoconjugate comprises a saccharide structure selected from the group consisting of Formula O1A, Formula O2, Formula O6, Formula O4, Formula O4:K52, Formula O4:K6, Formula O8, Formula O9, O9a, Formula O11, Formula O13, Formula O15, Formula O16, Formula O17, Formula O18, Formula O18A, Formula O18ac, Formula O18A1, Formula O18B, Formula O18B1, Formula O21, Formula O75, and Formula O86, and wherein at least one of the additional glycoconjugates comprises SCP, a functional fragment thereof, or CRM₁₉₇.

6-10. (canceled)

11. The composition of claim 1, wherein n is an integer consisting of 31 to 100 in the Formula for each saccharide molecule according to Table 1.

12. The composition of claim 1, wherein:
(i) the SCP, or functional fragment thereof, is an enzymatically inactive SCP; or
(ii) the SCP, or functional fragment thereof, is present in at least one Group B streptococcus (SCPB) bacterial strain.

13. (canceled)

14. The composition of claim 1, wherein the carrier protein is a functional fragment of SCP, and wherein the SCP fragment comprises:
(i) the protease domain, the protease-associated domain (PA domain) and the three fibronectin type III (Fn) domains but does not comprise the export signal presequence, the pro-sequence, or the cell wall anchor domain; or
(ii) the protease domain, the protease-associated domain (PA domain) and the three fibronectin type III (Fn) domains but does not comprise the export signal presequence, the pro-sequence, or the cell wall anchor domain; wherein the SCP is an enzymatically inactive SCP and said inactivation is accomplished by replacing at least one amino acid of the wild type sequence, and wherein said replacement is selected from the group consisting of D130A, H193A, N295A and S512A; or
(iii) the protease domain, the protease-associated domain (PA domain) and the three fibronectin type III (Fn) domains but does not comprise the export signal presequence, the pro-sequence, and the cell wall anchor domain, wherein the SCP is an enzymatically inactive SCP and said inactivation is accomplished by replacing at least two amino acids of the wild type sequence, wherein said at least two amino acids replacements are D130A and S512A.

15. (canceled)

16. The composition of claim 1, wherein:
(i) the SCP, or functional fragment thereof, is an enzymatically inactive SCP fragment and comprises a polypeptide with at least 95%, 96%, 97%, 98%, or 99% sequence identity with SEQ ID NO: 113; or
(ii) the SCP, or functional fragment thereof, is an enzymatically inactive SCP fragment and comprises the sequence of SEQ ID NO: 113; or
(iii) the SCP, or functional fragment thereof, is an enzymatically inactive SCP fragment and comprises a polypeptide with at least 95%, 96%, 97%, 98%, or 99% sequence identity with SEQ ID NO: 114; or
(iv) the SCP, or functional fragment thereof, is an enzymatically inactive SCP fragment and comprises the sequence of SEQ ID NO: 114.

17-19. (canceled)

20. The composition of claim 1, further comprising a polypeptide derived from FimH, a functional fragment thereof, or a nucleic acid encoding a polypeptide derived from FimH, or a functional fragment thereof.

21. The composition of claim 20, wherein the polypeptide comprises each of the mutations of G15A, G16A, and V27A, wherein the amino acid positions are numbered according to SEQ ID NO: 59.

22. The composition of claim 20, wherein the nucleic acid is RNA and wherein:
(i) the RNA comprises at least one open reading frame (ORF) encoding FimH, a 5′ untranslated region (5′ UTR), a 3′ untranslated region (3′ UTR) and a polyA tail: or
(ii) the RNA comprises at least one modified nucleotide, wherein the modified nucleotide is pseudouridine (Ψ) or N¹-methylpseudouridine (m1Ψ): or
(iii) the RNA is formulated in a lipid nanoparticle (LNP) comprising a cationic lipid, a steroid or steroid analog, a neutral lipid, and a PEGylated lipid.

23-28. (canceled)

29. The composition of claim 1, further comprising a liposomal adjuvant comprising monophosphoryl lipid A (MPLA) and QS-21 or a CpG oligonucleotide adjuvant.

30-33. (canceled)

34. A pharmaceutical composition comprising a glycoconjugate and a CpG oligonucleotide, wherein the glycoconjugate comprises a streptococcal C5a peptidase (SCP) carrier protein, or a functional fragment thereof, covalently bound to an E. coli saccharide, and wherein the saccharide comprises the structure of Formula O25b.

35-36. (canceled)

37. The composition of claim 1, wherein the glycoconjugate(s) comprising SCP, or functional fragment thereof, are produced by a click chemistry reaction.

38. The composition of claim 37, wherein the glycoconjugate(s) comprising SCP, or functional fragment thereof, are produced by an azide-alkyne cycloaddition reaction.

39. The composition of claim 37, wherein the reaction is mediated by copper.

40. The composition of claim 37, wherein the glycoconjugate(s) comprising SCP, or functional fragment thereof, is produced by a method comprising the steps of:
(a) reacting an isolated saccharide with a carbonic acid derivative and an agent comprising an azide to produce an activated saccharide with an azido linker,
(b) reacting SCP, or functional fragment thereof, with an agent comprising an N-Hydroxysuccinimide (NHS) ester to produce an activated alkyne-SCP, and
(c) reacting the activated saccharide with an azido linker of step (a) with the activated alkyne-SCP of step (b) by azide-alkyne cycloaddition reaction to form the glycoconjugate(s).

41. The composition of 40, wherein:
(i) in step a), the isolated saccharide is reacted with a carbonic acid derivative in an aprotic solvent, and wherein the aprotic solvent is DMSO; or
(ii) the carbonic acid derivative is selected from the group consisting of 1,1′-carbonyldiimidazole (CDI) and 1,1′-carbonyl-di-(1,2,4-triazole) (CDT); or
(iii) the agent comprising an azide comprises the structure of Formula I,
H₂—N—X—N₃ (Formula I),
wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ, and O(CH₂CH₂O)ₘCH₂CH₂, and wherein n ranges from 1 to 10, and m ranges from 1 to 4; or
(iv) the agent comprising an azide comprises 3-azido-1-propylamine: or
(v) the agent comprising an N-Hydroxysuccinimide (NHS) ester comprises the structure of Formula II, 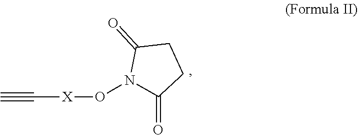
wherein X is selected from the group consisting of CH₂O(CH₂)ₙCH₂C═O and CH₂O(CH₂CH₂O)ₘ(CH₂)ₙCH₂C═O, and wherein n ranges from 0 to 10, and m ranges from 0 to 4; or
(vi) the agent comprising an N-Hydroxysuccinimide (NHS) ester comprises 3-propargyloxy-propanoic acid NHS ester.

42-46. (canceled)

47. The composition of claim 40, wherein:
(i) the method further comprises a step of capping the unreacted azido groups retained in the conjugate with an azido group capping agent, and wherein the azido group capping agent is propargyl alcohol; or
(ii) the method further comprises a step of capping the unreacted alkyne groups retained in the conjugate with an alkyne group capping agent, and wherein the alkyne group capping agent is 3-azido-1-propanol; or
(iii) the cycloaddition reaction is mediated by Cu+1.

48-50. (canceled)

51. The composition of claim 40, wherein the degree of activation (DoA) of the activated SCP, or functional fragment thereof, is between about 15% and about 25%.

52. A method of eliciting an immune response against an O25b expressing E. coli in a subject, comprising administering to the subject an effective amount of the composition of claim 1.

53-68. (canceled)
